(19) United States
(12) Patent Application Publication
(10) Pub. No.: US 2017/0233413 A1
(43) Pub. Date: Aug. 17, 2017

(54) SUBSTITUTED ANNULATED PYRIMIDINES AND USE THEREOF

(71) Applicant: Bayer Pharma Aktiengesellschaft, Berlin (DE)

(72) Inventors: Walter HÜBSCH, Wuppertal (DE); Alexandros VAKALOPOULOS, Hilden (DE); Markus FOLLMANN, Köln (DE); Gaelle VALOT, Wuppertal (DE); Johannes-Peter STASCH, Grottaferrata (RM) (IT); Damian BROCKSCHNIEDER, Haan (DE); Tobias MARQUARDT, Wuppertal (DE); Frank WUNDER, Wuppertal (DE); Adrian TERSTEEGEN, Wuppertal (DE); Lisa DIETZ, Wuppertal (DE); Dieter LANG, Velbert (DE); Niels LINDNER, Wuppertal (DE)

(73) Assignee: Bayer Pharma Aktiengesellschaft, Berlin (DE)

(21) Appl. No.: 15/503,448

(22) PCT Filed: Aug. 25, 2015
(86) PCT No.: PCT/EP2015/069428

§ 371 Date: Feb. 13, 2017

(30) Foreign Application Priority Data
Aug. 29, 2014 (EP) 14182877.2

(51) Int. Cl. C07D 519/00; C07D 487/04; A61K 9/08; A61K 31/519; A61K 9/20; A61K 9/00

(52) U.S. Cl. CPC C07D 519/00; A61K 9/20; A61K 9/0053; A61K 9/08; A61K 31/519; C07D 487/04

(57) ABSTRACT

The present application relates to novel substituted fused pyrimidines, to processes for their preparation, to their use alone or in combinations for the treatment and/or prophylaxis of diseases, and to their use for producing medicaments for the treatment and/or prophylaxis of diseases, in particular for the treatment and/or prophylaxis of cardiovascular disorders.

The present application relates to novel substituted fused pyrimidines, to processes for their preparation, to their use alone or in combinations for the treatment and/or prophylaxis of diseases, and to their use for producing medicaments for the treatment and/or prophylaxis of diseases, in particular for the treatment and/or prophylaxis of cardiovascular disorders.

One of the most important cellular transmission systems in mammalian cells is cyclic guanosine monophosphate (cGMP). Together with nitrogen monoxide (NO), which is released from the endothelium and transmits hormonal and mechanical signals, it forms the NO/cGMP system. Guanylate cyclases catalyse the biosynthesis of cGMP from guanosine triphosphate (GTP). The representatives of this family known to date can be classified into two groups either by structural features or by the type of ligands: the particulate guanylate cyclases which can be stimulated by natriuretic peptides, and the soluble guanylate cyclases which can be stimulated by NO. The soluble guanylate cyclases consist of two subunits and very probably contain one heme per heterodimer, which is part of the regulatory centre. This is of central importance for the activation mechanism. NO is able to bind to the iron atom of heme and thus markedly increase the activity of the enzyme. Heme-free preparations cannot, by contrast, be stimulated by NO. Carbon monoxide (CO) is also able to bind to the central iron atom of heme, but the stimulation by CO is much less than that by NO.

By forming cGMP, and owing to the resulting regulation of phosphodiesterases, ion channels and protein kinases, guanylate cyclase plays an important role in various physiological processes, in particular in the relaxation and proliferation of smooth muscle cells, in platelet aggregation and platelet adhesion and in neuronal signal transmission, and also in disorders which are based on a disruption of the aforementioned processes. Under pathophysiological conditions, the NO/cGMP system can be suppressed, which can lead, for example, to hypertension, platelet activation, increased cell proliferation, endothelial dysfunction, arteriosclerosis, angina pectoris, heart failure, myocardial infarction, thromboses, stroke and sexual dysfunction.

Owing to the expected high efficiency and low level of side effects, a possible NO-independent treatment for such disorders by targeting the influence of the cGMP signal pathway in organisms is a promising approach.

Hitherto, for the therapeutic stimulation of the soluble guanylate cyclase, use has exclusively been made of compounds such as organic nitrates whose effect is based on NO. The latter is formed by bioconversion and activates soluble guanylate cyclase by attacking the central iron atom of heme. In addition to the side effects, the development of tolerance is one of the crucial disadvantages of this mode of treatment.

A few years ago, some substances which stimulate soluble guanylate cyclase directly, i.e. without prior release of NO, were described, for example 3-(5′-hydroxymethyl-2′-furyl)-1-benzylindazole [YC-1; Wu et al., Blood 84 1994, 4226; Mülsch et al., Brit. J Pharmacol. 1997, 120, 681]. The more recent stimulators of soluble guanylate cyclase include BAY 41-2272, BAY 41-8543 and riociguat (BAY 63-2521) (see, for example, Stasch J.-P. et al., Nat. Rev. Drug Disc. 2006, 5: 755-768; Stasch J.-P. et al., ChemMedChem 2009, 4: 853-865; Stasch J.-P. et al., Circulation 2011, 123, 2263-2273]. Interestingly, some of these sGC stimulators, for example YC-1 or BAY 41-2272, also exhibit PDE5-inhibitory action in addition to direct guanylate cyclase stimulation. In order to maximize the cGMP pathway, it is pharmacologically desirable to stimulate the synthesis of cGMP and simultaneously to inhibit degradation via PDE-5. This dual principle is particularly advantageous in pharmacological terms (see, for example, Oudout et al., Eur. Urol. 2011, 60, 1020-1026; Albersen et al., J Sex Med. 2013; 10, 1268-1277].

The dual principle is fulfilled in the context of the present invention when the inventive compounds exhibit an effect on recombinant guanylate cyclase reporter cell lines according to the study in B-2 as the minimal effective concentration (MEC) of ≦3 μM and exhibit inhibition of human phosphodiesterase-5 (PDE5) according to the study in B-3 as IC₅₀<100 nM.

Phosphodiesterase-5 (PDE5) is the name of one of the enzymes which cleave the phosphoric ester bond in cGMP, forming 5′-guanosine monophosphate (5′-GMP). In humans, phosphodiesterase-5 occurs predominantly in the smooth musculature of the corpus cavernosum penis and the pulmonary arteries. Blockage of cGMP degradation by inhibition of PDE5 (with, for example, sildenafil, vardenafil or tadalafil) leads to increased signals of the relaxation signaling pathways and specifically to increased blood supply in the corpus cavernosum penis and lower pressure in the pulmonary blood vessels. They are used for treatment of erectile dysfunction and of pulmonary arterial hypertension. As well as PDE5, there are further cGMP-cleaving phosphodiesterases [Stasch et al., Circulation 2011, 123, 2263-2273].

As stimulators of soluble guanylate cyclase, WO 00/06568 and WO 00/06569 disclose fused pyrazole derivatives, and WO 03/095451 discloses carbamate-substituted 3-pyrimidinylpyrazolopyridines. 3-Pyrimidinylpyrazolopyridines with phenylamide substituents are described in E. M. Becker et al., BMC Pharmacology, 2001, 1 (13). WO 2004/009590 describes pyrazolopyridines with substituted 4-aminopyrimidines for the treatment of CNS disorders. WO 2010/065275 and WO 2011/149921 disclose substituted pyrrolo- and dihydropyridopyrimidines as sGC activators. As sGC stimulators, WO 2012/004259 describes fused aminopyrimidines, and WO 2012/004258, WO 2012/143510 and WO 2012/152629 fused pyrimidines and triazines. WO 2012/28647 discloses pyrazolopyridines with various azaheterocycles for treatment of cardiovascular disorders.

It was an object of the present invention to provide novel substances which act as stimulators of soluble guanylate cyclase and also as stimulators of soluble guanylate cyclase and phosphodiesterase-5 inhibitors (dual principle) and have an identical or improved therapeutic profile compared to the compounds known from the prior art, for example with respect to their in vivo properties, for example their pharmacokinetic and pharmacodynamic characteristics and/or their metabolic profile and/or their dose-activity relationship.

The present invention relates to compounds of the general formula (I)

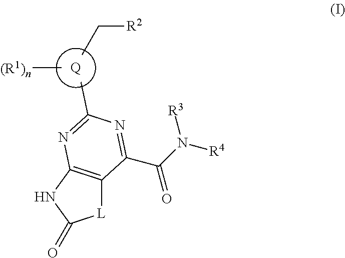

in which
the ring Q represents 5- or 6-membered monocyclic heteroaryl or 8- or 9-membered bicyclic heteroaryl,
L represents a #¹-CR^{5A}R^{5B}—(CR^{6A}R^{6B})ₘ-#² group, where
   #¹ represents the point of attachment to the carbonyl group,
   #² represents the attachment site to the pyrimidine ring,
   m represents a number 0, 1 or 2,
   R^{5A} represents hydrogen, fluorine, (C₁-C₄)-alkyl, hydroxy or amino,
      in which (C₁-C₄)-alkyl may be substituted by 1 to 3 substituents independently of one another selected from the group consisting of fluorine, trifluoromethyl, hydroxy, hydroxycarbonyl, (C₁-C₄)-alkoxycarbonyl and amino,
   R^{5B} represents hydrogen, fluorine, difluoromethyl, trifluoromethyl, (C₁-C₆)-alkyl, (C₁-C₄)alkoxycarbonylamino, cyano, (C₃-C₇)-cycloalkyl, difluoromethoxy, trifluoromethoxy, phenyl or a group of the formula -M-R⁷,
      in which (C₁-C₆)-alkyl may be substituted by 1 to 3 substituents independently of one another selected from the group consisting of fluorine, cyano, trifluoromethyl, (C₃-C₇)cycloalkyl, hydroxy, difluoromethoxy, trifluoromethoxy, (C₁-C₄)-alkoxy, hydroxycarbonyl, (C₁-C₄)-alkoxycarbonyl and amino,
      and in which
      M represents a bond or (C₁-C₄)-alkanediyl,
      R⁷ represents —(C═O)ᵣ—OR⁸, —(C═O)ᵣ—NR⁹R¹⁰, —C(═S)—NR⁹R¹⁰, —NR⁸—(C═O)—R¹¹, —NR⁸—(C═O)—NR⁹R¹⁰, —NR⁸—SO₂—NR⁹R¹⁰, —NR⁸—SO₂—R¹¹, —S(O)ₛ—R¹¹, —SO₂—NR⁹R¹⁰, 4- to 7-membered heterocyclyl, phenyl or 5- or 6-membered heteroaryl in which
         r represents the number 0 or 1,
         s represents the number 0, 1 or 2,
         R⁸, R⁹ and R¹⁰ independently of one another each represent hydrogen, (C₁-C₆)-alkyl, (C₃-C₈)-cycloalkyl, 4- to 7-membered heterocyclyl, phenyl or 5- or 6-membered heteroaryl,
         or
         R⁹ and R¹⁰ together with the atom(s) to which they are respectively attached form a 4- to 7-membered heterocycle,
         R¹¹ represents (C₁-C₆)-alkyl or (C₃-C₇)-cycloalkyl,
         or
         R⁸ and R¹¹ together with the atom(s) to which they are respectively attached form a 4- to 7-membered heterocycle,
      and
      in which the (C₁-C₆)-alkyl, (C₃-C₇)-cycloalkyl, (C₃-C₈)-cycloalkyl and 4- to 7-membered heterocyclyl groups mentioned above may each independently of one another additionally be substituted by 1 to 3 substituents independently of one another selected from the group consisting of fluorine, difluoromethyl, trifluoromethyl, (C₁-C₄)-alkyl, (C₃-C₇)cycloalkyl, hydroxy, difluoromethoxy, trifluoromethoxy, (C₁-C₄)-alkoxy, hydroxycarbonyl, (C₁-C₄)-alkoxycarbonyl, amino, phenyl, 4- to 7-membered heterocyclyl and 5- or 6-membered heteroaryl,
   or
   R^{5A} and R^{5B} together with the carbon atom to which they are attached form a (C₂-C₄)alkenyl group, an oxo group, a 3- to 6-membered carbocycle or a 4- to 7-membered heterocycle,
      in which the 3- to 6-membered carbocycle and the 4- to 7-membered heterocycle may be substituted by 1 or 2 substituents independently of one another selected from the group consisting of fluorine, hydroxy, methoxy and (C₁-C₄)-alkyl,
   R^{6A} represents hydrogen, fluorine, (C₁-C₄)-alkyl or hydroxy,
   R^{6B} represents hydrogen, fluorine, (C₁-C₄)-alkyl or trifluoromethyl,
R¹ represents hydrogen, halogen, cyano, difluoromethyl, trifluoromethyl, (C₁-C₄)-alkyl, (C₃-C₇)cycloalkyl, hydroxy, (C₁-C₄)-alkoxy, phenyl or 5- or 6-membered heterocyclyl,
n represents a number 0, 1, 2 or 3,
R² represents trifluoromethyl, (C₁-C₆)-alkyl, (C₃-C₈)-cycloalkyl, phenyl or 5- or 6-membered heteroaryl,
   where (C₁-C₆)-alkyl is substituted by a substituent selected from the group consisting of difluoromethyl and trifluoromethyl and may furthermore be up to trisubstituted by fluorine,
   and where (C₃-C₈)-cycloalkyl may be substituted by 1 or 2 substituents independently of one another selected from the group consisting of fluorine, methyl and methoxy,
   and where phenyl may be substituted by 1 to 3 halogen substituents and furthermore by 1 or 2 substituents independently of one another selected from the group consisting of (C₁-C₄)-alkyl, (C₁-C₄)-alkoxy and cyano,
   and where 5- or 6-membered heteroaryl may be substituted by 1 or 2 substituents selected from the group consisting of trifluoromethyl and methyl and furthermore up to three times by fluorine,
R³ represents hydrogen, (C₁-C₄)-alkyl or (C₃-C₈)-cycloalkyl,
R⁴ represents hydrogen, (C₁-C₁₀)-alkyl, (C₃-C₈)-cycloalkyl, (C₂-C₆)-alkenyl, 4- to 7-membered heterocyclyl, phenyl, 5- or 6-membered heteroaryl, —NR¹²R¹³ or —OR¹⁴,
   where (C₁-C₁₀)-alkyl, (C₃-C₈)-cycloalkyl, (C₂-C₆)-alkenyl and 4- to 7-membered heterocyclyl may be substituted by 1 to 3 substituents independently of one another selected from the group consisting of fluorine, difluoromethyl, trifluoromethyl, methyl, ethyl, hydroxy, oxo, (C₁-C₆)alkyl, (C₃-C₇)-cycloalkyl, difluoromethoxy, trifluoromethoxy, —OR¹⁵, —NR¹⁶—(C═O)—R¹⁷, —NR¹⁶—(C═O)—NR¹⁸R¹⁹, —NR¹⁸R¹⁹, —(C═O)—NR¹⁸R¹⁹, —S(O)ₚ—R²⁰, —NR¹⁸—SO₂—R¹⁹, —SO₂—NR¹⁸R¹⁹, —(C═O)—OR²¹, —NR¹⁶—(C═O)—OR²¹, phenyl, 4- to 7-membered heterocyclyl and 5- or 6-membered heteroaryl, in which
      p represents the number 0, 1 or 2,
      R¹⁵ and R²⁰ independently of one another each represent (C₁-C₆)-alkyl, phenyl or (C₃-C₈)-cycloalkyl,
      R¹⁶, R¹⁷, R¹⁸ and R¹⁹ independently of one another each represent hydrogen, (CIC₆)-alkyl or (C₃-C₈)-cycloalkyl,
      or
      R¹⁶ and R¹⁷ together with the nitrogen atom to which they are attached form a 4- to 7-membered heterocycle,
      or
      R¹⁸ and R¹⁹ together with the nitrogen atom to which they are attached form a 4- to 7-membered heterocycle,
      R²¹ represents hydrogen, (C₁-C₆)-alkyl or (C₃-C₈)-cycloalkyl,
   and
   where 5- or 6-membered heteroaryl and phenyl may each be substituted by 1 to 3 substituents independently of one another selected from the group consisting of halogen, difluoromethyl, trifluoromethyl, (C₁-C₄)-alkyl, (C₁-C₄)-alkoxy, difluoromethoxy, trifluoromethoxy, cyano, hydroxy and (C₃-C₇)-cycloalkyl,
   and where
   R¹² and R¹³ independently of one another represent hydrogen or (C₁-C₄)-alkyl, in which (C₁-C₄)-alkyl may be substituted by 1 to 3 substituents selected from the group consisting of fluorine, hydroxy and (C₁-C₄)-alkoxy,
   or
   R¹² and R¹³ together with the nitrogen atom to which they are attached form a 4- to 7-membered heterocycle,
   and where
   R¹⁴ represents (C₁-C₆)-alkyl, (C₃-C₇)-cycloalkyl or (C₃-C₆)-alkenyl,
or
R³ and R⁴ together with the nitrogen atom to which they are attached form a 4- to 7-membered heterocycle,
   where the 4- to 7-membered heterocycle may be substituted by 1 to 3 substituents independently of one another selected from the group consisting of fluorine, difluoromethyl, trifluoromethyl, cyano, (C₁-C₄)-alkyl, (C₃-C₇)-cycloalkyl, hydroxy, oxo, (C₁-C₄)-alkoxy, difluoromethoxy, trifluoromethoxy and amino,
and
   where the (C₁-C₄)-alkyl, (C₁-C₆)-alkyl, (C₃-C₈)-cycloalkyl, (C₃-C₇)-Cycloalkyl, (C₂-C₆)-alkenyl, (C₃-C₆)-alkenyl and 4- to 7-membered heterocyclyl groups mentioned above, unless stated otherwise, may each independently of one another additionally be substituted by 1 to 3 substituents independently of one another selected from the group consisting of fluorine, difluoromethyl, trifluoromethyl, (C₁-C₄)-alkyl, (C₃-C₇)-cycloalkyl, hydroxy, difluoromethoxy, trifluoromethoxy, (C₁-C₄)-alkoxy, hydroxycarbonyl, (C₁-C₄)-alkoxycarbonyl, amino, phenyl, 4- to 7-membered heterocyclyl and 5- or 6-membered heteroaryl,
   and the N-oxides, salts, solvates, salts of the N-oxides and solvates of the N-oxides and salts thereof.

Compounds according to the invention are the compounds of the formula (I) and the N-oxides, salts, solvates and solvates of the N-oxides and salts thereof, the compounds, encompassed by formula (I), of the formulae specified hereinafter and the N-oxides, salts, solvates and solvates of the N-oxides and salts thereof, and the compounds encompassed by formula (I) and specified hereinafter as working ing examples and the N-oxides, salts, solvates and solvates of the N-oxides and salts thereof, to the extent that the compounds encompassed by formula (I) and specified hereinafter are not already N-oxides, salts, solvates and solvates of the N-oxides and salts.

Preferred salts in the context of the present invention are physiologically acceptable salts of the compounds of the invention. Also encompassed are salts which are not themselves suitable for pharmaceutical applications but can be used, for example, for the isolation, purification or storage of the compounds of the invention.

Physiologically acceptable salts of the compounds of the invention include acid addition salts of mineral acids, carboxylic acids and sulfonic acids, for example salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, formic acid, acetic acid, trifluoroacetic acid, propionic acid, lactic acid, tartaric acid, malic acid, citric acid, fumaric acid, maleic acid and benzoic acid.

Physiologically acceptable salts of the inventive compounds also include salts of conventional bases, by way of example and with preference alkali metal salts (e.g. sodium and potassium salts), alkaline earth metal salts (e.g. calcium and magnesium salts) and ammonium salts derived from ammonia or organic amines having 1 to 16 carbon atoms, by way of example and with preference ethylamine, diethylamine, triethylamine, N,N-ethyldiisopropylamine, monoethanolamine, diethanolamine, triethanolamine, dimethylaminoethanol, diethylaminoethanol, procaine, dicyclohexylamine, dibenzylamine, N-methylpiperidine, N-methylmorpholine, arginine, lysine, choline and 1,2-ethylenediamine.

Solvates in the context of the invention are described as those forms of the compounds of the invention which form a complex in the solid or liquid state by coordination with solvent molecules. Hydrates are a specific form of the solvates in which the coordination is with water. Solvates preferred in the context of the present invention are hydrates.

The compounds of the invention may, depending on their structure, exist in different stereoisomeric forms, i.e. in the form of configurational isomers or else, if appropriate, as conformational isomers (enantiomers and/or diastereomers, including those in the case of atropisomers). The present invention therefore encompasses the enantiomers and diastereomers, and the respective mixtures thereof.

The stereoisomerically homogeneous constituents can be isolated from such mixtures of enantiomers and/or diastereomers in a known manner; chromatographic processes are preferably used for this purpose, especially HPLC chromatography on an achiral or chiral phase.

If the compounds of the invention can occur in tautomeric forms, the present invention encompasses all the tautomeric forms.

The present invention also encompasses all suitable isotopic variants of the compounds of the invention. An isotopic variant of a compound of the invention is understood here to mean a compound in which at least one atom within the compound of the invention has been exchanged for another atom of the same atomic number, but with a different atomic mass from the atomic mass which usually or predominantly occurs in nature. Examples of isotopes which can be incorporated into a compound of the invention are those of hydrogen, carbon, nitrogen, oxygen, phosphorus, sulfur, fluorine, chlorine, bromine and iodine, such as ²H (deuterium), ³H (tritium), ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I. Particular isotopic variants of a compound of the invention, especially those in which one or more radioactive isotopes have been incorporated, may be beneficial, for example, for the examination of the mechanism of action or of the active compound distribution in the body; due to the comparatively easy preparability and detectability, especially compounds labeled with ³H or ¹⁴C isotopes are suitable for this purpose. In addition, the incorporation of isotopes, for example of deuterium, may lead to particular therapeutic benefits as a consequence of greater metabolic stability of the compound, for example an extension of the half-life in the body or a reduction in the active dose required; such modifications of the compounds of the invention may therefore in some cases also constitute a preferred embodiment of the present invention. Isotopic variants of the compounds of the invention can be prepared by the processes known to those skilled in the art, for example by the methods described further down and the procedures described in the working examples, by using corresponding isotopic modifications of the respective reagents and/or starting materials.

The present invention additionally also encompasses prodrugs of the compounds of the invention. The term “prodrugs” in this context refers to compounds which may themselves be biologically active or inactive but are reacted (for example metabolically or hydrolytically) to give compounds of the invention during their residence time in the body.

In the context of the present invention, unless specified otherwise, the substituents are defined as follows:

Alkyl in the context of the invention is a straight-chain or branched alkyl radical having the particular number of carbon atoms specified. By way of example and with preference, mention may be made of the following: methyl, ethyl, n-propyl, isopropyl, n-butyl, 2-methylprop-1-yl, 1-methylpropyl, tert-butyl, n-pentyl, isopentyl, 2-methylbutyl, 3-methylbutyl, n-hexyl.

Alkoxy in the context of the invention is a straight-chain or branched alkoxy radical having 1 to 4 carbon atoms. The following may be mentioned by way of example: methoxy, ethoxy, n-propoxy, isopropoxy, 1-methylprop-1-oxy, n-butoxy, 2-methylprop-1-oxy, tert-butoxy.

Cycloalkyl or carbocycle in the context of the invention is a monocyclic saturated alkyl radical having the number of carbon atoms specified in each case. By way of example and with preference, mention may be made of the following: cyclopropyl, cyclobutyl, cyclopentyl, cyclohexyl, cycloheptyl and cyclooctyl.

5- to 7-membered saturated or partly unsaturated carbocycle in the context of the present invention is a saturated or partly unsaturated cyclic alkyl radical having the number of carbon atoms specified in each case. By way of example and with preference, mention may be made of the following: cyclopentyl, cyclohexyl, cycloheptyl, cyclopentenyl, cyclohexenyl and cycloheptenyl. Alkanediyl in the context of the invention is a straight-chain or branched divalent alkyl radical having 1 to 4 carbon atoms. By way of example and with preference, mention may be made of the following: methylene, ethane-1,2-diyl, ethane-1,1-diyl, propane-1,3-diyl, propane-1,1-diyl, propane-1,2-diyl, propane-2,2-diyl, butane-1,4-diyl, butane-1,2-diyl, butane-1,3-diyl and butane-2,3-diyl.

Alkenyl in the context of the invention is a straight-chain or branched alkenyl radical having 2 to 6 carbon atoms and a double bond. By way of example and with preference, mention may be made of the following: allyl, isopropenyl, n-but-2-en-1-yl and 3-methylbut-2-en-1-yl.

Alkoxycarbonyl in the context of the invention is a straight-chain or branched alkoxy radical having 1 to 4 carbon atoms and a carbonyl group attached to the oxygen. By way of example and with preference, mention may be made of the following: methoxycarbonyl, ethoxycarbonyl, n-propoxycarbonyl, isopropoxycarbonyl and tert-butoxycarbonyl.

Alkoxycarbonylamino in the context of the invention is an amino group having a straight-chain or branched alkoxycarbonyl substituent which has 1 to 4 carbon atoms in the alkyl chain and is attached to the nitrogen atom via the carbonyl group. By way of example and with preference, mention may be made of the following: methoxycarbonylamino, ethoxycarbonylamino, propoxycarbonylamino, nbutoxycarbonylamino, isobutoxycarbonylamino and tert-butoxycarbonylamino.

Alkylthio in the context of the invention is a thio group having a straight-chain or branched alkyl substituent having 1 to 4 carbon atoms. By way of example and with preference, mention may be made of the following: methylthio, ethylthio, n-propylthio, isopropylthio, n-butylthio and tert-butylthio.

Alkylsulfonyl in the context of the invention is a straight-chain or branched alkyl radical which has 1 to 4 carbon atoms and is attached via a sulfonyl group. The following may be mentioned by way of example and by way of preference: methylsulfonyl, ethylsulfonyl, n-propylsulfonyl, isopropylsulfonyl, n-butylsulfonyl and tert-butylsulfonyl.

Monoalkylamino in the context of the invention is an amino group having a straight-chain or branched alkyl substituent having 1 to 6 carbon atoms. By way of example and with preference, mention may be made of the following: methylamino, ethylamino, n-propylamino, isopropylamino and tert-butylamino.

Dialkylamino in the context of the invention is an amino group having two identical or different straight-chain or branched alkyl substituents each having 1 to 6 carbon atoms. By way of example and with preference, mention may be made of the following: N,N-dimethylamino, N,N-diethylamino, N-ethyl-N-methyl-amino, N-methyl-N-n-propylamino, N-isopropyl-N-n-propylamino, N-tert-butyl-N-methylamino, N-ethyl-N-n-pentylamino and N-n-hexyl-N-methylamino.

5- to 7-membered saturated or partly unsaturated heterocycle in the context of the invention is a saturated or partly unsaturated heterocycle which has a total of 5 to 7 ring atoms and contains one ring heteroatom from the series N, O, S, SO and/or SO₂. The following may be mentioned by way of example: pyrrolidinyl, tetrahydrofuranyl, piperidinyl, tetrahydropyranyl, dihydropyrrolyl, dihydropyridyl.

Heterocyclyl or heterocycle in the context of the invention is a saturated heterocycle which has a total of 4 to 7 ring atoms and contains one or two ring heteroatoms from the group consisting of N, O, S, SO and/or SO₂. The following may be mentioned by way of example: azetidinyl, pyrrolidinyl, pyrazolidinyl, imidazolinyl, tetrahydrofuranyl, piperidinyl, piperazinyl, tetrahydropyranyl, morpholinyl, thiomorpholinyl and dioxidothiomorpholinyl. Preference is given to oxetanyl, pyrrolidinyl, tetrahydrofuranyl, piperidinyl and tetrahydropyranyl.

Heteroaryl in the context of the invention is a monocyclic or bicyclic aromatic heterocycle (heteroaromatic) which has a total of 5 to 10 ring atoms, contains up to four identical or different ring heteroatoms from the group consisting of N, O and S and is attached via a ring carbon atom or optionally via a ring nitrogen atom. The following may be mentioned by way of example: furyl, pyrrolyl, thienyl, pyrazolyl, imidazolyl, thiazolyl, oxazolyl, isoxazolyl, isothiazolyl, triazolyl, oxadiazolyl, thiadiazolyl, pyridyl, pyrimidinyl, pyridazinyl, pyrazinyl, triazinyl, benzofuranyl, benzothienyl, benzimidazolyl, benzoxazolyl, benzothiazolyl, benzotriazolyl, indolyl, indazolyl, imidazopyridazinyl, quinolinyl, isoquinolinyl, naphthyridinyl, quinazolinyl, quinoxalinyl, phthalazinyl, dihydrothienopyrazolyl, thienopyrazolyl, pyrazolopyrazolyl, imidazothiazolyl, tetrahydrocyclopentapyrazolyl, dihydrocyclopentapyrazolyl, tetrahydroindazolyl, dihydroindazolyl, pyrazolopyridyl, tetrahydropyrazolopyridyl, pyrazolopyrimidinyl and imidazopyridyl. Preferred in the definition of ring Q are 5- or 6-membered monocyclic heteroaryl radicals having up to three ring nitrogen atoms, such as pyrazolyl, imidazolyl, triazolyl, pyridyl, pyrimidinyl and pyridazinyl, and 8- or 9-membered bicyclic heteroaryl radicals having up to four ring nitrogen atoms, such as indazol-3-yl, indazol-1-yl, pyrazolo[3,4-b]pyridin-3-yl, pyrazolo[4,3-b]pyridin-1-yl, imidazo[1,5-b]pyridazin-5-yl, imidazo[1,5-a]pyridin-1-yl, pyrazolo[3,4-d]pyrimidin-3-yl. Particular preference is given to 8- or 9-membered bicyclic heteroaryl radicals having 2 or 3 ring nitrogen atoms, such as pyrazolo[3,4-b]pyridin-3-yl and indazol-3-yl. Preferred in the definition of the radical R¹ are thienyl, pyridyl, thiazolyl, oxazolyl, isoxazolyl. Preferred in the definition of the radical R² are pyridyl, pyrimidinyl, pyrazinyl or pyridazinyl. Preferred in the definition of the radical R⁴ are pyridyl, pyrimidinyl, pyrazinyl, furanyl, 2,3,5-triazol-1-yl, thiazolin-2-yl, 1,3,4-oxadiazol-2-yl, 1,3,4-thiadiazol-2-yl.

Halogen in the context of the invention is fluorine, chlorine, bromine and iodine. Preference is given to fluorine and chlorine.

An oxo group in the context of the invention is an oxygen atom attached to a carbon atom via a double bond.

A thiooxo group in the context of the invention is a sulfur atom attached via a double bond to a carbon atom.

In the formula of the group that L, Q or R² may represent, the end point of the line marked by the symbol #, #¹, #², * and ** does not represent a carbon atom or a CH₂ group but is part of the bond to the respective atom to which L, Q or R² is attached.

When radicals in the compounds of the invention are substituted, the radicals may be mono- or polysubstituted, unless specified otherwise. In the context of the present invention, all radicals which occur more than once are defined independently of one another. Substitution by one, two or three identical or different substituents is preferred. Substitution by one or two identical or different substituents is preferred.

In the context of the present invention, the term “treatment” or “treating” includes inhibition, retardation, checking, alleviating, attenuating, restricting, reducing, suppressing, repelling or healing of a disease, a condition, a disorder, an injury or a health problem, or the development, the course or the progression of such states and/or the symptoms of such states. The term “therapy” is understood here to be synonymous with the term “treatment”.

The terms “prevention”, “prophylaxis” and “preclusion” are used synonymously in the context of the present invention and refer to the avoidance or reduction of the risk of contracting, experiencing, suffering from or having a disease, a condition, a disorder, an injury or a health problem, or a development or advancement of such states and/or the symptoms of such states.

The treatment or prevention of a disease, a condition, a disorder, an injury or a health problem may be partial or complete.

A particular embodiment of the present invention encompasses compounds of the formula (I) in which the ring Q represents a group of the formula

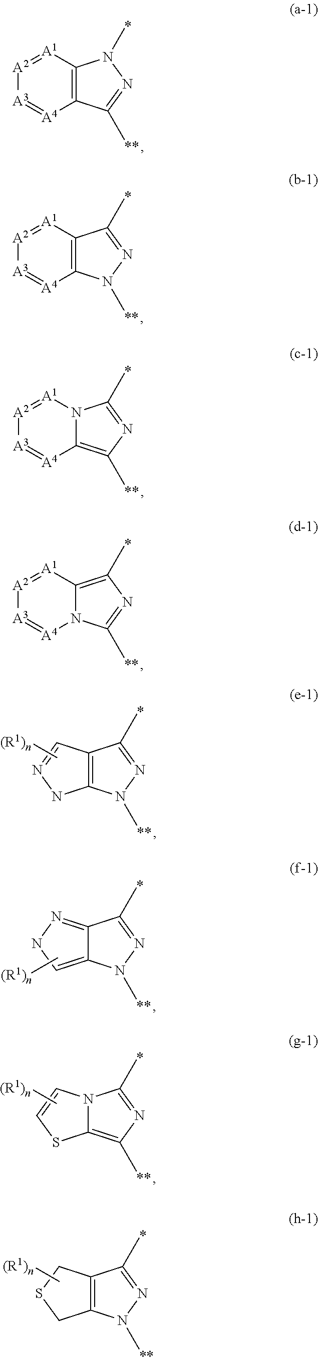

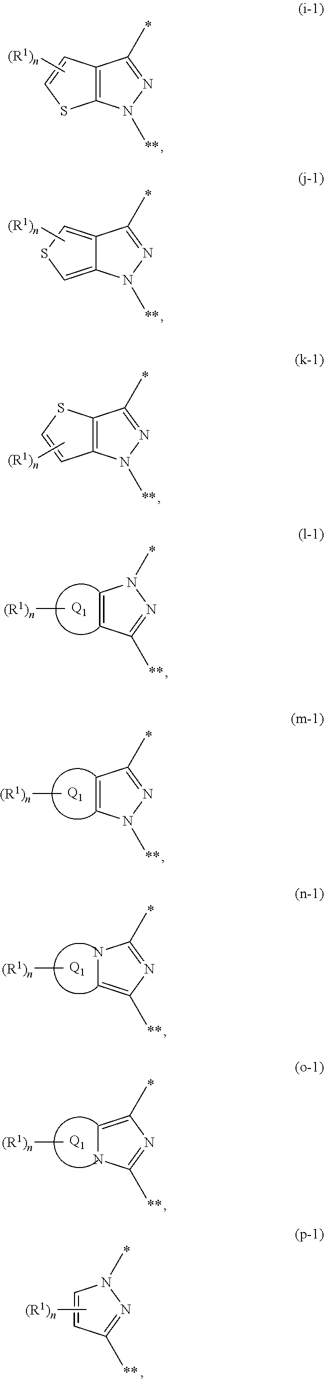

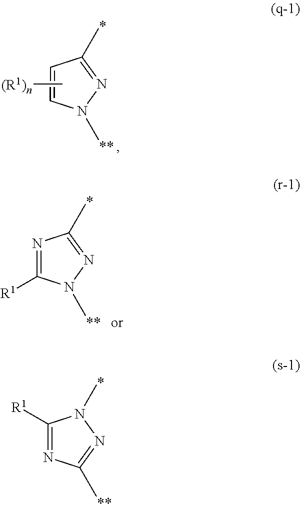

where
* represents the point of attachment to —CH₂—R²,
** represents the point of attachment to the pyrimidine ring,
n represents a number 0, 1 or 2,
the ring Q₁ together with the atoms to which it is attached forms a 5- to 7-membered saturated or partially unsaturated carbocycle or a 5- to 7-membered saturated or partially unsaturated heterocycle,
A¹, A², A³ and A⁴ independently of one another each represent N, C—H or C—R¹, with the proviso that not more than two of the A¹, A², A³ and A⁴ groups represent N.

Preference is given in the context of the present invention to compounds of the formula (I) in which the ring Q represents a group of the formula

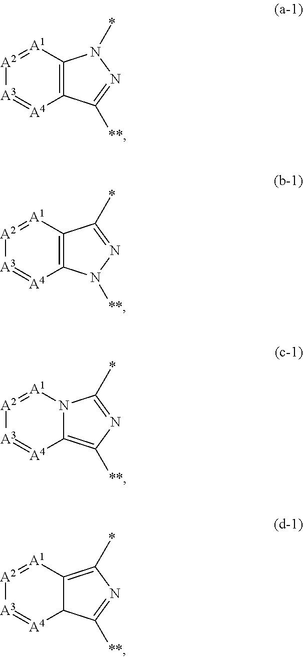

where
   * represents the point of attachment to —CH₂—R²,
   ** represents the point of attachment to the pyrimidine ring,
n represents a number 0, 1 or 2,
A¹, A², A³ and A⁴ independently of one another each represent N, C—H or C—R¹, with the proviso that not more than two of the A¹, A², A³ and A⁴ groups represent N,
L represents a #¹-CR^{5A}R^{5B}—(CR^{6A}R^{6B})ₘ-#² group, where
   #¹ represents the point of attachment to the carbonyl group,
   #² represents the attachment site to the pyrimidine ring,
   m represents a number 0 or 1,
   R^{5A} represents hydrogen, fluorine, trifluoromethyl or (C₁-C₄)-alkyl,
   R^{5B} represents hydrogen, fluorine, trifluoromethyl, (C₁-C₄)-alkyl, (C₃-C₇)-cycloalkyl or a group of the formula -M-R⁷,
      in which (C₁-C₄)-alkyl may be substituted by 1 to 3 substituents independently of one another selected from the group consisting of fluorine, cyano, trifluoromethyl, (C₃-C₇)cycloalkyl, difluoromethoxy and trifluoromethoxy,
      M represents a bond or methylene,
      R⁷ represents —(C═O)—OR⁸ or —(C═O)—NR⁹R¹⁰, in which
         R⁸ represents hydrogen, (C₁-C₄)-alkyl, (C₃-C₆)-cycloalkyl or 4- or 7-membered heterocyclyl,
         R⁹ and R¹⁰ independently of one another each represent hydrogen, (C₁-C₄)alkyl, (C₃-C₆)-cycloalkyl, 4- to 7-membered heterocyclyl, phenyl or 5- or 6-membered heteroaryl,
         or
         R⁹ and R¹⁰ together with the atom(s) to which they are respectively attached form a 4- to 7-membered heterocycle,
   or
   R^{5A} and R^{5B} together with the carbon atom to which they are attached form a (C₂-C₄)alkenyl group, a 3- to 6-membered carbocycle or a 4- to 7-membered heterocycle, where the 3- to 6-membered carbocycle may be monosubstituted by hydroxy and up to disubstituted by fluorine,
   R^{6A} represents hydrogen, fluorine, (C₁-C₄)-alkyl or hydroxy,
   R⁶ represents hydrogen, fluorine, (C₁-C₄)-alkyl or trifluoromethyl,
R¹ represents fluorine, chlorine, cyano, difluoromethyl, trifluoromethyl, (C₁-C₄)-alkyl, (C₃-C₅)cycloalkyl or (C₁-C₄)-alkoxy,
n represents a number 0, 1 or 2,
R² represents (C₁-C₆)-alkyl, phenyl or 5- or 6-membered heteroaryl,
   where (C₁-C₆)-alkyl is substituted by a substituent selected from the group consisting of difluoromethyl and trifluoromethyl and may furthermore be up to trisubstituted by fluorine, and where phenyl is substituted by 1 to 3 fluorine substituents and may furthermore be substituted by 1 or 2 substituents independently of one another selected from the group consisting of methyl and methoxy,
   and where 5- or 6-membered heteroaryl is up to disubstituted by fluorine,
R³ represents hydrogen, (C₁-C₄)-alkyl or cyclopropyl,
R⁴ represents hydrogen, (C₁-C₁₀)-alkyl, (C₃-C₇)-cycloalkyl, (C₂-C₆)-alkenyl, 4- to 7-membered heterocyclyl, phenyl, 5- or 6-membered heteroaryl, —NR¹²R¹³ or —OR¹⁴,
   where (C₁-C₁₀)-alkyl may be substituted by 1 to 3 substituents independently of one another selected from the group consisting of fluorine, difluoromethyl, trifluoromethyl, (C₃-C₇)cycloalkyl, hydroxy, oxo, —OR¹⁵, —NR¹⁶—(C═O)—R¹⁷, —NR¹⁸R¹⁹, —(C═O)—NR¹⁸R¹⁹, —S(O)ₚ—R²⁰, —NR¹⁸—SO₂—R¹⁹, phenyl, 4- to 7-membered heterocyclyl and 5- or 6-membered heteroaryl,
      in which (C₃-C₇)-cycloalkyl and 4- to 7-membered heterocyclyl independently of one another may each be substituted by a substituent selected from the group consisting of (C₁-C₄)-alkyl, oxo, hydroxy, amino and furthermore up to tetrasubstituted by fluorine, and
      in which phenyl and 5- or 6-membered heteroaryl independently of one another may each be substituted by (C₁-C₄)-alkyl and furthermore up to trisubstituted by fluorine,
      p represents the number 0, 1 or 2,
      R¹⁵ and R²⁰ independently of one another represent (C₁-C₄)-alkyl which may be up to pentasubstituted by fluorine, represent phenyl or (C₃-C₇)-cycloalkyl,
      R¹⁶ and R¹⁷ independently of one another each represent hydrogen, (C₁-C₄)-alkyl or (C₃-C₇)-cycloalkyl,
      R¹⁸ and R¹⁹ independently of one another represent hydrogen, (C₁-C₆)-alkyl which may be up to pentasubstituted by fluorine, or represent (C₃-C₇)-cycloalkyl, or
      R¹⁸ and R¹⁹ together with the nitrogen atom to which they are attached form a 4- to 7-membered heterocycle,
         in which the 4- to 7-membered heterocycle may be up to tetrasubstituted by fluorine,
   where (C₃-C₇)-cycloalkyl may be substituted by a substituent selected from the group consisting of (C₁-C₄)-alkyl, hydroxy, amino, cyano and furthermore up to tetrasubstituted by fluorine, and where (C₂-C₆)-alkenyl may be substituted by (C₁-C₄)-alkyl and furthermore up to pentasubstituted by fluorine,
   and where 4- to 7-membered heterocyclyl may be substituted by 1 to 4 substituents independently of one another selected from the group consisting of fluorine, trifluoromethyl, oxo, (C₁-C₄)-alkyl, hydroxy and amino,
   and where 5- or 6-membered heteroaryl and phenyl may each be substituted by 1 to 3 substituents independently of one another selected from the group consisting of halogen, (C₁-C₄)-alkyl, (C₁-C₄)-alkoxy, cyano and (C₃-C₅)-cycloalkyl,
   and where
   R¹² and R¹³ independently of one another represent hydrogen or (C₁-C₄)-alkyl,
   or
   R¹² and R¹³ together with the nitrogen atom to which they are attached form a 4- to 7-membered heterocycle,
   and where
   R¹⁴ represents (C₁-C₆)-alkyl which may be up to pentasubstituted by fluorine, represents (C₃-C₇)-cycloalkyl or (C₃-C₆)-alkenyl,
or
R³ and R⁴ together with the nitrogen atom to which they are attached form a 4- to 7-membered heterocycle,
   where the 4- to 7-membered heterocycle may be substituted by 1 to 3 substituents independently of one another selected from the group consisting of trifluoromethyl, (C₁-C₄)-alkyl, (C₃-C₇)cycloalkyl, hydroxy, (C₁-C₄)-alkoxy, trifluoromethoxy and amino and furthermore up to tetrasubstituted by fluorine,
and the salts, solvates and solvates of the salts thereof.

Also particularly preferred in the context of the present invention are compounds of the formula (I) in which the ring Q represents a group of the formula

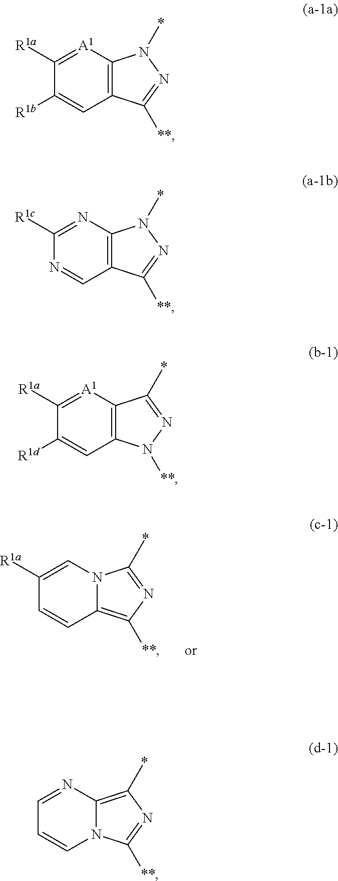

where
* represents the point of attachment to —CH₂—R²,
** represents the point of attachment to the pyrimidine ring, in which
A¹ represents N or C—H,
R¹ represents hydrogen or methyl if A¹ represents nitrogen,
or
R^{1a} represents hydrogen, fluorine or chlorine if A¹ represents C—H,
R^{1b} represents hydrogen or fluorine,
R^{1c} represents hydrogen or methyl,
R^{1d} represents hydrogen, methyl or fluorine,
R^{1e} represents hydrogen or chlorine,
L represents a #¹-CR^{5A}R^{5B}—(CR^{6A}R^{6B})ₘ—#² group, where
   #¹ represents the point of attachment to the carbonyl group,
   #² represents the attachment site to the pyrimidine ring,
   m represents a number 0,
   R^{5A} represents hydrogen, methyl or ethyl,
   R^{5B} represents hydrogen, fluorine, trifluoromethyl, methyl, ethyl or ethoxycarbonyl,
      where methyl, ethyl or ethoxycarbonyl may be up to trisubstituted by fluorine, or
   R^{5A} and R^{5B} together with the carbon atom to which they are attached form a cyclopropyl ring,
R² represents (C₁-C₄)-alkyl, phenyl or 6-membered heteroaryl,
   where (C₁-C₄)-alkyl is substituted by a substituent selected from the group consisting of difluoromethyl and trifluoromethyl and may furthermore be up to disubstituted by fluorine,
   and where phenyl is substituted by 1 to 3 fluorine substituents and may furthermore be substituted by 1 or 2 substituents independently of one another selected from the group consisting of methyl and methoxy,
   and where 6-membered heteroaryl is up to disubstituted by fluorine,
R³ represents hydrogen, (C₁-C₄)-alkyl or cyclopropyl,
R⁴ represents hydrogen, (C₁-C₁₀)-alkyl, (C₃-C₇)-cycloalkyl, (C₂-C₆)-alkenyl, 4- to 7-membered heterocyclyl, phenyl, 5- or 6-membered heteroaryl, —NR¹²R¹³ or —OR¹⁴,
   where (C₁-C₁₀)-alkyl may be substituted by 1 to 3 substituents independently of one another selected from the group consisting of fluorine, difluoromethyl, trifluoromethyl, (C₃-C₇)cycloalkyl, hydroxy, oxo, —OR¹⁵, —NR¹⁶—(C═O)—R¹⁷, —NR¹⁸R¹⁹, —(C═O)—NR¹⁸R¹⁹, —S(O)ₚ—R²⁰, phenyl, 4- to 7-membered heterocyclyl and 5- or 6-membered heteroaryl,
      in which (C₃-C₇)-cycloalkyl and 4- to 7-membered heterocyclyl independently of one another may each be substituted by a substituent selected from the group consisting of (C₁-C₄)-alkyl, oxo, hydroxy, amino and furthermore up to tetrasubstituted by fluorine, and
      in which phenyl and 5- or 6-membered heteroaryl independently of one another may each be substituted by (C₁-C₄)-alkyl and furthermore up to trisubstituted by fluorine,
      p represents the number 0, 1 or 2,
      R¹⁵ and R²⁰ independently of one another each represent (C₁-C₄)-alkyl, phenyl or (C₃-C₇)-cycloalkyl,
         in which (C₁-C₄)-alkyl may be substituted up to five times by fluorine,
      R¹⁶ and R¹⁷ independently of one another each represent hydrogen, (C₁-C₄)-alkyl or (C₃-C₇)-cycloalkyl,
      R¹⁸ and R¹⁹ independently of one another represent hydrogen, (C₁-C₆)-alkyl
         which may be up to pentasubstituted by fluorine, or represent (C₃-C₆)-cycloalkyl, or
      R¹⁸ and R¹⁹ together with the nitrogen atom to which they are attached form a 4- to 6-membered heterocycle,
   where (C₃-C₇)-cycloalkyl may be substituted by a substituent selected from the group consisting of (C₁-C₄)-alkyl, hydroxy, amino, cyano and furthermore up to tetrasubstituted by fluorine,
   and
   where (C₂-C₆)-alkenyl may be up to pentasubstituted by fluorine,
   and
   where 4- to 7-membered heterocyclyl may be substituted by 1 or 2 substituents independently of one another selected from the group consisting of oxo, (C₁-C₄)-alkyl, hydroxy, amino and furthermore up to tetrasubstituted by fluorine,
   and
   where 5- or 6-membered heteroaryl and phenyl may each be substituted by 1 to 3 substituents independently of one another selected from the group consisting of halogen, (C₁-C₄)-alkyl, (C₁-C₄)-alkoxy, cyano and (C₃-C₅)-cycloalkyl,
   and where
   R¹² and R¹³ independently of one another represent hydrogen or (C₁-C₄)-alkyl,
   or
   R¹² and R¹³ together with the nitrogen atom to which they are attached form a 4- to 7-membered heterocycle,
   and where
   R¹⁴ represents (C₁-C₆)-alkyl which may be up to pentasubstituted by fluorine, represents (C₃-C₇)-cycloalkyl or (C₃-C₆)-alkenyl,
or
R³ and R⁴ together with the nitrogen atom to which they are attached form a 4- to 7-membered heterocycle,
   where the 4- to 7-membered heterocycle may be substituted by 1 or 2 substituents independently of one another selected from the group consisting of trifluoromethyl, (C₁-C₄)-alkyl, (C₃-C₇)cycloalkyl, hydroxy, (C₁-C₄)-alkoxy, trifluoromethoxy and amino and furthermore up to tetrasubstituted by fluorine,
      and the salts, solvates and solvates of the salts thereof.

Also particularly preferred in the context of the present invention are compounds of the formula (I) in which the ring Q represents a group of the formula

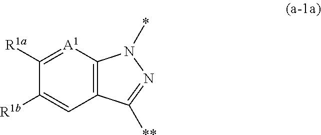

where
* represents the point of attachment to —CH₂—R²,
** represents the point of attachment to the pyrimidine ring, in which
A¹ represents N or C—H,
R^{1a} represents hydrogen or methyl if A¹ represents nitrogen,
or
R^{1a} represents hydrogen, fluorine or chlorine if A¹ represents C—H,
R^{1b} represents hydrogen or fluorine,
L represents a #¹-CR^{5A}R^{5B}—(CR^{6A}R^{6B})ₘ—#² group, where
   #¹ represents the point of attachment to the carbonyl group,
   #² represents the attachment site to the pyrimidine ring,
   m represents a number 0,
   R^{5A} represents hydrogen, methyl or ethyl,
   R^{5B} represents hydrogen, fluorine, trifluoromethyl, methyl or ethyl,
      where methyl and ethyl may be up to trisubstituted by fluorine,
   or
   R^{5A} and R^{5B} together with the carbon atom to which they are attached form a cyclopropyl ring,
R² represents 2,2,2-trifluoroeth-1-yl, phenyl or pyridyl,
   where phenyl is substituted by 1 to 3 fluorine substituents,
   and
   where pyridyl is monosubstituted by fluorine,
R³ represents hydrogen, (C₁-C₄)-alkyl or cyclopropyl,
R⁴ represents hydrogen, (C₁-C₆)-alkyl, (C₃-C₆)-cycloalkyl, (C₂-C₆)-alkenyl, 5- or 6-membered heterocyclyl, phenyl, 5- or 6-membered heteroaryl or —OR¹⁴,
   where (C₁-C₆)-alkyl may be substituted by 1 or 2 substituents independently of one another selected from the group consisting of difluoromethyl, trifluoromethyl, (C₃-C₆)-cycloalkyl, hydroxy, oxo, —OR¹⁵, —NR¹⁶—(C═O)—R¹⁷, —NR¹⁸R¹⁹, —(C═O)—NR¹⁸R¹⁹, —S(O)ₚ—R²⁰, phenyl, 4- to 6-membered heterocyclyl and 5- or 6-membered heteroaryl and furthermore up to trisubstituted by fluorine,
      in which (C₃-C₆)-cycloalkyl and 4- to 6-membered heterocyclyl independently of one another may each be substituted by a substituent selected from the group consisting of (C₁-C₄)-alkyl, oxo, hydroxy, amino and furthermore up to tetrasubstituted by fluorine,
      and
      in which phenyl and 5- or 6-membered heteroaryl independently of one another may each be substituted by (C₁-C₄)-alkyl and furthermore up to trisubstituted by fluorine,
      p represents the number 0, 1 or 2,
      R¹⁵ and R²⁰ each independently of one another represent (C₁-C₄)-alkyl,
         in which (C₁-C₄)-alkyl may be substituted up to five times by fluorine,
      R¹⁶ represents hydrogen or (C₁-C₄)-alkyl,
      R¹⁷ represents (C₁-C₄)-alkyl or (C₃-C₆)-cycloalkyl,
      R¹⁸ and R¹⁹ independently of one another represent hydrogen or (C₁-C₄)-alkyl which may be up to pentasubstituted by fluorine,
      or
      R¹⁸ and R¹⁹ together with the nitrogen atom to which they are attached form a 5- or 6-membered heterocycle,
   where (C₃-C₆)-cycloalkyl may be substituted by a substituent selected from the group consisting of (C₁-C₄)-alkyl, hydroxy, amino, cyano and furthermore up to tetrasubstituted by fluorine, and where (C₂-C₆)-alkenyl may be up to trisubstituted by fluorine,
   and where 5- or 6-membered heterocyclyl may be substituted by a substituent selected from the group consisting of oxo, (C₁-C₄)-alkyl, hydroxy and amino and furthermore up to tetrasubstituted by fluorine,
   and where 5- or 6-membered heteroaryl and phenyl independently of one another may each be substituted by a substituent selected from the group consisting of halogen, (C₁-C₄)-alkyl, cyano and (C₃-C₅)-cycloalkyl,
   and where
   R¹⁴ represents (C₁-C₆)-alkyl which may be up to pentasubstituted by fluorine, or represents (C₃-C₆)-alkenyl,
or
R³ and R⁴ together with the nitrogen atom to which they are attached form a 5- or 6-membered heterocycle,
   where the 5- or 6-membered heterocycle may be substituted by a substituent selected from the group consisting of (C₁-C₄)-alkyl, oxo, hydroxy and furthermore up to tetrasubstituted by fluorine,
      and the salts, solvates and solvates of the salts thereof.

Especially preferred in the context of the present invention are compounds of the formula (I) in which the ring Q represents a group of the formula

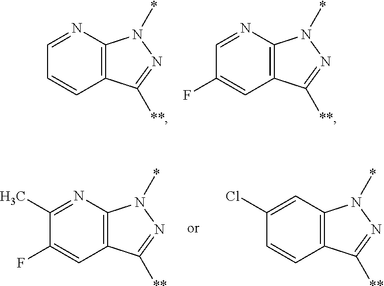

where
* represents the point of attachment to —CH₂—R²,
** represents the point of attachment to the pyrimidine ring, in which
L represents a #¹-CR^{5A}R^{5B}—(CR^{6A}R^{6B})ₘ-#² group, where
   #¹ represents the point of attachment to the carbonyl group,
   #² represents the attachment site to the pyrimidine ring,
   m represents a number 0,
   R^{5A} represents methyl,
   R^{5B} represents methyl or trifluoromethyl,
R² represents a phenyl group of the formula

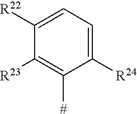

where
# represents the point of attachment to the methylene group,
R²² and R²⁴ independently of one another each represent hydrogen or fluorine,
R²³ represents fluorine,
or
R² represents 3-fluoropyrid-2-yl,
R³ represents hydrogen or methyl,
R⁴ represents hydrogen, (C₁-C₄)-alkyl or cyclopropyl,
   where (C₁-C₄)-alkyl may be substituted by a substituent selected from the group consisting of hydroxy, amino, methoxy, 2,2,2-trifluoroethoxy and cyclopropyl, and furthermore up to trisubstituted by fluorine,
   and where cyclopropyl may be substituted by cyano,
      and the salts, solvates and solvates of the salts thereof.

A particular embodiment of the present invention comprises compounds of the formula (I) in which
L represents a #¹-CR^{5A}R^{5B}—(CR^{6A}R^{6B})ₘ-#² group, where
   #¹ represents the point of attachment to the carbonyl group,
   #² represents the attachment site to the pyrimidine ring,
   m represents a number 0,
   R^{5A} represents hydrogen, methyl or ethyl,
   R^{5B} represents hydrogen, fluorine, trifluoromethyl, methyl or ethyl,
      where methyl and ethyl may be up to trisubstituted by fluorine,
   or
   R^{5A} and R^{5B} together with the carbon atom to which they are attached form a cyclopropyl ring,
R² represents 2,2,2-trifluoroeth-1-yl, phenyl or pyridyl,
   where phenyl is substituted by 1 to 3 fluorine substituents,
   and
   where pyridyl is monosubstituted by fluorine.

A particular embodiment of the present invention comprises compounds of the formula (I) in which
L represents a #¹-CR^{5A}R^{5B}—(CR^{6A}R^{6B})ₘ-#² group, where
   #¹ represents the point of attachment to the carbonyl group,
   #² represents the attachment site to the pyrimidine ring,
   m represents a number 0,
   R^{5A} represents methyl,
   R^{5B} represents methyl or trifluoromethyl.

A particular embodiment of the present invention comprises compounds of the formula (I) in which
L represents a #¹-CR^{5A}R^{5B}—(CR^{6A}R^{6B})ₘ#² group, where
   #¹ represents the point of attachment to the carbonyl group,
   #² represents the attachment site to the pyrimidine ring,
   m represents a number 0,
   R^{5A} represents methyl,
   R^{5A} represents methyl.

A particular embodiment of the present invention comprises compounds of the formula (I) in which
L represents a #¹-CR^{5A}R^{5B}—(CR^{6A}R^{6B})ₘ-#² group, where
   #¹ represents the point of attachment to the carbonyl group,
   #² represents the attachment site to the pyrimidine ring,
   m represents a number 0,
   R^{5A} represents methyl,
   R^{5B} represents trifluoromethyl.

A particular embodiment of the present invention comprises compounds of the formula (I) in which
R² represents a phenyl group of the formula

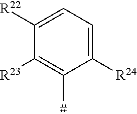

where
# represents the point of attachment to the methylene group,
R²² and R²⁴ independently of one another each represent hydrogen or fluorine,
R²³ represents fluorine.

A particular embodiment of the present invention comprises compounds of the formula (I) in which
R² represents 3-fluoropyrid-2-yl.

A particular embodiment of the present invention comprises compounds of the formula (I) in which
R³ represents hydrogen, (C₁-C₄)-alkyl or cyclopropyl,
R⁴ represents hydrogen, (C₁-C₆)-alkyl, (C₃-C₆)-cycloalkyl, (C₂-C₆)-alkenyl, 5- or 6-membered heterocyclyl, phenyl, 5- or 6-membered heteroaryl or —OR¹⁴,
   where (C₁-C₆)-alkyl may be substituted by 1 or 2 substituents independently of one another selected from the group consisting of difluoromethyl, trifluoromethyl, (C₃-C₆)-cycloalkyl, hydroxy, oxo, —OR¹⁵, —NR¹⁶—(C═O)—R¹⁷, —NR¹⁸R¹⁹, —(C═O)—NR¹⁸R¹⁹, —S(O)ₚ—R²⁰, phenyl, 4- to 6-membered heterocyclyl and 5- or 6-membered heteroaryl and furthermore up to trisubstituted by fluorine,
      in which (C₃-C₆)-cycloalkyl and 4- to 6-membered heterocyclyl independently of one another may each be substituted by a substituent selected from the group consisting of (C₁-C₄)-alkyl, oxo, hydroxy, amino and furthermore up to tetrasubstituted by fluorine, and
      in which phenyl and 5- or 6-membered heteroaryl independently of one another may each be substituted by (C₁-C₄)-alkyl and furthermore up to trisubstituted by fluorine,
      p represents the number 0, 1 or 2,
      R¹⁵ and R²⁰ each independently of one another represent (C₁-C₄)-alkyl, in which (C₁-C₄)-alkyl may be substituted up to five times by fluorine,
      R¹⁶ represents hydrogen or (C₁-C₄)-alkyl,
      R¹⁷ represents (C₁-C₄)-alkyl or (C₃-C₆)-cycloalkyl,
      R¹⁸ and R¹⁹ independently of one another represent hydrogen or (C₁-C₄)-alkyl which may be up to pentasubstituted by fluorine,
      or
      R¹⁸ and R¹⁹ together with the nitrogen atom to which they are attached form a 5- or 6-membered heterocycle,
   where (C₃-C₆)-cycloalkyl may be substituted by a substituent selected from the group consisting of (C₁-C₄)-alkyl, hydroxy, amino, cyano and furthermore up to tetrasubstituted by fluorine, and where (C₂-C₆)-alkenyl may be up to trisubstituted by fluorine,
   and where 5- or 6-membered heterocyclyl may be substituted by a substituent selected from the group consisting of oxo, (C₁-C₄)-alkyl, hydroxy and amino and furthermore up to tetrasubstituted by fluorine,
   and where 5- or 6-membered heteroaryl and phenyl independently of one another may each be substituted by a substituent selected from the group consisting of halogen, (C₁-C₄)-alkyl, cyano and (C₃-C₅)-cycloalkyl,
   and where
   R¹⁴ represents (C₁-C₆)-alkyl which may be up to pentasubstituted by fluorine, or represents (C₃-C₆)-alkenyl,
or
R³ and R⁴ together with the nitrogen atom to which they are attached form a 5- or 6-membered heterocycle,
   where the 5- or 6-membered heterocycle may be substituted by a substituent selected from the group consisting of (C₁-C₄)-alkyl, oxo, hydroxy and furthermore up to tetrasubstituted by fluorine.

A particular embodiment of the present invention comprises compounds of the formula (I) in which
R³ represents hydrogen,
R⁴ represents hydrogen, (C₁-C₆)-alkyl, (C₃-C₆)-cycloalkyl, (C₂-C₆)-alkenyl, 5- or 6-membered heterocyclyl, phenyl, 5- or 6-membered heteroaryl or —OR¹⁴,
   where (C₁-C₆)-alkyl may be substituted by 1 or 2 substituents independently of one another selected from the group consisting of difluoromethyl, trifluoromethyl, (C₃-C₆)-cycloalkyl, hydroxy, oxo, —OR¹⁵, —NR¹⁶—(C═O)—R¹⁷, —NR¹⁸R¹⁹, —(C═O)—NR¹⁸R¹⁹, —S(O)ₚ—R²⁰, phenyl, 4- to 6-membered heterocyclyl and 5- or 6-membered heteroaryl and furthermore up to trisubstituted by fluorine,
      in which (C₃-C₆)-cycloalkyl and 4- to 6-membered heterocyclyl independently of one another may each be substituted by a substituent selected from the group consisting of (C₁-C₄)-alkyl, oxo, hydroxy, amino and furthermore up to tetrasubstituted by fluorine, and
      in which phenyl and 5- or 6-membered heteroaryl independently of one another may each be substituted by (C₁-C₄)-alkyl and furthermore up to trisubstituted by fluorine,
      p represents the number 0, 1 or 2,
      R¹⁵ and R²⁰ each independently of one another represent (C₁-C₄)-alkyl,
         in which (C₁-C₄)-alkyl may be substituted up to five times by fluorine,
      R¹⁶ represents hydrogen or (C₁-C₄)-alkyl,
      R¹⁷ represents (C₁-C₄)-alkyl or (C₃-C₆)-cycloalkyl,
      R¹⁸ and R¹⁹ independently of one another represent hydrogen or (C₁-C₄)-alkyl which may be up to pentasubstituted by fluorine,
      or
      R¹⁸ and R¹⁹ together with the nitrogen atom to which they are attached form a 5- or 6-membered heterocycle,
   where (C₃-C₆)-cycloalkyl may be substituted by a substituent selected from the group consisting of (C₁-C₄)-alkyl, hydroxy, amino, cyano and furthermore up to tetrasubstituted by fluorine,
   and where (C₂-C₆)-alkenyl may be up to trisubstituted by fluorine,
   and where 5- or 6-membered heterocyclyl may be substituted by a substituent selected from the group consisting of oxo, (C₁-C₄)-alkyl, hydroxy and amino and furthermore up to tetrasubstituted by fluorine,
   and where 5- or 6-membered heteroaryl and phenyl independently of one another may each be substituted by a substituent selected from the group consisting of halogen, (C₁-C₄)-alkyl, cyano and (C₃-C₅)-cycloalkyl,
   and where
   R¹⁴ represents (C₁-C₆)-alkyl which may be up to pentasubstituted by fluorine, or represents (C₃-C₆)-alkenyl,
or
R³ and R⁴ together with the nitrogen atom to which they are attached form a 5- or 6-membered heterocycle,
   where the 5- or 6-membered heterocycle may be substituted by a substituent selected from the group consisting of (C₁-C₄)-alkyl, oxo, hydroxy and furthermore up to tetrasubstituted by fluorine.

A particular embodiment of the present invention comprises compounds of the formula (I) in which
R³ represents hydrogen or methyl,
R⁴ represents hydrogen, (C₁-C₄)-alkyl or cyclopropyl,
   where (C₁-C₄)-alkyl may be substituted by a substituent selected from the group consisting of hydroxy, amino, methoxy, 2,2,2-trifluoroethoxy and cyclopropyl, and furthermore up to trisubstituted by fluorine,
   and where cyclopropyl may be substituted by cyano.

A particular embodiment of the present invention comprises compounds of the formula (I) in which
R³ represents hydrogen,
R⁴ represents hydrogen, (C₁-C₄)-alkyl or cyclopropyl,
   where (C₁-C₄)-alkyl may be substituted by a substituent selected from the group consisting of hydroxy, amino, methoxy, 2,2,2-trifluoroethoxy and cyclopropyl, and furthermore up to trisubstituted by fluorine.

A particular embodiment of the present invention comprises compounds of the formula (I) in which
R³ and R⁴ represent hydrogen.

A particular embodiment of the present invention comprises compounds of the formula (I) in which the ring Q represents a group of the formula

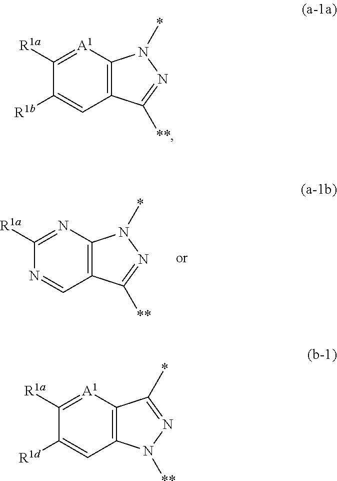

where
* represents the point of attachment to —CH₂—R²,
** represents the point of attachment to the pyrimidine ring, in which
A¹ represents N or C—H,
R^{1a} represents hydrogen or methyl if A¹ represents nitrogen,
or
R^{1a} represents hydrogen, fluorine or chlorine if A¹ represents C—H,
R^{1b} represents hydrogen or fluorine,
R^{1c} represents hydrogen or methyl,
R^{1d} represents hydrogen, methyl or fluorine,
R^{1e} represents hydrogen or chlorine.

A particular embodiment of the present invention encompasses compounds of the formula (I) in which the ring Q represents a group of the formula

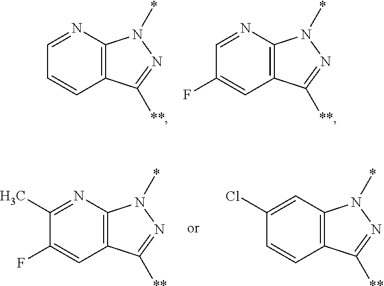

where
* represents the point of attachment to —CH₂—R²,
** represents the point of attachment to the pyrimidine ring.

A particular embodiment of the present invention encompasses compounds of the formula (I) in which the ring Q represents a group of the formula

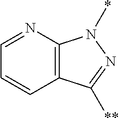

where
* represents the point of attachment to —CH₂—R²,
** represents the point of attachment to the pyrimidine ring.

A particular embodiment of the present invention encompasses compounds of the formula (I) in which the ring Q represents a group of the formula

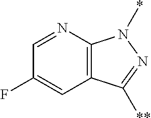

where
* represents the point of attachment to —CH₂—R²,
** represents the point of attachment to the pyrimidine ring.

A particular embodiment of the present invention encompasses compounds of the formula (I) in which the ring Q represents a group of the formula

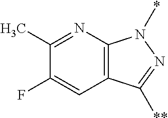

where
* represents the point of attachment to —CH₂—R²,
** represents the point of attachment to the pyrimidine ring.

A particular embodiment of the present invention encompasses compounds of the formula (I) in which the ring Q represents a group of the formula

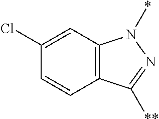

where
* represents the point of attachment to —CH₂—R²,
** represents the point of attachment to the pyrimidine ring.

The individual radical definitions specified in the respective combinations or preferred combinations of radicals are, independently of the respective combinations of the radicals specified, also replaced as desired by radical definitions of other combinations.

Very particular preference is given to combinations of two or more of the abovementioned preferred ranges.

The radical definitions specified as preferred, particularly preferred and very particularly preferred and also the particular embodiments apply both to the compounds of the formula (I) and correspondingly to all starting materials and intermediates.

The invention furthermore provides a process for preparing compounds of the formula (I) according to the invention, characterized in that a compound of the formula (II)

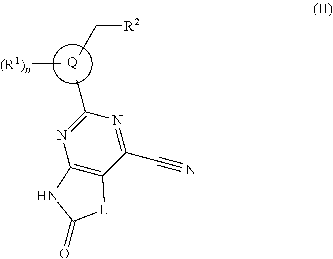

in which n, L, Q, R¹ and R² each have the meanings given above,
is reacted in a first step in the presence of a suitable aqueous base or acid to give the carboxamide of the formula (I-A) according to the invention

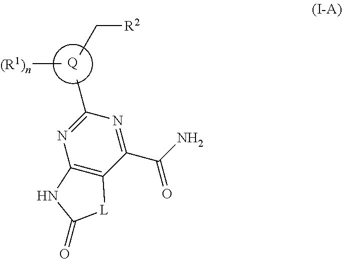

in which n, L, Q, R¹ and R² each have the meanings given above,
and the carboxamide (I-A) is optionally converted in a second step in an inert solvent in the presence of a suitable aqueous acid or base into a carboxylic acid of the formula (III)

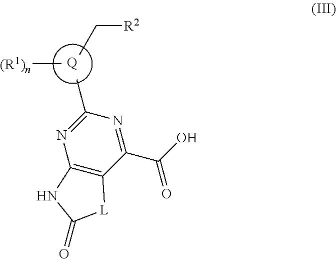

in which n, L, Q, R¹ and R² each have the meanings given above,
and these are subsequently in a third step reacted, with activation of the carboxylic acid function, with an amine compound of the formula (IV)

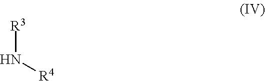

in which R³ and R⁴ each have the meanings given above, to give the carboxamide of the formula (I-B) according to the invention

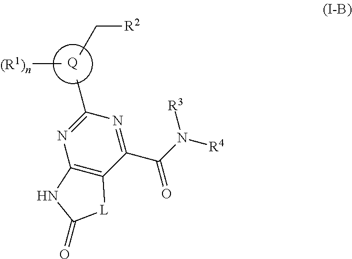

in which n, L, Q, R¹, R², R³ and R⁴ each have the meanings given above,
then any protective groups present are detached, and the resulting compounds of the formulae (I-A) and (I-B) are optionally converted, optionally with the appropriate (i) solvents and/or (ii) acids or bases, to the solvates, salts and/or solvates of the salts thereof.

Together, the compounds of the formulae (I-A) and (I-B) form the group of the compounds of the formula (I) according to the invention.

The hydrolysis of the nitrile group of the compounds (II) to give compounds of the formula (I-A) in the first step is preferably carried out in the presence of an aqueous base. Suitable bases for the hydrolysis of the nitrile group are, in general, alkali metal or alkaline earth metal hydroxides such as, for example, sodium hydroxide, lithium hydroxide, potassium hydroxide or barium hydroxide or alkali metal or alkaline earth metal carbonates such as sodium carbonate, potassium carbonate or calcium carbonate. Preference is given to using sodium hydroxide (aqueous sodium hydroxide solution).

The reaction (II)→(I-A) is generally carried out in inert solvents in a temperature range of from +20° C. to +100° C., preferably from +75° C. to +100° C. The reaction can take place at atmospheric, elevated or reduced pressure (e.g. from 0.5 to 5 bar). In general, the reaction is carried out at atmospheric pressure.

Suitable inert solvents for the reaction (II)→(I-A) are water, tetrahydrofuran, 1,4-dioxane or glycol dimethyl ether, or other solvents such as dimethylformamide or dimethyl sulfoxide. It is also possible to use mixtures of the solvents mentioned. Preference is given to using dioxane or dimethyl sulfoxide.

The hydrolysis of the amide group of the compounds (I-A) to give compounds of the formula (III) in the second step is preferably carried out in the presence of an aqueous acid.

Suitable acids for the reaction (I-A)→(III) are, in general, sulfuric acid, hydrogen chloride/hydrochloric acid, hydrogen bromide/hydrobromic acid or acetic acid or mixtures thereof, optionally with addition of water. Preference is given to using hydrochloric acid or a mixture of hydrochloric acid and acetic acid.

The reaction (I-A)→(III) can be carried out in an inert solvent such as, for example, water, THF, 1,4-dioxane, DMF or DMSO, or in the absence of a solvent. It is also possible to use mixtures of the solvents mentioned. The reaction can generally be carried out in a temperature range of from +20° C. to +100° C. The conversion can be carried out under atmospheric, elevated or reduced pressure (for example from 0.5 to 5 bar). Preferably, the reaction is carried out in the absence of a solvent, preferably in a temperature range of from 75-100° C. at atmospheric pressure.

The coupling reaction (III)+(IV)→(I-B) [amide formation] can be effected either by a direct route with the aid of a condensing or activating agent or via the intermediate stage of a carbonyl chloride or carbonyl imidazolide obtainable from (III).

Suitable condensing or activating agents of this kind are, for example, carbodiimides such as N,N′-diethyl-, N,N′-dipropyl-, N,N′-diisopropyl-, N,N′-dicyclohexylcarbodiimide (DCC) or N-(3-dimethylaminopropyl)-N′-ethylcarbodiimide hydrochloride (EDC), phosgene derivatives such as N,N′-carbonyldiimidazole (CDI) or isobutyl chloroformate, 1,2-oxazolium compounds such as 2-ethyl-5-phenyl-1,2-oxazolium 3-sulfate or 2-tert-butyl-5-methylisoxazolium perchlorate, acylamino compounds such as 2-ethoxy-1-ethoxycarbonyl-1,2-dihydroquinoline, α-chlorenamines such as 1-chloro-N,N,2-trimethylprop-1-en-1-amine, 1,3,5-triazine derivatives such as 4-(4,6-dimethoxy-1,3,5-triazin-2-yl)-4-methylmorpholinium chloride, phosphorus compounds such as n-propanephosphonic anhydride (PPA, T3P), diethyl cyanophosphonate, diphenylphosphoryl azide (DPPA), bis(2-oxo-3-oxazolidinyl)phosphoryl chloride, benzotriazol-1-yloxytris(dimethylamino)phosphonium hexafluorophosphate or benzotriazol-1-yloxytris(pyrrolidino)phosphonium hexafluorophosphate (PyBOP), or uronium compounds such as O-(benzotriazol-1-yl)-N,N,N′,N′-tetramethyluronium tetrafluoroborate (TBTU), O-(benzotriazol-1-yl)-N,N,N′,N′-tetramethyluronium hexafluorophosphate (HBTU), O-(1H-6-chlorobenzotriazol-1-yl)-1,1,3,3-tetramethyluronium tetrafluoroborate (TCTU), O-(7-azabenzotriazol-1-yl)-N,N,N′,N′-tetramethyluronium hexafluorophosphate (HATU) or 2-(2-oxo-1-(2H)-pyridyl)-1,1,3,3-tetramethyluronium tetrafluoroborate (TPTU), optionally in combination with further auxiliaries such as 1-hydroxybenzotriazole (HOBt) or N-hydroxysuccinimide (HOSu), and, as bases, alkali metal carbonates, e.g. sodium or potassium carbonate, or tertiary amine bases such as triethylamine, N-methylmorpholine (NMM), N-methylpiperidine (NMP), N,N-diisopropylethylamine, pyridine or 4-N,N-dimethylaminopyridine (DMAP). The condensing or activating agent preferably employed is n-propanephosphonic anhydride in combination with N,N-diisopropylethylamine or triethylamine as base.

In the case of a two-stage reaction regime via the carbonyl chlorides or carbonyl imidazolines obtainable from (III), the coupling with the amine component (IV) is conducted in the presence of a customary base, for example sodium carbonate or potassium carbonate, triethylamine, N,N-diisopropylethylamine, N-methylmorpholine (NMM), N-methylpiperidine (NMP), pyridine, 2,6-dimethylpyridine, 4-N,N-dimethylaminopyridine (DMAP), 1,8-diazabicyclo[5.4.0]undec-7-ene (DBU), 1,5-diazabicyclo[4.3.0]non-5-ene (DBN), sodium methoxide or potassium methoxide, sodium ethoxide or potassium ethoxide, sodium tert-butoxide or potassium tert-butoxide, or sodium hydride or potassium hydride. In the case of the carbonyl chlorides, the base used is preferably N,N-diisopropylethylamine.

Inert solvents for the coupling reactions mentioned are—according to the method used—for example ethers such as diethyl ether, diisopropyl ether, methyl tert-butyl ether, tetrahydrofuran, 1,4-dioxane, 1,2-dimethoxyethane or bis(2-methoxyethyl) ether, hydrocarbons such as benzene, toluene, xylene, pentane, hexane or cyclohexane, halohydrocarbons such as dichloromethane, trichloromethane, carbon tetrachloride, 1,2-dichloroethane, trichloroethylene or chlorobenzene, or polar aprotic solvents such as acetone, methyl ethyl ketone, ethyl acetate, acetonitrile, butyronitrile, pyridine, dimethyl sulfoxide (DMSO), N,N-dimethylformamide (DMF), N,N′-dimethylpropyleneurea (DMPU) or N-methylpyrrolidinone (NMP). It is also possible to use mixtures of such solvents. Preference is given to using 1,2-dichloroethane, tetrahydrofuran and N,N-dimethylformamide or mixtures of these solvents. The couplings are generally conducted within a temperature range from −20° C. to +60° C., preferably at 0° C. to +60° C.

The carbonyl chlorides are prepared in a customary manner by treating (III) with thionyl chloride or oxalyl chloride, optionally in an inert solvent such as dichloromethane, trichloromethane or 1,2-dichloroethane, optionally with use of a small amount of N,N-dimethylformamide as catalyst. The reaction is generally conducted at a temperature of 0° C. to +30° C.

The preferred coupling method is the reaction of a carbonyl chloride derived from (III) with the amine compound (IV).

The preparation process described can be illustrated by way of example by the following synthesis schemes (Schemes 1 and 2):

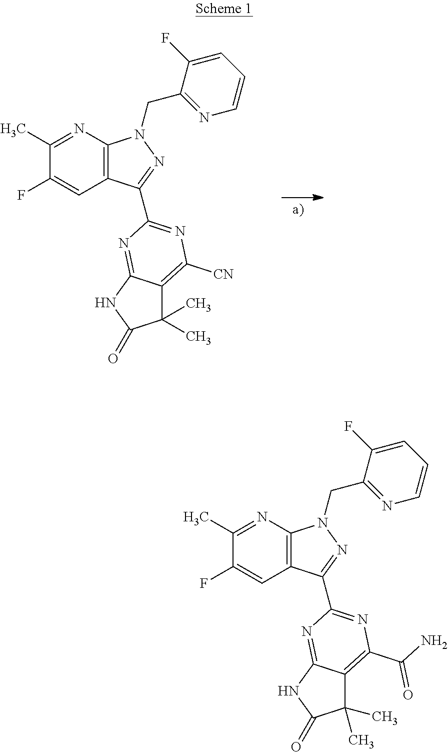 [SMILES: Cc1nc2c(cc1F)c(-c1nc(C#N)c3c(n1)NC(=O)C3(C)C)nn2Cc1ncccc1F ; Cc1nc2c(cc1F)c(-c1nc3c(c(C(N)=O)n1)C(C)(C)C(=O)N3)nn2Cc1ncccc1F]

[a): aqueous sodium hydroxide solution, dioxane, 80-90° C.].

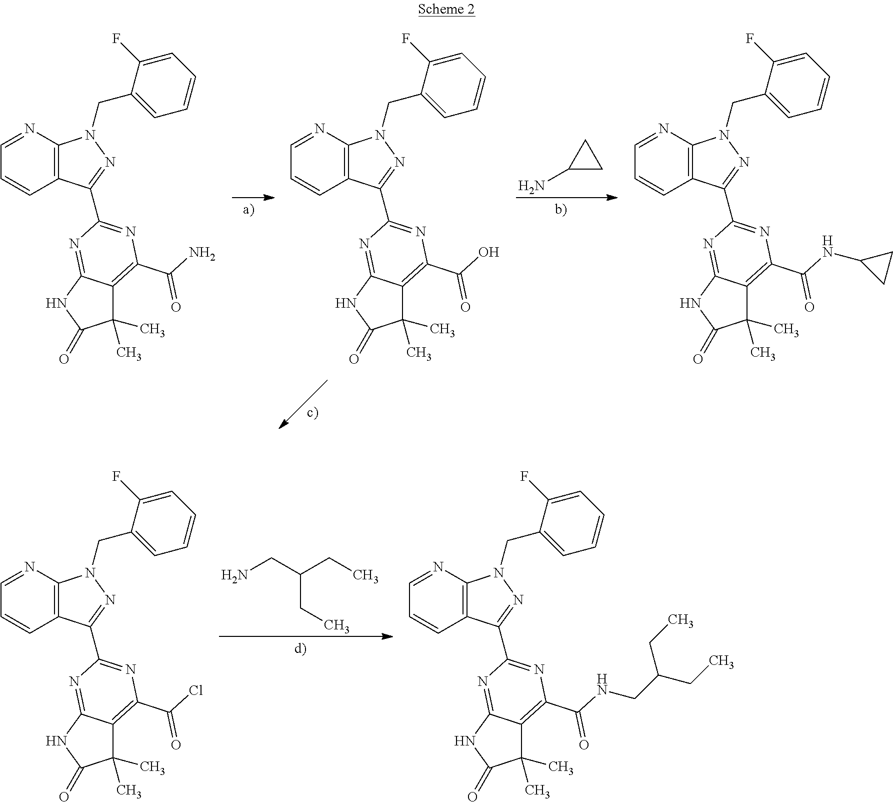

[a): conc. hydrochloric acid, 80-95° C.; b): propanephosphonic anhydride (T3P), N,N-diisopropylethylamine, DMF, RT−50° C.; c): SOCl₂, 0° C.->RT; d): N,N-diisopropylethylamine, dichloroethane, RT]

The compounds of the formula (II) are known from the literature (see, for example, WO 2013/104703) or can be prepared in analogy to processes known from the literature.

The compounds of the formula (II) can be prepared by converting a compound of the formula (V)

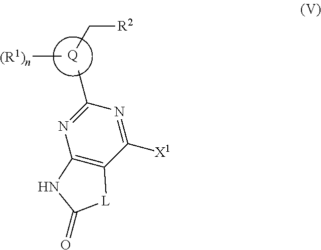

in which n, L, Q, R¹ and R² are each as defined above and
X¹ represents chlorine, bromine or iodine,
by reaction with copper(I) cyanide in an inert solvent, optionally in the presence of a suitable base, into a compound of the formula (II)

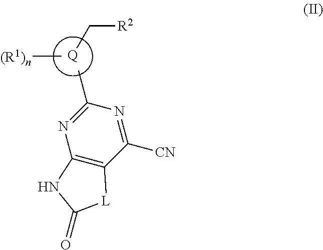

in which n, L, Q, R¹ and R² each have the meanings given above.

Process step (V)+copper cyanide→(II) is carried out in a solvent which is inert under the reaction conditions. Suitable solvents are, for example, ethers such as diethyl ether, dioxane, dimethoxyethane, tetrahydrofuran, glycol dimethyl ether or diethylene glycol dimethyl ether, hydrocarbons such as benzene, xylene, toluene, hexane, cyclohexane or mineral oil fractions, or other solvents such as dimethylformamide (DMF), dimethyl sulfoxide (DMSO), N,N′-dimethylpropyleneurea (DMPU), N-methylpyrrolidone (NMP), pyridine, acetonitrile or sulfolane. It is also possible to use mixtures of the solvents mentioned. Preference is given to DMSO.

The reaction (V)→(II) is generally conducted within a temperature range of 0° C. to +200° C., preferably at +120° C. to +180° C., optionally in a microwave. The conversion can be carried out under atmospheric, elevated or reduced pressure (for example from 0.5 to 5 bar). In general, the reaction is carried out at atmospheric pressure.

The compounds of the formula (V) are known from the literature (see, for example WO 2013/104703, WO 2013/030288) or can be prepared analogously to processes known from the literature.

The compounds of the formula (V) can be prepared by reacting, in a first step, a compound of the formula (VI)

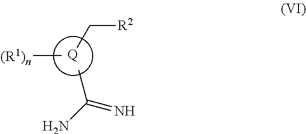

in which n, Q, R¹ and R² each have the meanings given above,
in an inert solvent in the presence of a suitable base with a compound of the formula (VII)

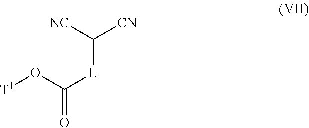

in which L has the meaning given above and
T¹ represents (C₁-C₄)-alkyl
to give a compound of the formula (VIII)

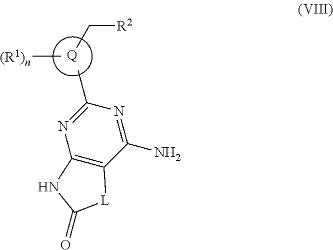

in which n, L, Q, R¹ and R² each have the meanings given above,
then converting this, in a second step, using isopentyl nitrite and a halogen equivalent into a compound of the formula (V)

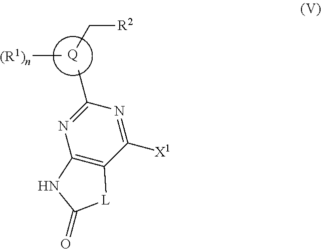

in which n, L, Q, R¹ and R² each have the meanings given above
and
X¹ represents chlorine, bromine or iodine.

Preferably, X¹ in (V) represents iodine.

Inert solvents for the process step (VI)+(VII)→(VIII) are, for example, alcohols such as methanol, ethanol, n-propanol, isopropanol, n-butanol or tert-butanol, ethers such as diethyl ether, dioxane, dimethoxyethane, tetrahydrofuran, glycol dimethyl ether or diethylene glycol dimethyl ether, hydrocarbons such as benzene, xylene, toluene, hexane, cyclohexane or mineral oil fractions, or other solvents such as dimethylformamide (DMF), dimethyl sulfoxide (DMSO), N,N′-dimethylpropyleneurea (DMPU), N-methylpyrrolidone (NMP), pyridine, acetonitrile, sulfolane or else water. It is also possible to use mixtures of the solvents mentioned. Preference is given to tert-butanol or methanol.

Suitable bases for the process step (VI)+(VII)→(VIII) are alkali metal hydroxides such as, for example, lithium hydroxide, sodium hydroxide or potassium hydroxide, alkali metal carbonates such as lithium carbonate, sodium carbonate, potassium carbonate or cesium carbonate, alkali metal bicarbonates such as sodium bicarbonate or potassium bicarbonate, alkali metal alkoxides such as sodium methoxide or potassium methoxide, sodium ethoxide or potassium ethoxide or potassium tert-butoxide, or organic amines such as triethylamine, diisopropylethylamine, pyridine, 1,8-diazabicyclo[5.4.0]undec-7-ene (DBU) or 1,5-diazabicyclo[4.3.0]non-5-ene (DBN). Preference is given to potassium tert-butoxide or sodium methoxide.

The reaction (VI)+(VII)→(VIII) is generally carried out within a temperature range of +20° C. to +150° C., preferably at +75° C. to +100° C., optionally in a microwave. The conversion can be carried out under atmospheric, elevated or reduced pressure (for example from 0.5 to 5 bar). In general, the reaction is carried out at atmospheric pressure.

Process step (VIII)→(V) is carried out with or without solvent. Suitable solvents are all organic solvents which are inert under the reaction conditions. The preferred solvent is dimethoxyethane.

The reaction (VIII)→(V) is generally carried out within a temperature range from +20° C. to +100° C., preferably within the range from +50° C. to +100° C., optionally in a microwave. The conversion can be carried out at atmospheric, elevated or reduced pressure (for example in the range from 0.5 to 5 bar). In general, the reaction is carried out at atmospheric pressure.

Suitable halogen sources in the conversion (VIII)→(V) are, for example, diiodomethane, a mixture of cesium iodide, iodine and copper(I) iodide or copper(II) bromide.

Process step (IV)→(V), in the case of diiodomethane as the halogen source, is carried out with a molar ratio of 10 to 30 mol of isopentyl nitrite and 10 to 30 mol of the iodine equivalent based on 1 mol of the compound of the formula (IV).

The preparation process described above can be illustrated in an exemplary manner by the following synthesis schemes (Scheme 3 and Scheme 4):

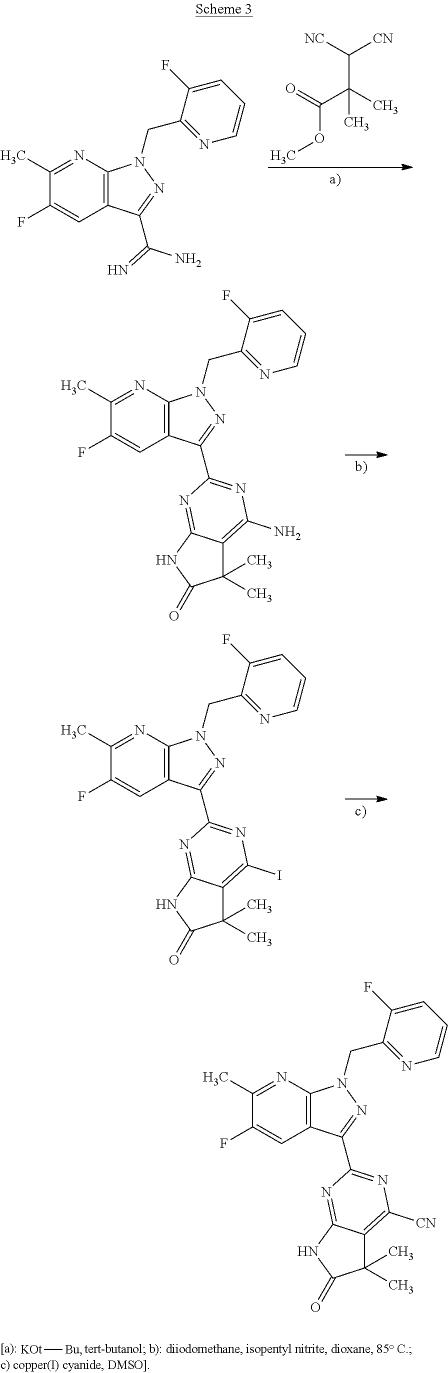

The compounds of the formula (VI) are known from the literature (see, for example, WO 03/095451, Example 6A; WO2013/104703, Example 52A; WO2013/104598, Example 54A) or can be prepared as in the synthesis scheme below (Scheme 4).

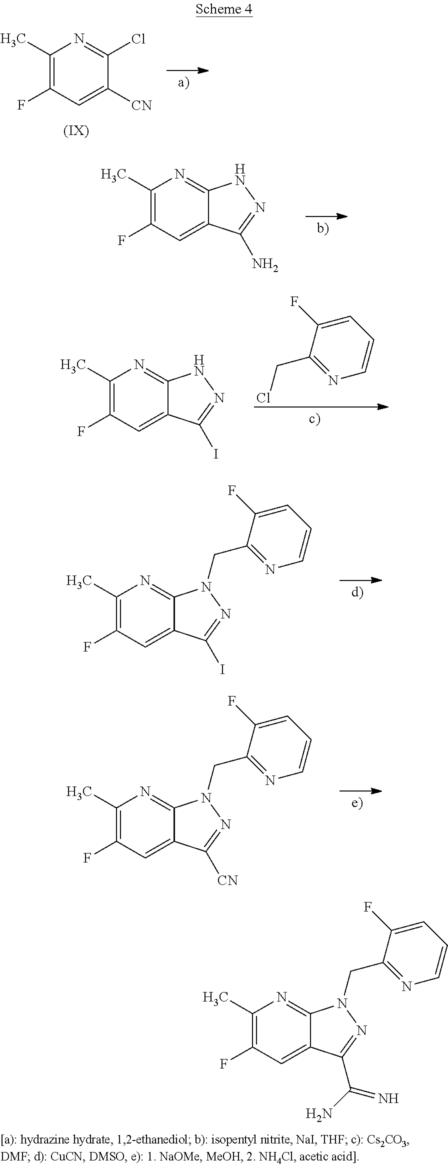

The compound of the formula (IX) is known from the literature [WO 2007/041052] or can be prepared analogously to processes known from the literature [WO2013/004785 and WO 2011/149921].

The compounds of the formula (VII) are commercially available, known from the literature or can be prepared in analogy to literature processes.

Detailed procedures and further literature references can also be found in the experimental section, in the section on the preparation of the starting compounds and intermediates.

The compounds of the invention have valuable pharmacological properties and can be used for treatment and/or prophylaxis of disorders in humans and animals.

The compounds of the invention act as potent stimulators of soluble guanylate cyclase and inhibitors of phosphodiesterase-5, have useful pharmacological properties and have an improved therapeutic profile, for example with respect to the in vivo properties thereof and/or the pharmacokinetic characteristics and/or metabolic profile thereof. They are therefore suitable for the treatment and/or prophylaxis of diseases in humans and animals.

The compounds of the invention bring about vasorelaxation and inhibition of platelet aggregation, and lead to a decrease in blood pressure and to a rise in coronary blood flow. These effects are mediated by a direct stimulation of soluble guanylate cyclase and an intracellular rise in cGMP. In addition, the compounds of the invention enhance the action of substances which increase the cGMP level, for example EDRF (endothelium-derived relaxing factor), NO donors, protoporphyrin IX, arachidonic acid or phenylhydrazine derivatives.

The compounds of the invention are suitable for the treatment and/or prophylaxis of cardiovascular, pulmonary, thromboembolic and fibrotic disorders.

Accordingly, the compounds of the invention can be used in medicaments for the treatment and/or prophylaxis of cardiovascular disorders such as, for example, high blood pressure (hypertension), resistant hypertension, acute and chronic heart failure, coronary heart disease, stable and unstable angina pectoris, peripheral and cardiac vascular disorders, arrhythmias, atrial and ventricular arrhythmias and impaired conduction such as, for example, atrioventricular blocks degrees I-III (AB block I-III), supraventricular tachyarrhythmia, atrial fibrillation, atrial flutter, ventricular fibrillation, ventricular flutter, ventricular tachyarrhythmia, Torsade de pointes tachycardia, atrial and ventricular extrasystoles, AV-junctional extrasystoles, sick sinus syndrome, syncopes, AV-nodal re-entry tachycardia, Wolff-Parkinson-White syndrome, of acute coronary syndrome (ACS), autoimmune cardiac disorders (pericarditis, endocarditis, valvolitis, aortitis, cardiomyopathies), shock such as cardiogenic shock, septic shock and anaphylactic shock, aneurysms, boxer cardiomyopathy (premature ventricular contraction (PVC)), for the treatment and/or prophylaxis of thromboembolic disorders and ischemias such as myocardial ischemia, myocardial infarction, stroke, cardiac hypertrophy, transient and ischemic attacks, preeclampsia, inflammatory cardiovascular disorders, spasms of the coronary arteries and peripheral arteries, edema formation such as, for example, pulmonary edema, cerebral edema, renal edema or edema caused by heart failure, peripheral circulatory disturbances, reperfusion damage, arterial and venous thromboses, microalbuminuria, myocardial insufficiency, endothelial dysfunction, to prevent restenoses, for example after thrombolysis therapies, percutaneous transluminal angioplasties (PTA), transluminal coronary angioplasties (PTCA), heart transplants and bypass operations, and also micro- and macrovascular damage (vasculitis), increased levels of fibrinogen and of low-density lipoprotein (LDL) and increased concentrations of plasminogen activator inhibitor 1 (PAI-1), and also for the treatment and/or prophylaxis of erectile dysfunction and female sexual dysfunction.

In the context of the present invention, the term “heart failure” encompasses both acute and chronic forms of heart failure, and also more specific or related types of disease, such as acute decompensated heart failure, right heart failure, left heart failure, global failure, ischemic cardiomyopathy, dilated cardiomyopathy, hypertrophic cardiomyopathy, idiopathic cardiomyopathy, congenital heart defects, heart failure associated with heart valve defects, mitral valve stenosis, mitral valve insufficiency, aortic valve stenosis, aortic valve insufficiency, tricuspid valve stenosis, tricuspid valve insufficiency, pulmonary valve stenosis, pulmonary valve insufficiency, combined heart valve defects, myocardial inflammation (myocarditis), chronic myocarditis, acute myocarditis, viral myocarditis, diabetic heart failure, alcoholic cardiomyopathy, cardiac storage disorders, diastolic heart failure and systolic heart failure, and acute phases of worsening of existing chronic heart failure (worsening heart failure).

In addition, the compounds of the invention can also be used for the treatment and/or prophylaxis of arteriosclerosis, impaired lipid metabolism, hypolipoproteinemias, dyslipidemias, hypertriglyceridemias, hyperlipidemias, hypercholesterolemias, abetelipoproteinemia, sitosterolemia, xanthomatosis, Tangier disease, adiposity, obesity and of combined hyperlipidemias and metabolic syndrome.

The compounds of the invention can additionally be used for the treatment and/or prophylaxis of primary and secondary Raynaud's phenomenon, of microcirculation impairments, claudication, peripheral and autonomic neuropathies, diabetic microangiopathies, diabetic retinopathy, diabetic ulcers on the extremities, gangrene, CREST syndrome, erythematosis, onychomycosis, rheumatic disorders and for promoting wound healing. The compounds of the invention are also suitable for the treatment of muscular dystrophy, such as Becker-Kiener muscular dystrophy (BMD) and Duchenne muscular dystrophy (DMD).

The compounds of the invention are furthermore suitable for treating urological disorders, for example benign prostate syndrome (BPS), benign prostate hyperplasia (BPH), benign prostate enlargement (BPE), bladder outlet obstruction (BOO), lower urinary tract syndromes (LUTS, including Feline Urological Syndrome (FUS)), disorders of the urogenital system including neurogenic over-active bladder (OAB) and (IC), incontinence (UI), for example mixed urinary incontinence, urge urinary incontinence, stress urinary incontinence or overflow urinary incontinence (MUI, UUI, SUI, OUI), pelvic pain, benign and malignant disorders of the organs of the male and female urogenital system.

The compounds of the invention are also suitable for the treatment and/or prophylaxis of kidney disorders, in particular of acute and chronic renal insufficiency and acute and chronic renal failure. In the context of the present invention, the term “renal insufficiency” encompasses both acute and chronic manifestations of renal insufficiency, and also underlying or related renal disorders such as renal hypoperfusion, intradialytic hypotension, obstructive uropathy, glomerulopathies, glomerulonephritis, acute glomerulonephritis, glomerulosclerosis, tubulointerstitial diseases, nephropathic disorders such as primary and congenital kidney disease, nephritis, immunological kidney disorders such as kidney transplant rejection and immunocomplex-induced kidney disorders, nephropathy induced by toxic substances, nephropathy induced by contrast agents, diabetic and non-diabetic nephropathy, pyelonephritis, renal cysts, nephrosclerosis, hypertensive nephrosclerosis and nephrotic syndrome which can be characterized diagnostically, for example by abnormally reduced creatinine and/or water excretion, abnormally elevated blood concentrations of urea, nitrogen, potassium and/or creatinine, altered activity of renal enzymes, for example glutamyl synthetase, altered urine osmolarity or urine volume, elevated microalbuminuria, macroalbuminuria, lesions on glomerulae and arterioles, tubular dilatation, hyperphosphatemia and/or need for dialysis. The present invention also encompasses the use of the compounds of the invention for the treatment and/or prophylaxis of sequelae of renal insufficiency, for example pulmonary edema, heart failure, uremia, anemia, electrolyte disorders (for example hyperkalemia, hyponatremia) and disorders in bone and carbohydrate metabolism.

In addition, the compounds of the invention are also suitable for the treatment and/or prophylaxis of asthmatic disorders, pulmonary arterial hypertension (PAH) and other forms of pulmonary hypertension (PH) including left-heart disease-, HIV-, sickle cell anemia-, thromboembolism (CTEPH), sarcoidosis-, COPD- or pulmonary fibrosis-associated pulmonary hypertension, chronic-obstructive pulmonary disease (COPD), acute respiratory distress syndrome (ARDS), acute lung injury (ALI), alpha-1-antitrypsin deficiency (AATD), pulmonary fibrosis, pulmonary emphysema (for example pulmonary emphysema induced by cigarette smoke) and cystic fibrosis (CF). In addition, the compounds mentioned can be used as bronchodilators.

The compounds described in the present invention are also active compounds for control of central nervous system disorders characterized by disturbances of the NO/cGMP system. They are suitable in particular for improving perception, concentration, learning or memory after cognitive impairments like those occurring in particular in association with situations/diseases/syndromes such as mild cognitive impairment, age-associated learning and memory impairments, age-associated memory losses, vascular dementia, craniocerebral trauma, stroke, dementia occurring after strokes (post-stroke dementia), post-traumatic craniocerebral trauma, general concentration impairments, concentration impairments in children with learning and memory problems, Alzheimer's disease, Lewy body dementia, dementia with degeneration of the frontal lobes including Pick's syndrome, Parkinson's disease, progressive nuclear palsy, dementia with corticobasal degeneration, amyolateral sclerosis (ALS), Huntington's disease, demyelinization, multiple sclerosis, thalamic degeneration, Creutzfeldt-Jakob dementia, HIV dementia, schizophrenia with dementia or Korsakoff's psychosis. They are also suitable for the treatment and/or prophylaxis of central nervous system disorders such as states of anxiety, tension and depression, CNS-related sexual dysfunctions and sleep disturbances, and for controlling pathological disturbances of the intake of food, stimulants and addictive substances.

In addition, the compounds of the invention are also suitable for controlling cerebral blood flow and are effective agents for controlling migraine. They are also suitable for the prophylaxis and control of sequelae of cerebral infarct (Apoplexia cerebri) such as stroke, cerebral ischemias and skull-brain trauma. The compounds of the invention can likewise be used for controlling states of pain and tinnitus.

In addition, the compounds of the invention have anti-inflammatory action and can therefore be used as anti-inflammatory agents for the treatment and/or prophylaxis of sepsis (SIRS), multiple organ failure (MODS, MOF), inflammatory disorders of the kidney, chronic intestinal inflammations (IBD, Crohn's disease, UC), pancreatitis, peritonitis, rheumatoid disorders, inflammatory skin disorders and inflammatory eye disorders.

Furthermore, the compounds of the invention can also be used for the treatment and/or prophylaxis of autoimmune diseases.

The compounds of the invention are also suitable for the treatment and/or prophylaxis of fibrotic disorders of the internal organs, for example the lung, the heart, the kidney, the bone marrow and in particular the liver, and also dermatological fibroses and fibrotic eye disorders. In the context of the present invention, the term fibrotic disorders includes in particular the following terms: hepatic fibrosis, cirrhosis of the liver, pulmonary fibrosis, endomyocardial fibrosis, nephropathy, glomerulonephritis, interstitial renal fibrosis, fibrotic damage resulting from diabetes, bone marrow fibrosis and similar fibrotic disorders, scleroderma, morphea, keloids, hypertrophic scarring (also following surgical procedures), naevi, diabetic retinopathy, proliferative vitroretinopathy and disorders of the connective tissue (for example sarcoidosis).

The compounds of the invention are also suitable for controlling postoperative scarring, for example as a result of glaucoma operations.

The compounds of the invention can also be used cosmetically for ageing and keratinizing skin.

Moreover, the compounds of the invention are suitable for the treatment and/or prophylaxis of hepatitis, neoplasms, osteoporosis, glaucoma and gastroparesis.

The present invention further provides for the use of the compounds of the invention for the treatment and/or prophylaxis of disorders, especially the disorders mentioned above.

The present invention further provides for the use of the compounds of the invention for the treatment and/or prophylaxis of heart failure, angina pectoris, hypertension, pulmonary hypertension, ischemias, vascular disorders, renal insufficiency, thromboembolic disorders, fibrotic disorders, arteriosclerosis, dementia disorders and erectile dysfunction.

The present invention further provides the compounds of the invention for use in a method for the treatment and/or prophylaxis of heart failure, angina pectoris, hypertension, pulmonary hypertension, ischemias, vascular disorders, renal insufficiency, thromboembolic disorders, fibrotic disorders, arteriosclerosis, dementia disorders and erectile dysfunction.

The present invention further provides for the use of the compounds of the invention for production of a medicament for the treatment and/or prophylaxis of disorders, especially the disorders mentioned above.

The present invention further provides for the use of the compounds of the invention for preparing a medicament for the treatment and/or prophylaxis of heart failure, angina pectoris, hypertension, pulmonary hypertension, ischemias, vascular disorders, renal insufficiency, thromboembolic disorders, fibrotic disorders, arteriosclerosis, dementia disorders and erectile dysfunction.

The present invention further provides a method for the treatment and/or prophylaxis of disorders, in particular the disorders mentioned above, using an effective amount of at least one of the compounds of the invention.

The present invention further provides a method for the treatment and/or prophylaxis of heart failure, angina pectoris, hypertension, pulmonary hypertension, ischemias, vascular disorders, renal insufficiency, thromboembolic disorders, fibrotic disorders, arteriosclerosis, dementia disorders and erectile dysfunction using an effective amount of at least one of the compounds of the invention.

The compounds of the invention can be used alone or, if required, in combination with other active compounds. The present invention further provides medicaments comprising at least one of the compounds of the invention and one or more further active compounds, especially for the treatment and/or prophylaxis of the aforementioned disorders. Preferred examples of active compounds suitable for combinations include:
organic nitrates and NO donors, for example sodium nitroprusside, nitroglycerin, isosorbide mononitrate, isosorbide dinitrate, molsidomine or SIN-1, and inhaled NO;
compounds which inhibit the breakdown of cyclic guanosine monophosphate (cGMP), for example inhibitors of phosphodiesterases (PDE) 1, 2 and/or 5, especially PDE 5 inhibitors such as sildenafil, vardenafil and tadalafil;
antithrombotic agents, by way of example and with preference from the group of the platelet aggregation inhibitors, the anticoagulants or the profibrinolytic substances;
hypotensive active compounds, by way of example and with preference from the group of the calcium antagonists, angiotensin AII antagonists, ACE inhibitors, endothelin antagonists, renin inhibitors, alpha-receptor blockers, beta-receptor blockers, mineralocorticoid receptor antagonists, and the diuretics; and/or
active compounds altering lipid metabolism, by way of example and with preference from the group of the thyroid receptor agonists, cholesterol synthesis inhibitors such as, by way of example and preferably, HMG-CoA reductase inhibitors or squalene synthesis inhibitors, the ACAT inhibitors, CETP inhibitors, MTP inhibitors, PPAR-alpha, PPAR-gamma and/or PPAR-delta agonists, cholesterol absorption inhibitors, lipase inhibitors, polymeric bile acid adsorbents, bile acid reabsorption inhibitors and lipoprotein(a) antagonists.

Antithrombotic agents are preferably understood to mean compounds from the group of the platelet aggregation inhibitors, the anticoagulants or the profibrinolytic substances.

In a preferred embodiment of the invention, the compounds of the invention are administered in combination with a platelet aggregation inhibitor, by way of example and with preference aspirin, clopidogrel, ticlopidine or dipyridamole.

In a preferred embodiment of the invention, the compounds of the invention are administered in combination with a thrombin inhibitor, by way of example and with preference ximelagatran, dabigatran, melagatran, bivalirudin or clexane.

In a preferred embodiment of the invention, the compounds of the invention are administered in combination with a GPIIb/IIIa antagonist, by way of example and with preference tirofiban or abciximab.

In a preferred embodiment of the invention, the compounds of the invention are administered in combination with a factor Xa inhibitor, by way of example and with preference rivaroxaban, DU-176b, apixaban, otamixaban, fidexaban, razaxaban, fondaparinux, idraparinux, PMD-3112, YM-150, KFA-1982, EMD-503982, MCM-17, MLN-1021, DX 9065a, DPC 906, JTV 803, SSR-126512 or SSR128428.

In a preferred embodiment of the invention, the compounds of the invention are administered in combination with heparin or with a low molecular weight (LMW) heparin derivative.

In a preferred embodiment of the invention, the compounds of the invention are administered in combination with a vitamin K antagonist, by way of example and with preference coumarin.

Hypotensive agents are preferably understood to mean compounds from the group of the calcium antagonists, angiotensin AII antagonists, ACE inhibitors, endothelin antagonists, renin inhibitors, alpha-receptor blockers, beta-receptor blockers, mineralocorticoid receptor antagonists, and the diuretics.

In a preferred embodiment of the invention, the compounds of the invention are administered in combination with a calcium antagonist, by way of example and with preference nifedipine, amlodipine, verapamil or diltiazem.

In a preferred embodiment of the invention, the compounds of the invention are administered in combination with an alpha-1-receptor blocker, by way of example and with preference prazosin.

In a preferred embodiment of the invention, the compounds of the invention are administered in combination with a beta-receptor blocker, by way of example and with preference propranolol, atenolol, timolol, pindolol, alprenolol, oxprenolol, penbutolol, bupranolol, metipranolol, nadolol, mepindolol, carazalol, sotalol, metoprolol, betaxolol, celiprolol, bisoprolol, carteolol, esmolol, labetalol, carvedilol, adaprolol, landiolol, nebivolol, epanolol or bucindolol.

In a preferred embodiment of the invention, the inventive compounds are administered in combination with an angiotensin AII antagonist, preferred examples being losartan, candesartan, valsartan, telmisartan or embusartan.

In a preferred embodiment of the invention, the compounds of the invention are administered in combination with an ACE inhibitor, by way of example and with preference enalapril, captopril, lisinopril, ramipril, delapril, fosinopril, quinopril, perindopril or trandopril.

In a preferred embodiment of the invention, the compounds of the invention are administered in combination with an endothelin antagonist, by way of example and with preference bosentan, darusentan, ambrisentan or sitaxsentan.

In a preferred embodiment of the invention, the compounds of the invention are administered in combination with a renin inhibitor, by way of example and with preference aliskiren, SPP-600 or SPP-800.

In a preferred embodiment of the invention, the compounds of the invention are administered in combination with a mineralocorticoid receptor antagonist, by way of example and with preference spironolactone or eplerenone.

In a preferred embodiment of the invention, the compounds of the invention are administered in combination with a loop diuretic, for example furosemide, torasemide, bumetanide and piretanide, with potassium-sparing diuretics, for example amiloride and triamterene, with aldosterone antagonists, for example spironolactone, potassium canrenoate and eplerenone, and also thiazide diuretics, for example hydrochlorothiazide, chlorthalidone, xipamide and indapamide.

Lipid metabolism modifiers are preferably understood to mean compounds from the group of the CETP inhibitors, thyroid receptor agonists, cholesterol synthesis inhibitors such as HMG-CoA reductase inhibitors or squalene synthesis inhibitors, the ACAT inhibitors, MTP inhibitors, PPAR-alpha, PPAR-gamma and/or PPAR-delta agonists, cholesterol absorption inhibitors, polymeric bile acid adsorbers, bile acid reabsorption inhibitors, lipase inhibitors and the lipoprotein(a) antagonists. In a preferred embodiment of the invention, the compounds of the invention are administered in combination with a CETP inhibitor, by way of example and with preference dalcetrapib, BAY 60-5521, anacetrapib or CETP vaccine (CETi-1).

In a preferred embodiment of the invention, the compounds of the invention are administered in combination with a thyroid receptor agonist, by way of example and with preference D-thyroxine, 3,5,3′-triiodothyronine (T3), CGS 23425 or axitirome (CGS 26214).

In a preferred embodiment of the invention, the compounds of the invention are administered in combination with an HMG-CoA reductase inhibitor from the class of statins, by way of example and with preference lovastatin, simvastatin, pravastatin, fluvastatin, atorvastatin, rosuvastatin or pitavastatin.

In a preferred embodiment of the invention, the compounds of the invention are administered in combination with a squalene synthesis inhibitor, by way of example and with preference BMS-188494 or TAK-475.

In a preferred embodiment of the invention, the compounds of the invention are administered in combination with an ACAT inhibitor, by way of example and with preference avasimibe, melinamide, pactimibe, eflucimibe or SMP-797.

In a preferred embodiment of the invention, the compounds of the invention are administered in combination with an MTP inhibitor, by way of example and with preference implitapide, BMS-201038, R-103757 or JTT-130.

In a preferred embodiment of the invention, the compounds of the invention are administered in combination with a PPAR-gamma agonist, by way of example and with preference pioglitazone or rosiglitazone.

In a preferred embodiment of the invention, the compounds of the invention are administered in combination with a PPAR-delta agonist, by way of example and with preference GW 501516 or BAY 68-5042.

In a preferred embodiment of the invention, the compounds of the invention are administered in combination with a cholesterol absorption inhibitor, by way of example and with preference ezetimibe, tiqueside or pamaqueside.

In a preferred embodiment of the invention, the compounds of the invention are administered in combination with a lipase inhibitor, by way of example and with preference orlistat.

In a preferred embodiment of the invention, the compounds of the invention are administered in combination with a polymeric bile acid adsorber, by way of example and with preference cholestyramine, colestipol, colesolvam, CholestaGel or colestimide.

In a preferred embodiment of the invention, the compounds of the invention are administered in combination with a bile acid reabsorption inhibitor, by way of example and with preference ASBT (=IBAT) inhibitors, for example AZD-7806, S-8921, AK-105, BARI-1741, SC-435 or SC-635.

In a preferred embodiment of the invention, the compounds of the invention are administered in combination with a lipoprotein(a) antagonist, by way of example and with preference gemcabene calcium (CI-1027) or nicotinic acid.

The present invention further provides medicaments which comprise at least one compound of the invention, typically together with one or more inert, non- toxic, pharmaceutically suitable excipients, and for the use thereof for the aforementioned purposes.

The compounds of the invention can act systemically and/or locally. For this purpose, they can be administered in a suitable manner, for example by the oral, parenteral, pulmonal, nasal, sublingual, lingual, buccal, rectal, dermal, transdermal, conjunctival or otic route, or as an implant or stent.

The compounds of the invention can be administered in administration forms suitable for these administration routes.

Suitable administration forms for oral administration are those which work according to the prior art and release the compounds of the invention rapidly and/or in a modified manner and which contain the compounds of the invention in crystalline and/or amorphized and/or dissolved form, for example tablets (uncoated or coated tablets, for example with gastric juice-resistant or retarded-dissolution or insoluble coatings which control the release of the compound of the invention), tablets or films/oblates which disintegrate rapidly in the oral cavity, films/lyophilizates, capsules (for example hard or soft gelatin capsules), sugar-coated tablets, granules, pellets, powders, emulsions, suspensions, aerosols or solutions.

Parenteral administration can be accomplished with avoidance of a resorption step (for example by an intravenous, intraarterial, intracardiac, intraspinal or intralumbar route) or with inclusion of a resorption (for example by an intramuscular, subcutaneous, intracutaneous, percutaneous or intraperitoneal route). Administration forms suitable for parenteral administration include preparations for injection and infusion in the form of solutions, suspensions, emulsions, lyophilizates or sterile powders.

For the other administration routes, suitable examples are inhalable medicament forms (including powder inhalers, nebulizers), nasal drops, solutions or sprays, tablets, films/oblates or capsules for lingual, sublingual or buccal administration, suppositories, ear or eye preparations, vaginal capsules, aqueous suspensions (lotions, shaking mixtures), lipophilic suspensions, ointments, creams, transdermal therapeutic systems (e.g. patches), milk, pastes, foams, sprinkling powders, implants or stents.

Preference is given to oral or parenteral administration, especially oral administration.

The compounds of the invention can be converted to the administration forms mentioned. This can be accomplished in a manner known per se by mixing with inert, non- toxic, pharmaceutically suitable excipients. These excipients include carriers (for example microcrystalline cellulose, lactose, mannitol), solvents (e.g. liquid polyethylene glycols), emulsifiers and dispersing or wetting agents (for example sodium dodecylsulfate, polyoxysorbitan oleate), binders (for example polyvinylpyrrolidone), synthetic and natural polymers (for example albumin), stabilizers (e.g. antioxidants, for example ascorbic acid), colorants (e.g. inorganic pigments, for example iron oxides) and flavor and/or odor correctants.

In general, it has been found to be advantageous in the case of parenteral administration to administer amounts of about 0.001 to 1 mg/kg, preferably about 0.01 to 0.5 mg/kg, of body weight to achieve effective results. In the case of oral administration, the dose is about 0.001 to 2 mg/kg, preferably about 0.001 to 1 mg/kg, of body weight.

It may nevertheless be necessary in some cases to deviate from the stated amounts, specifically as a function of body weight, route of administration, individual response to the active ingredient, nature of the preparation and time or interval over which administration takes place. Thus in some cases it may be sufficient to manage with less than the abovementioned minimum amount, while in other cases the upper limit mentioned must be exceeded. In the case of administration of greater amounts, it may be advisable to divide them into several individual doses over the day.

The working examples which follow illustrate the invention. The invention is not restricted to the examples.

Unless stated otherwise, the percentages in the tests and examples which follow are percentages by weight; parts are parts by weight. Solvent ratios, dilution ratios and concentration data for liquid/liquid solutions, unless indicated otherwise, are based in each case on volume.

### A. Examples

### Abbreviations and Acronyms

| | |
|---|---|
| abs. | absolute |
| aq. | aqueous solution |
| calc. | calculated |
| Boc | tert-butyloxycarbonyl |
| br. s | broad singlet (in NMR) |
| Cbz | benzyloxycarbonyl |
| δ | shift in the NMR spectrum (stated in ppm) |
| d | doublet (NMR coupling pattern) |
| DAD | diode array detectors (for UV detection) |
| TLC | thin-layer chromatography |
| DCI | direct chemical ionization (in MS) |
| dd | doublet of doublet (NMR coupling pattern) |
| ddt | doublet of doublet of |
| | triplet (NMR coupling pattern) |
| DMF | N,N-dimethylformamide |
| DMSO | dimethyl sulfoxide |
| ent | enantiomerically pure; enantiomer |
| eq. | equivalent(s) |
| ESI | electrospray ionization (in MS) |
| Et | ethyl |
| h | hour(s) |
| HATU | (1-[bis(dimethylamino)methylene]-1H-1,2, |
| | 3-triazolo[4,5-b]pyridinium 3-oxide |
| | hexafluorophosphate) |
| HPLC | high-pressure, high-performance liquid |
| | chromatography |
| HRMS | high-resolution mass spectrometry |
| conc. | concentrated (in the case of a solution) |
| LC-MS | liquid chromatography-coupled mass spectrometry |
| m | multiplet |
| M | molar (in solution) |
| Me | methyl |
| min | minute(s) |
| MS | mass spectrometry |
| N | normal (in the case of a solution) |
| NMR | nuclear magnetic resonance spectrometry |
| PdCl₂(dppf)CH₂Cl₂ | 1,1′-bis(diphenylphosphino) |
| | ferrocenepalladium(II) dichloride/dichloromethane |
| | complex |
| Ph | phenyl |
| q | quartet (NMR coupling pattern) |
| quint. | quintet (NMR coupling pattern) |
| rac | racemic; racemate |
| rel | relative stereochemistry |
| RT | room temperature (about 20-25° C.) |
| Rₜ | retention time (in HPLC, LC/MS) |
| s | singlet (NMR coupling pattern) |
| SFC | supercritical fluid chromatography |
| t | triplet (NMR coupling pattern) |
| TBTU | (benzotriazol-1-yloxy)bisdimethylaminomethylium |
| | fluoroborate |
| TFA | trifluoroacetic acid |
| THF | tetrahydrofuran |
| UV | ultraviolet spectrometry |
| v/v | volume to volume ratio (of a solution) |

### LC/MS and MS Methods:

### Method 1 (LC-MS):

Instrument: Waters Acquity SQD UPLC System; column: Waters Acquity UPLC HSS T3 1.8μ, 50×1 mm; mobile phase A: 1 l of water+0.25 ml of 99% strength formic acid, mobile phase B: 1 l of acetonitrile+0.25 ml of 99% strength formic acid; gradient: 0.0 min 90% A→1.2 min 5% A→2.0 min 5% A; oven: 50° C.; flow rate: 0.40 ml/min; UV detection: 208-400 nm.

### Method 2 (LC-MS):

Instrument: Waters Acquity SQD UPLC System; column: Waters Acquity UPLC HSS T3 1.8μ, 50×1 mm; mobile phase A: 1 l of water+0.25 ml of 99% strength formic acid, mobile phase B: 1 l of acetonitrile+0.25 ml of 99% strength formic acid; gradient: 0.0 min 95% A→6.0 min 5% A→7.5 min 5% A; oven: 50° C.; flow rate: 0.35 ml/min; UV detection: 210-400 nm.

### Method 3 (LC-MS):

Instrument: Micromass Quattro Premier with Waters UPLC Acquity; column: Thermo Hypersil GOLD 1.9μ, 50×1 mm; mobile phase A: 1 l of water+0.5 ml of 50% strength formic acid, mobile phase B: 1 l of acetonitrile+0.5 ml of 50% strength formic acid; gradient: 0.0 min 97% A→0.5 min 97% A→3.2 min 5% A→4.0 min 5% A; oven: 50° C.; flow rate: 0.3 ml/min; UV detection: 210 nm.

### Method 4 (LC-MS):

MS instrument: Waters Micromass Quattro Micro; HPLC instrument: Agilent 1100 series; column: YMC-Triart C18 3μ 50×3 mm; mobile phase A: 1 l of water+0.01 mol of ammonium carbonate, mobile phase B: 1 l of acetonitrile; gradient: 0.0 min 100% A→2.75 min 5% A→4.5 min 5% A; oven: 40° C.; flow rate: 1.25 ml/min; UV detection: 210 nm.

### Method 5 (LC-MS):

MS instrument: Waters (Micromass) QM; HPLC instrument: Agilent 1100 series; column: Agilent Zorbax Extend-C18 3.5μ, 3.0×50 mm; mobile phase A: 1 l of water+0.01 mol of ammonium carbonate, mobile phase B: 1 l of acetonitrile; gradient: 0.0 min 98% A→0.2 min 98% A→3.0 min 5% A→4.5 min 5% A; oven: 40° C.; flow rate: 1.75 ml/min; UV detection: 210 nm.

### Method 6 (GC-MS):

Instrument: Micromass GCT, GC6890; column: Restek RTX-35, 15 m×200 μm×0.33 μm; constant helium flow rate: 0.88 ml/min; oven: 70° C.; inlet: 250° C.; gradient: 70° C., 30° C./min→310° C. (maintain for 3 min).

### Method 7 (LC-MS):

MS instrument: Agilent MS Quad 6150; HPLC instrument: Agilent 1290; column: Waters Acquity UPLC HSS T3 1.8μ, 50×2.1 mm; mobile phase A: 1 l of water+0.25 ml of 99% strength formic acid, mobile phase B: 1 l of acetonitrile+0.25 ml of 99% strength formic acid; gradient: 0.0 min 90% A→0.3 min 90% A→1.7 min 5% A→3.0 min 5% A; oven: 50° C.; flow rate: 1.20 ml/min; UV detection: 205-305 nm.

### Method 8 (GC-MS):

Instrument: Thermo Scientific DSQII, Thermo Scientific Trace GC Ultra; column: Restek RTX-35MS, 15 m×200 μm×0.33 μm; constant flow rate with helium: 1.20 ml/min; oven: 60° C.; inlet: 220° C.; gradient: 60° C., 30° C./min→300° C. (maintain for 3.33 min).

### Method 9 (LC-MS):

MS instrument: Waters SQD; HPLC instrument: Waters UPLC; column: Agilent Zorbax SB-Aq, 1.8 μm, 50×2.1 mm; mobile phase A: water+0.025% formic acid, mobile phase B: acetonitrile (ULC)+0.025% formic acid; gradient: 0.0 min 98% A-0.9 min 25% A-1.0 min 5% A-1.4 min 5% A-1.41 min 98% A-1.5 min 98% A; oven: 40° C.; flow rate: 0,600 ml/min; UV detection: DAD; 210 nm.

Method 10 (preparative HPLC):

Variant A): MS instrument: Waters, HPLC instrument: Waters; column: Waters X-Bridge C18 5 μm, 19×50 mm; mobile phase A: water+0.05% ammonia, mobile phase B: acetonitrile (ULC) with gradient; flow rate: 40 ml/min; UV detection: DAD; 210-400 nm).

Variant B): MS instrument: Waters, HPLC instrument: Waters (column Phenomenex Luna C18(2) 100 Å, AXIA Tech., 5 μm, 50 mm×21.2 mm; mobile phase A: water+0.05% formic acid, mobile phase B: acetonitrile (ULC) with gradient; flow rate: 40 ml/min; UV detection: DAD; 210-400 nm).

### Method 11 (LC-MS):

MS instrument: ThermoFisherScientific LTQ-Orbitrap-XL; HPLC instrument: Agilent 1200SL; column: Agilent, Poroshell 120 SB-C18 2.7 μm 3×150 mm; mobile phase A: 1 l of water+0.1% trifluoroacetic acid; mobile phase B: 1 l of acetonitrile+0.1% trifluoroacetic acid; gradient: 0.0 min 2% B→1.5 min 2% B→15.5 min 95% B→18.0 min 95% B; oven: 40° C.; flow rate: 0.75 ml/min; UV detection: 210 nm.

### Further Details:

In the case of purifications of compounds of the invention by preparative HPLC by the above-described methods in which the eluents contain additives, for example trifluoroacetic acid, formic acid or ammonia, the compounds of the invention can be obtained in salt form, for example as trifluoroacetate, formate or ammonium salt, if the compounds of the invention contain a sufficiently basic or acidic functionality. Such a salt can be converted to the corresponding free base or acid by various methods known to the person skilled in the art.

Furthermore, amidines can be present as free compounds or partially (depending on the preparation if acetic acid is involved) as acetate salts or acetate solvates.

In the case of the synthesis intermediates and working examples of the invention described hereinafter, any compound specified in the form of a salt of the corresponding base or acid is generally a salt of unknown exact stoichiometric composition, as obtained by the respective preparation and/or purification process. Unless specified in more detail, additions to names and structural formulae, such as “hydrochloride”, “trifluoroacetate”, “sodium salt” or “x HCl”, “x CF₃COOH”, “x Na⁺” should not therefore be understood in a stoichiometric sense in the case of such salts, but have merely descriptive character with regard to the salt-forming components present therein.

This applies correspondingly if synthesis intermediates or working examples or salts thereof were obtained in the form of solvates, for example hydrates, of unknown stoichiometric composition (if they are of a defined type) by the preparation and/or purification processes described.

Furthermore, the secondary amides according to the invention may be present as rotational isomers/isomer mixtures, in particular in NMR studies. Purity figures are generally based on corresponding peak integrations in the LC/MS chromatogram, but may additionally also have been determined with the aid of the ¹H NMR spectrum. If no purity is indicated, the purity is generally 100% according to automated peak integration in the LC/MS chromatogram, or the purity has not been determined explicitly.

Stated yields in % of theory are generally corrected for purity if a purity of <100% is indicated. In solvent-containing or contaminated batches, the formal yield may be “>100%”; in these cases the yield is not corrected for solvent or purity.

In all ¹H NMR spectra data, the chemical shifts δ are stated in ppm.

The multiplicities of proton signals in 1H NMR spectra reported in the paragraphs which follow represent the signal form observed in each case and do not take account of any higher-order signal phenomena. In general, the stated chemical shift refers to the center of the signal in question. In the case of broad multiplets, an interval is given. Signals obscured by solvent or water were either tentatively assigned or have not been listed. Significantly broadened signals—caused, for example, by rapid rotation of molecular moieties or because of exchanging protons—were likewise assigned tentatively (often referred to as a broad multiplet or broad singlet) or are not listed.

Melting points and melting-point ranges, if stated, are uncorrected.

All reactants or reagents whose preparation is not described explicitly hereinafter were purchased commercially from generally accessible sources. For all other reactants or reagents whose preparation likewise is not described hereinafter and which were not commercially obtainable or were obtained from sources which are not generally accessible, a reference is given to the published literature in which their preparation is described.

### STARTING COMPOUNDS AND INTERMEDIATES

### Example 1A

### 5-Fluoro-6-methyl-1H-pyrazolo[3,4-b]pyridine-3-amine

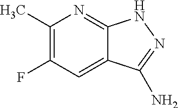

58 g (340.03 mmol) of 2-chloro-5-fluoro-6-methylnicotinonitrile (preparation described in WO2007/041052, Example U-2, page 80) were initially charged in 1,2-ethanediol (580 ml), and hydrazine hydrate (24.81 ml) and 56.09 ml (340.03 mmol) of N,N-diisopropylethylamine were then added. The mixture was stirred at 80° C. for 16 h and then at 120° C. for 6 h. After cooling to RT, water (2.5 l) and ethyl acetate (2.5 l) were added and the resulting solid was filtered off with suction. The solid obtained was dried under reduced pressure. This gave 28.4 g (47% of theory) of the target compound.

LC-MS (Method 4): Rₜ=1.77 min

MS (ESIpos): m/z=167 [M+H]⁺

### Example 2A

### 5-Fluoro-3-iodo-6-methyl-1H-pyrazolo[3,4-b]pyridine

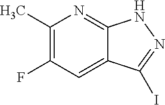

28 g (168.5 mmol) of 5-fluoro-6-methyl-1H-pyrazolo[3,4-b]pyridine-3-amine from Example 1A were initially charged in 1.32 l of THF, and the mixture was cooled to 0° C. 41.45 ml (337.03 mmol) of boron trifluoride diethyl ether complex were then added slowly. The reaction mixture was cooled to −10° C. A solution of 25.66 g (219.07 mmol) of isopentyl nitrite in 166 ml of THF was then added slowly, and the mixture was subsequently stirred for a further 30 min. The reaction solution was then concentrated to about a third of its volume. 988 ml of acetone were then added, and the solution was cooled to 0° C. A solution of 32.84 g (219.07 mmol) of sodium iodide in 412 ml of acetone was added dropwise to this solution, and the mixture was then stirred at RT for 2 h. The reaction mixture was poured into 5 l of ice-water and extracted three times with in each case 750 ml of ethyl acetate. The combined organic phases were washed with 750 ml of saturated aqueous sodium chloride solution, dried and then concentrated under reduced pressure. The crude product was purified using silica gel (silica gel, mobile phase: cyclohexane/ethyl acetate, gradient 9:1 to 1:1). This gave 14.90 g (32% of theory) of the title compound.

LC-MS (Method 1): Rₜ=0.84 min

MS (ESIpos): m/z=278 [M+H]⁺

### Example 3A

### 1-(2,3-Difluorobenzyl)-5-fluoro-3-iodo-6-methyl-1H-pyrazolo[3,4-b]pyridine

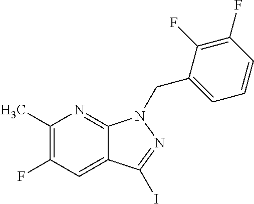 [SMILES: Cc1nc2c(cc1F)c(I)nn2Cc1cccc(F)c1F]

2.60 g (9.37 mmol) of 5-fluoro-3-iodo-6-methyl-1H-pyrazolo[3,4-b]pyridine from Example 2A were initially charged in 35 ml of DMF. A solution of 3.67 g (11.26 mmol) of cesium carbonate and 1.94 g (9.37 mmol) of 1-(bromomethyl)-2,3-difluorobenzene in 10 ml of DMF was then added, and the mixture was subsequently stirred at RT overnight. The reaction mixture was added to 200 ml of water and extracted twice with ethyl acetate. The collected organic phases were dried over sodium sulfate, filtered and concentrated. The residue was purified by column chromatography (silica gel, mobile phase: petroleum ether/ethyl acetate=10/1) and the product fractions were concentrated. Further purification was carried out by preparative HPLC (column: Sunfire C18, 5 μm, 250×20 mm; mobile phase: 12% water+85% methanol+3% 1% strength aqueous TFA solution; flow rate: 25 ml/min; temperature: 40° C.; wavelength: 210 nm). This gave 2.67 g (71% of theory) of the title compound.

LC-MS (Method 1): Rₜ=1.29 min

MS (ESIpos): m/z=404 [M+H]⁺

Analogously to Example 3A, the exemplary compounds shown in Table 1A were prepared by reacting 5-fluoro-3-iodo-6-methyl-1H-pyrazolo[3,4-b]pyridine from Example 2A with 1-(bromomethyl)-2-fluorobenzene, 2-(bromomethyl)-1,3,4-trifluorobenzene or 2-(chloromethyl)-3-fluoropyridine hydrochloride (1.1-1.5 equivalents) and cesium carbonate (1.2-2 equivalents) under the reaction conditions described (reaction time: 2-72 h; temperature: RT to 60° C.) in DMF.

Exemplary Work-Up of the Reaction Mixture:

Method A: The reaction mixture was added to water and then stirred at room temperature for about 1 h. The solid formed was filtered off, washed with water and dried under high vacuum.

Method B: Alternatively, the reaction mixture was added to water and extracted with ethyl acetate. The collected organic phases were dried over sodium sulfate, filtered and concentrated. The residue was purified by column chromatography on silica gel (mobile phase: petroleum ether/ethyl acetate or dichloromethane/methanol).

Method C: Alternatively, the reaction mixture was diluted with acetonitrile and purified by preparative HPLC (RP18 column, mobile phase: acetonitrile/water gradient with addition of 0.1% TFA or 0.05% formic acid).

TABLE 1A

| Ex- | IUPAC name/structure | |
|---|---|---|
| ample | (Yield) | Analytical data |
| 4A | 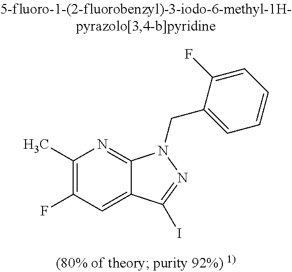 [SMILES: Cc1nc2c(cc1F)c(I)nn2Cc1ccccc1F] | ¹H-NMR (400 MHz, DMSO-d₆) δ = 2.60 (d, 3H), 5.68 (s, 2H), 7.13- 7.25 (m, 3H), 7.33-7.40 (m, 1H), 7.81 (d, 1H). LC-MS (Method 5): Rₜ = 3.02 min MS (ESIpos): m/z = 386 [M + H]⁺ |
| 5A | 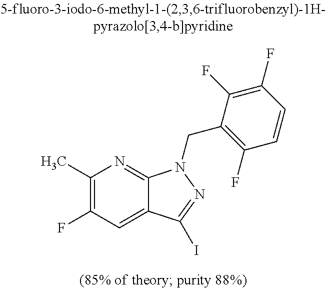 [SMILES: Cc1nc2c(cc1F)c(I)nn2Cc1c(F)ccc(F)c1F] | ¹H-NMR (400 MHz, DMSO-d₆) δ = 2.61 (d, 3H), 5.70 (s, 2H), 7.18 (ddt, 1H), 7.54 (ddt, 1H), 7.80 (d, 1H). LC-MS (Method 5): Rₜ = 3.03 min MS (ESIpos): m/z = 422 [M + H]⁺ |
| 6A | 5-fluoro-1-[(3-fluoropyridin-2-yl)methyl]-3-iodo-6- | LC-MS (Method 1): Rₜ = 1.10 min |
| | methyl-1H-pyrazolo[3,4-b]pyridine | MS (ESIpos): m/z = 387 [M + H]⁺ |
| | 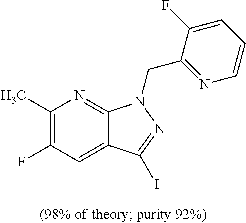 [SMILES: Cc1nc2c(cc1F)c(I)nn2Cc1ncccc1F] | |

¹⁾ This starting material has already been described in WO2013/104703 (Example 50A).

### Example 7A

### 1-(2,3-Difluorobenzyl)-5-fluoro-6-methyl-1H-pyrazolo[3,4-b]pyridine-3-carbonitrile

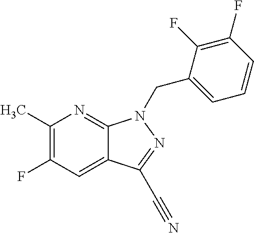 [SMILES: Cc1nc2c(cc1F)c(C#N)nn2Cc1cccc(F)c1F]

A mixture of 2.47 g (6.13 mmol) of 1-(2,3-difluorobenzyl)-5-fluoro-3-iodo-6-methyl-1H-pyrazolo[3,4-b]pyridine from Example 3A and 0.576 g (6.43 mmol) of copper(I) cyanide was initially charged in 12.1 ml of abs. DMSO in a flask which had been dried by heating, and the mixture was stirred at 150° C. for 3 h. Ethyl acetate was added to the cooled reaction solution, and the mixture was washed three times with a mixture of semisaturated aqueous ammonium chloride solution and aqueous concentrated ammonia solution (3/1). The organic phase was dried over sodium sulfate, filtered and concentrated by evaporation. The crude product was purified by flash chromatography (silica gel, mobile phase: cyclohexane/ethyl acetate gradient: 15/1 to 10/1; then dichloromethane/methanol: 10/1). This gave 780 mg of the target compound (42% of theory).

LC-MS (Method 1): Rₜ=1.19 min

MS (ESIpos): m/z=303 [M+H]⁺

¹H-NMR (400 MHz, DMSO-d₆): δ [ppm]=2.65 (d, 3H), 5.87 (s, 2H), 7.10-7.25 (m, 2H), 7.39-7.48 (m, 1H), 8.41 (d, 1H).

The exemplary compounds shown in Table 2A were prepared analogously to Example 7A by reacting the appropriate iodides with copper(I) cyanide (1.1-1.5 equivalents) under the reaction conditions described (reaction time: 1-5 h; temperature: 150° C.) in DMSO.

Exemplary Work-Up of the Reaction Mixture:

Method A: After cooling, ethyl acetate was added to the reaction mixture, and the mixture was washed three times with a mixture of semisaturated aqueous ammonium chloride solution and aqueous concentrated ammonia solution (3/1). The organic phase was dried over sodium sulfate and filtered and the solvent was removed under reduced pressure. The crude product was purified by column chromatography (silica gel, mobile phase: cyclohexane/ethyl acetate gradient: or dichloromethane/methanol gradient).

Method B: Alternatively, the reaction mixture was diluted with acetonitrile and purified by preparative HPLC (RP18 column, mobile phase: acetonitrile/water gradient with addition of 0.1% TFA or 0.05% formic acid).

TABLE 2A

| Ex- | IUPAC name/structure | |
|---|---|---|
| ample | (Yield) | Analytical data |
| 8A | 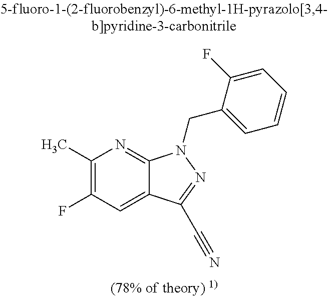 [SMILES: Cc1nc2c(cc1F)c(C#N)nn2Cc1ccccc1F] | ¹H-NMR (400 MHz, DMSO-d₆) δ = 2.65 (d, 3H), 5.82 (s, 2H), 7.18 (dt, 1H), 7.21-7.27 (m, 1H), 7.31 (dt, 1H), 7.37-7.44 (m, 1H), 8.38 (d, 1H). LC-MS (Method 1): Rₜ = 1.15 min MS (ESIpos): m/z = 285 [M + H]⁺ |
| 9A | 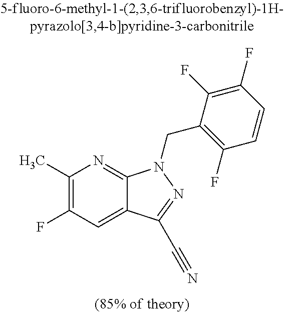 [SMILES: Cc1nc2c(cc1F)c(C#N)nn2Cc1c(F)ccc(F)c1F] | ¹H-NMR (400 MHz, DMSO-d₆) δ = 2.65 (d, 3H), 5.85 (s, 2H), 7.21 (ddt, 1H), 7.58 (ddt, 1H), 8.37 (d, 1H). LC-MS (Method 1): Rₜ = 1.15 min MS (ESIpos): m/z = 321 [M + H]⁺ |
| 10A | 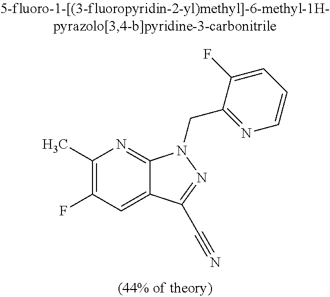 [SMILES: Cc1nc2c(cc1F)c(C#N)nn2Cc1ncccc1F] | LC-MS (Method 5): Rₜ = 2.44 min MS (ESIpos): m/z = 286 [M + H]⁺ |

¹⁾ This starting material has already been described in WO2013/104703 (Example 51A).

### Example 11A

### 1-(2,3-Difluorobenzyl)-5-fluoro-6-methyl-1H-pyrazolo[3,4-b]pyridine-3-carboximidamide

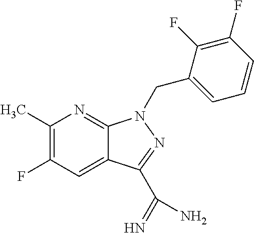 [SMILES: Cc1nc2c(cc1F)c(C(=N)N)nn2Cc1cccc(F)c1F]

960 mg (3.18 mmol) of 1-(2,3-difluorobenzyl)-5-fluoro-6-methyl-1H-pyrazolo[3,4-b]pyridine-3-carbonitrile from Example 7A were initially charged in 9.47 ml of methanol. 0.69 ml (3.18 mmol) of sodium methoxide in methanol was added, and the mixture was subsequently stirred at RT for 1 h. Another 10 ml of methanol were then added, and the reaction mixture was subsequently stirred at 60° C. for 1 h. 204 mg (3.81 mmol) of ammonium chloride and 0.71 ml (12.39 mmol) of acetic acid were added and the reaction mixture was stirred under reflux for 7 h. The solvent was removed under reduced pressure and the residue was stirred with 38 ml of 1 N aqueous sodium hydroxide solution at room temperature for 1 h. The precipitate was then filtered off and washed with water. This gave 1.0 g of the target compound (90% of theory, purity 90%).

LC-MS (Method 1): Rₜ=0.68 min

MS (ESIpos): m/z=320 [M+H]⁺

¹H-NMR (400 MHz, DMSO-d₆): δ [ppm]=2.60 (d, 3H), 5.77 (s, 2H), 6.62 (br. s, 3H), 6.91-6.98 (m, 1H), 7.11-7.20 (m, 1H), 7.34-7.44 (m, 1H), 8.29 (d, 1H).

The exemplary compounds shown in Table 3A were prepared analogously to Example 11A by reacting the appropriate nitriles with sodium methoxide (1.0-1.2 equivalents) in methanol and subsequently with ammonium chloride (1.2-1.5 equivalente) and acetic acid (3.5-5 equivalents) under the reaction conditions described (reaction time after addition of ammonium chloride and acetic acid: 5-24 h; temperature: reflux).

Exemplary Work-Up of the Reaction Mixture:

The solvent was evaporated and the residue was stirred with 1 N aqueous sodium hydroxide solution at room temperature for 0.5-2 h. The precipitate was then filtered off and washed with water and subsequently dried.

The target compounds obtained may, if appropriate partially, be present as acetate salt or acetate solvate.

TABLE 3A

| Ex- | IUPAC name/structure | |
|---|---|---|
| ample | (Yield) | Analytical data |
| 12A | 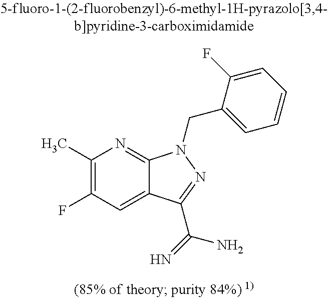 [SMILES: Cc1nc2c(cc1F)c(C(=N)N)nn2Cc1ccccc1F] | ¹H-NMR (400 MHz, DMSO-d₆) δ = 2.59 (d, 3H), 5.73 (s, 2H), 6.51 (br. s, 3H), 7.07-7.17 (m, 2H), 7.20- 7.27 (m, 1H), 7.32-7.39 (m, 1H), 8.29 (d, 1H). LC-MS (Method 7): Rₜ = 0.83 min MS (ESIpos): m/z = 302 [M + H]⁺ |
| 13A | 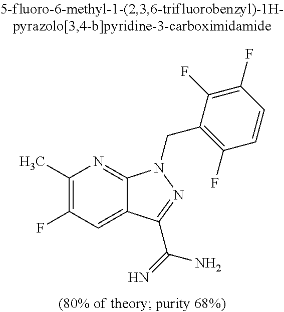 [SMILES: Cc1nc2c(cc1F)c(C(=N)N)nn2Cc1c(F)ccc(F)c1F] | ¹H-NMR (400 MHz, DMSO-d₆) δ = 2.60 (d, 3H), 5.75 (s, 2H), 6.36 (br. s, 3H), 7.17 (ddt, 1H), 7.53 (ddt, 1H), 8.25 (d, 2H). LC-MS (Method 5): Rₜ = 2.14 min MS (ESIpos): m/z = 338 [M + H]⁺ |
| 14A | 5-fluoro-1-[(3-fluoropyridin-2-yl)methyl]-6-methyl-1H- | LC-MS (Method 1): Rₜ = 0.55 min |
| | pyrazolo[3,4-b]pyridine-3-carboximidamide | MS (ESIpos): m/z = 303 [M + H]⁺ |
| | 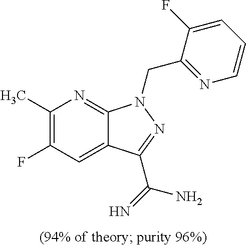 [SMILES: Cc1nc2c(cc1F)c(C(=N)N)nn2Cc1ncccc1F] | |

1) This starting material has already been described as acetate salt in WO 2013/104703 (Example 52A).

### Example 15A

### 5-Fluoro-1-[(3-fluoropyridin-2-yl)methyl]-1H-pyrazolo[3,4-b]pyridine-3-carboximidamide acetate

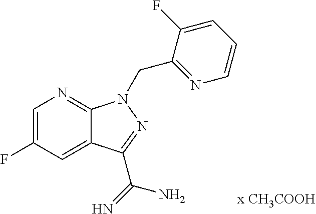 [SMILES: N=C(N)c1nn(Cc2ncccc2F)c2ncc(F)cc12]

The preparation of the compound is described in WO 2013/004785, example 14A, pp. 69-70.

### Example 16A

### 6-Chloro-1-(2-fluorobenzyl)-1H-indazole-3-carboximidamide acetate

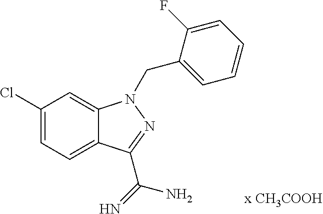 [SMILES: N=C(N)c1nn(Cc2ccccc2F)c2cc(Cl)ccc12]

The preparation of the compound is described in WO2013/104598, example 54A, pp. 97-98.

### Example 17A

### 4-Amino-2-[1-(2,3-difluorobenzyl)-5-fluoro-6-methyl-1H-pyrazolo[3,4-b]pyridin-3-yl]-5,5-dimethyl-5,7-dihydro-6H-pyrrolo[2,3-d]pyrimidin-6-one

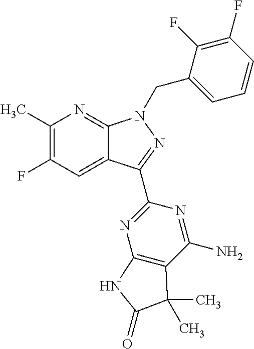 [SMILES: Cc1nc2c(cc1F)c(-c1nc(N)c3c(n1)NC(=O)C3(C)C)nn2Cc1cccc(F)c1F]

2.34 g (6.67 mmol; purity 90%) of 1-(2,3-difluorobenzyl)-5-fluoro-6-methyl-1H-pyrazolo[3,4-b]pyridine-3-carboximidamide from Example 11A were initially charged in 50.5 ml of tert-butanol. 1.33 g (8.00 mmol) of methyl 3,3-dicyanopivalate were then added, and the mixture was subsequently stirred under reflux for 6 h. Another 8 ml of tert-butanol were added and the mixture was then heated under reflux overnight. After cooling to RT, water was added and the reaction mixture was stirred at room temperature for 30 min. The precipitate formed was filtered off and washed with water. The solid was dried under high vacuum. This gave 3.25 g (99% of theory; purity: 92%) of the title compound.

LC-MS (Method 1): Rₜ=1.03 min

MS (ESIpos): m/z=454 [M+H]⁺

The exemplary compounds shown in Table 4A were prepared analogously to Example 17A by reacting the appropriate carboximidamides (amidines) with methyl 3,3-dicyanopivalate (1.1-1.5 equivalents) in tert-butanol [0.2-1.4 equivalents of potassium tert-butoxide were added to amidines present as acetate salt or acetate solvate] under the reaction conditions described (reaction time: 4-24 h).

Exemplary Work-Up of the Reaction Mixture:

Water was added to the reaction mixture and the mixture was stirred at room temperature for 30 min. The precipitate formed was filtered off and washed with water.

TABLE 4A

| Ex- | IUPAC name/structure | |
|---|---|---|
| ample | (Yield) | Analytical data |
| 18A | 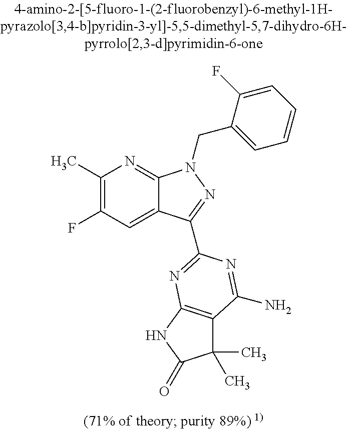 [SMILES: Cc1nc2c(cc1F)c(-c1nc(N)c3c(n1)NC(=O)C3(C)C)nn2Cc1ccccc1F] | LC-MS (Method 1): Rₜ = 1.01 min MS (ESIpos): m/z = 436 [M + H]⁺ |
| 19A | 4-amino-2-[5-fluoro-6-methyl-1-(2,3,6-trifluorobenzyl)- | LC-MS (Method 1): Rₜ = 1.03 min |
| | 1H-pyrazolo[3,4-b]pyridin-3-yl]-5,5-dimethyl-5,7-dihydro- | MS (ESIpos): m/z = 472 [M + H]⁺ |
| | 6H-pyrrolo[2,3-d]pyrimidin-6-one | |
| | 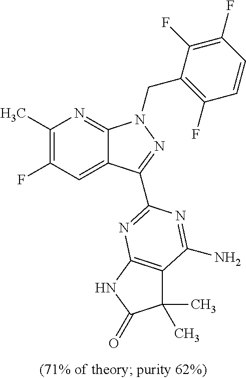 [SMILES: Cc1nc2c(cc1F)c(-c1nc(N)c3c(n1)NC(=O)C3(C)C)nn2Cc1c(F)ccc(F)c1F] | |
| 20A | 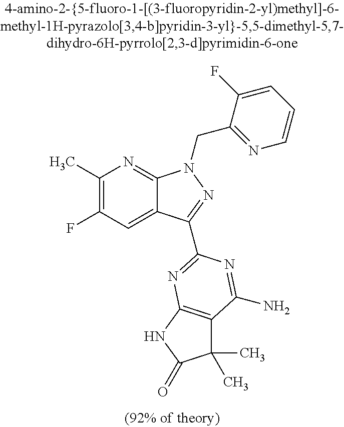 [SMILES: Cc1nc2c(cc1F)c(-c1nc(N)c3c(n1)NC(=O)C3(C)C)nn2Cc1ncccc1F] | ¹H-NMR (400 MHz, DMSO-d₆) δ = 1.34 (s, 6H), 2.61 (d, 3H), 5.89 (s, 2H), 6.81 (br. s, 2H), 7.40-7.47 (m, 1H), 7.77 (t, 1H), 8.29 (d, 1H), 8.72 (d, 1H), 10.91 (br. s, 1H). LC-MS (Method 5): Rₜ = 2.16 min MS (ESIpos): m/z = 437 [M + H]⁺ |
| 21A | 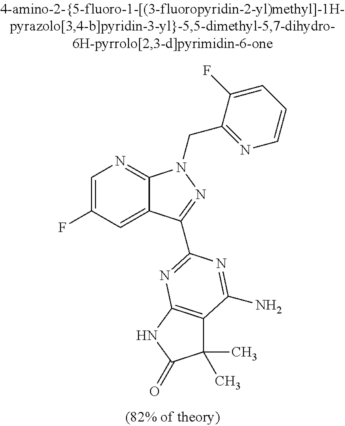 [SMILES: CC1(C)C(=O)Nc2nc(-c3nn(Cc4ncccc4F)c4ncc(F)cc34)nc(N)c21] | ¹H-NMR (400 MHz, DMSO-d₆) δ = 1.34 (s, 6H), 5.95 (s, 2H), 6.87 (br. s, 2H), 7.41-7.48 (m, 1H), 7.78 (t, 1H), 8.28 (d, 1H), 8.64-8.70 (m, 1H), 8.81-8.87 (m, 1H), 10.97 (br. s, 1H). LC-MS (Method 1): Rₜ = 0.80 min MS (ESIpos): m/z = 423 [M + H]⁺ |
| 22A | 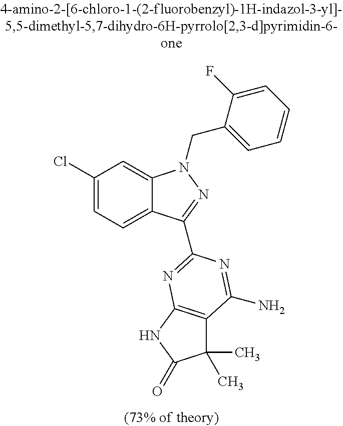 [SMILES: CC1(C)C(=O)Nc2nc(-c3nn(Cc4ccccc4F)c4cc(Cl)ccc34)nc(N)c21] | ¹H-NMR (400 MHz, DMSO-d₆) δ = ppm 1.34 (s, 6H), 5.79 (s, 2H), 6.79 (br. s, 2H), 7.06-7.32 (m, 4H), 7.32- 7.42 (m, 1H), 7.99 (s, 1H), 8.69 (d, 1H), 10.97 (br. s, 1H) LC-MS (Method 1): Rₜ = 1.03 min MS (ESIpos): m/z = 437 [M + H]⁺ |

¹⁾ This starting material has already been described in WO 2013/104703 (Example 55A).

### Example 23A

### Methyl 3,3-dicyano-2-(trifluoromethyl)acrylate

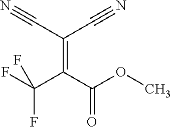

The synthesis of this compound is described in Journal of Fluorine Chemistry 1991, vol. 51, 3, pp. 323-334.

### Example 24A

### Methyl 2-(dicyanomethyl)-3,3,3-trifluoro-2-methylpropanoate

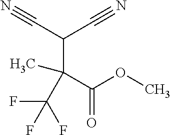

3.00 g (14.70 mmol) of Example 23A were dissolved in tetrahydrofuran (30 ml) and the solution was cooled to 0° C. 7.35 ml (22.05 mmol) of methylmagnesium chloride (3 M in THF) were then added dropwise such that the temperature did not exceed 5° C. After the addition had ended, the mixture was stirred for another 10 min. 1 N aqueous hydrochloric acid was then added to the mixture, and the mixture was subsequently extracted with ethyl acetate. The phases were separated and the aqueous phase was extracted twice more with ethyl acetate. The combined organic phases were washed with saturated aqueous sodium chloride solution, dried over sodium sulfate, filtered and concentrated. The crude product was then purified by column chromatography (silica gel, mobile phase: cyclohexane, then cyclohexane:ethyl acetate 9:1 (v:v)). Concentration gave 3.24 g (63% of theory) of the title compound.

¹H-NMR (400 MHz, CDCl₃): δ [ppm]=1.81 (s, 3H), 3.95 (s, 3H), 4.48 (s, 1H).

### Example 25A

### rac-4-Amino-2-{5-fluoro-1-[(3-fluoropyridin-2-yl)methyl]-1H-pyrazolo[3,4-b]pyridin-3-yl}-5-methyl-5-(trifluoromethyl)-5,7-dihydro-6H-pyrrolo[2,3-d]pyrimidin-6-one

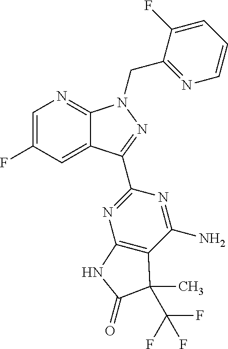 [SMILES: CC1(C(F)(F)F)C(=O)Nc2nc(-c3nn(Cc4ncccc4F)c4ncc(F)cc34)nc(N)c21]

23.0 g (66.02 mmol) of 5-fluoro-1-[(3-fluoropyridin-2-yl)methyl]-1H-pyrazolo[3,4-b]pyridine-3-carboximidamide acetate from Example 15A were initially charged in tert-butanol (400 ml), and 13.43 g (119.68 mmol) of potassium tert-butoxide were added. Subsequently, 21.08 g (95.75 mmol) of methyl 2-(dicyanomethyl)-3,3,3-trifluoro-2-methylpropanoate from Example 24A in tert-butanol (100 ml) were added, and the mixture was heated under reflux overnight. After cooling to RT, water was added and the reaction mixture was stirred at room temperature for a further 30 min. The precipitate formed was filtered off and washed with water and a little diethyl ether. The solid was dried under high vacuum. This gave 16.1 g of the title compound (51% of theory).

LC-MS (Method 1): Rₜ=0.95 min;

MS (ESIpos): m/z=477 [M+H]⁺

¹H-NMR (400 MHz, DMSO-d₆): δ [ppm]=1.72 (s, 3H), 5.96 (s, 2H), 7.10 (br. s, 2H), 7.42-7.48 (m, 1H), 7.75-7.80 (m, 1H), 8.27 (d, 1H), 8.68 (dd, 1H), 8.86 (dd, 1H), 11.60 (br. s, 1H).

The exemplary compounds shown in Table 5A were prepared analogously to Example 25A by reacting the appropriate carboximidamides (amidines) with methyl 2-(dicyanomethyl)-3,3,3-trifluoro-2-methylpropanoate (1.1-1.5 equivalents) in tert-butanol [0.2-1.4 equivalents of potassium tert-butoxide were added to amidines present as acetate salt or acetate solvate] under the reaction conditions described (reaction time: 0.5-24 h).

Alternatively, the reactions can be carried out in the microwave [0.5-10 h, 100° C.]

Exemplary Work-Up of the Reaction Mixture:

Water was added, and the reaction mixture was stirred at room temperature for 30 min. The precipitate formed was filtered off and washed with water.

TABLE 5A

| Ex- | IUPAC name/structure | |
|---|---|---|
| ample | (Yield) | Analytical data |
| 26A | 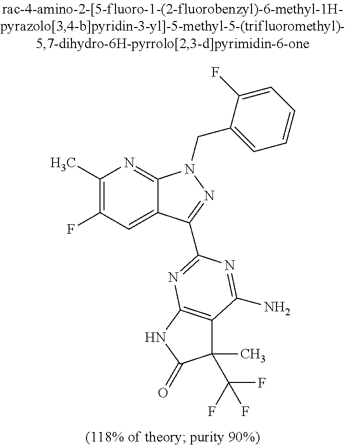 [SMILES: Cc1nc2c(cc1F)c(-c1nc(N)c3c(n1)NC(=O)C3(C)C(F)(F)F)nn2Cc1ccccc1F] | ¹H-NMR (400 MHz, DMSO-d₆) δ = 1.72 (s, 3H), 2.63 (d, 3H), 5.78 (s, 2H), 7.07 (br. m, 2H), 7.12-7.27 (m, 3H), 7.33-7.40 (m, 1H), 8.77 (d, 1H), 11.60 (s, 1H). LC-MS (Method 1): Rₜ = 1.09 min MS (ESIpos): m/z = 490 [M + H]⁺ |
| 27A | 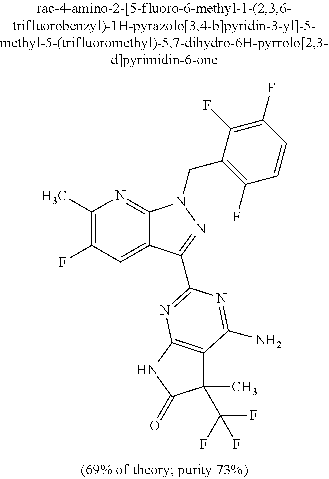 [SMILES: Cc1nc2c(cc1F)c(-c1nc(N)c3c(n1)NC(=O)C3(C)C(F)(F)F)nn2Cc1c(F)ccc(F)c1F] | ¹H-NMR (400 MHz, DMSO-d₆) δ = 1.71 (s, 3H), 2.64 (d, 3H), 5.81 (s, 2H), 7.07 (br. s, 2H), 7.15-7.25 (m, 1H), 7.48-7.61 (m, 1H), 8.77 (d, 1H), 11.60 (s, 1H). LC-MS (Method 1): Rₜ = 1.10 min MS (ESIpos): m/z = 526 [M + H]⁺ |
| 28A | rac-4-amino-2-[1-(2,3-difluorobenzyl)-5-fluoro-6-methyl- | LC-MS (Method 1): Rₜ = 1.14 min |
| | 1H-pyrazolo[3,4-b]pyridin-3-yl]-5-methyl-5- | MS (ESIpos): m/z = 508 [M + H]⁺ |
| | (trifluoromethyl)-5,7-dihydro-6H-pyrrolo[2,3-d]pyrimidin- | |
| | 6-one | |
| | 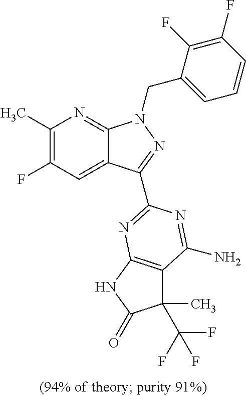 [SMILES: Cc1nc2c(cc1F)c(-c1nc(N)c3c(n1)NC(=O)C3(C)C(F)(F)F)nn2Cc1cccc(F)c1F] | |
| 29A | rac-4-amino-2-{5-fluoro-1-[(3-fluoropyridin-2-yl)methyl]- | LC-MS (Method 1): Rₜ = 0.99 min |
| | 6-methyl-1H-pyrazolo[3,4-b]pyridin-3-yl}-5-methyl-5- | MS (ESIpos): m/z = 491 [M + H]⁺ |
| | (trifluoromethyl)-5,7-dihydro-6H-pyrrolo[2,3-d]pyrimidin- | |
| | 6-one | |
| | 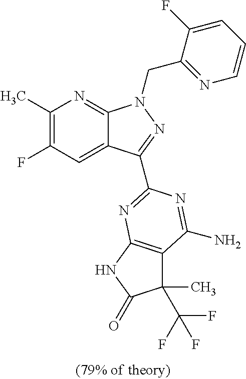 [SMILES: Cc1nc2c(cc1F)c(-c1nc(N)c3c(n1)NC(=O)C3(C)C(F)(F)F)nn2Cc1ncccc1F] | |

### Example 30A

### 2-[1-(2,3-Difluorobenzyl)-5-fluoro-6-methyl-1H-pyrazolo[3,4-b]pyridin-3-yl]-4-iodo-5,5-dimethyl-5,7-dihydro-6H-pyrrolo[2,3-d]pyrimidin-6-one

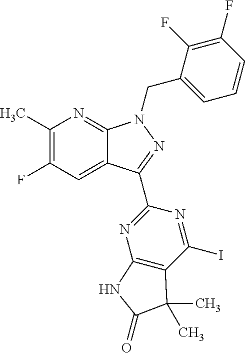 [SMILES: Cc1nc2c(cc1F)c(-c1nc(I)c3c(n1)NC(=O)C3(C)C)nn2Cc1cccc(F)c1F]

3.25 g (6.61 mmol; purity 92%) of 4-amino-2-[1-(2,3-difluorobenzyl)-5-fluoro-6-methyl-1H-pyrazolo[3,4-b]pyridin-3-yl]-5,5-dimethyl-5,7-dihydro-6H-pyrrolo[2,3-d]pyrimidin-6-one from Example 17A were initially charged in 64 ml of dioxane, 4.42 ml (33.04 mmol) of isopentyl nitrite and 2.66 ml (33.04 mmol) of diiodomethane were added and the mixture was then heated at 85° C. for 3 h. After cooling, the mixture was concentrated under reduced pressure and the residue was chromatographed on silica gel (mobile phase: dichloromethane/methanol gradient). Removal of the solvent under reduced pressure gave 2.32 g (51% of theory, purity 82%) of the title compound.

LC-MS (Method 1): Rₜ=1.34 min

MS (ESIpos): m/z=565 [M+H]⁺

The exemplary compounds shown in Table 6A were prepared analogously to Example 30A by reacting the appropriate anilines with diiodomethane (3-18 equivalents) and isopentyl nitrite (3-10 equivalents) in dioxane under the reaction conditions described (temperature: 85° C.; reaction time: 2-10 h).

Exemplary Work-Up of the Reaction Mixture:

The reaction mixture was concentrated [if appropriate partitioned between water and an organic solvent and then concentrated] and the residue was chromatographed on silica gel (mobile phase: dichloromethane/methanol or cyclohexane/ethyl acetate gradient]. Optionally, further purification was carried out by preparative HPLC [column: Sunfire C18, 5 μM, 100×30 mm; mobile phase: water/acetonitrile+0.2% strength formic acid].

TABLE 6A

| Ex- | IUPAC name/structure | |
|---|---|---|
| ample | (Yield) | Analytical data |
| 31A | 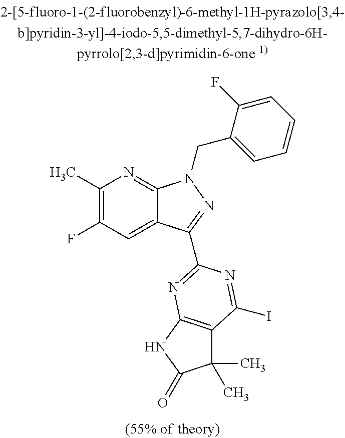 [SMILES: Cc1nc2c(cc1F)c(-c1nc(I)c3c(n1)NC(=O)C3(C)C)nn2Cc1ccccc1F] | ¹H-NMR (400 MHz, DMSO-d₆) δ = 1.42 (s, 6H), 2.64 (d, 3H), 5.82 (s, 2H), 7.12-7.20 (m, 2H), 7.20- 7.27 (m, 1H), 7.34-7.41 (m, 1H), 8.37 (d, 1H), 11.73 (s, 1H). LC-MS (Method 7): Rₜ = 1.64 min MS (ESIpos): m/z = 547 [M + H]⁺ |
| 32A | 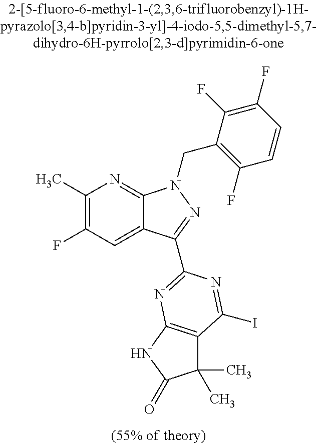 [SMILES: Cc1nc2c(cc1F)c(-c1nc(I)c3c(n1)NC(=O)C3(C)C)nn2Cc1c(F)ccc(F)c1F] | ¹H-NMR (400 MHz, DMSO-d₆) δ = 1.41 (s, 6H), 2.65 (d, 3H), 5.85 (s, 2H), 7.20 (ddt, 1H), 7.55 (ddt, 1H), 8.36 (d, 1H), 11.73 (s, 1H). LC-MS (Method 7): Rₜ = 1.64 min MS (ESIpos): m/z = 583 [M + H]⁺ |
| 33A | 2-{5-fluoro-1-[(3-fluoropyridin-2-yl)methyl]-6-methyl-1H- | LC-MS (Method 1): Rₜ = 1.15 min |
| | pyrazolo[3,4-b]pyridin-3-yl}-4-iodo-5,5-dimethyl-5,7- | MS (ESIpos): m/z = 548 [M + H]⁺ |
| | dihydro-6H-pyrrolo[2,3-d]pyrimidin-6-one | |
| | 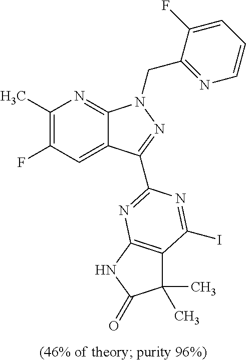 [SMILES: Cc1nc2c(cc1F)c(-c1nc(I)c3c(n1)NC(=O)C3(C)C)nn2Cc1ncccc1F] | |
| 34A | 2-{5-fluoro-1-[(3-fluoropyridin-2-yl)methyl]-1H- | LC-MS (Method 7): Rₜ = 1.36 min |
| | pyrazolo[3,4-b]pyridin-3-yl}-4-iodo-5,5-dimethyl-5,7- | MS (ESIpos): m/z = 534 [M + H]⁺ |
| | dihydro-6H-pyrrolo[2,3-d]pyrimidin-6-one | |
| | 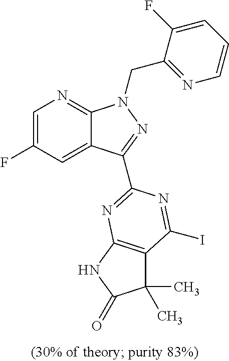 [SMILES: CC1(C)C(=O)Nc2nc(-c3nn(Cc4ncccc4F)c4ncc(F)cc34)nc(I)c21] | |
| 35A | 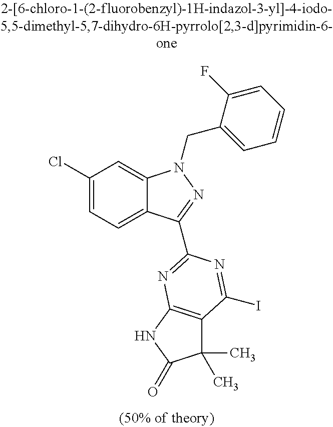 [SMILES: CC1(C)C(=O)Nc2nc(-c3nn(Cc4ccccc4F)c4cc(Cl)ccc34)nc(I)c21] | ¹H-NMR (400 MHz, DMSO-d₆) δ = 1.41 (s, 6H), 5.86 (s, 2H), 7.10- 7.29 (m, 3H), 7.31-7.44 (m, 2H), 8.06 (d, 1H), 8.47 (d, 1H), 11.75 (s, 1H). LC-MS (Method 1): Rₜ = 1.32 min MS (ESIpos): m/z = 548 [M + H]⁺ |

¹⁾ This starting material has already been described in WO 2013/104703 (Example 56A).

### Example 36A

### rac-2-{5-Fluoro-1-[(3-fluoropyridin-2-yl)methyl]-1H-pyrazolo[3,4-b]pyridin-3-yl}-4-iodo-5-methyl-5-(trifluoromethyl)-5,7-dihydro-6H-pyrrolo[2,3-d]pyrimidin-6-one

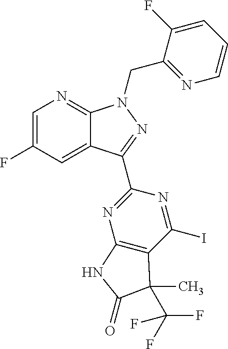 [SMILES: CC1(C(F)(F)F)C(=O)Nc2nc(-c3nn(Cc4ncccc4F)c4ncc(F)cc34)nc(I)c21]

798 μl (5.93 mmol) of isopentyl nitrite and 286 μl (3.56 mmol) of diiodomethane were added to 565 mg (1.19 mmol) of rac-4-amino-2-{5-fluoro-1-[(3-fluoropyridin-2-yl)methyl]-1H-pyrazolo[3,4-b]pyridin-3-yl}-5-methyl-5-(trifluoromethyl)-5,7-dihydro-6H-pyrrolo[2,3-d]pyrimidin-6-one from Example 25A in 15 ml of dioxane, and the mixture was heated to 85° C. for 4 h. After cooling, the mixture was concentrated under reduced pressure, the residue was taken up in dichloromethane, kieselguhr was added and the mixture was then concentrated under reduced pressure. The crude compound adsorbed on kieselguhr was then purified by column chromatography (silica gel, mobile phase: cyclohexane/ethyl acetate gradient). Concentration gave 297 mg (42% of theory) of the title compound.

LC-MS (Method 1): Rₜ=1.19 min;

MS (ESIpos): m/z=588 [M+H]⁺

¹H-NMR (400 MHz, DMSO-d₆): δ [ppm]=1.81 (s, 3H), 6.04 (s, 2H), 7.43-7.47 (m, 1H), 7.77-7.82 (m, 1H), 8.26 (d, 1H), 8.47 (dd, 1H), 8.76 (dd, 1H), 12.41 (br. s, 1H).

The exemplary compounds shown in Table 7A were prepared analogously to Example 36A by reacting the appropriate anilines with diiodomethane (4-18 equivalents) and isopentyl nitrite (4-12 equivalents) in dioxane under the reaction conditions described (temperature: 85° C.; reaction time: 2-10 h).

Exemplary Work-Up of the Reaction Mixture:

The reaction mixture was concentrated and the residue was chromatographed on silica gel (mobile phase: dichloromethane/methanol gradient). Optionally, further purification was carried out by preparative HPLC [column: Kinetex C18, 5 μM, 100×300 mm; mobile phase: water/acetonitrile 35:65].

TABLE 7A

| Ex- | IUPAC name/structure | |
|---|---|---|
| ample | (Yield) | Analytical data |
| 37A | 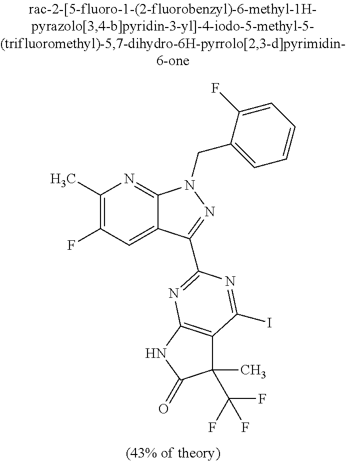 [SMILES: Cc1nc2c(cc1F)c(-c1nc(I)c3c(n1)NC(=O)C3(C)C(F)(F)F)nn2Cc1ccccc1F] | ¹H-NMR (400 MHz, DMSO-d₆) δ = 1.81 (s, 3H), 2.64 (d, 3H), 5.84 (s, 2H), 7.13-7.27 (m, 3H), 7.34-7.41 (m, 1H), 8.37 (d, 1H), 12.39 (s, 1H). LC-MS (Method 7): Rₜ = 1.64 min MS (ESIpos): m/z = 601 [M + H]⁺ |
| 38A | 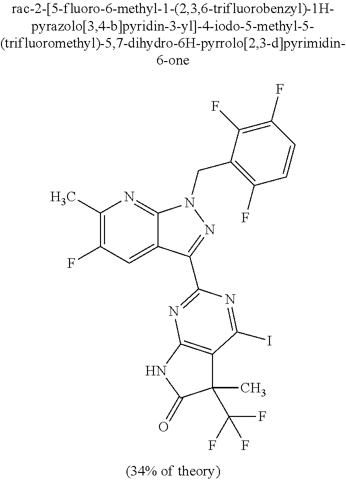 [SMILES: Cc1nc2c(cc1F)c(-c1nc(I)c3c(n1)NC(=O)C3(C)C(F)(F)F)nn2Cc1c(F)ccc(F)c1F] | ¹H-NMR (400 MHz, DMSO-d₆) δ = 1.80 (s, 3H), 2.65 (d, 3H), 5.87 (s, 2H), 7.21 (ddt, 1H), 7.56 (ddt, 1H), 8.36 (d, 1H), 12.39 (s, 1H). LC-MS (Method 2): Rₜ = 4.45 min MS (ESIpos): m/z = 637 [M + H]⁺ |
| 39A | rac-2-[1-(2,3-difluorobenzyl)-5-fluoro-6-methyl-1H- | LC-MS (Method 1): Rₜ = 1.35 min |
| | pyrazolo[3,4-b]pyridin-3-yl]-4-iodo-5-methyl-5- | MS (ESIpos): m/z = 619 [M + H]⁺ |
| | (trifluoromethyl)-5,7-dihydro-6H-pyrrolo[2,3-d]pyrimidin- | |
| | 6-one | |
| | 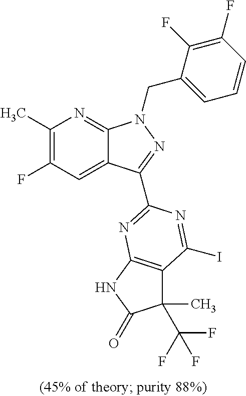 [SMILES: Cc1nc2c(cc1F)c(-c1nc(I)c3c(n1)NC(=O)C3(C)C(F)(F)F)nn2Cc1cccc(F)c1F] | |
| 40A | rac-2-{5-fluoro-1-[(3-fluoropyridin-2-yl)methyl]-6- | LC-MS (Method 1): Rₜ = 1.26 min |
| | methyl-1H-pyrazolo[3,4-b]pyridin-3-yl}-4-iodo-5-methyl- | MS (ESIpos): m/z = 602 [M + H]⁺ |
| | 5-(trifluoromethyl)-5,7-dihydro-6H-pyrrolo[2,3- | |
| | d]pyrimidin-6-one | |
| | 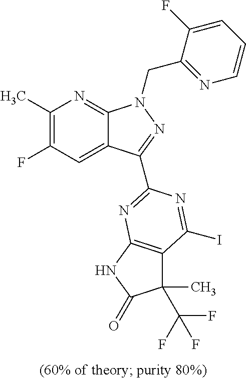 [SMILES: Cc1nc2c(cc1F)c(-c1nc(I)c3c(n1)NC(=O)C3(C)C(F)(F)F)nn2Cc1ncccc1F] | |

### Example 41A

### 2-[1-(2-Fluorobenzyl)-1H-pyrazolo[3,4-b]pyridin-3-yl]-5,5-dimethyl-6-oxo-6,7-dihydro-5H-pyrrolo[2,3-d]pyrimidine-4-carbonitrile

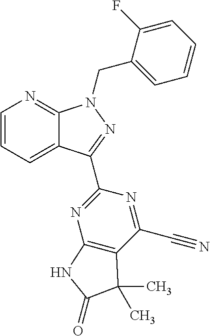 [SMILES: CC1(C)C(=O)Nc2nc(-c3nn(Cc4ccccc4F)c4ncccc34)nc(C#N)c21]

This substance has already been described in WO 2013/104703.

Alternative Preparation Method:

27 g (52.5 mmol) of 2-[1-(2-fluorobenzyl)-1H-pyrazolo[3,4-b]pyridin-3-yl]-4-iodo-5,5-dimethyl-5,7-dihydro-6H-pyrrolo[2,3-d]pyrimidin-6-one [described in WO 2013/030288, Ex. 15A] and 5.17 g (57.75 mmol) copper(I) cyanide in 200 ml of DMSO were stirred at 150° C. for 2 h. After cooling to 40° C., the reaction mixture was poured into a mixture of water, aqueous conc. ammonia solution and ethyl acetate, stirred and filtered through kieselguhr. The phases were separated, the org. phase was washed twice with sat. sodium chloride solution, dried and concentrated and dried under high vacuum. The crude product was purified by column chromatography (silica gel, mobile phase: dichloromethane/methanol (2%)). Mixed fractions were subsequently purified by a second column chromatography (silica gel, dichloromethane/1-2% methanol). This gave a total of 12.0 g (55% of th.) of the title compound.

LC-MS (Method 7): Rₜ=1.35 min

MS (ESIpos): m/z=414 [M+H]⁺

¹H-NMR (400 MHz, DMSO-d₆): δ [ppm]=1.48 (s, 6H), 5.89 (s, 2H), 7.09-7.29 (m, 3H), 7.32-7.41 (m, 1H), 7.44-7.56 (m, 1H), 8.71 (d, 1H), 8.82 (d, 1H), 12.17 (br. s, 1H).

### Example 42A

### 2-[5-Fluoro-1-(2-fluorobenzyl)-1H-pyrazolo[3,4-b]pyridin-3-yl]-5,5-dimethyl-6-oxo-6,7-dihydro-5H-pyrrolo[2,3-d]pyrimidine-4-carbonitrile

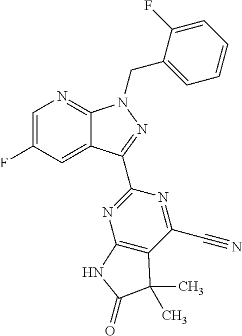 [SMILES: CC1(C)C(=O)Nc2nc(-c3nn(Cc4ccccc4F)c4ncc(F)cc34)nc(C#N)c21]

The substance has been described in WO 2013/104703 Example 81A, p. 163.

### Example 43A

### 2-[1-(2,3-Difluorobenzyl)-5-fluoro-6-methyl-1H-pyrazolo[3,4-b]pyridin-3-yl]-5,5-dimethyl-6-oxo-6,7-dihydro-5H-pyrrolo[2,3-d]pyrimidine-4-carbonitrile

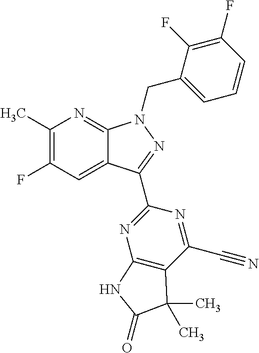 [SMILES: Cc1nc2c(cc1F)c(-c1nc(C#N)c3c(n1)NC(=O)C3(C)C)nn2Cc1cccc(F)c1F]

In a flask which had been dried by heating, 150 mg (0.22 mmol; purity 82%) of 2-[1-(2,3-difluorobenzyl)-5-fluoro-6-methyl-1H-pyrazolo[3,4-b]pyridin-3-yl]-4-iodo-5,5-dimethyl-5,7-dihydro-6H-pyrrolo[2,3-d]pyrimidin-6-one from Example 30A were initially charged in 2 ml of abs. DMSO, 27 mg (0.30 mmol) of copper(I) cyanide were added and the mixture was heated at 150° C. for 2 h. The reaction solution was filtered through Celite, rinsed with about 14 ml of ethyl acetate and washed three times with a mixture of semiconcentrated aqueous ammonium chloride solution/concentrated aqueous ammonium chloride solution (3/1) and once with saturated aqueous sodium chloride solution. The organic phase was dried over sodium sulfate, filtered and the solvent was removed under reduced pressure. The crude product was purified by column chromatography (silica gel, mobile phase: dichloromethane to dichloromethane/methanol=100/1). The crude product obtained was then purified by a second column chromatography (silica gel, mobile phase: cyclohexane/ethyl acetate=5/1). Removal of the solvent under reduced pressure gave 104 mg (95% of theory; purity 93%) of the title compound.

LC-MS (Method 1): Rₜ=1.20 min

MS (ESIpos): m/z=464 [M+H]⁺

The exemplary compounds shown in Table 8A were prepared analogously to Example 43A by reacting the appropriate iodides with copper(I) cyanide (1.0-1.5 equivalents) in DMSO under the reaction conditions described (temperature: 150° C.; reaction time: 0.25-3 h).

Exemplary Work-Up of the Reaction Mixture:

Method A: The reaction solution was, if appropriate, filtered through Celite, rinsed with ethyl acetate and washed three times with a mixture of semiconcentrated aqueous ammonium chloride solution/concentrated aqueous ammonium chloride solution (3/1) and once with saturated aqueous sodium chloride solution. The organic phase was dried over sodium sulfate, filtered and the solvent was removed under reduced pressure. The crude product was purified by column chromatography (silica gel, mobile phase: dichloromethane/methanol or cyclohexane/ethyl acetate gradient) or preparative HPLC (RP18 column, mobile phase: acetonitrile/water gradient with addition of 0.1% TFA).

Method B: Alternatively or additionally, water/acetonitrile was added and the reaction mixture was purified by preparative HPLC (RP18 column, mobile phase: acetonitrile/water gradient or methanol/water gradient with addition of 0.1% TFA).

TABLE 8A

| Ex- | IUPAC name/structure | |
|---|---|---|
| ample | (Yield) | Analytical data |
| 44A | 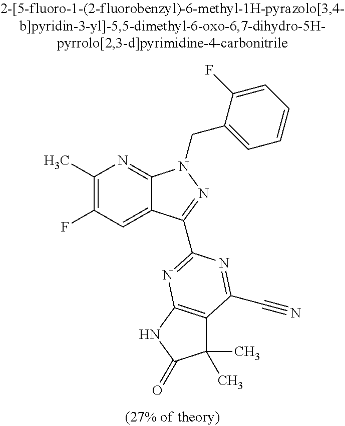 [SMILES: Cc1nc2c(cc1F)c(-c1nc(C#N)c3c(n1)NC(=O)C3(C)C)nn2Cc1ccccc1F] | ¹H-NMR (400 MHz, DMSO-d₆) δ = 1.48 (s, 6H), 2.65 (d, 3H), 5.84 (s, 2H), 7.13-7.27 (m, 3H), 7.34-7.41 (m, 1H), 8.42 (d, 1H), 12.13 (s, 1H). LC-MS (Method 7): Rₜ = 1.52 min MS (ESIpos): m/z = 446 [M + H]⁺ |
| 45A | 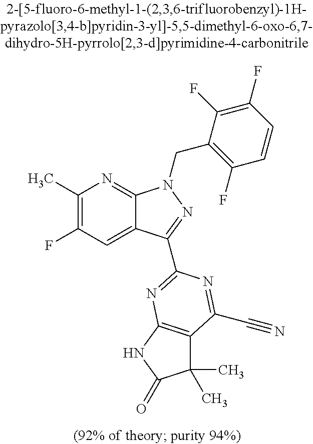 [SMILES: Cc1nc2c(cc1F)c(-c1nc(C#N)c3c(n1)NC(=O)C3(C)C)nn2Cc1c(F)ccc(F)c1F] | ¹H-NMR (400 MHz, DMSO-d₆) δ = 1.39 (s, 6H), 2.65 (d, 3H), 5.85 (s, 2H), 7.20 (t, 1H), 7.55 (ddt, 1H), 8.41 (d, 1H), 12.05 (s, 1H). LC-MS (Method 1): Rₜ = 1.20 min MS (ESIpos): m/z = 482 [M + H]⁺ |
| 46A | 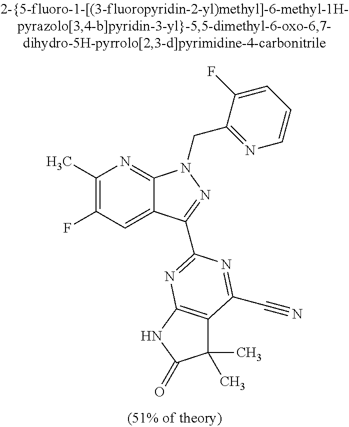 [SMILES: Cc1nc2c(cc1F)c(-c1nc(C#N)c3c(n1)NC(=O)C3(C)C)nn2Cc1ncccc1F] | ¹H-NMR (400 MHz, DMSO-d₆) δ = 1.48 (s, 6H), 2.63 (d, 3H), 5.98 (s, 2H), 7.39-7.47 (m, 1H), 7.78 (t, 1H), 8.28 (d, 1H), 8.42 (d, 1H), 12.10 (s, 1H). LC-MS (Method 1): Rₜ = 1.07 min MS (ESIpos): m/z = 447 [M + H]⁺ |
| 47A | 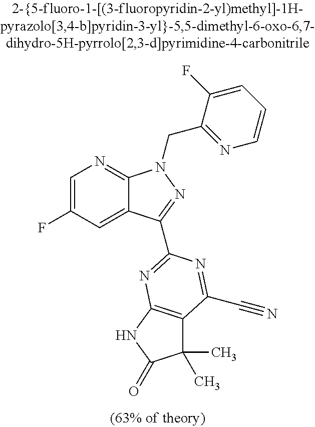 [SMILES: CC1(C)C(=O)Nc2nc(-c3nn(Cc4ncccc4F)c4ncc(F)cc34)nc(C#N)c21] | ¹H-NMR (500 MHz, DMSO-d₆) δ = 1.48 (s, 6H), 6.03 (s, 2H), 7.40- 7.47 (m, 1H), 7.78 (t, 1H), 8.26 (d, 1H), 8.50-8.55 (m, 1H), 8.72-8.76 (m, 1H), 12.13 (s, 1H). LC-MS (Method 1): Rₜ = 0.99 min MS (ESIpos): m/z = 433 [M + H]⁺ |
| 48A | 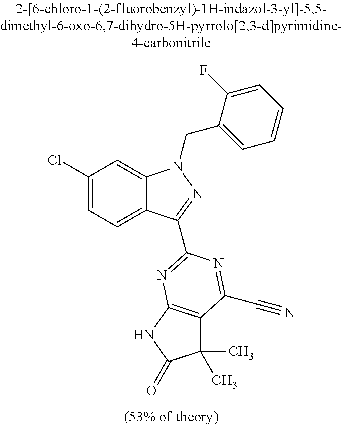 [SMILES: CC1(C)C(=O)Nc2nc(-c3nn(Cc4ccccc4F)c4cc(Cl)ccc34)nc(C#N)c21] | ¹H-NMR (400 MHz, DMSO-d₆) δ = 1.38-1.59 (m, 7H), 5.80-5.94 (m, 2H), 7.08-7.31 (m, 3H), 7.31-7.52 (m, 2H), 8.00-8.18 (m, 1H), 8.40- 8.57 (m, 1H), 12.01-12.26 (m, 1H). LC-MS (Method 1): Rₜ = 1.23 min MS (ESIpos): m/z = 447 |

### Example 49A

### rac-2-{5-Fluoro-1-[(3-fluoropyridin-2-yl)methyl]-1H-pyrazolo[3,4-b]pyridin-3-yl}-5-methyl-6-oxo-5-(trifluoromethyl)-6,7-dihydro-5H-pyrrolo[2,3-d]pyrimidine-4-carbonitrile

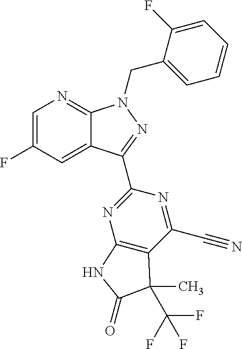 [SMILES: CC1(C(F)(F)F)C(=O)Nc2nc(-c3nn(Cc4ccccc4F)c4ncc(F)cc34)nc(C#N)c21]

In a flask which had been dried by heating, 560 mg (0.84 mmol) of rac-2-{5-fluoro-1-[(3-fluoropyridin-2-yl)methyl]-1H-pyrazolo[3,4-b]pyridin-3-yl}-4-iodo-5-methyl-5-(trifluoromethyl)-5,7-dihydro-6H-pyrrolo[2,3-d]pyrimidin-6-one from Example 36A were initially charged in 9 ml of abs. DMSO, 83 mg (0.92 mmol) of copper(I) cyanide were added and the mixture was then heated at 150° C. for 1.5 h. The reaction solution was cooled, water/acetonitrile were added and the mixture was purified by preparative HPLC (RP18 column, mobile phase: acetonitrile/water gradient with addition of 0.1% TFA). Evaporation gave 80 mg (20% of theory) of the title compound.

LC-MS (Method 1): Rₜ=1.07 min

MS (ESIpos): m/z=487 [M+H]⁺

The exemplary compounds shown in Table 9A were prepared analogously to Example 49A by reacting the appropriate iodides with copper(I) cyanide (1.0-1.5 equivalents) in DMSO under the reaction conditions described (temperature: 150° C.; reaction time: 0.25-3 h).

Exemplary Work-Up of the Reaction Mixture:

The reaction solution was, if appropriate, filtered through Celite, rinsed with ethyl acetate and washed three times with a mixture of semiconcentrated aqueous ammonium chloride solution/concentrated aqueous ammonium chloride solution (3/1) and once with saturated aqueous sodium chloride solution. The organic phase was dried over sodium sulfate, filtered and the solvent was removed under reduced pressure. The crude product was purified by column chromatography (silica gel, mobile phase: dichloromethane/methanol gradient or cyclohexane/ethyl acetate gradient) preparative HPLC (RP18 column, mobile phase: acetonitrile/water gradient with addition of 0.1% TFA). Alternatively or additionally, water/acetonitrile was added and the reaction mixture was purified by preparative HPLC (RP18 column, mobile phase: acetonitrile/water gradient with addition of 0.1% TFA).

TABLE 9A

| Ex- | IUPAC name/structure | |
|---|---|---|
| ample | (Yield) | Analytical data |
| 50A | 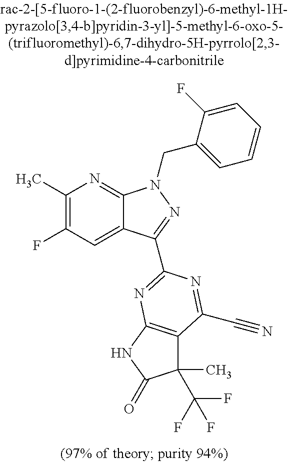 [SMILES: Cc1nc2c(cc1F)c(-c1nc(C#N)c3c(n1)NC(=O)C3(C)C(F)(F)F)nn2Cc1ccccc1F] | ¹H-NMR (400 MHz, DMSO-d₆) δ = 1.56 (s, 3H), 2.63 (d, 3H), 5.81 (s, 2H), 7.12-7.18 (m, 2H), 7.20-7.27 (m, 1H), 7.33-7.40 (m, 1H), 8.42 (d, 1H). LC-MS (Method 1): Rₜ = 1.23 min MS (ESIpos): m/z = 500 [M + H]⁺ |
| 51A | 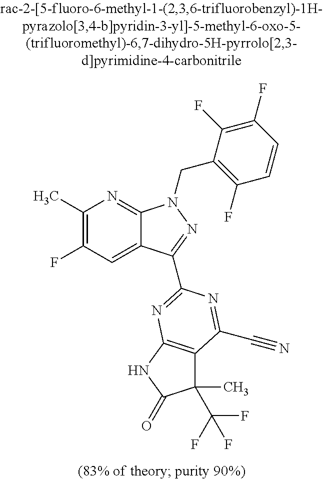 [SMILES: Cc1nc2c(cc1F)c(-c1nc(C#N)c3c(n1)NC(=O)C3(C)C(F)(F)F)nn2Cc1c(F)ccc(F)c1F] | ¹H-NMR (400 MHz, DMSO-d₆) δ = 1.56 (s, 3H), 2.64 (d, 3H), 5.83 (s, 2H), 7.19 (ddt, 1H), 7.54 (ddt, 1H), 8.39 (d, 1H). LC-MS (Method 1): Rₜ = 1.24 min MS (ESIpos): m/z = 536 [M + H]⁺ |
| 52A | 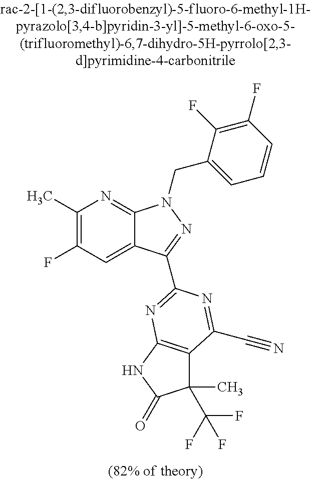 [SMILES: Cc1nc2c(cc1F)c(-c1nc(C#N)c3c(n1)NC(=O)C3(C)C(F)(F)F)nn2Cc1cccc(F)c1F] | ¹H-NMR (400 MHz, DMSO-d₆) δ = 1.82 (s, 3H), 2.66 (d, 3H), 5.90 (s, 2H), 7.04-7.09 (m, 1H), 7.14-7.23 (m, 1H), 7.36-7.45 (m, 1H), 8.42 (d, 1H), 12.85 (br. s, 1H). LC-MS (Method 1): Rₜ = 1.25 min MS (ESIpos): m/z = 518 [M + H]⁺ |
| 53A | 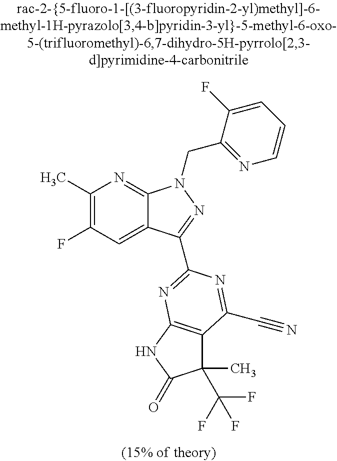 [SMILES: Cc1nc2c(cc1F)c(-c1nc(C#N)c3c(n1)NC(=O)C3(C)C(F)(F)F)nn2Cc1ncccc1F] | LC-MS (Method 1): Rₜ = 1.13 min MS (ESIpos): m/z = 501 [M + H]⁺ |

### Example 54A

### 2-[1-(2-Fluorobenzyl)-1H-pyrazolo[3,4-b]pyridin-3-yl]-5,5-dimethyl-6-oxo-6,7-dihydro-5H-pyrrolo[2,3-d]pyrimidine-4-carboxylic acid

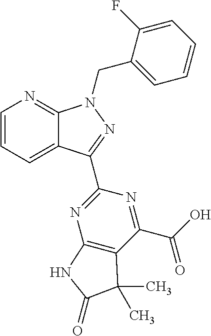 [SMILES: CC1(C)C(=O)Nc2nc(-c3nn(Cc4ccccc4F)c4ncccc34)nc(C(=O)O)c21]

A suspension of 9.0 g (20.86 mmol) of 2-[1-(2-fluorobenzyl)-1H-pyrazolo[3,4-b]pyridin-3-yl]-5,5-dimethyl-6-oxo-6,7-dihydro-5H-pyrrolo[2,3-d]pyrimidine-4-carboxamide (Example 1) in 180 ml of conc. hydrochloric acid was stirred at 80° C. for 20 h. Water and ethyl acetate were then added, the pH was adjusted to 2-3 using 20% strength aqueous sodium hydroxide solution and the phases were separated. The aqueous phase was extracted with ethyl acetate and the combined organic phases were dried and concentrated under reduced pressure. The residue was dissolved in dichloromethane/methanol (9:1) and purified by column chromatography (silica gel, dichloromethane and dichloromethane/methanol (5-20%) as mobile phase). The crude product obtained was suspended in diethyl ether and the resulting solid was filtered off with suction and dried under high vacuum. 7.50 g (83% of theory) of the title compound were obtained.

LC-MS (Method 7): Rₜ=1.13 min

MS (ESIpos): m/z=433 [M+H]⁺

¹H-NMR (400 MHz, DMSO-d₆): δ [ppm]=1.47 (s, 6H), 5.90 (s, 2H), 7.08-7.27 (m, 3H), 7.31-7.39 (m, 1H), 7.46 (dd, 1H), 8.68 (dd, 1H), 8.96 (dd, 1H), 11.80 (br. s, 1H), 14.10 (br. s, 1H).

### Example 55A

### 2-[5-Fluoro-1-(2-fluorobenzyl)-1H-pyrazolo[3,4-b]pyridin-3-yl]-5,5-dimethyl-6-oxo-6,7-dihydro-5H-pyrrolo[2,3-d]pyrimidine-4-carboxylic acid

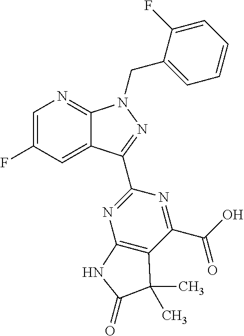 [SMILES: CC1(C)C(=O)Nc2nc(-c3nn(Cc4ccccc4F)c4ncc(F)cc34)nc(C(=O)O)c21]

A suspension of 2.02 g (4.5 mmol) of 2-[5-fluoro-1-(2-fluorobenzyl)-1H-pyrazolo[3,4-b]pyridin-3-yl]-5,5-dimethyl-6-oxo-6,7-dihydro-5H-pyrrolo[2,3-d]pyrimidine-4-carboxamide (Example 2) in 40 ml of conc. hydrochloric acid was stirred at 80° C. for 10 h. After cooling, the solid formed was filtered off with suction, washed with water and dried. 1.41 g (66% of theory) of the title compound were obtained.

LC-MS (Method 1): Rₜ=0.95 min

MS (ESIpos): m/z=451 [M+H]⁺

¹H-NMR (400 MHz, DMSO-d₆): δ [ppm]=1.49 (s, 6H), 5.87 (s, 2H), 7.11-7.29 (m, 3H), 7.31-7.44 (m, 1H), 8.69 (dd, 1H), 8.76 (dd, 1H), 11.90 (s, 1H), 14.05 (br. s, 1H).

### Example 56A

### 2-[1-(2,3-Difluorobenzyl)-5-fluoro-6-methyl-1H-pyrazolo[3,4-b]pyridin-3-yl]-5,5-dimethyl-6-oxo-6,7-dihydro-5H-pyrrolo[2,3-d]pyrimidine-4-carboxylic acid

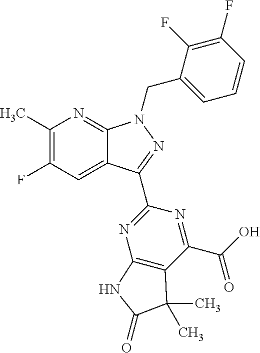 [SMILES: Cc1nc2c(cc1F)c(-c1nc3c(c(C(=O)O)n1)C(C)(C)C(=O)N3)nn2Cc1cccc(F)c1F]

A mixture of 386 mg (0.80 mmol) of 2-[1-(2,3-difluorobenzyl)-5-fluoro-6-methyl-1H-pyrazolo[3,4-b]pyridin-3-yl]-5,5-dimethyl-6-oxo-6,7-dihydro-5H-pyrrolo[2,3-d]pyrimidine-4-carboxamide from Example 4 in 19 ml of conc. hydrochloric acid and 19 ml of conc. acetic acid was stirred at 95° C. for 24 h. After cooling to RT, water was added to the mixture and the suspension formed was then stirred at RT for 30 min. The resulting solid was then filtered off, washed with water and dried under reduced pressure. This gave 416 mg (crude product; purity about 93%) of the title compound.

LC-MS (Method 11): Rₜ=12.19 min

MS (ESIpos): m/z=483 [M+H]⁺

¹H-NMR (400 MHz, DMSO-d₆): δ [ppm]=1.48 (s, 6H), 2.65 (d, 3H), 5.87 (s, 2H), 6.99-7.05 (m, 1H), 7.13-7.21 (m, 1H), 7.35-7.42 (m, 1H), 8.59 (d, 1H), 11.88 (s, 1H), 14.02 (br. s, 1H).

### Example 57A

### 2-{5-Fluoro-1-[(3-fluoropyridin-2-yl)methyl]-1H-pyrazolo[3,4-b]pyridin-3-yl}-5,5-dimethyl-6-oxo-6,7-dihydro-5H-pyrrolo[2,3-d]pyrimidine-4-carboxylic acid

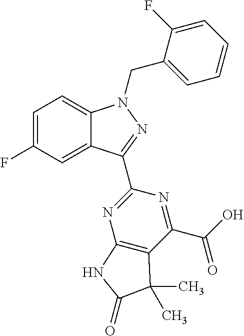 [SMILES: CC1(C)C(=O)Nc2nc(-c3nn(Cc4ccccc4F)c4ccc(F)cc34)nc(C(=O)O)c21]

A suspension of 245 mg (0.54 mmol) of 2-{5-fluoro-1-[(3-fluoropyridin-2-yl)methyl]-1H-pyrazolo[3,4-b]pyridin-3-yl}-5,5-dimethyl-6-oxo-6,7-dihydro-5H-pyrrolo[2,3-d]pyrimidine-4-carboxamide from Example 8 in 6.4 ml of conc. hydrochloric acid was stirred at 80° C. for 11 h. After cooling to RT, the solvent was removed under reduced pressure. The residue was taken up in water and a little acetonitrile and the mixture was stirred at 50° C. for 30 min. The resulting solid was filtered off, washed with water and dried. This gave 269 mg (96% of theory; purity 86%) of the title compound.

LC-MS (Method 11): Rₜ=9.89 min

MS (ESIpos): m/z=452 [M+H]⁺

¹H-NMR (400 MHz, DMSO-d₆): δ [ppm]=1.48 (s, 6H), 6.02 (s, 2H), 7.39-7.47 (m, 1H), 7.74-7.81 (m, 1H), 8.23-8.30 (m, 1H), 8.66-8.78 (m, 2H), 11.88 (s, 1H), 14.02 (br. s, 1H).

### Example 58A

### 2-[6-Chloro-1-(2-fluorobenzyl)-1H-indazol-3-yl]-5,5-dimethyl-6-oxo-6,7-dihydro-5H-pyrrolo[2,3-d]pyrimidine-4-carboxylic acid

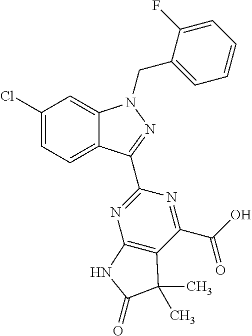 [SMILES: CC1(C)C(=O)Nc2nc(-c3nn(Cc4ccccc4F)c4cc(Cl)ccc34)nc(C(=O)O)c21]

1.83 g (3.94 mmol) of 2-[6-chloro-1-(2-fluorobenzyl)-1H-indazol-3-yl]-5,5-dimethyl-6-oxo-6,7-dihydro-5H-pyrrolo[2,3-d]pyrimidine-4-carboxamide (Example 3) were stirred in 20 ml of concentrated hydrochloric acid and 20 ml of conc. acetic acid at 95° C. for 18 h. With stirring, the warm reaction mixture was then carefully introduced into 250 ml of warm water at 70° C. After cool-cooling of the mixture, the solid formed was filtered off with suction, washed with water and dried. 1.57 g (86% of theory) of the title compound were obtained.

LC-MS (Method 2): Rₜ=3.19 min

MS (ESIpos): m/z=466 [M+H]⁺

¹H-NMR (400 MHz, DMSO-d₆): δ [ppm]=1.47 (s, 6H), 5.86 (s, 2H), 7.12-7.28 (m, 3H), 7.32-7.44 (m, 2H), 8.06 (d, 1H), 8.65 (d, 1H), 11.87 (s, 1H), 14.0 (br. s, 1H).

### Example 59A

### rac-2-[(Diphenylmethylene)amino]-4,4-difluorobutanonitrile

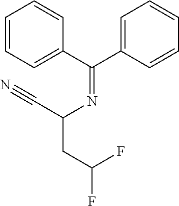 [SMILES: N#CC(CC(F)F)N=C(c1ccccc1)c1ccccc1]

18 g (81.72 mmol) of [(diphenylmethylene)amino]acetonitrile were initially charged in 500 ml of abs. THF, and 39.22 ml (98.06 mmol) of n-butyllithium (2.5 N in hexane) were added at −78° C. under argon, and the mixture was then stirred at −78° C. for 15 min. Subsequently, the reaction solution was brought to 0° C. 17.25 g (89.89 mmol) of 1,1-difluoro-2-iodoethane were added dropwise, and the mixture was then stirred at 0° C. for 15 min. At 0° C., first water and then ethyl acetate were then added to the reaction solution, and the mixture was washed three times with semisaturated aqueous sodium chloride solution. The combined aqueous phases were furthermore extracted twice with ethyl acetate. The combined organic phases were dried over sodium sulfate, filtered and concentrated. The residue was purified by means of column chromatography (silica gel, mobile phase: dichloromethane/cyclohexane=1/1). This gave 13.57 g of the target compound (49% of theory, purity 84%).

LC-MS (Method 3): Rt=2.48 min

MS (ESIpos): m/z=285 [M+H]+

¹H-NMR (400 MHz, DMSO-d₆): δ=2.53-2.61 (m, 2H; partially superposed by solvent peak), 4.50 (t, 1H), 6.08-6.41 (m, 1H), 7.23-7.33 (m, 2H), 7.38-7.47 (m, 2H), 7.49-7.67 (m, 6H).

### Example 60A

### rac-2-[(Diphenylmethylene)amino]-4,4-difluoro-2-methylbutanenitrile

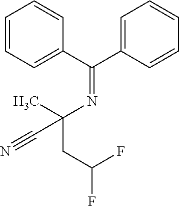 [SMILES: CC(C#N)(CC(F)F)N=C(c1ccccc1)c1ccccc1]

To an initial charge of 13.07 g (38.62 mmol) of rac-2-[(diphenylmethylene)amino]-4,4-difluorobutanonitrile from Example 59A in 255 ml of abs. THF were added 15.6 ml (39.0 mmol) of n-butyllithium (2.5 N in hexane) at −78° C. under argon, and the mixture was then stirred at −78° C. for 10 min. Subsequently, 22.6 g (154.46 mmol) of iodomethane were added to the reaction solution at −78° C. The reaction mixture was gradually brought to 0° C. over 3.5 h. At 0° C., first water and then ethyl acetate were then added, and the mixture was washed twice with saturated aqueous sodium chloride solution. The organic phase was dried over sodium sulfate, filtered and concentrated. The residue was purified by means of column chromatography (silica gel, mobile phase: cyclohexane/ethyl acetate=15/1). This gave 11.4 g of the target compound (91% of theory, purity 92%).

LC-MS (Method 3): Rₜ=2.52 min

MS (ESIpos): m/z=299 [M+H]⁺

¹H-NMR (400 MHz, DMSO-d₆): δ=1.67 (s, 3H), 2.55-2.77 (m, 2H), 6.14-6.48 (m, 1H), 7.28-7.34 (m, 2H), 7.36-7.44 (m, 2H), 7.44-7.54 (m, 6H).

### Example 61A

### rac-2-Amino-4,4-difluoro-2-methylbutanonitrile hydrochloride

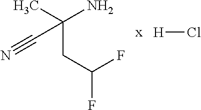

10.84 g (33.43 mmol; 92% purity) of rac-2-[(diphenylmethylene)amino]-4,4-difluoro-2-methylbutanonitrile from Example 60A were dissolved in 156 ml of tetrahydrofuran and 6 ml of water, 73.5 ml (36.77 mmol) of hydrogen chloride solution (0.5 N in diethyl ether) were added and the mixture was stirred at room temperature overnight. 16.71 ml (33.43 mmol) of hydrogen chloride solution (2 N in diethyl ether) were then added to the reaction solution, and the mixture was concentrated. The isolated crude product was reacted further directly without further purification.

LC-MS (Method 3): Rₜ=0.32 min

MS (ESIpos): m/z=135 (M−HCl+H)⁺

### Example 62A

### rac-Benzyl (2-cyano-4,4-difluorobutan-2-yl)carbamate

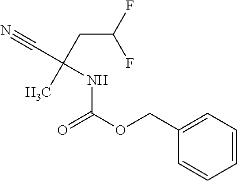 [SMILES: CC(C#N)(CC(F)F)NC(=O)OCc1ccccc1]

The crude product rac-2-amino-4,4-difluoro-2-methylbutanonitrile hydrochloride from Example 61A was initially charged in 109 ml of tetrahydrofuran/water (1:1), and 18.94 g (137.06 mmol) of potassium carbonate and 6.27 g (36.77 mmol) of benzyl chloroformate were added. The reaction mixture was stirred at room temperature overnight. Another 1.14 g (6.69 mmol) of benzyl chloroformate were added to the reaction and the mixture was stirred at room temperature for a further 2 h. The phases were then separated and the aqueous phase was extracted twice with ethyl acetate. The combined organic phases were washed once with saturated aqueous sodium chloride solution, and then dried over sodium sulfate, filtered and concentrated. The residue was purified by column chromatography (mobile phase: cyclohexane/ethyl acetate gradient 20/1 to 5/1). This gave 7.68 g of the target compound (61% of theory over two steps, purity 71%).

LC-MS (Method 3): Rₜ=2.04 min

MS (ESIpos): m/z=269 [M+H]⁺

¹H-NMR (400 MHz, DMSO-d₆): δ [ppm]=1.65 (s, 3H), 2.51-2.65 (m, 2H), 5.10 (s, 2H), 6.08-6.41 (m, 1H), 7.27-7.44 (m, 5H), 8.24 (br. s, 1H).

### Example 63A

### ent-Benzyl (2-cyano-4,4-difluorobutan-2-yl)carbamate (enantiomer A)

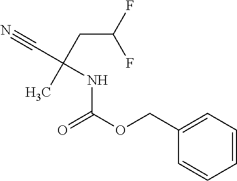 [SMILES: CC(C#N)(CC(F)F)NC(=O)OCc1ccccc1]

7.68 g (20.33 mmol, purity 71%) of rac-benzyl (2-cyano-4,4-difluorobutan-2-yl)carbamate from Example 62A were separated into the enantiomers by preparative separation on the chiral phase [column: Daicel Chiralpak AY-H, 5 μm, 250×20 mm, mobile phase: 80% isohexane, 20% isopropanol; flow rate: 25 ml/min; temperature: 22° C., detection: 210 nm].

Enantiomer A: yield: 2.64 g (>99% ee)

Rₜ=6.67 min [Chiralpak AY-H, 5 μm, 250×4.6 mm; mobile phase: 80% isohexane, 20% isopropanol; flow rate: 3 ml/min; detection: 220 nm].

### Example 64A

### ent-Benzyl (2-cyano-4,4-difluorobutan-2-yl)carbamate (enantiomer B)

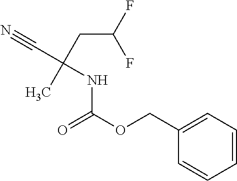 [SMILES: CC(C#N)(CC(F)F)NC(=O)OCc1ccccc1]

7.68 g (20.33 mmol, purity 71%) of rac-benzyl (2-cyano-4,4-difluorobutan-2-yl)carbamate from Example 62A were separated into the enantiomers by preparative separation on the chiral phase [column: Daicel Chiralpak AY-H, 5 μm, 250×20 mm, mobile phase: 80% isohexane, 20% isopropanol; flow rate: 25 ml/min; temperature: 22° C., detection: 210 nm].

Enantiomer B: yield: 2.76 g (93% ee)

Rₜ=7.66 min [Chiralpak AY-H, 5 μm, 250×4.6 mm; mobile phase: 80% isohexane, 20% isopropanol; flow rate: 3 ml/min; detection: 220 nm].

### Example 65A

### ent-Benzyl (1-amino-4,4-difluoro-2-methylbutan-2-yl)carbamate (enantiomer A)

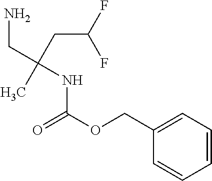 [SMILES: CC(CN)(CC(F)F)NC(=O)OCc1ccccc1]

2.3 g (8.57 mmol) of ent-benzyl (2-cyano-4,4-difluorobutan-2-yl)carbamate (enantiomer A) from Example 63A were dissolved in 75 ml of methanolic ammonia solution (7 N in methanol), and 2.66 g of Raney nickel (50% aqueous slurry) were added under argon. The reaction mixture was hydrogenated in an autoclave at 20-30 bar for 1.5 h. The reaction mixture was filtered through Celite, rinsed with methanol and methanolic ammonia solution (2 N in methanol) and concentrated. This gave 2.23 g of the target compound (94% of theory).

LC-MS (Method 3): Rₜ=1.48 min

MS (ESIpos): m/z=273 [M+H]⁺

¹H-NMR (400 MHz, DMSO-d₆): δ [ppm]=1.19 (s, 3H), 1.48 (br. s, 2H), 2.08-2.40 (m, 2H), 2.53 2.72 (m, 2H; partially superposed by solvent peak), 5.00 (s, 2H), 5.90-6.23 (m, 1H), 6.95 (br. s, 1H), 7.25-7.41 (m, 5H).

### Example 66A

### ent-Benzyl (1-amino-4,4-difluoro-2-methylbutan-2-yl)carbamate (enantiomer B)

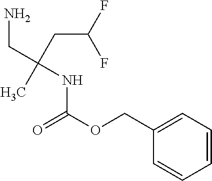 [SMILES: CC(CN)(CC(F)F)NC(=O)OCc1ccccc1]

2.76 g (10.29 mmol) of ent-benzyl (2-cyano-4,4-difluorobutan-2-yl)carbamate (enantiomer B) from Example 64A were dissolved in 90 ml of methanolic ammonia solution (7 N in methanol), and 3.19 g of Raney nickel (50% aqueous slurry) were added under argon. The reaction mixture was hydrogenated in an autoclave at 20-30 bar of hydrogen for 1.5 h. The reaction mixture was filtered through Celite and rinsed with methanol and methanolic ammonia solution (2 N in methanol), and the mixture was concentrated. This gave 2.64 g of the target compound (88% of theory, purity 93%).

LC-MS (Method 3): Rₜ=1.49 min

MS (ESIpos): m/z=273 [M+H]⁺

¹H-NMR (400 MHz, DMSO-d₆): δ [ppm]=1.19 (s, 3H), 1.48 (br. s, 2H), 2.08-2.40 (m, 2H), 2.53 2.73 (m, 2H; partially superposed by solvent peak), 5.00 (s, 2H), 5.90-6.24 (m, 1H), 6.95 (br. s, 1H), 7.25-7.41 (m, 5H).

### Example 67A

### ent-Benzyl (4,4-difluoro-1-{[(2-{5-fluoro-1-[(3-fluoropyridin-2-yl)methyl]-1H-pyrazolo[3,4-b]pyridin-3-yl}-5,5-dimethyl-6-oxo-6,7-dihydro-5H-pyrrolo[2,3-d]pyrimidin-4-yl)carbonyl]amino}-2-methylbutan-2-yl)carbamate (enantiomer A)

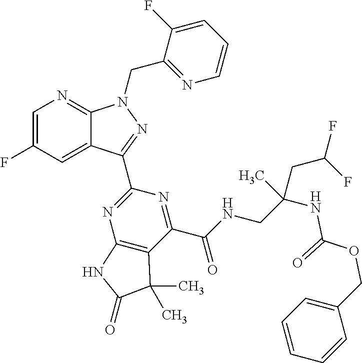 [SMILES: CC(CNC(=O)c1nc(-c2nn(Cc3ncccc3F)c3ncc(F)cc23)nc2c1C(C)(C)C(=O)N2)(CC(F)F)NC(=O)OCc1ccccc1]

25 mg (0.05 mmol) of 2-{5-fluoro-1-[(3-fluoropyridin-2-yl)methyl]-1H-pyrazolo[3,4-b]pyridin-3-yl}-5,5-dimethyl-6-oxo-6,7-dihydro-5H-pyrrolo[2,3-d]pyrimidine-4-carboxylic acid from Example 57A, 39 mg (0.14 mmol) of ent-benzyl (1-amino-4,4-difluoro-2-methylbutan-2-yl)carbamate (enantiomer A) from Example 65A and 40 μl (0.29 mmol) of triethylamine were dissolved in 0.3 ml of DMF, 43 μl (0.07 mmol) of 2,4,6-tripropyl-1,3,5,2,4,6-trioxatriphosphinane 2,4,6-trioxide (T3P, 50% solution in ethyl acetate) were added and the mixture was stirred at RT for 3 h. Another 39 mg (0.14 mmol) ent-benzyl (1-amino-4,4-difluoro-2-methylbutan-2-yl)carbamate (enantiomer A), 20 μl (0.14 mmol) of triethylamine and 23 μl (0.04 mmol) of 2,4,6-tripropyl-1,3,5,2,4,6-trioxatriphosphinane 2,4,6-trioxide (T3P, 50% solution in ethyl acetate) were added and the reaction mixture was stirred at RT overnight. Acetonitrile/water and TFA were then added and the reaction mixture was purified by preparative HPLC (RP18 column, mobile phase: acetonitrile/water gradient with addition of 0.1% TFA). This gave 26 mg of the target compound (63% of theory, purity 80%).

LC-MS (Method 1): Rₜ=1.19 min

MS (ESIpos): m/z=706.5 [M+H]⁺

The exemplary compounds listed in Table 10A were prepared analogously to the procedure from Example 67A from the acids of the starting materials 56A, 57A and the appropriate amines (Examples 65A and 66A). If appropriate, further amine (1-3 equivalents), 2,4,6-tripropyl-1,3,5,2,4,6-trioxatriphosphinane 2,4,6-trioxide (50% in ethyl acetate) (0.5-1.0 equivalent) and triethylamine (2-4 equivalents) were added to the reaction mixtures and stirring was continued until the reaction had gone to completion (1-24 h). Purifications were carried out by preparative HPLC (RP18 column, mobile phase: acetonitrile/water gradient with addition of 0.1% formic acid or 0.1% TFA).

TABLE 10 A

| Ex- | IUPAC name/structure | Workup, |
|---|---|---|
| ample | (Yield) | Analytical data |
| 68A | ent-Benzyl (4,4-difluoro-1-{[(2-{5-fluoro-1-[(3- | LC-MS (Method 1): Rₜ = 1.19 min |
| | fluoropyridin-2-yl)methyl]-1H-pyrazolo[3,4-b]pyridin-3- | MS (ESIpos): m/z = 706.5 [M + H]⁺ |
| | yl}-5,5-dimethyl-6-oxo-6,7-dihydro-5H-pyrrolo[2,3- | |
| | d]pyrimidin-4-yl)carbonyl]amino}-2-methylbutan-2- | |
| | yl)carbamate (enantiomer B) ¹⁾ | |
| | 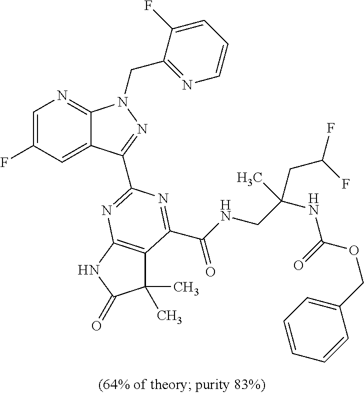 [SMILES: CC(CNC(=O)c1nc(-c2nn(Cc3ncccc3F)c3ncc(F)cc23)nc2c1C(C)(C)C(=O)N2)(CC(F)F)NC(=O)OCc1ccccc1] | |
| 69A | ent-Benzyl {1-[({2-[1-(2,3-difluorobenzyl)-5-fluoro-6- | LC-MS (Method 1): Rₜ = 1.28 min |
| | methyl-1H-pyrazolo[3,4-b]pyridin-3-yl]-5,5-dimethyl-6- | MS (ESIpos): m/z = 737 [M + H]⁺ |
| | oxo-6,7-dihydro-5H-pyrrolo[2,3-d]pyrimidin-4- | |
| | yl}carbonyl)amino]-4,4-difluoro-2-methylbutan-2- | |
| | yl}carbamate (enantiomer A) ²⁾ | |
| | 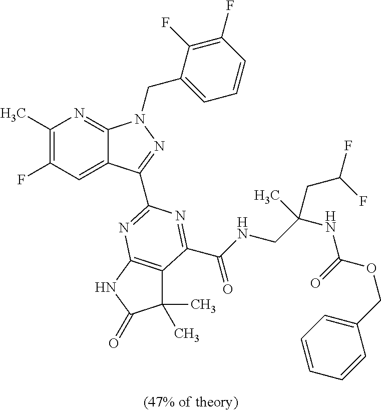 [SMILES: Cc1nc2c(cc1F)c(-c1nc3c(c(C(=O)NCC(C)(CC(F)F)NC(=O)OCc4ccccc4)n1)C(C)(C)C(=O)N3)nn2Cc1cccc(F)c1F] | |
| 70A | ent-Benzyl {1-[({2-[1-(2,3-difluorobenzyl)-5-fluoro-6- | LC-MS (Method 1): Rₜ = 1.28 min |
| | methyl-1H-pyrazolo[3,4-b]pyridin-3-yl]-5,5-dimethyl-6- | MS (ESIpos): m/z = 737 [M + H]⁺ |
| | oxo-6,7-dihydro-5H-pyrrolo[2,3-d]pyrimidin-4- | |
| | yl}carbonyl)amino]-4,4-difluoro-2-methylbutan-2- | |
| | yl}carbamate (enantiomer B) ¹⁾ | |
| | 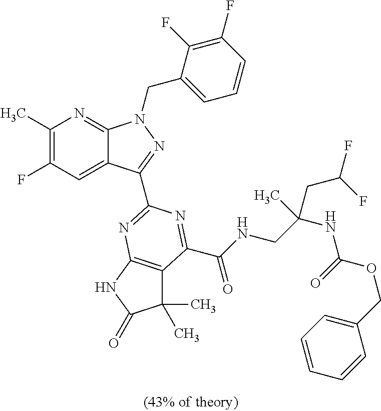 [SMILES: Cc1nc2c(cc1F)c(-c1nc3c(c(C(=O)NCC(C)(CC(F)F)NC(=O)OCc4ccccc4)n1)C(C)(C)C(=O)N3)nn2Cc1cccc(F)c1F] | |

¹⁾ ent-Benzyl (1-amino-4,4-difluoro-2-methylbutan-2-yl)carbamate (enantiomer B) from Example 66A was employed.
²⁾ ent-Benzyl (1-amino-4,4-difluoro-2-methylbutan-2-yl)carbamate (enantiomer A) from Example 65A was employed.

### WORKING EXAMPLES

### Example 1

### 2-[1-(2-Fluorobenzyl)-1H-pyrazolo[3,4-b]pyridin-3-yl]-5,5-dimethyl-6-oxo-6,7-dihydro-5H-pyrrolo[2,3-d]pyrimidine-4-carboxamide

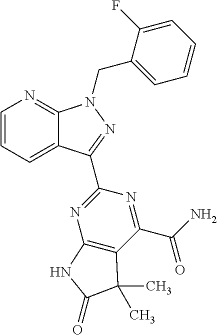 [SMILES: CC1(C)C(=O)Nc2nc(-c3nn(Cc4ccccc4F)c4ncccc34)nc(C(N)=O)c21]

11.5 g (27.8 mmol) of 2-[1-(2-fluorobenzyl)-1H-pyrazolo[3,4-b]pyridin-3-yl]-5,5-dimethyl-6-oxo-6,7-dihydro-5H-pyrrolo[2,3-d]pyrimidine-4-carbonitrile (Ex. 41A) in 100 ml of dioxane and 35 ml of 2 M aqueous sodium hydroxide solution were stirred at 80° C. overnight. The reaction mixture was poured into a mixture of 10% aqueous sodium chloride solution and ethyl acetate and, with stirring, adjusted to pH 3 using semiconcentrated hydrochloric acid. The resulting precipitate was filtered off, washed with ethyl acetate and dried. This gave 9.0 g (75% of theory) of the title compound. The phases of the fitrate were separated, the aqueous phase was re-extracted once with ethyl acetate, the combined organic phases were dried and the solvent was removed under reduced pressure, giving a further 3.1 g of crude product (15% of theory, purity 59%).

LC-MS (Method 7): Rₜ=1.12 min

MS (ESIpos): m/z=432 [M+H]⁺

¹H-NMR (400 MHz, DMSO-d₆) δ [ppm]=1.50 (s, 6H), 5.88 (s, 2H), 7.10-7.27 (m, 3H), 7.32-7.41 (m, 1H), 7.46 (dd, 1H), 8.05 (br. s, 1H), 8.10 (br. s, 1H), 8.69 (dd, 1H), 8.93 (dd, 1H), 11.86 (s, 1H)

### Example 2

### 2-[5-Fluoro-1-(2-fluorobenzyl)-1H-pyrazolo[3,4-b]pyridin-3-yl]-5,5-dimethyl-6-oxo-6,7-dihydro-5H-pyrrolo[2,3-d]pyrimidine-4-carboxamide

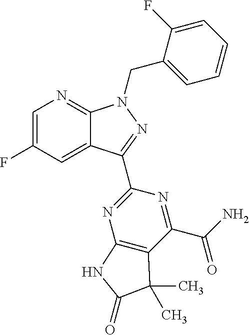 [SMILES: CC1(C)C(=O)Nc2nc(-c3nn(Cc4ccccc4F)c4ncc(F)cc34)nc(C(N)=O)c21]

2.11 g (purity 75%, 3.67 mmol) of 2-[5-fluoro-1-(2-fluorobenzyl)-1H-pyrazolo[3,4-b]pyridin-3-yl]-5,5-dimethyl-6-oxo-6,7-dihydro-5H-pyrrolo[2,3-d]pyrimidine-4-carbonitrile (described in WO 2013/104703, Ex. 81A) in 70 ml of dioxane and 24 ml of 2 M aqueous sodium hydroxide solution were stirred at 80° C. for 6 h. The reaction mixture was then adjusted to pH 5 using formic acid and concentrated under reduced pressure, and the residue was subsequently diluted with 100 ml of water. The precipitate formed was then filtered off with suction and dried. The resulting solid was suspended in 50 ml of petroleum ether and 2 ml of dichloromethane and then filtered off with suction and dried. This gave 2.23 g of crude product which was reacted further to give the compound from Example 55A. Pure material was obtained by preparative HPLC (RP18, gradient of water+0.1% formic acid/acetonitrile (5-95%)).

LC-MS (Method 1): Rₜ=0.94 min

MS (ESIpos): m/z=450 [M+H]⁺

¹H-NMR (400 MHz, DMSO-d₆): δ [ppm]=1.50 (s, 6H), 5.88 (s, 2H), 7.11-7.29 (m, 3H), 7.32-7.42 (m, 1H), 8.01 (br. s, 1H), 8.20 (br. s, 1H), 8.69 (dd, 1H), 8.75 (dd, 1H), 11.84 (s, 1H).

### Example 3

### 2-[6-Chloro-1-(2-fluorobenzyl)-1H-indazol-3-yl]-5,5-dimethyl-6-oxo-6,7-dihydro-5H-pyrrolo[2,3-d]pyrimidine-4-carboxamide

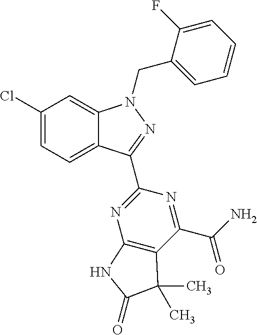 [SMILES: CC1(C)C(=O)Nc2nc(-c3nn(Cc4ccccc4F)c4cc(Cl)ccc34)nc(C(N)=O)c21]

1.69 g (3.77 mmol) of 2-[6-chloro-1-(2-fluorobenzyl)-1H-indazol-3-yl]-5,5-dimethyl-6-oxo-6,7-dihydro-5H-pyrrolo[2,3-d]pyrimidine-4-carbonitrile (Ex. 48A) were stirred in 12 ml of dioxane and 4 ml of 2 M aqueous sodium hydroxide solution at 80° C. for 5 h. The reaction mixture was then adjusted to pH 5 using formic acid, the reaction mixture was concentrated under reduced pressure, water was then added to the residue and the resulting suspension was stirred at 50° C. After cooling to RT, the precipitate formed was filtered off with suction and dried. This gave 1.83 g of crude product which was reacted further to give the compound from Example 58A. Pure material was obtained by preparative HPLC (RP18, gradient of water+0.1% formic acid/acetonitrile (5-95%)).

LC-MS (Method 1): Rₜ=1.05 min

¹H-NMR (400 MHz, DMSO-d₆): δ [ppm]=1.49 (s, 6H), 5.86 (s, 2H), 7.12-7.27 (m, 3H), 7.33-7.42 (m, 2H), 8.01-8.08 (m, 1H), 8.54 (d, 1H), 11.83 (s, 1H).

### Example 4

### 2-[1-(2,3-Difluorobenzyl)-5-fluoro-6-methyl-1H-pyrazolo[3,4-b]pyridin-3-yl]-5,5-dimethyl-6-oxo-6,7-dihydro-5H-pyrrolo[2,3-d]pyrimidine-4-carboxamide

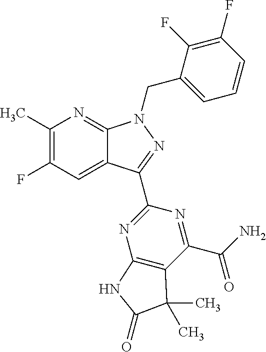 [SMILES: Cc1nc2c(cc1F)c(-c1nc3c(c(C(N)=O)n1)C(C)(C)C(=O)N3)nn2Cc1cccc(F)c1F]

424 mg (0.86 mmol; purity 93%) of 2-[1-(2,3-difluorobenzyl)-5-fluoro-6-methyl-1H-pyrazolo[3,4-b]pyridin-3-yl]-5,5-dimethyl-6-oxo-6,7-dihydro-5H-pyrrolo[2,3-d]pyrimidine-4-carbonitrile from Example 43A were initially charged in 13 ml of abs. dioxane, 3.23 ml (6.46 mmol) of 2 N aqueous sodium hydroxide solution were added and the mixture was stirred at 90° C. for 10 h. The reaction solution was cooled to RT and diluted with 1 ml of 1 M aqueous sodium hydroxide solution. A further 1.08 ml (2.16 mmol) of 2 N sodium hydroxide solution were added and the mixture was then stirred at 90° C. for a further 8 h. The mixture was adjusted to pH 3 using 1 N hydrochloric acid. The suspension was freed from the dioxane on a rotary evaporator. The solid obtained was then filtered off. This gave 413 mg (95% of theory, purity 95%) of the title compound.

LC-MS (Method 1): Rₜ=0.97 min

MS (ESIpos): m/z=482 [M+H]⁺

¹H-NMR (500 MHz, DMSO-d₆): δ [ppm]=1.49 (s, 6H), 2.64 (d, 3H), 5.88 (s, 2H), 6.99-7.05 (m, 1H), 7.13-7.20 (m, 1H), 7.37-7.42 (m, 1H), 8.01 (br. s, 1H), 8.19 (br. s, 1H), 8.59 (d, 1H), 11.82 (br. s, 1H).

### Example 5

### 2-[5-Fluoro-1-(2-fluorobenzyl)-6-methyl-1H-pyrazolo[3,4-b]pyridin-3-yl]-5,5-dimethyl-6-oxo-6,7-dihydro-5H-pyrrolo[2,3-d]pyrimidine-4-carboxamide

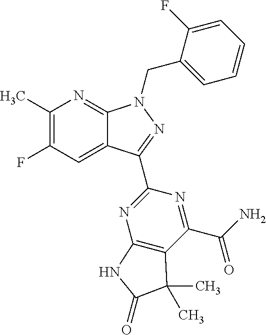 [SMILES: Cc1nc2c(cc1F)c(-c1nc3c(c(C(N)=O)n1)C(C)(C)C(=O)N3)nn2Cc1ccccc1F]

39 mg (0.09 mmol) of 2-[5-fluoro-1-(2-fluorobenzyl)-6-methyl-1H-pyrazolo[3,4-b]pyridin-3-yl]-5,5-dimethyl-6-oxo-6,7-dihydro-5H-pyrrolo[2,3-d]pyrimidine-4-carbonitrile from Example 44A were initially charged in 1.7 ml of abs. dioxane, 0.70 ml (1.40 mmol) of 2 N aqueous sodium hydroxide solution were added and the mixture was stirred at 80° C. for 6 h. The reaction solution was cooled and diluted with 5 ml of 1 N aqueous sodium hydroxide solution. The mixture was subsequently adjusted to pH 5 using saturated aqueous ammonium chloride solution. The solid obtained was filtered off, washed with water and dried under high vacuum. This gave 36 mg (85% of theory; purity 96%) of the title compound.

LC-MS (Method 7): Rₜ=1.29 min

MS (ESIpos): m/z=464 [M+H]⁺

¹H-NMR (400 MHz, DMSO-d₆): δ [ppm]=1.46 (s, 6H), 2.64 (d, 3H), 5.83 (s, 2H), 7.12-7.20 (m, 2H), 7.20-7.28 (m, 1H), 7.33-7.40 (m, 1H), 7.95 (br. s, 1H), 8.15 (br. s, 1H), 8.58 (d, 1H), 10.83 (br. s, 1H).

### Example 6

### 2-[5-Fluoro-6-methyl-1-(2,3,6-trifluorobenzyl)-1H-pyrazolo[3,4-b]pyridin-3-yl]-5,5-dimethyl-6-oxo-6,7-dihydro-5H-pyrrolo[2,3-d]pyrimidine-4-carboxamide

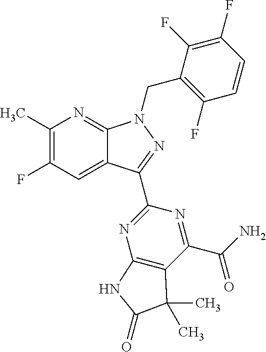 [SMILES: Cc1nc2c(cc1F)c(-c1nc3c(c(C(N)=O)n1)C(C)(C)C(=O)N3)nn2Cc1c(F)ccc(F)c1F]

180 mg (0.37 mmol) of 2-[5-fluoro-6-methyl-1-(2,3,6-trifluorobenzyl)-1H-pyrazolo[3,4-b]pyridin-3-yl]-5,5-dimethyl-6-oxo-6,7-dihydro-5H-pyrrolo[2,3-d]pyrimidine-4-carbonitrile from Example 45A were initially charged in 7 ml of abs. dioxane, 3.0 ml (6.00 mmol) of 2 N aqueous sodium hydroxide solution were added and the mixture was stirred at RT overnight and at 80° C. for 5 h. The reaction solution was cooled and diluted with 5 ml of 1 N aqueous sodium hydroxide solution. The mixture was subsequently adjusted to pH 5 using saturated aqueous ammonium chloride solution. The suspension was freed from the dioxane on a rotary evaporator. The solid obtained was then filtered off. This solid was washed with water and dried under high vacuum. This gave 149 mg (73% of theory; purity 92%) of the title compound.

LC-MS (Method 1): Rₜ=1.02 min

MS (ESIpos): m/z=500 [M+H]⁺

¹H-NMR (400 MHz, DMSO-d₆): δ [ppm]=1.48 (s, 6H), 2.65 (d, 3H), 5.86 (s, 2H), 7.16-7.24 (m, 1H), 7.55 (ddt, 1H), 8.00 (br. s, 1H), 8.17 (br. s, 1H), 8.56 (d, 1H), 11.82 (br. s, 1H).

### Example 7

### 2-{5-Fluoro-1-[(3-fluoropyridin-2-yl)methyl]-6-methyl-1H-pyrazolo[3,4-b]pyridin-3-yl}-5,5-dimethyl-6-oxo-6,7-dihydro-5H-pyrrolo[2,3-d]pyrimidine-4-carboxamide

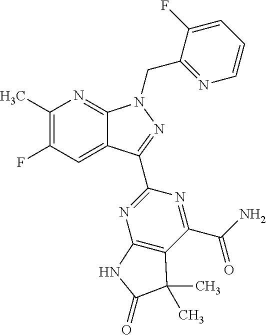 [SMILES: Cc1nc2c(cc1F)c(-c1nc3c(c(C(N)=O)n1)C(C)(C)C(=O)N3)nn2Cc1ncccc1F]

55 mg (0.12 mmol) of 2-{5-fluoro-1-[(3-fluoropyridin-2-yl)methyl]-6-methyl-1H-pyrazolo[3,4-b]pyridin-3-yl}-5,5-dimethyl-6-oxo-6,7-dihydro-5H-pyrrolo[2,3-d]pyrimidine-4-carbonitrile from Example 46A were initially charged in 2.4 ml of abs. dioxane, 0.305 ml (0.61 mmol) of 2 N aqueous sodium hydroxide solution were added and the mixture was stirred at 90° C. for 13 h. A further 0.061 ml (0.122 mmol) of 2 N sodium hydroxide solution were added and the mixture was then stirred at 90° C. for 5 h. A further 0.091 ml (0.182 mmol) of 2 N aqueous sodium hydroxide solution was added and the mixture was then stirred at 90° C. for 4 h. The reaction solution was concentrated by evaporation, water/acetonitrile was added and the mixture was purified by preparative HPLC (column: RP18, mobile phase: acetonitrile/water gradient with addition of 0.1% TFA). 43 mg (75% of theory) of the title compound were obtained.

LC-MS (Method 1): Rₜ=0.86 min

MS (ESIpos): m/z=465 [M+H]⁺

¹H-NMR (400 MHz, DMSO-d₆): δ [ppm]=1.49 (s, 6H), 2.60 (d, 3H), 5.97 (s, 2H), 7.39-7.46 (m, 1H), 7.72-7.82 (m, 1H), 7.99 (br. s, 1H), 8.19 (br. s, 1H), 8.28 (d, 1H), 8.59 (d, 1H), 11.80 (br. s, 1H).

### Example 8

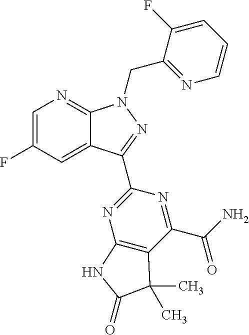 [SMILES: CC1(C)C(=O)Nc2nc(-c3nn(Cc4ncccc4F)c4ncc(F)cc34)nc(C(N)=O)c21]

319 mg (0.74 mmol) of 2-{5-fluoro-1-[(3-fluoropyridin-2-yl)methyl]-1H-pyrazolo[3,4-b]pyridin-3-yl}-5,5-dimethyl-6-oxo-6,7-dihydro-5H-pyrrolo[2,3-d]pyrimidine-4-carbonitrile from Example 47A were initially charged in 10.5 ml of abs. dioxane, 1.85 ml (3.70 mmol) of 2 N aqueous sodium hydroxide solution were added and the mixture was stirred at 90° C. for 13 h. The reaction solution was cooled and the organic solvent was evaporated. Ethyl acetate was then added and the mixture was adjusted to pH 3 using 1 N hydrochloric acid. The solid obtained was filtered off and washed with water. This gave 258 mg (77% of theory) of the title compound.

LC-MS (Method 1): Rₜ=0.80 min

MS (ESIpos): m/z=451 [M+H]⁺

¹H-NMR (400 MHz, DMSO-d₆): δ [ppm]=1.50 (s, 6H), 6.02 (s, 2H), 7.41-7.47 (m, 1H), 7.74-7.81 (m, 1H), 8.01 (br. s, 1H), 8.20 (br. s, 1H), 8.24-8.29 (m, 1H), 8.66-8.73 (m, 2H), 11.82 (br. s, 1H).

### Example 9

### rac-2-[1-(2,3-difluorobenzyl)-5-fluoro-6-methyl-1H-pyrazolo[3,4-b]pyridin-3-yl]-5-methyl-6-oxo-5-(trifluoromethyl)-6,7-dihydro-5H-pyrrolo[2,3-d]pyrimidine-4-carboxamide

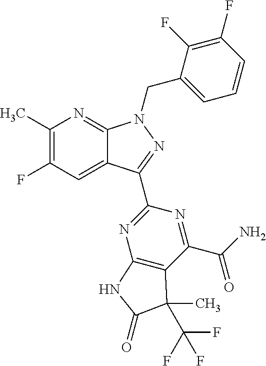 [SMILES: Cc1nc2c(cc1F)c(-c1nc3c(c(C(N)=O)n1)C(C)(C(F)(F)F)C(=O)N3)nn2Cc1cccc(F)c1F]

340 mg (0.66 mmol) of rac-2-[1-(2,3-difluorobenzyl)-5-fluoro-6-methyl-1H-pyrazolo[3,4-b]pyridin-3-yl]-5-methyl-6-oxo-5-(trifluoromethyl)-6,7-dihydro-5H-pyrrolo[2,3-d]pyrimidine-4-carbonitrile from Example 52A were initially charged in 10 ml of abs. dioxane, 1.64 ml (3.28 mmol) of 2 N aqueous sodium hydroxide solution were added and the mixture was stirred at 90° C. for 5.5 h. A further 0.82 ml (0.164 mmol) of 2 N sodium hydroxide solution were added and the mixture was then stirred at 90° C. for 4 h. The volatile constituents were removed under reduced pressure, and water/acetonitrile/TFA and a little methanol were then added to the residue. The precipitate formed was filtered off and dried. This gave 333 mg (93% of theory) of the title compound.

LC-MS (Method 1): Rₜ=1.06 min

MS (ESIpos): m/z=536 [M+H]⁺

¹H-NMR (500 MHz, DMSO-d₆): δ [ppm]=1.90 (s, 3H), 2.63 (d, 3H), 5.89 (s, 2H), 7.02-7.08 (m, 1H), 7.14-7.21 (m, 1H), 7.37-7.43 (m, 1H), 7.98 (br. s, 1H), 8.29 (br. s, 1H), 8.55 (d, 1H), 12.48 (br. s, 1H).

### Example 10

### ent-2-[1-(2,3-Difluorobenzyl)-5-fluoro-6-methyl-1H-pyrazolo[3,4-b]pyridin-3-yl]-5-methyl-6-oxo-5-(trifluoromethyl)-6,7-dihydro-5H-pyrrolo[2,3-d]pyrimidine-4-carboxamide (enantiomer A)

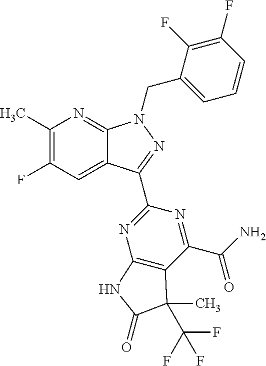 [SMILES: Cc1nc2c(cc1F)c(-c1nc3c(c(C(N)=O)n1)C(C)(C(F)(F)F)C(=O)N3)nn2Cc1cccc(F)c1F]

300 mg of rac-2-[1-(2,3-difluorobenzyl)-5-fluoro-6-methyl-1H-pyrazolo[3,4-b]pyridin-3-yl]-5-methyl-6-oxo-5-(trifluoromethyl)-6,7-dihydro-5H-pyrrolo[2,3-d]pyrimidine-4-carboxamide (Example 9) were separated on a chiral phase into the enantiomers [SFC column: Daicel Chiralpak IB, 5 μm, 250×20 mm, mobile phase: 82% CO₂, 18% ethanol, flow rate 50 ml/min; 40° C., detection: 210 nm].

Enantiomer A: 107 mg (>99% ee)

Rₜ=2.07 min [SFC: Daicel Chiralpak IB, 5 μm, 250×4.6 mm; mobile phase: 5→60% ethanol; flow rate 3.0 ml/min; detection: 220 nm].

### Example 11

### ent-2-[1-(2,3-Difluorobenzyl)-5-fluoro-6-methyl-1H-pyrazolo[3,4-b]pyridin-3-yl]-5-methyl-6-oxo-5-(trifluoromethyl)-6,7-dihydro-5H-pyrrolo[2,3-d]pyrimidine-4-carboxamide (enantiomer B)

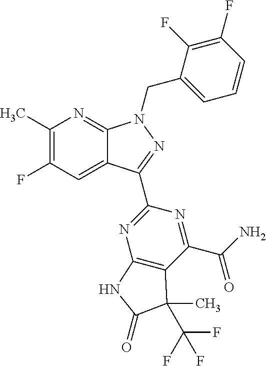 [SMILES: Cc1nc2c(cc1F)c(-c1nc3c(c(C(N)=O)n1)C(C)(C(F)(F)F)C(=O)N3)nn2Cc1cccc(F)c1F]

300 mg of rac-2-[1-(2,3-difluorobenzyl)-5-fluoro-6-methyl-1H-pyrazolo[3,4-b]pyridin-3-yl]-5-methyl-6-oxo-5-(trifluoromethyl)-6,7-dihydro-5H-pyrrolo[2,3-d]pyrimidine-4-carboxamide (Example 9) were separated on a chiral phase into the enantiomers [SFC column: Daicel Chiralpak IB, 5 μm, 250×20 mm, mobile phase: 82% CO₂, 18% ethanol, flow rate 50 ml/min; 40° C., detection: 210 nm].

Enantiomer B: 105 mg (96% ee)

Rₜ=2.16 min [SFC: Daicel Chiralpak IB, 5 μm, 250×4.6 mm; mobile phase: 5→60% ethanol; flow rate 3.0 ml/min; detection: 220 nm].

### Example 12

### rac-2-[5-fluoro-1-(2-fluorobenzyl)-6-methyl-1H-pyrazolo[3,4-b]pyridin-3-yl]-5-methyl-6-oxo-5-(trifluoromethyl)-6,7-dihydro-5H-pyrrolo[2,3-d]pyrimidine-4-carboxamide

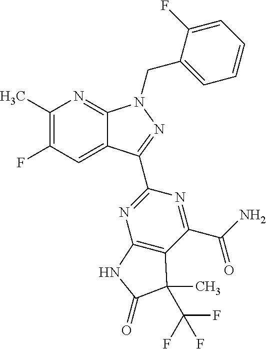 [SMILES: Cc1nc2c(cc1F)c(-c1nc3c(c(C(N)=O)n1)C(C)(C(F)(F)F)C(=O)N3)nn2Cc1ccccc1F]

138 mg (0.26 mmol; purity 94%) of rac-2-[5-fluoro-1-(2-fluorobenzyl)-6-methyl-1H-pyrazolo[3,4-b]pyridin-3-yl]-5-methyl-6-oxo-5-(trifluoromethyl)-6,7-dihydro-5H-pyrrolo[2,3-d]pyrimidine-4-carbonitrile from Example 50A were initially charged in 4 ml of abs. dioxane, 1.5 ml (3.00 mmol) of 2 N aqueous sodium hydroxide solution were added and the mixture was stirred at RT overnight and at 80° C. for 5 h. The reaction solution was cooled to RT and diluted with 5 ml of 1 N aqueous sodium hydroxide solution. The mixture was subsequently adjusted to pH 5 using saturated aqueous ammonium chloride solution. The suspension was freed from the dioxane on a rotary evaporator. The solid obtained was then filtered off. This solid was washed with water and dried under high vacuum. This gave 116 mg (83% of theory, purity 96%) of the title compound.

LC-MS (Method 1): Rₜ=1.03 min

MS (ESIpos): m/z=518 [M+H]⁺

¹H-NMR (400 MHz, DMSO-d₆): δ [ppm]=1.91 (s, 3H), 2.65 (d, 3H), 5.84 (s, 2H), 7.13-7.27 (m, 3H), 7.34-7.41 (m, 1H), 7.99 (s, 1H), 8.29 (s, 1H), 8.54 (d, 1H), 12.46 (br. s, 1H).

### Example 13

### ent-2-[5-fluoro-1-(2-fluorobenzyl)-6-methyl-1H-pyrazolo[3,4-b]pyridin-3-yl]-5-methyl-6-oxo-5-(trifluoromethyl)-6,7-dihydro-5H-pyrrolo[2,3-d]pyrimidine-4-carboxamide (enantiomer A)

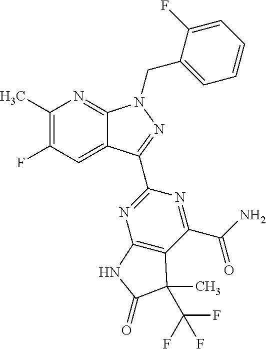 [SMILES: Cc1nc2c(cc1F)c(-c1nc3c(c(C(N)=O)n1)C(C)(C(F)(F)F)C(=O)N3)nn2Cc1ccccc1F]

102 mg of rac-2-[5-fluoro-1-(2-fluorobenzyl)-6-methyl-1H-pyrazolo[3,4-b]pyridin-3-yl]-5-methyl-6-oxo-5-(trifluoromethyl)-6,7-dihydro-5H-pyrrolo[2,3-d]pyrimidine-4-carboxamide (Example 12) were separated on a chiral phase into the enantiomers [SFC column: Daicel Chiralcel OJ-H, 5 μm, SFC 250×20 mm, mobile phase 85% CO₂, 15% isopropanol, flow rate 100 ml/min; 40° C., detection: 210 nm].

Enantiomer A: 37 mg (purity >99%, >99% ee)

Rₜ=2.09 min [SFC: Daicel Chiralcel OJ-H, 5 μm, 250×4.6 mm; mobile phase: 5→50% isopropanol gradient; flow rate 3.0 ml/min; detection: 220 nm].

### Example 14

### ent-2-[5-fluoro-1-(2-fluorobenzyl)-6-methyl-1H-pyrazolo[3,4-b]pyridin-3-yl]-5-methyl-6-oxo-5-(trifluoromethyl)-6,7-dihydro-5H-pyrrolo[2,3-d]pyrimidine-4-carboxamide (enantiomer B)

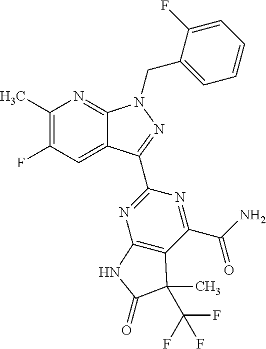 [SMILES: Cc1nc2c(cc1F)c(-c1nc3c(c(C(N)=O)n1)C(C)(C(F)(F)F)C(=O)N3)nn2Cc1ccccc1F]

102 mg of rac-2-[5-fluoro-1-(2-fluorobenzyl)-6-methyl-1H-pyrazolo[3,4-b]pyridin-3-yl]-5-methyl-6-oxo-5-(trifluoromethyl)-6,7-dihydro-5H-pyrrolo[2,3-d]pyrimidine-4-carboxamide (Example 12) were separated on a chiral phase into the enantiomers [SFC column: Daicel Chiralcel OJ-H, 5 μm, SFC 250×20 mm, mobile phase 85% CO₂, 15% isopropanol, flow rate 100 ml/min; 40° C., detection: 210 nm].

Enantiomer B: 38 mg (purity >99%, >99% ee)

Rₜ=2.54 min [SFC: Daicel Chiralcel OJ-H, 5 μm, 250×4.6 mm; mobile phase: 5→50% isopropanol gradient; flow rate 3.0 ml/min; detection: 220 nm].

### Example 15

### rac-2-[5-fluoro-6-methyl-1-(2,3,6-trifluorobenzyl)-1H-pyrazolo[3,4-b]pyridin-3-yl]-5-methyl-6-oxo-5-(trifluoromethyl)-6,7-dihydro-5H-pyrrolo[2,3-d]pyrimidine-4-carboxamide

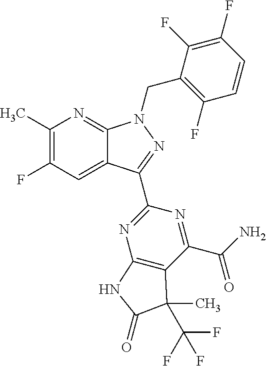 [SMILES: Cc1nc2c(cc1F)c(-c1nc3c(c(C(N)=O)n1)C(C)(C(F)(F)F)C(=O)N3)nn2Cc1c(F)ccc(F)c1F]

121 mg (0.20 mmol; purity 90%) of rac-2-[5-fluoro-6-methyl-1-(2,3,6-trifluorobenzyl)-1H-pyrazol-3,4-b]pyridin-3-yl-5-methyl-6-oxo-5-(trifluoromethyl)-6,7-dihydro-5H-pyrrolo[2,3-d]pyrimidine-4-carbonitrile from Example 51A were initially charged in 4 ml of abs. dioxane, 1.5 ml (3.00 mmol) of 2 N aqueous sodium hydroxide solution were added and the mixture was stirred at RT overnight and then at 80° C. for 5 h. The reaction solution was then cooled to RT and diluted with 5 ml of 1 N aqueous sodium hydroxide solution. The mixture was subsequently adjusted to pH 5 using saturated aqueous ammonium chloride solution. The suspension was freed from the dioxane on a rotary evaporator. The solid obtained was then filtered off. This solid was washed with water and dried under high vacuum. This gave 116 mg (83% of theory, purity 96%) of the title compound.

LC-MS (Method 1): Rₜ=1.04 min

MS (ESIpos): m/z=554 [M+H]⁺

¹H-NMR (400 MHz, DMSO-d₆): δ [ppm]=1.90 (s, 3H), 2.66 (d, 3H), 5.87 (s, 2H), 7.20 (ddt, 1H), 7.55 (ddt, 1H), 7.99 (s, 1H), 8.29 (s, 1H), 8.54 (d, 1H), 12.45 (br. s, 1H).

### Example 16

### ent-2-[5-Fluoro-6-methyl-1-(2,3,6-trifluorobenzyl)-1H-pyrazolo[3,4-b]pyridin-3-yl]-5-methyl-6-oxo-5-(trifluoromethyl)-6,7-dihydro-5H-pyrrolo[2,3-d]pyrimidine-4-carboxamide (enantiomer A)

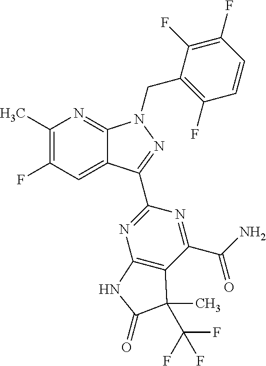 [SMILES: Cc1nc2c(cc1F)c(-c1nc3c(c(C(N)=O)n1)C(C)(C(F)(F)F)C(=O)N3)nn2Cc1c(F)ccc(F)c1F]

85 mg of rac-2-[5-fluoro-6-methyl-1-(2,3,6-trifluorobenzyl)-1H-pyrazolo[3,4-b]pyridin-3-yl]-5-methyl-6-oxo-5-(trifluoromethyl)-6,7-dihydro-5H-pyrrolo[2,3-d]pyrimidine-4-carboxamide (Example 15) were separated on a chiral phase into the enantiomers [SFC column: Daicel Chiralcel OJ-H, 5 μm, 250×20 mm, mobile phase: 85% CO₂, 15% isopropanol, flow rate 80 ml/min; 40° C., detection: 210 nm].

Enantiomer A: 36 mg (purity >99%, >99% ee)

Rₜ=2.04 min [SFC: Daicel Chiralcel OJ-H, 5 μm, 250×4.6 mm; mobile phase: 5→60% isopropanol; flow rate 3.0 ml/min; detection: 220 nm].

### Example 17

### ent-2-[5-Fluoro-6-methyl-1-(2,3,6-trifluorobenzyl)-1H-pyrazolo[3,4-b]pyridin-3-yl]-5-methyl-6-oxo-5-(trifluoromethyl)-6,7-dihydro-5H-pyrrolo[2,3-d]pyrimidine-4-carboxamide (enantiomer B)

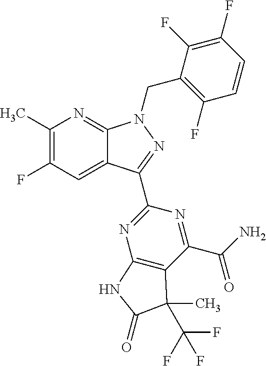 [SMILES: Cc1nc2c(cc1F)c(-c1nc3c(c(C(N)=O)n1)C(C)(C(F)(F)F)C(=O)N3)nn2Cc1c(F)ccc(F)c1F]

85 mg of rac-2-[5-fluoro-6-methyl-1-(2,3,6-trifluorobenzyl)-1H-pyrazolo[3,4-b]pyridin-3-yl]-5-methyl-6-oxo-5-(trifluoromethyl)-6,7-dihydro-5H-pyrrolo[2,3-d]pyrimidine-4-carboxamide (Example 15) were separated on a chiral phase into the enantiomers [SFC column: Daicel Chiralcel OJ-H, 5 μm, 250×20 mm, mobile phase: 85% CO₂, 15% isopropanol, flow rate 80 ml/min; 40° C., detection: 210 nm].

Enantiomer B: 36 mg (purity >99%, >99% ee)

Rₜ=2.57 min [SFC: Daicel Chiralcel OJ-H, 5 μm, 250×4.6 mm; mobile phase: 5-60% isopropanol; flow rate 3.0 ml/min; detection: 220 nm].

### Example 18

### rac-2-{5-Fluoro-1-[(3-fluoropyridin-2-yl)methyl]-6-methyl-1H-pyrazolo[3,4-b]pyridin-3-yl}-5-methyl-6-oxo-5-(trifluoromethyl)-6,7-dihydro-5H-pyrrolo[2,3-d]pyrimidine-4-carboxamide

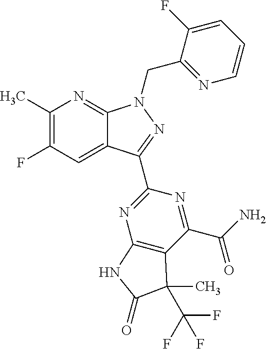 [SMILES: Cc1nc2c(cc1F)c(-c1nc3c(c(C(N)=O)n1)C(C)(C(F)(F)F)C(=O)N3)nn2Cc1ncccc1F]

100 mg (0.20 mmol) of rac-2-{5-fluoro-1-[(3-fluoropyridin-2-yl)methyl]-6-methyl-1H-pyrazolo[3,4-b]pyridin-3-yl}-5-methyl-6-oxo-5-(trifluoromethyl)-6,7-dihydro-5H-pyrrolo[2,3-d]pyrimidine-4-carbonitrile from Example 53A were initially charged in 3.0 ml of abs. dioxane, 0.50 ml (1.00 mmol) of 2 N aqueous sodium hydroxide solution were added and the mixture was stirred at 90° C. for 7 h. The reaction solution was cooled to RT and 1.20 ml (1.2 mmol) of 1 M hydrochloric acid were added. Water/acetonitrile were then added and the mixture was purified by preparative HPLC (RP18 column, mobile phase: acetonitrile/water gradient with addition of 0.1% TFA). 67 mg (63% of theory) of the title compound were obtained.

LC-MS (Method 1): Rₜ=0.92 min

MS (ESIpos): m/z=519 [M+H]⁺

¹H-NMR (500 MHz, DMSO-d₆): δ [ppm]=1.89 (s, 3H), 2.63 (d, 3H), 5.98 (s, 2H), 7.39-7.44 (m, 1H), 7.75-7.81 (m, 1H), 7.98 (br. s, 1H) 8.24-8.33 (m, 2H), 8.55 (d, 1H), 12.43 (br. s, 1H).

### Example 19

### ent-2-{5-Fluoro-1-[(3-fluoropyridin-2-yl)methyl]-6-methyl-1H-pyrazolo[3,4-b]pyridin-3-yl}-5-methyl-6-oxo-5-(trifluoromethyl)-6,7-dihydro-5H-pyrrolo[2,3-d]pyrimidine-4-carboxamide (enantiomer A)

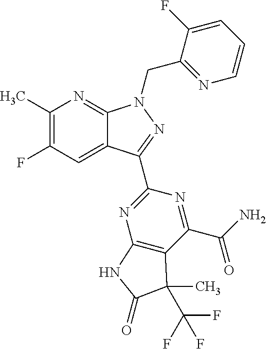 [SMILES: Cc1nc2c(cc1F)c(-c1nc3c(c(C(N)=O)n1)C(C)(C(F)(F)F)C(=O)N3)nn2Cc1ncccc1F]

67 mg of rac-2-{5-fluoro-1-[(3-fluoropyridin-2-yl)methyl]-6-methyl-1H-pyrazolo[3,4-b]pyridin-3-yl}-5-methyl-6-oxo-5-(trifluoromethyl)-6,7-dihydro-5H-pyrrolo[2,3-d]pyrimidine-4-carboxamide (Example 18) were separated on a chiral phase into the enantiomers [SFC column: Daicel Chiralpak OJ-H, 5 μm, 250×20 mm, mobile phase: 80% CO₂, 20% methanol, flow rate 100 ml/min; 30° C., detection: 210 nm].

Enantiomer A: 26 mg (purity 98%, >99% ee)

Rₜ=1.99 min [SFC: Daicel Chiralpak OJ-H, 5 μm, 250×4.6 mm, mobile phase: 5→50% methanol; flow rate 3.0 ml/min; detection: 220 nm].

### Example 20

### ent-2-{5-Fluoro-1-[(3-fluoropyridin-2-yl)methyl]-6-methyl-1H-pyrazolo[3,4-b]pyridin-3-yl}-5-methyl-6-oxo-5-(trifluoromethyl)-6,7-dihydro-5H-pyrrolo[2,3-d]pyrimidine-4-carboxamide (enantiomer B)

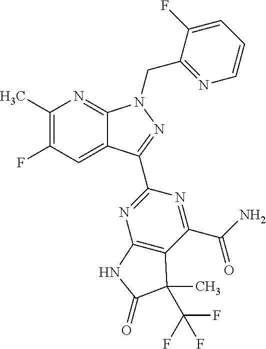 [SMILES: Cc1nc2c(cc1F)c(-c1nc3c(c(C(N)=O)n1)C(C)(C(F)(F)F)C(=O)N3)nn2Cc1ncccc1F]

67 mg of rac-2-{5-fluoro-1-[(3-fluoropyridin-2-yl)methyl]-6-methyl-1H-pyrazolo[3,4-b]pyridin-3-yl}-5-methyl-6-oxo-5-(trifluoromethyl)-6,7-dihydro-5H-pyrrolo[2,3-d]pyrimidine-4-carboxamide (Example 18) were separated on a chiral phase into the enantiomers [SFC column: Daicel Chiralpak OJ-H, 5 μm, 250×20 mm, mobile phase: 80% CO₂, 20% methanol, flow rate 100 ml/min; 30° C., dedetection: 210 nm].

Enantiomer B: 29 mg (purity 98%, 99% ee)

Rₜ=2.59 min [SFC: Daicel Chiralpak OJ-H, 5 μm, 250×4.6 mm, mobile phase: 5→50% methanol; flow rate 3.0 ml/min; detection: 220 nm].

### Example 21

### rac-2-{5-Fluoro-1-[(3-fluoropyridin-2-yl)methyl]-1H-pyrazolo[3,4-b]pyridin-3-yl}-5-methyl-6-oxo-5-(trifluoromethyl)-6,7-dihydro-5H-pyrrolo[2,3-d]pyrimidine-4-carboxamide

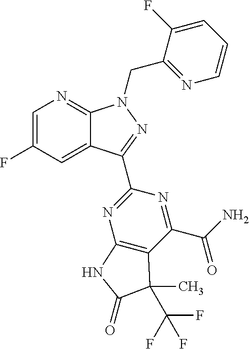 [SMILES: CC1(C(F)(F)F)C(=O)Nc2nc(-c3nn(Cc4ncccc4F)c4ncc(F)cc34)nc(C(N)=O)c21]

80 mg (0.16 mmol) of 2-{5-fluoro-1-[(3-fluoropyridin-2-yl)methyl]-1H-pyrazolo[3,4-b]pyridin-3-yl}-5-methyl-6-oxo-5-(trifluoromethyl)-6,7-dihydro-5H-pyrrolo[2,3-d]pyrimidine-4-carbonitrile from Example 49A were initially charged in 2.5 ml of abs. dioxane, 0.41 ml (0.82 mmol) of 2 N aqueous sodium hydroxide solution were added and the mixture was stirred at 90° C. for 7 h. The reaction solution was cooled to RT and diluted with 1.00 ml (1.00 mmol) of 1 M hydrochloric acid. Water/acetonitrile were then added and the mixture was purified by preparative HPLC (RP18 column, mobile phase: acetonitrile/water gradient with addition of 0.1% TFA). This gave 54 mg (63% of theory; purity 97%) of the title compound.

LC-MS (Method 1): Rₜ=0.86 min

MS (ESIpos): m/z=505 [M+H]⁺

¹H-NMR (500 MHz, DMSO-d₆): δ [ppm]=1.92 (s, 3H), 6.04 (s, 2H), 7.41-7.47 (m, 1H), 7.74-7.81 (m, 1H), 7.98 (br. s, 1H) 8.24-8.28 (m, 1H), 8.31 (s, 1H), 8.63-8.68 (m, 1H), 8.72-8.75 (m, 1H), 12.46 (br. s, 1H).

### Example 22

### ent-2-{5-Fluoro-1-[(3-fluoropyridin-2-yl)methyl]-1H-pyrazolo[3,4-b]pyridin-3-yl}-5-methyl-6-oxo-5-(trifluoromethyl)-6,7-dihydro-5H-pyrrolo[2,3-d]pyrimidine-4-carboxamide (enantiomer A)

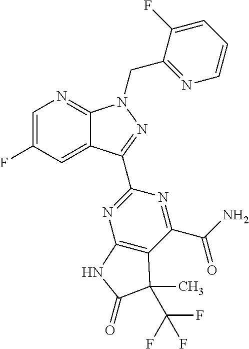 [SMILES: CC1(C(F)(F)F)C(=O)Nc2nc(-c3nn(Cc4ncccc4F)c4ncc(F)cc34)nc(C(N)=O)c21]

48 mg of rac-2-{5-fluoro-1-[(3-fluoropyridin-2-yl)methyl]-1H-pyrazolo[3,4-b]pyridin-3-yl}-5-methyl-6-oxo-5-(trifluoromethyl)-6,7-dihydro-5H-pyrrolo[2,3-d]pyrimidine-4-carboxamide (Example 21) were separated on a chiral phase into the enantiomers [SFC column: Daicel Chiralpak IB, 5 μm, 250×30 mm, mobile phase: 80% CO₂, 20% ethanol, flow rate 80 ml/min; 40° C., detection: 210 nm].

Enantiomer A: 16 mg (purity 97%, >99% ee)

Rₜ=3.26 min [SFC: Daicel Chiralpak IB, 5 μm, 250×4.6 mm; mobile phase: 5→50% ethanol; flow rate 3.0 ml/min; detection: 220 nm].

### Example 23

### ent-2-{5-Fluoro-1-[(3-fluoropyridin-2-yl)methyl]-1H-pyrazolo[3,4-b]pyridin-3-yl}-5-methyl-6-oxo-5-(trifluoromethyl)-6,7-dihydro-5H-pyrrolo[2,3-d]pyrimidine-4-carboxamide (enantiomer B)

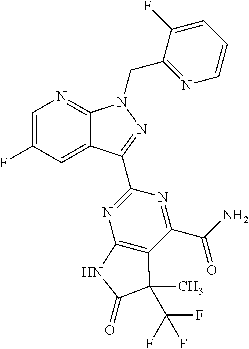 [SMILES: CC1(C(F)(F)F)C(=O)Nc2nc(-c3nn(Cc4ncccc4F)c4ncc(F)cc34)nc(C(N)=O)c21]

48 mg of rac-2-{5-fluoro-1-[(3-fluoropyridin-2-yl)methyl]-1H-pyrazolo[3,4-b]pyridin-3-yl}-5-methyl-6-oxo-5-(trifluoromethyl)-6,7-dihydro-5H-pyrrolo[2,3-d]pyrimidine-4-carboxamide (Example 21) were separated on a chiral phase into the enantiomers [SFC column: Daicel Chiralpak IB, 5 μm, 250×30 mm, mobile phase: 80% CO₂, 20% ethanol, flow rate 80 ml/min; 40° C., detection: 210 nm].

Enantiomer B: 18 mg (purity 97%, 93% ee)

Rₜ=3.84 min [SFC: Daicel Chiralpak IB, 5 μm, 250×4.6 mm; mobile phase: 5→50% ethanol; flow rate 3.0 ml/min; detection: 220 nm].

### Example 24

### N-Cyclopropyl-2-[1-(2-fluorobenzyl)-1H-pyrazolo[3,4-b]pyridin-3-yl]-5,5-dimethyl-6-oxo-6,7-dihydro-5H-pyrrolo[2,3-d]pyrimidine-4-carboxamide

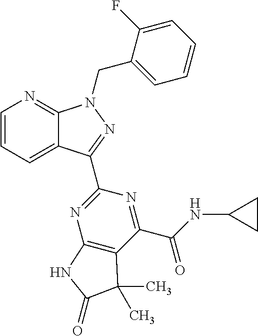 [SMILES: CC1(C)C(=O)Nc2nc(-c3nn(Cc4ccccc4F)c4ncccc34)nc(C(=O)NC3CC3)c21]

38 mg (0.09 mmol) of the compound from Example 54A, 10 mg (0.18 mmol) of cyclopropylamine and 46 μl (34 mg, 0.26 mmol) of diisopropylethylamine were dissolved in 0.8 ml of DMF, 78.5 μl (0.13 mmol) of 2,4,6-tripropyl-1,3,5,2,4,6-trioxatriphosphinane 2,4,6-trioxide (T3P, 50% solution in ethyl acetate) were added and the mixture was stirred at RT for 10 h. A further 5 mg (0.09 mmol) of cyclopropylamine and 42 μl (0.07 mmol) of 2,4,6-tripropyl-1,3,5,2,4,6-trioxatriphosphinane 2,4,6-trioxide (T3P, 50% solution in ethyl acetate) were added and the mixture was stirred at 50° C. for 5 h. The reaction mixture was concentrated under reduced pressure, dissolved in DMSO and acetonitrile, acidified slightly with 5 M formic acid and purified by preparative HPLC (RP18, mobile phase: 0.1% aqueous formic acid—acetonitrile, 5-95%). The residue was purified on silica gel (mobile phase: gradient of cyclohexane/ethyl acetate 5-65%). This gave 19 mg (46% of theory) of the title compound.

LC-MS (Method 1): Rₜ=1.02 min

MS (ESIpos): m/z=472 [M+H]⁺

¹H-NMR (400 MHz, DMSO-d₆): δ [ppm]=0.63-0.72 (m, 2H), 0.75-0.85 (m, 2H), 1.49 (s, 6H), 2.87-2.98 (m, 1H), 5.88 (s, 2H), 7.10-7.29 (m, 3H), 7.32-7.41 (m, 1H), 7.48 (dd, 1H), 8.65-8.76 (m, 2H), 8.84 (dd, 1H), 11.86 (br. s, 1H).

The exemplary compounds listed in Table 1 were prepared analogously to the procedure from Example 24 from the acid of Example 58A and the appropriate amines.

TABLE 1

| Ex- | IUPAC name/structure | |
|---|---|---|
| ample | (Yield) | Analytical data |
| 25 | 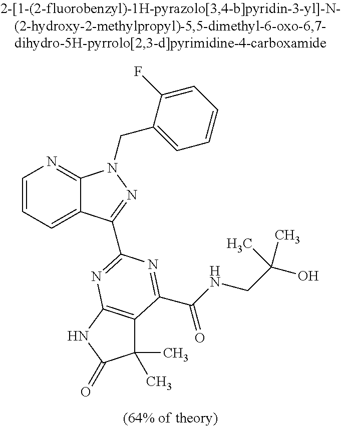 [SMILES: CC(C)(O)CNC(=O)c1nc(-c2nn(Cc3ccccc3F)c3ncccc23)nc2c1C(C)(C)C(=O)N2] | ¹H-NMR (400 MHz, DMSO-d₆): δ [ppm] = 1.20 (s, 6H), 1.52 (s, 6H), 4.92 (br. s, 1H), 5.89 (s, 2H), 7.10- 7.28 (m, 3H), 7.32-7.41 (m, 1H), 7.44 (dd, 1H), 8.63-8.75 (m, 2H), 8.94 (d, 1H), 11.93 (s, 1H). LC-MS (Method 1): Rₜ = 0.98 min MS (ESIpos): m/z = 504 [M + H]⁺ |
| 26 | 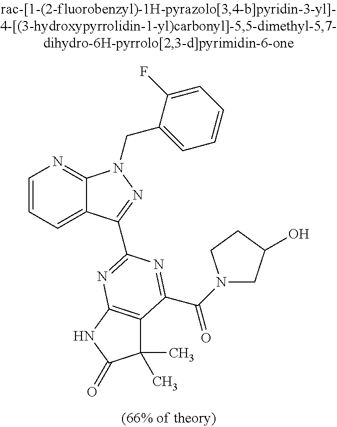 [SMILES: CC1(C)C(=O)Nc2nc(-c3nn(Cc4ccccc4F)c4ncccc34)nc(C(=O)N3CCC(O)C3)c21] | ¹H-NMR (400 MHz, DMSO-d₆): δ [ppm] = 1.28-1.43 (m, 6H), 1.72- 2.06 (m, 2H), 3.15-3.24 (m, 1H), 3.16-3.70 (m, 4H) superposed by water signal, 4.19-4.41 (m, 1H), 4.88-5.16 (m, 1H), 5.88 (s, 2H), 7.10-7.28 (m, 3H), 7.32-7.41 (m, 1H), 7.42-7.49 (m, 1H), 8.68 (dd, 1H), 8.78-8.86 (m, 1H), 11.81 (s, 1H). LC-MS (Method 1): Rₜ = 0.84 min MS (ESIpos): m/z = 502 [M + H]⁺ |

### Example 27

### 2-[5-Fluoro-1-(2-fluorobenzyl)-1H-pyrazolo[3,4-b]pyridin-3-yl]-N-[(1-hydroxycyclopropyl)methyl]-5,5-dimethyl-6-oxo-6,7-dihydro-5H-pyrrolo[2,3-d]pyrimidine-4-carboxamide

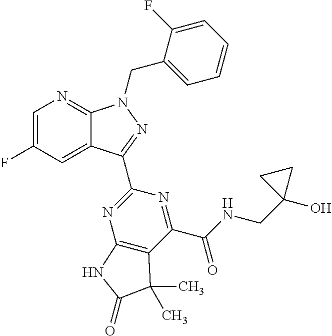 [SMILES: CC1(C)C(=O)Nc2nc(-c3nn(Cc4ccccc4F)c4ncc(F)cc34)nc(C(=O)NCC3(O)CC3)c21]

50 mg (0.11 mmol) of the compound from Example 55A, 19 mg (0.22 mmol) of 1-(aminomethyl)cyclopropanol and 93 μl (0.67 mmol) of triethylamine were dissolved in 0.7 ml of DMF, 99 μl (0.17 mmol) of 2,4,6-tripropyl-1,3,5,2,4,6-trioxatriphosphinane 2,4,6-trioxide (T3P, 50% solution in ethyl acetate) were added and the mixture was stirred at RT for 9 h. The reaction mixture was concentrated under reduced pressure, dissolved in DMSO and acetonitrile, acidified slightly with formic acid and purified by preparative HPLC (RP 18, mobile phase: 0.1% aqueous formic acid-acetonitrile, 5-95%). 33 mg (55% of theory) of the title compound were obtained.

LC-MS (Method 1): Rₜ=1.01 min

MS (ESIpos): m/z=520 [M+H]⁺

¹H-NMR (400 MHz, DMSO-d₆): δ [ppm]=0.60-0.70 (m, 4H), 1.50 (s, 6H), 3.51 (d, 2H), 5.89 (s, 2H), 7.12-7.30 (m, 3H), 7.33-7.42 (m, 1H), 8.64 (d, 1H), 8.70-8.80 (m, 2H), 11.89 (s, 1H).

The exemplary compounds listed in Table 2 were prepared analogously to the procedure from Example 27 from the acids of the starting materials 55A, 56A and 57A and the appropriate amines. If appropriate, further amine (1-3 equivalents), 2,4,6-tripropyl-1,3,5,2,4,6-trioxatriphosphinane 2,4,6-trioxide (50% in ethyl acetate) (0.5-1.0 equivalent) and triethylamine (2-4 equivalents) were added to the reaction mixtures and stirring was continued until the reaction had gone to completion (1-24 h). Purifications were carried out by preparative HPLC (RP18 column, mobile phase: acetonitrile/water gradient with addition of 0.1% formic acid or 0.1% TFA).

TABLE 2

| Ex- | IUPAC name/structure | Workup, |
|---|---|---|
| ample | (Yield) | analytical data |
| 28 | 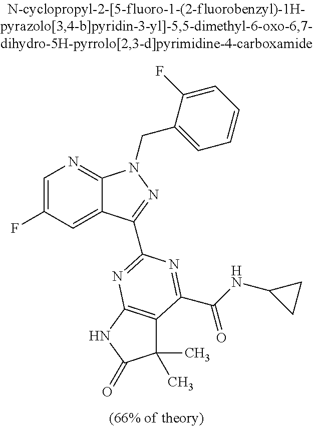 [SMILES: CC1(C)C(=O)Nc2nc(-c3nn(Cc4ccccc4F)c4ncc(F)cc34)nc(C(=O)NC3CC3)c21] | ¹H-NMR (400 MHz, DMSO-d₆): δ [ppm] = 0.63-0.70 (m, 2H), 0.76- 0.84 (m, 2H), 1.49 (s, 6H), 2.94 (m, 1H), 5.88 (s, 2H), 7.12-7.27 (m, 3H), 7.33-7.41 (m, 1H), 8.59 (dd, 1H), 8.72-8.79 (m, 2H), 11.84 (s, 1H). LC-MS (Method 1): Rₜ = 1.11 min MS (ESIpos): m/z = 490 [M + H]⁺ |
| 29 | 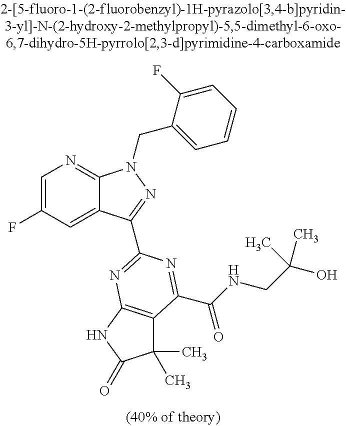 [SMILES: CC(C)(O)CNC(=O)c1nc(-c2nn(Cc3ccccc3F)c3ncc(F)cc23)nc2c1C(C)(C)C(=O)N2] | Additional purification by stirring with water/methanol/sat. potassium carbonate solution ¹H-NMR (400 MHz, DMSO-d₆): δ [ppm] = 1.19 (s, 6H), 1.50 (s, 6H), 3.35 (d, 2H), 4.77 (s, 1H), 5.88 (s, 2H), 7.12-7.29 (m, 3H), 7.33-7.41 (m, 1H), 8.59-8.67 (m, 2H), 8.76 (s, 1H), 11.90 (br. s, 1H). LC-MS (Method 5): Rₜ = 2.42 min MS (ESIpos): m/z = 522 [M + H]⁺ |
| 30 | 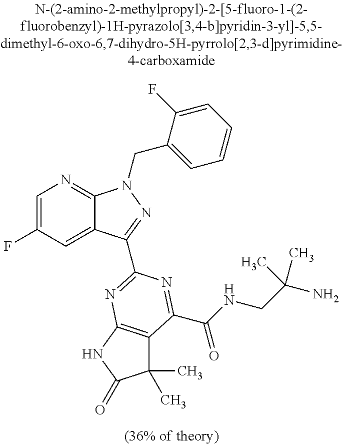 [SMILES: CC(C)(N)CNC(=O)c1nc(-c2nn(Cc3ccccc3F)c3ncc(F)cc23)nc2c1C(C)(C)C(=O)N2] | ¹H-NMR (400 MHz, DMSO- d₆/D₂O): δ [ppm] = 1.15 (s, 6H), 1.51 (s, 6H), 3.28 (s, 2H), 5.88 (s, 2H), 7.12-7.31 (m, 3H), 7.34-7.42 (m, 1H), 8.74-8.78 (m, 1H), 8.81 (dd, 1H). LC-MS (Method 1): Rₜ = 0.75 min MS (ESIpos): m/z = 521 [M + H]+ |
| 31 | 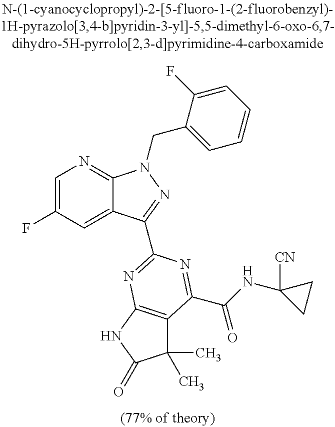 [SMILES: CC1(C)C(=O)Nc2nc(-c3nn(Cc4ccccc4F)c4ncc(F)cc34)nc(C(=O)NC3(C#N)CC3)c21] | ¹H-NMR (400 MHz, DMSO-d₆): δ [ppm] = 1.32-1.41 (m, 2H), 1.49 (s, 6H), 1.63-1.71 (m, 2H), 5.88 (s, 2H), 7.11-7.27 (m, 3H), 7.33-7.42 (m, 1H), 8.65 (dd, 1H), 8.77 (dd, 1H), 9.66 (s, 1H), 11.92 (s, 1H). LC-MS (Method 5): Rₜ = 1.07 min MS (ESIpos): m/z = 515 [M + H]⁺ |
| 32 | 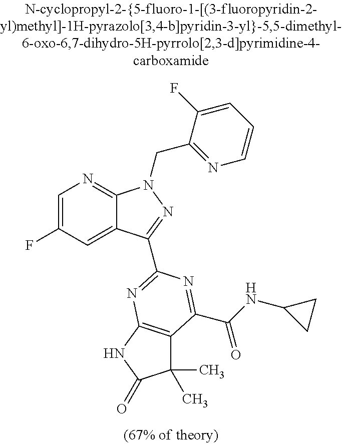 [SMILES: CC1(C)C(=O)Nc2nc(-c3nn(Cc4ncccc4F)c4ncc(F)cc34)nc(C(=O)NC3CC3)c21] | ¹H-NMR (400 MHz, DMSO-d₆): δ [ppm] = 0.65-0.70 (m, 2H), 0.76- 0.83 (m, 2H), 1.50 (s, 6H), 2.89- 2.98 (m, 1H), 6.02 (s, 2H), 7.40- 7.47 (m, 1H), 7.74-7.81 (m, 1H), 8.24 (d, 1H), 8.57-8.62 (m, 1H), 8.68-8.78 (m, 2H), 11.81 (s, 1H). LC-MS (Method 1): Rₜ = 0.95 min MS (ESIpos): m/z = 491 [M + H]⁺ |
| 33 | 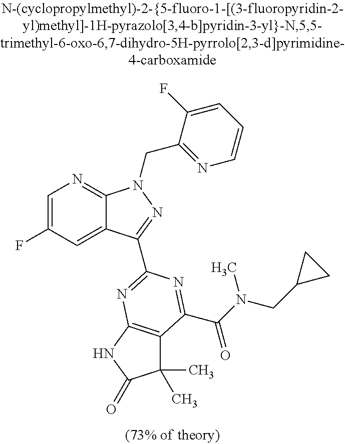 [SMILES: CN(CC1CC1)C(=O)c1nc(-c2nn(Cc3ncccc3F)c3ncc(F)cc23)nc2c1C(C)(C)C(=O)N2] | ¹H-NMR (400 MHz, DMSO-d₆): δ [ppm] = 0.10-0.17 (m, 1H), 0.30- 0.40 (m, 2H), 0.53-0.60 (m, 1H), 0.95-1.08 and 1.12-1.22 (2 m, together 1H), 1.31 (s, 3H), 1.36 (s, 3H), 2.92 and 3.13 (2 s, together 3H), 3.06 and 3.44 (2 d, together 2H), 5.98-6.05 (m, 2H), 7.40-7.47 (m, 1H), 7.74-7.81 (m, 1H), 8.25- 8.28 (m, 1H), 8.58-8.63 (m, 1H), 8.69-8.74 (m, 1H), 11.73-11.79 (m, 1H). (~1:1 mixture of amide rotational isomers). LC-MS (Method 1): Rₜ = 0.93 min MS (ESIpos): m/z = 519 [M + H]⁺ |
| 34 | 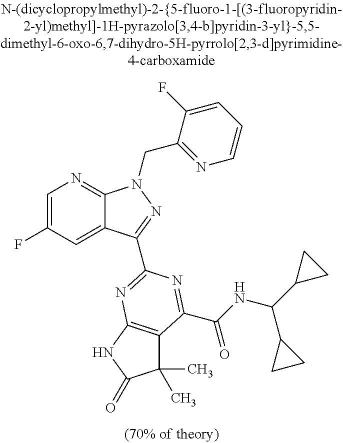 [SMILES: CC1(C)C(=O)Nc2nc(-c3nn(Cc4ncccc4F)c4ncc(F)cc34)nc(C(=O)NC(C3CC3)C3CC3)c21] | ¹H-NMR (400 MHz, DMSO-d₆): δ [ppm] = 0.34-0.48 (m, 6H), 0.53- 0.62 (m, 2H), 1.13-1.24 (m, 2H), 1.49 (s, 6H), 3.08-3.14 (m, 1H), 6.02 (s, 2H), 7.40-7.47 (m, 1H), 7.74-7.81 (m, 1H), 8.26 (d, 1H), 8.60-8.66 (m, 1H), 8.68-8.78 (m, 2H), 11.81 (s, 1H). LC-MS (Method 1): Rₜ = 1.11 min MS (ESIpos): m/z = 545 [M + H]⁺ |
| 35 | 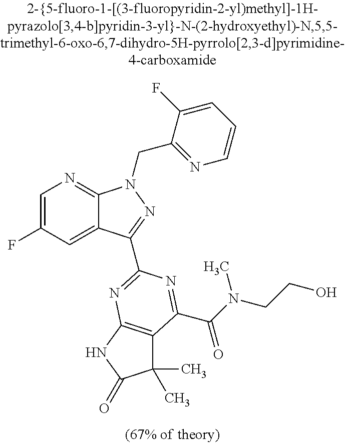 [SMILES: CN(CCO)C(=O)c1nc(-c2nn(Cc3ncccc3F)c3ncc(F)cc23)nc2c1C(C)(C)C(=O)N2] | ¹H-NMR (400 MHz, DMSO-d₆): δ [ppm] = 1.35/1.36 (2 s, together 6H), 2.91/3.12 (2 s, together 3H), 3.48-3.56 (m, 1H), 3.59-3.65 (m, 1H), 3.67-3.72 (m, 1H), 4.62 and 4.82 (2 br. s, together 1H), 6.02 (s, 2H), 7.40-7.47 (m, 1H), 7.74-7.81 (m, 1H), 8.28 (d, 1H), 8.48-8.58 (m, 1H), 8.69-8.73 (m, 1H), 11.74 (s, 1H) [further signal under solvent peak]). (~1:1 mixture of amide rotational isomers). LC-MS (Method 1): Rₜ = 0.70 min MS (ESIpos): m/z = 509 [M + H]⁺ |
| 36 | 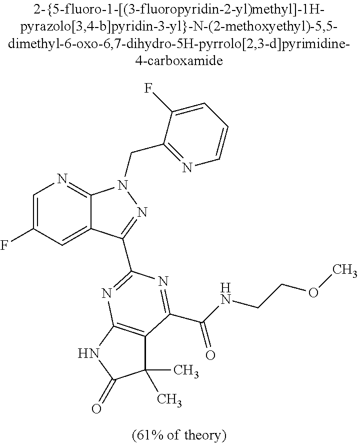 [SMILES: COCCNC(=O)c1nc(-c2nn(Cc3ncccc3F)c3ncc(F)cc23)nc2c1C(C)(C)C(=O)N2] | ¹H-NMR (400 MHz, DMSO-d₆): δ [ppm] = 1.50 (s, 6H), 3.35 (s, 3H; superposed by solvent peak), 3.52- 3.60 (m, 4H), 6.02 (s, 2H), 7.40- 7.47 (m, 1H), 7.74-7.81 (m, 1H), 8.27 (d, 1H), 8.57-8.62 (m, 1H), 8.70-8.78 (m, 2H), 11.85 (s, 1H). LC-MS (Method 1): Rₜ = 0.88 min MS (ESIpos): m/z = 509 [M + H]⁺ |
| 37 | 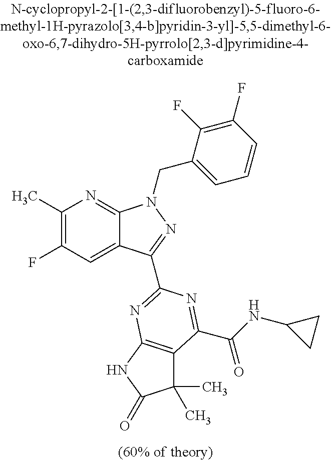 [SMILES: Cc1nc2c(cc1F)c(-c1nc3c(c(C(=O)NC4CC4)n1)C(C)(C)C(=O)N3)nn2Cc1cccc(F)c1F] | ¹H-NMR (400 MHz, DMSO-d₆): δ [ppm] = 0.64-0.69 (m, 2H), 0.78- 0.83 (m, 2H), 1.49 (s, 6H), 2.67 (d, 3H), 2.89-2.98 (m, 1H), 5.88 (s, 2H), 6.98-7.05 (m, 1H), 7.13-7.21 (m, 1H), 7.36-7.45 (m, 1H), 8.48 (d, 1H), 8.73 (d, 1H), 11.80 (br. s, 1H). LC-MS (Method 1): Rₜ = 1.19 min MS (ESIpos): m/z = 522 [M + H]⁺ |
| 38 | 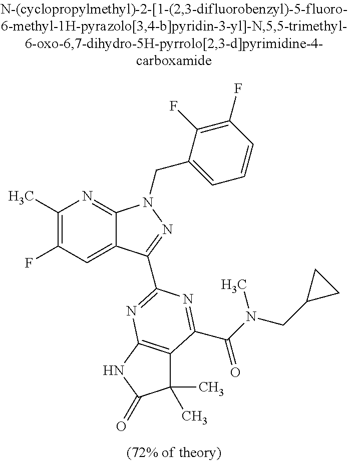 [SMILES: Cc1nc2c(cc1F)c(-c1nc3c(c(C(=O)N(C)CC4CC4)n1)C(C)(C)C(=O)N3)nn2Cc1cccc(F)c1F] | ¹H-NMR (400 MHz, DMSO-d₆): δ [ppm] = 0.12-0.17 (m, 1H), 0.30- 0.40 (m, 2H), 0.53-0.60 (m, 1H), 0.98-1.10 and 1.12-1.22 (2 m, together 1H), 1.31 (s, 3H), 1.36 (s, 3H), 2.62-2.66 (m, 3H), 2.91 and 3.13 (2 s, together 3H), 3.05 and 3.46 (2 d, together 2H), 5.84-5.88 (m, 2H), 6.99-7.10 (m, 1H), 7.12- 7.20 (m, 1H), 7.35-7.44 (m, 1H), 8.40 (d, 1H), 11.71-11.78 (m, 1H). (~1:1 mixture of amide rotational isomers). LC-MS (Method 1): Rₜ = 1.13 min MS (ESIpos): m/z = 550 [M + H]⁺ |
| 39 | 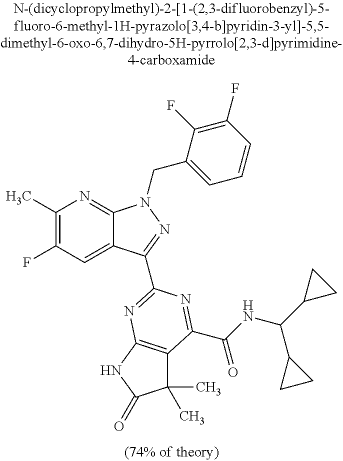 [SMILES: Cc1nc2c(cc1F)c(-c1nc3c(c(C(=O)NC(C4CC4)C4CC4)n1)C(C)(C)C(=O)N3)nn2Cc1cccc(F)c1F] | ¹H-NMR (400 MHz, DMSO-d₆): δ [ppm] = 0.33-0.48 (m, 6H), 0.52- 0.61 (m, 2H), 1.13-1.23 (m, 2H), 1.48 (s, 6H), 2.67 (d, 3H), 3.08- 3.16 (m, 1H), 5.88 (s, 2H), 7.02- 7.08 (m, 1H), 7.13-7.22 (m, 1H), 7.36-7.45 (m, 1H), 8.52 (d, 1H), 8.69 (d, 1H), 11.82 (s, 1H). LC-MS (Method 1): Rₜ = 1.34 min MS (ESIpos): m/z = 576 [M + H]⁺ |
| 40 | 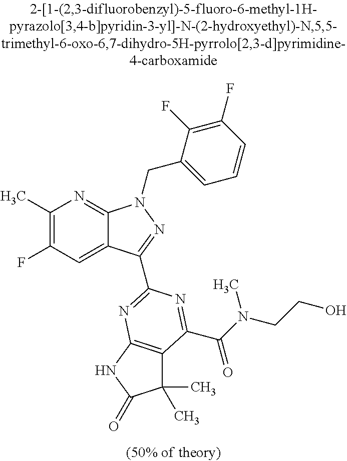 [SMILES: Cc1nc2c(cc1F)c(-c1nc3c(c(C(=O)N(C)CCO)n1)C(C)(C)C(=O)N3)nn2Cc1cccc(F)c1F] | ¹H-NMR (400 MHz, DMSO-d₆): δ [ppm] = 1.35/1.36 (2 s, together 6H), 2.65 d, 3H), 2.92/3.12 (2 s, together 3H), 3.48-3.56 (m, 1H), 3.59-3.65 (m, 1H), 3.67-3.72 (m, 1H), 4.70 (br. s, 1H), 5.88 (s, 2H), 6.98-7.08 (m, 1H), 7.12-7.22 (m, 1H), 7.35-7.45 (m, 1H), 8.38-8.48 (m, 1H), 11.75 (s, 1H). [further signal under solvent peak]). (~1:1 1:1 mixture of amide rotational isomers) LC-MS (Method 1): Rₜ = 0.91 min MS (ESIpos): m/z = 540 [M + H]⁺ |
| 41 | 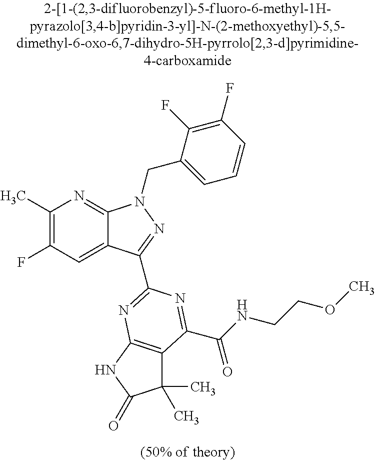 [SMILES: COCCNC(=O)c1nc(-c2nn(Cc3cccc(F)c3F)c3nc(C)c(F)cc23)nc2c1C(C)(C)C(=O)N2] | ¹H-NMR (400 MHz, DMSO-d₆): δ [ppm] = 1.50 (s, 6H), 2.67 (d, 3H), 3.35 (s, 3H), 3.53-3.61 (m, 4H), 5.88 (s, 2H), 6.98-7.05 (m, 1H), 7.13-7.21 (m, 1H), 7.36-7.45 (m, 1H), 8.49 (d, 1H), 8.69-8.76 (m, 1H), 11.86 (s, 1H). LC-MS (Method 1): Rₜ = 1.10 min MS (ESIpos): m/z = 540 [M + H]⁺ |

### Example 42

### 2-[6-Chloro-1-(2-fluorobenzyl)-1H-indazol-3-yl]-N-(cyclopropylmethyl)-5,5-dimethyl-6-oxo-6,7-dihydro-5H-pyrrolo[2,3-d]pyrimidine-4-carboxamide

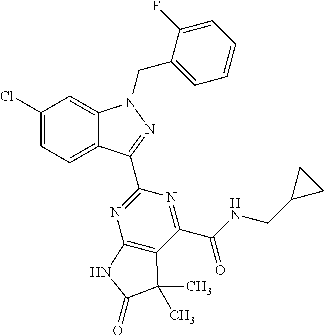 [SMILES: CC1(C)C(=O)Nc2nc(-c3nn(Cc4ccccc4F)c4cc(Cl)ccc34)nc(C(=O)NCC3CC3)c21]

70 mg (0.15 mmol) of the compound from Example 58A, 21 mg (0.3 mmol) of 1-cyclopropylmethanamine and 61 mg (0.6 mmol) of triethylamine in 1 ml of THF were heated to 60° C., 0.18 ml (0.3 mmol) of 2,4,6-tripropyl-1,3,5,2,4,6-trioxatriphosphinane 2,4,6-trioxide (T3P, 50% solution in ethyl acetate) were then added and the mixture was stirred at this temperature for 30 min. The reaction mixture was partitioned between water and ethyl acetate (extraction), and the organic phase was washed with sat. sodium chloride solution, dried and concentrated. The residue was purified by means of column chromatography (silica gel, mobile phase: gradient of cyclohexane/ethyl acetate 5-65%). This gave 59 mg (76% of theory) of the title compound.

LC-MS (Method 1): Rₜ=1.23 min

MS (ESIpos): m/z=519 [M+H]⁺

¹H-NMR (400 MHz, DMSO-d₆): δ [ppm]=0.29-0.36 (m, 1H), 0.48-0.54 (m, 1H), 1.07-1.17 (m, 1H), 1.50 (s, 6H), 3.26 (t, 2H), 5.87 (s, 2H), 7.14-7.28 (m, 3H), 7.33-7.42 (m, 2H), 8.09 (s, 1H), 8.53 (d, 1H), 8.70 (t, 1H), 11.85 (s, 1H).

The exemplary compounds listed in Table 3 were prepared analogously to the procedure from Example 42 from the acids of Example 58A and Example 55A, respectively, and the appropriate amines. If the amine was employed as a salt, 2 equivalents of triethylamine were additionally employed. If appropriate, further amine, 2,4,6-tripropyl-1,3,5,2,4,6-trioxatriphosphinane 2,4,6-trioxide (50% solution in ethyl acetate) and triethylamine were added and stirring was continued until the reaction had gone to completion.

Work-Up:

Method a): extraction and column chromatography on silica gel as described in Example 42.

Method b): water, acetonitrile and formic acid are added to the reaction mixture (pH 3-4), and the precipitate formed is filtered off and washed with water/acetonitrile.

Method c): the reaction mixture is concentrated, the residue is dissolved in DMSO/acetonitrile/aq. formic acid and purified by preparative HPLC (column: RP 18, gradient of water+0.1% formic acid/acetonitrile (5-95%)).

TABLE 3

| Ex- | IUPAC name/structure | Workup, |
|---|---|---|
| ample | (Yield) | analytical data |
| 43 | 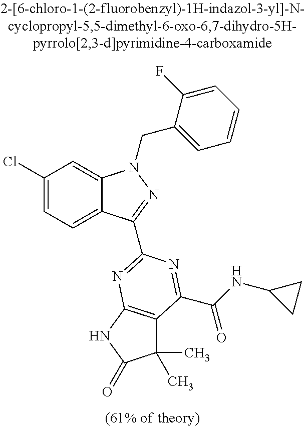 [SMILES: CC1(C)C(=O)Nc2nc(-c3nn(Cc4ccccc4F)c4cc(Cl)ccc34)nc(C(=O)NC3CC3)c21] | work-up Method a) ¹H-NMR (400 MHz, DMSO-d₆): δ [ppm] = 0.63-0.69 (m, 2H), 0.76- 0.83 (m, 2H), 1.48 (s, 6H), 2.88- 2.97 (m, 1H), 5.86 (s, 2H), 7.12- 7.28 (m, 3H), 7.33-7.43 (m, 2H), 8.06 (s, 1H), 8.46 (d, 1H), 8.64 (d, 1H), 11.83 (s, 1H). LC-MS (Method 1): Rₜ = 1.16 min MS (ESIpos): m/z = 505 [M + H]⁺ |
| 44 | 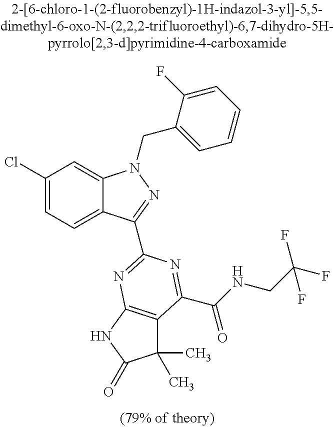 [SMILES: CC1(C)C(=O)Nc2nc(-c3nn(Cc4ccccc4F)c4cc(Cl)ccc34)nc(C(=O)NCC(F)(F)F)c21] | work-up Method b) ¹H-NMR (400 MHz, DMSO-d₆): δ [ppm] = 1.47 (s, 6H), 4.14-4.27 (m, 2H), 5.87 (s, 2H), 7.10-7.28 (m, 3H), 7.31-7.43 (m, 2H), 8.08 (s, 1H), 8.53 (d, 1H), 9.19 (t, 1H), 11.90 (s, 1H). LC-MS (Method 1): Rₜ = 1.17 min MS (ESIpos): m/z = 547 [M + H]⁺ |
| 45 | 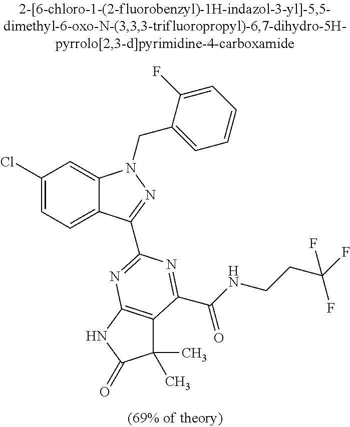 [SMILES: CC1(C)C(=O)Nc2nc(-c3nn(Cc4ccccc4F)c4cc(Cl)ccc34)nc(C(=O)NCCC(F)(F)F)c21] | work-up Method a) ¹H-NMR (400 MHz, DMSO-d₆): δ [ppm] = 1.50 (s, 6H), 2.56-2.72 (m, 2H), 3.64 (q, 2H), 5.87 (s, 2H), 7.10- 7.28 (m, 3H), 7.31-7.42 (m, 2H), 8.07 (s, 1H), 8.53 (d, 1H), 8.87 (t, 1H), 11.87 (s, 1H). LC-MS (Method 1): Rₜ = 1.22 min MS (ESIpos): m/z = 561 [M + H]⁺ |
| 46 | 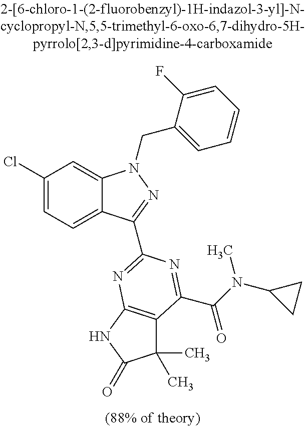 [SMILES: CN(C(=O)c1nc(-c2nn(Cc3ccccc3F)c3cc(Cl)ccc23)nc2c1C(C)(C)C(=O)N2)C1CC1] | work-up Method c) ¹H-NMR (400 MHz, DMSO-d₆): δ [ppm] = 0.41-0.58 (m, 3H), 0.74- 0.89 (m, 1H), 1.31 (s, 1.5H), 1.35 (s, 4.5H), 2.77 (s, 0.75H), 2.85-2.93 (m, 0.75H), 2.96-3.03 (m, 0.25H), 3.07 (s, 2.25H), 5.84 (s, 2H), 7.14- 7.28 (m, 3H), 7.34-7.42 (m, 2H), 8.06 (s, 1H), 8.45 (d, 0.25H), 8.50 (d, 0.75H), 11.77 (s, 1H). (~3:1 mixture of amide rotational isomers LC-MS (Method 3): Rₜ = 2.55 min MS (ESIpos): m/z = 519 [M + H]⁺ |
| 47 | 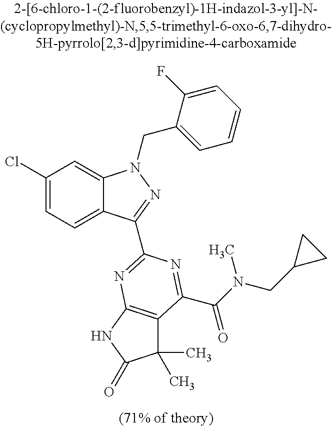 [SMILES: CN(CC1CC1)C(=O)c1nc(-c2nn(Cc3ccccc3F)c3cc(Cl)ccc23)nc2c1C(C)(C)C(=O)N2] | work-up Method c) ¹H-NMR (400 MHz, DMSO-d₆): δ [ppm] = 0.09-0.16 (m, 1H), 0.30- 0.40 (m, 2H), 0.52-0.60 (m, 1H), 0.97-1.08 (m, 1H), 1.11-1.21 (m, 1H), 1.31 (s, 3H), 1.36 (s, 3H), 2.92 (s, 1.5H), 3.05 (d, 1H), 3.13 (s, 1.5H), 3.43 (d, 1H), 5.84 (s, 1H), 5.86 (s, 1H), 7.13-7.27 (m, 3H), 7.33-7.41 (m, 2H), 8.06 (br. s., 1H), 8.43-8.52 (m, 1H), 11.77 (br. s., 1H). (~1:1 mixture of amide rotational isomers). LC-MS (Method 1): Rₜ = 1.17 min MS (ESIpos): m/z = 533 [M + H]⁺ |
| 48 | 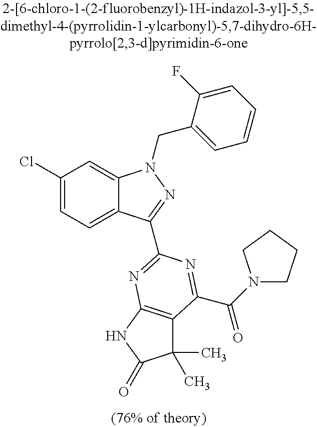 [SMILES: CC1(C)C(=O)Nc2nc(-c3nn(Cc4ccccc4F)c4cc(Cl)ccc34)nc(C(=O)N3CCCC3)c21] | work-up Method a) ¹H-NMR (400 MHz, DMSO-d₆): δ [ppm] = 1.76-1.86 (m, 2H), 1.86- 1.95 (m, 2H), 3.27-3.37 (m, superposed by water signal), 3.56 (t, 2H), 5.85 (s, 2H), 7.13-7.27 (m, 3H), 7.33-7.42 (m, 2H), 8.06 (s, 1H), 8.47 (d, 1H), 11.76 (s, 1H). LC-MS (Method 1): Rₜ = 1.11 min MS (ESIpos): m/z = 519 [M + H]⁺ |
| 49 | 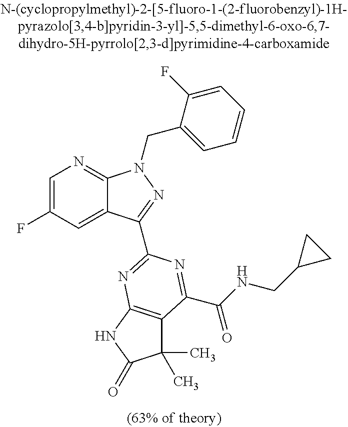 [SMILES: CC1(C)C(=O)Nc2nc(-c3nn(Cc4ccccc4F)c4ncc(F)cc34)nc(C(=O)NCC3CC3)c21] | work-up Method c) ¹H-NMR (400 MHz, DMSO-d₆): δ [ppm] = 0.29-0.40 (m, 2H), 0.45- 0.56 (m, 2H), 1.08-1.19 (m, 1H), 1.50 (s, 6H), 3.27 (t, 2H), 5.88 (s, 2H), 7.11-7.29 (m, 3H), 7.32-7.42 (m, 1H), 8.66 (dd, 1H), 8.74-8.86 (m, 2H), 11.83 (br. s, 1H). LC-MS (Method 1): Rₜ = 1.19 min MS (ESIpos): m/z = 504 [M + H]⁺ |
| 50 | 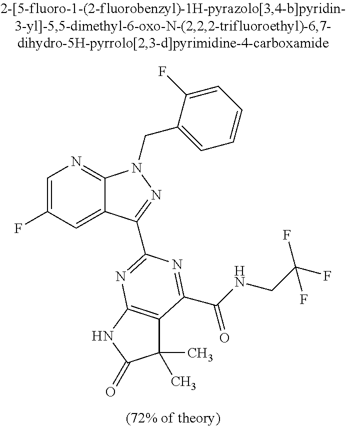 [SMILES: CC1(C)C(=O)Nc2nc(-c3nn(Cc4ccccc4F)c4ncc(F)cc34)nc(C(=O)NCC(F)(F)F)c21] | work-up Method c) ¹H-NMR (400 MHz, DMSO-d₆): δ [ppm] = 1.48 (s, 6H), 4.15-4.28 (m, 2H), 5.89 (s, 2H), 7.12-7.28 (m, 3H), 7.33-7.42 (m, 1H), 8.65 (dd, 1H), 8.77 (dd, 1H), 9.31 (t, 1H), 11.83-12.02 (m, 1H). LC-MS (Method 1): Rₜ = 1.17 min MS (ESIpos): m/z = 532 [M + H]⁺ |
| 51 | 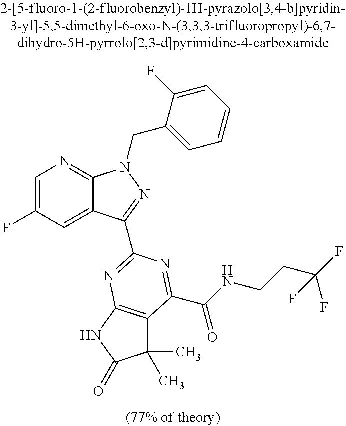 [SMILES: CC1(C)C(=O)Nc2nc(-c3nn(Cc4ccccc4F)c4ncc(F)cc34)nc(C(=O)NCCC(F)(F)F)c21] | work-up Method c) ¹H-NMR (400 MHz, DMSO-d₆): δ [ppm] = 1.50 (s, 6H), 2.57-2.73 (m, 2H), 3.65 (q, 2H), 5.88 (s, 2H), 7.11- 7.28 (m, 3H), 7.33-7.42 (m, 1H), 8.65 (dd, 1H), 8.73-8.79 (m, 1H), 8.97 (t, 1H), 11.89 (s, 1H). LC-MS (Method 1): Rₜ = 1.18 min MS (ESIpos): m/z = 546 [M + H]⁺ |
| 52 | 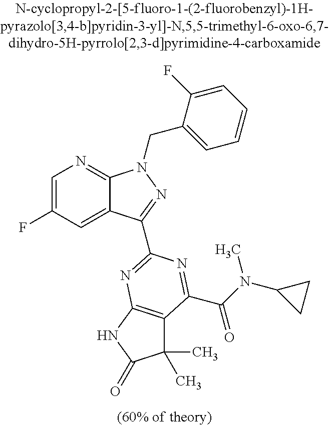 [SMILES: CN(C(=O)c1nc(-c2nn(Cc3ccccc3F)c3ncc(F)cc23)nc2c1C(C)(C)C(=O)N2)C1CC1] | work-up Method c) ¹H-NMR (400 MHz, DMSO-d₆): δ [ppm] = 0.42-0.58 (m, 3H), 0.74- 0.91 (m, 1H), 1.31 (s, 1.5H), 1.35 (s, 4.5H), 2.77 (s, 1H), 2.85-2.92 (m, 0.75H), 2.96-3.03 (m, 0.25 H), 3.07 (s, 2H), 5.87 (s, 2H), 7.12-7.30 (m, 3H), 7.33-7.41 (m, 1H), 8.48 (dd, 0.25H), 8.54 (dd, 0.75H), 8.71-8.79 (m, 1H), 11.79 (br. s, 1H). (~3:1 mixture of amide rotational isomers). LC-MS (Method 1): Rₜ = 1.07 min MS (ESIpos): m/z = 504 [M + H]⁺ |
| 53 | 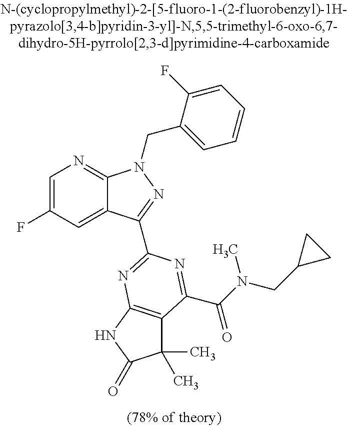 [SMILES: CN(CC1CC1)C(=O)c1nc(-c2nn(Cc3ccccc3F)c3ncc(F)cc23)nc2c1C(C)(C)C(=O)N2] | work-up Method c) ¹H-NMR (400 MHz, DMSO-d₆): δ [ppm] = 0.10-0.17 (m, 1H), 0.30- 0.40 (m, 2H), 0.52-0.59 (m, 1H), 0.98-1.08 (m, 0.5H), 1.10-1.22 (m, 0.5H), 1.32 (s, 3H), 1.36 (s, 3H), 2.92 (s, 1.5H), 3.06 (d, 1H), 3.14 (s, 1.5H), 3.44 (d, 1H), 5.84-5.90 (m, 2H), 7.12-7.31 (m, 3H), 7.33-7.41 (m, 1H), 8.47-8.53 (m, 1H), 8.74- 8.78 (m, 1H), 11.78 (br. s, 1H). (~1:1 mixture of amide rotational isomers). LC-MS (Method 1): Rₜ = 1.12 min MS (ESIpos): m/z = 518 [M + H]⁺ |
| 54 | 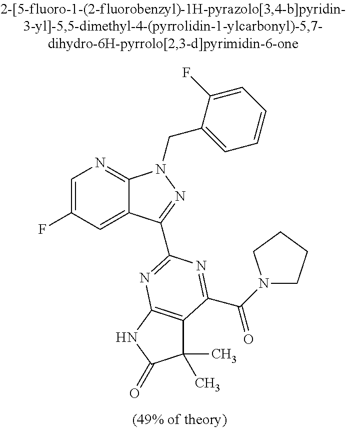 [SMILES: CC1(C)C(=O)Nc2nc(-c3nn(Cc4ccccc4F)c4ncc(F)cc34)nc(C(=O)N3CCCC3)c21] | work-up Method c) ¹H-NMR (400 MHz, DMSO-d₆): δ [ppm] = 1.36 (s, 6H), 1.75-1.96 (m, 4H), 3.32 signal superposed by water peak 3.56 (t, 2H), 5.87 (s, 2H), 7.17 (t, 1H), 7.19-7.28 (m, 2H), 7.33-7.42 (m, 1H), 8.51 (dd, 1H), 8.73-8.78 (m, 1H), 11.77 (s, 1H). LC-MS (Method 1): Rₜ = 1.04 min MS (ESIpos): m/z = 504 [M + H]⁺ |

### Example 55

### ent-N-(2-Amino-4,4-difluoro-2-methylbutyl)-2-{5-fluoro-1-[(3-fluoropyridin-2-yl)methyl]-1H-pyrazolo[3,4-b]pyridin-3-yl}-5,5-dimethyl-6-oxo-6,7-dihydro-5H-pyrrolo[2,3-d]pyrimidine-4-carboxamide (enantiomer A)

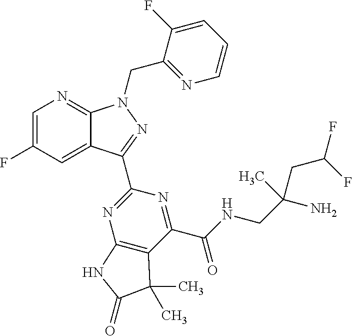 [SMILES: CC(N)(CNC(=O)c1nc(-c2nn(Cc3ncccc3F)c3ncc(F)cc23)nc2c1C(C)(C)C(=O)N2)CC(F)F]

26 mg (0.03 mmol) of ent-benzyl (4,4-difluoro-1-{[(2-{5-fluoro-1-[(3-fluoropyridin-2-yl)methyl]-1H-pyrazolo[3,4-b]pyridin-3-yl}-5,5-dimethyl-6-oxo-6,7-dihydro-5H-pyrrolo[2,3-d]pyrimidin-4-yl)carbonyl]amino}-2-methylbutan-2-yl)carbamate (enantiomer A) from Example 67A were dissolved in 0.8 ml of ethanol, 11 μl (0.15 mmol) of trifluoroacetic acid and 1 mg of palladium on activated carbon (10%) were added and the mixture was hydrogenated at atmospheric pressure and RT for 2 h. The reaction solution was subsequently filtered through a Millipore filter and the filtrate was concentrated under reduced pressure. The residue was taken up in dichloromethane/methanolic ammonia solution (2 N in methanol) and then purified by preparative thick-layer chromatography (mobile phase: dichloromethane/methanol=10/1). The product fractions were combined and concentrated. This gave 12 mg of the target compound (72% of theory).

LC-MS (Method 1): Rₜ=0.71 min

MS (ESIpos): m/z=572.5 [M+H]⁺

¹H-NMR (400 MHz, DMSO-d₆): δ [ppm]=1.13 (s, 3H), 1.51 (d, 6H), 1.93-2.07 (m, 2H), 3.25-3.42 (m, 2H; superposed by solvent peak), 6.02 (s, 2H), 6.13-6.46 (m, 1H), 7.41-7.47 (m, 1H), 7.74-7.81 (m, 1H), 8.24-8.28 (m, 1H), 8.71-8.74 (m, 1H), 8.75-8.79 (m, 1H), 8.84 (t, 1H).

The exemplary compounds listed in Table 4 were prepared analogously to the procedure from Example 55 from the appropriate starting materials. In each case, the reaction times were 0.5-3 h. Purifications were carried out by preparative thick-layer chromatography (mobile phase: dichloromethane/methanol=10/1 or 20/1).

TABLE 4

| Ex- | IUPAC name/structure | |
|---|---|---|
| ample | (Yield) | Work-up, analytical data |
| 56 | 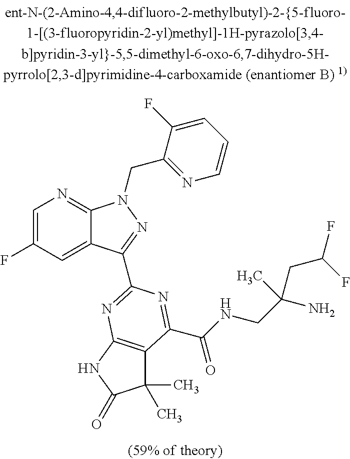 [SMILES: CC(N)(CNC(=O)c1nc(-c2nn(Cc3ncccc3F)c3ncc(F)cc23)nc2c1C(C)(C)C(=O)N2)CC(F)F] | ¹H-NMR (400 MHz, DMSO-d₆): δ [ppm] = 1.13 (s, 3H), 1.51 (d, 6H), 1.93-2.07 (m, 2H), 3.25-3.42 (m, 2H; superposed by solvent peak), 6.02 (s, 2H), 6.13-6.46 (m, 1H), 7.41-7.47 (m, 1H), 7.74-7.81 (m, 1H), 8.24-8.28 (m, 1H), 8.71-8.74 (m, 1H), 8.75-8.79 (m, 1H), 8.84 (t, 1H). LC-MS (Method 1): Rₜ = 0.71 min MS (ESIpos): m/z = 572.5 [M + H]⁺ |
| 57 | 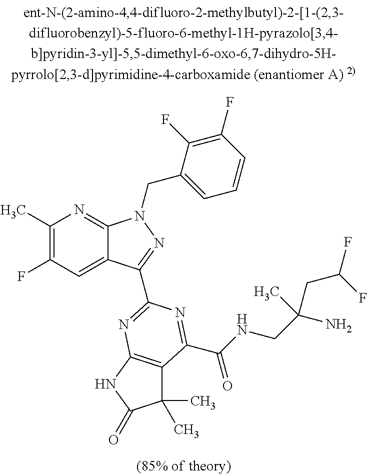 [SMILES: Cc1nc2c(cc1F)c(-c1nc3c(c(C(=O)NCC(C)(N)CC(F)F)n1)C(C)(C)C(=O)N3)nn2Cc1cccc(F)c1F] | ¹H-NMR (400 MHz, DMSO-d₆): δ [ppm] = 1.13 (s, 3H), 1.50 (d, 6H), 1.94-2.07 (m, 2H), 2.65 (d, 3H), 3.25-3.42 (m, 2H; superposed by solvent peak), 5.88 (s, 2H), 6.13- 6.46 (m, 1H), 7.01-7.08 (m, 1H), 7.13-7.21 (m, 1H), 7.36-7.45 (m, 1H), 8.67 (d, 1H), 8.83 (t, 1H). LC-MS (Method 1): Rₜ = 0.85 min MS (ESIpos): m/z = 603.5 [M + H]⁺ |
| 58 | 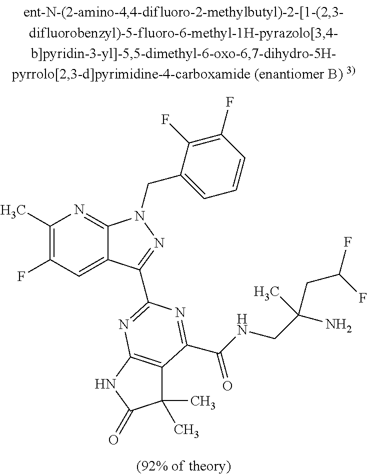 [SMILES: Cc1nc2c(cc1F)c(-c1nc3c(c(C(=O)NCC(C)(N)CC(F)F)n1)C(C)(C)C(=O)N3)nn2Cc1cccc(F)c1F] | ¹H-NMR (400 MHz, DMSO-d₆): δ [ppm] = 1.13 (s, 3H), 1.50 (d, 6H), 1.94-2.08 (m, 2H), 2.65 (d, 3H), 3.25-3.42 (m, 2H; superposed by solvent peak), 5.88 (s, 2H), 6.13- 6.46 (m, 1H), 7.01-7.08 (m, 1H), 7.13-7.21 (m, 1H), 7.35-7.44 (m, 1H), 8.67 (d, 1H), 8.83 (t, 1H). LC-MS (Method 1): Rₜ = 0.84 min MS (ESIpos): m/z = 603.5 [M + H]⁺ |

¹⁾ ent-Benzyl (4,4-difluoro-1-{[(2-{5-fluoro-1-[(3-fluoropyridin-2-yl)methyl]-1H-pyrazolo[3,4-b]pyridin-3-yl}-5,5-dimethyl-6-oxo-6,7-dihydro-5H-pyrrolo[2,3-d]pyrimidin-4-yl)carbonyl]amino}-2-methylbutan-2-yl)carbamate (enantiomer B) from Example 68A was employed.
²⁾ ent-Benzyl {1-[({2-[1-(2,3-difluorobenzyl)-5-fluoro-6-methyl-1H-pyrazolo[3,4-b]pyridin-3-yl]-5,5-dimethyl-6-oxo-6,7-dihydro-5H-pyrrolo[2,3-d]pyrimidin-4-yl}carbonyl)amino]-4,4-difluoro-2-methylbutan-2-yl}carbamate (enantiomer A) from Example 69A was employed.
³⁾ ent-Benzyl {1-[({2-[1-(2,3-difluorobenzyl)-5-fluoro-6-methyl-1H-pyrazolo[3,4-b]pyridin-3-yl]-5,5-dimethyl-6-oxo-6,7-dihydro-5H-pyrrolo[2,3-d]pyrimidin-4-yl}carbonyl)amino]-4,4-difluoro-2-methylbutan-2-yl}carbamate (enantiomer B) from Example 70A was employed.

### Example 59

### N-(2-Ethylbutyl)-2-[1-(2-fluorobenzyl)-1H-pyrazolo[3,4-b]pyridin-3-yl]-5,5-dimethyl-6-oxo-6,7-dihydro-5H-pyrrolo[2,3-d]pyrimidine-4-carboxamide

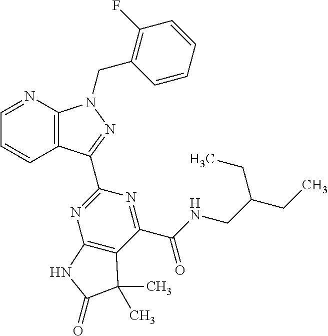 [SMILES: CCC(CC)CNC(=O)c1nc(-c2nn(Cc3ccccc3F)c3ncccc23)nc2c1C(C)(C)C(=O)N2]

### Step 1: 2-[1-(2-Fluorobenzyl)-1H-pyrazolo[3,4-b]pyridin-3-yl]-5,5-dimethyl-6-oxo-6,7-dihydro-5H-pyrrolo[2,3-d]pyrimidine-4-carbonyl chloride

At 0 C, 40.77 g (342.73 mmol) of thionyl chloride were added to 14.82 g (34.27 mmol) of 2-[1-(2-fluorobenzyl)-1H-pyrazolo[3,4-b]pyridin-3-yl]-5,5-dimethyl-6-oxo-6,7-dihydro-5H-pyrrolo[2,3-d]pyrimidin-4-carboxylic acid (Example 54A), and the mixture was stirred at RT for 3 h. The reaction solution was subsequently concentrated completely. 50 ml of toluene were then added to the residue and the solvent was subsequently removed under reduced pressure. This procedure was repeated twice.

### Step 2: N-(2-Ethylbutyl)-2-[1-(2-fluorobenzyl)-1H-pyrazolo[3,4-b]pyridin-3-yl]-5,5-dimethyl-6-oxo-6,7-dihydro-5H-pyrrolo[2,3-d]pyrimidine-4-carboxamide

10.12 mg (0.10 mmol) of 2-ethylbutane-1-amine were initially charged in a multititer plate (96 deep wells), and a solution of 45.09 mg (0.10 mmol) of 2-[1-(2-fluorobenzyl)-1H-pyrazolo[3,4-b]pyridin-3-yl]-5,5-dimethyl-6-oxo-6,7-dihydro-5H-pyrrolo[2,3-d]pyrimidine-4-carbonyl chloride (from step 1) in 0.6 ml of 1,2-dichloroethane was added. 64.62 mg (0.5 mol) of N,N-diisopropylethylamine were then added and the mixture was shaken at RT overnight. The solvent was then removed completely using a centrifugal drier, and 0.6 ml of DMF were then added to the residue. The reaction mixture was then filtered and the target compound was isolated from the filtrate by preparative LC-MS (Method 10). The product-containing fractions were concentrated under reduced pressure using a centrifugal dryer. The resulting residue of each product fraction was dissolved in 0.6 ml of DMSO. These fractions were then combined and finally freed of the solvent in a centrifugal dryer. 10.8 mg (21% of theory) of the title compound were obtained.

LC-MS (Method 9): Rₜ=1.26 min

MS (ESIpos): m/z=516 [M+H]⁺

The exemplary compounds shown in Table 5 were prepared analogously to Example 59 using the appropriate amines:

TABLE 5

| Ex- | IUPAC name/structure | |
|---|---|---|
| ample | (Yield) | Analytical data |
| 60 | 2-[1-(2-fluorobenzyl)-1H-pyrazolo[3,4-b]pyridin-3-yl]-N- | LC-MS (Method 9): Rₜ = 1.12 min |
| | [(1-hydroxycyclopropyl)methyl]-5,5-dimethyl-6-oxo-6,7- | MS (ESIpos): m/z = 506 [M + H]⁺ |
| | dihydro-5H-pyrrolo[2,3-d]pyrimidine-4-carboxamide | |
| | 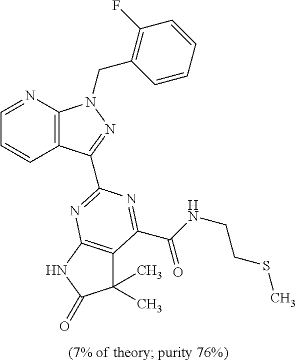 [SMILES: CSCCNC(=O)c1nc(-c2nn(Cc3ccccc3F)c3ncccc23)nc2c1C(C)(C)C(=O)N2] | |
| 61 | 2-[1-(2-fluorobenzyl)-1H-pyrazolo[3,4-b]pyridin-3-yl]-5,5- | LC-MS (Method 9): Rₜ = 1.16 min |
| | dimethyl-N-[3-(methylsulfanyl)propyl]-6-oxo-6,7-dihydro- | MS (ESIpos): m/z = 520 [M + H]⁺ |
| | 5H-pyrrolo[2,3-d]pyrimidine-4-carboxamide | |
| | 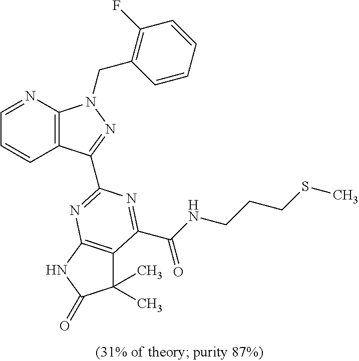 [SMILES: CSCCCNC(=O)c1nc(-c2nn(Cc3ccccc3F)c3ncccc23)nc2c1C(C)(C)C(=O)N2] | |
| 62 | 2-[1-(2-fluorobenzyl)-1H-pyrazolo[3,4-b]pyridin-3-yl]-5,5- | LC-MS (Method 9): Rₜ = 1.17 min |
| | dimethyl-6-oxo-N-(prop-2-en-1-yloxy)-6,7-dihydro-5H- | MS (ESIpos): m/z = 488 [M + H]⁺ |
| | pyrrolo[2,3-d]pyrimidine-4-carboxamide | |
| | 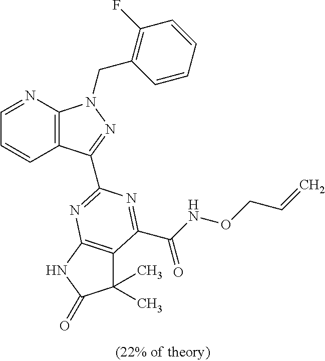 [SMILES: C=CCONC(=O)c1nc(-c2nn(Cc3ccccc3F)c3ncccc23)nc2c1C(C)(C)C(=O)N2] | |
| 63 | rac-N-(butan-2-yl)-2-[1-(2-fluorobenzyl)-1H-pyrazolo[3,4- | LC-MS (Method 9): Rₜ = 1.17 min |
| | b]pyridin-3-yl]-5,5-dimethyl-6-oxo-6,7-dihydro-5H- | MS (ESIpos): m/z = 488 [M + H]⁺ |
| | pyrrolo[2,3-d]pyrimidine-4-carboxamide | |
| | 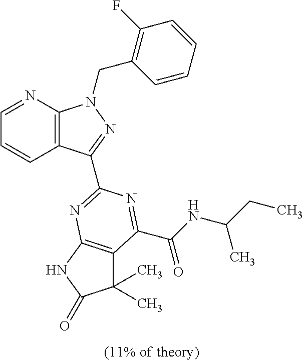 [SMILES: CCC(C)NC(=O)c1nc(-c2nn(Cc3ccccc3F)c3ncccc23)nc2c1C(C)(C)C(=O)N2] | |
| 64 | 2-[1-(2-fluorobenzyl)-1H-pyrazolo[3,4-b]pyridin-3-yl]-5,5- | LC-MS (Method 9): Rₜ = 0.96 min |
| | dimethyl-N-[2-(methylsulfinyl)ethyl]-6-oxo-6,7-dihydro- | MS (ESIpos): m/z = 522 [M + H]⁺ |
| | 5H-pyrrolo[2,3-d]pyrimidine-4-carboxamide | |
| | 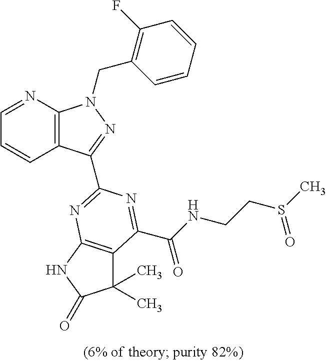 [SMILES: CS(=O)CCNC(=O)c1nc(-c2nn(Cc3ccccc3F)c3ncccc23)nc2c1C(C)(C)C(=O)N2] | |
| 65 | rac-2-[1-(2-fluorobenzyl)-1H-pyrazolo[3,4-b]pyridin-3- | LC-MS (Method 9): Rₜ = 1.07 min |
| | yl]-5,5-dimethyl-6-oxo-N-(tetrahydrofuran-3-yl)-6,7- | MS (ESIpos): m/z = 502 [M + H]⁺ |
| | dihydro-5H-pyrrolo[2,3-d]pyrimidine-4-carboxamide | |
| | 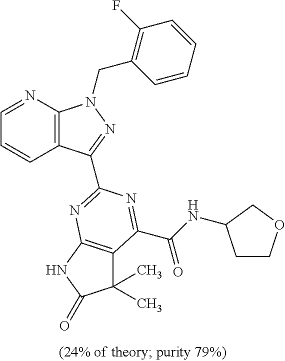 [SMILES: CC1(C)C(=O)Nc2nc(-c3nn(Cc4ccccc4F)c4ncccc34)nc(C(=O)NC3CCOC3)c21] | |
| 66 | rac-2-[1-(2-fluorobenzyl)-1H-pyrazolo[3,4-b]pyridin-3- | LC-MS (Method 9): Rₜ = 1.15 min |
| | yl]-5,5-dimethyl-6-oxo-N-(3-oxopentan-2-yl)-6,7-dihydro- | MS (ESIpos): m/z = 516 [M + H]⁺ |
| | 5H-pyrrolo[2,3-d]pyrimidine-4-carboxamide | |
| | 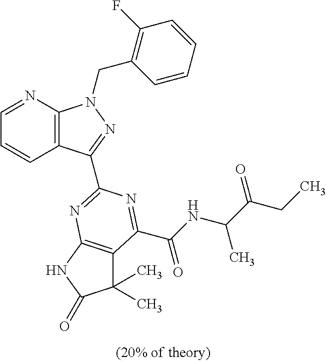 [SMILES: CCC(=O)C(C)NC(=O)c1nc(-c2nn(Cc3ccccc3F)c3ncccc23)nc2c1C(C)(C)C(=O)N2] | |
| 67 | 2-[1-(2-fluorobenzyl)-1H-pyrazolo[3,4-b]pyridin-3-yl]-5,5- | LC-MS (Method 9): Rₜ = 1.10 min |
| | dimethyl-N-(1-methyl-1H-pyrazol-5-yl)-6-oxo-6,7- | MS (ESIpos): m/z = 512 [M + H]⁺ |
| | dihydro-5H-pyrrolo[2,3-d]pyrimidine-4-carboxamide | |
| | 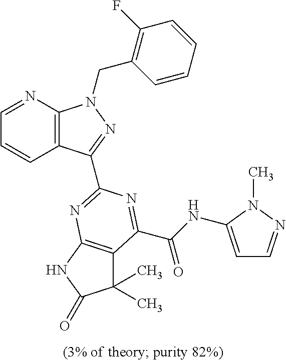 [SMILES: Cn1nccc1NC(=O)c1nc(-c2nn(Cc3ccccc3F)c3ncccc23)nc2c1C(C)(C)C(=O)N2] | |
| 68 | rac-N-(1-cyclopropylpropan-2-yl)-2-[1-(2-fluorobenzyl)- | LC-MS (Method 9): Rₜ = 1.21 min |
| | 1H-pyrazolo[3,4-b]pyridin-3-yl]-5,5-dimethyl-6-oxo-6,7- | MS (ESIpos): m/z = 514 [M + H]⁺ |
| | dihydro-5H-pyrrolo[2,3-d]pyrimidine-4-carboxamide | |
| | 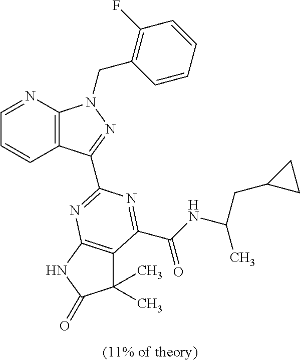 [SMILES: CC(CC1CC1)NC(=O)c1nc(-c2nn(Cc3ccccc3F)c3ncccc23)nc2c1C(C)(C)C(=O)N2] | |
| 69 | 2-[1-(2-fluorobenzyl)-1H-pyrazolo[3,4-b]pyridin-3-yl]-N- | LC-MS (Method 9): Rₜ = 1.14 min |
| | (furan-2-ylmethyl)-5,5-dimethyl-6-oxo-6,7-dihydro-5H- | MS (ESIpos): m/z = 512 [M + H]⁺ |
| | pyrrolo[2,3-d]pyrimidine-4-carboxamide | |
| | 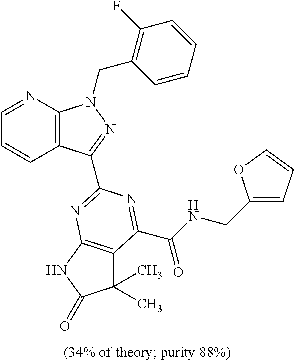 [SMILES: CC1(C)C(=O)Nc2nc(-c3nn(Cc4ccccc4F)c4ncccc34)nc(C(=O)NCc3ccco3)c21] | |
| 70 | 2-[1-(2-fluorobenzyl)-1H-pyrazolo[3,4-b]pyridin-3-yl]-5,5- | LC-MS (Method 9): Rₜ = 1.10 min |
| | dimethyl-N-(1-methyl-1H-pyrazol-3-yl)-6-oxo-6,7- | MS (ESIpos): m/z = 5 12 [M + H]⁺ |
| | dihydro-5H-pyrrolo[2,3-d]pyrimidine-4-carboxamide | |
| | 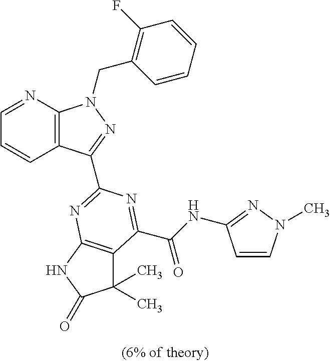 [SMILES: Cn1ccc(NC(=O)c2nc(-c3nn(Cc4ccccc4F)c4ncccc34)nc3c2C(C)(C)C(=O)N3)n1] | |
| 71 | 2-[1-(2-fluorobenzyl)-1H-pyrazolo[3,4-b]pyridin-3-yl]-5,5- | LC-MS (Method 9): Rₜ = 1.03 min |
| | dimethyl-N-(5-methyl-1,3,4-oxadiazol-2-yl)-6-oxo-6,7- | MS (ESIpos): m/z = 5 14 [M + H]⁺ |
| | dihydro-5H-pyrrolo[2,3-d]pyrimidine-4-carboxamide | |
| | 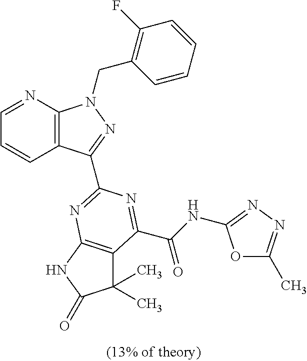 [SMILES: Cc1nnc(NC(=O)c2nc(-c3nn(Cc4ccccc4F)c4ncccc34)nc3c2C(C)(C)C(=O)N3)o1] | |
| 72 | 2-[1-(2-fluorobenzyl)-1H-pyrazolo[3,4-b]pyridin-3-yl]-5,5- | LC-MS (Method 9): Rₜ = 1.17 min |
| | dimethyl-6-oxo-N-(1,3-thiazol-2-yl)-6,7-dihydro-5H- | MS (ESIpos): m/z = 515 [M + H]⁺ |
| | pyrrolo[2,3-d]pyrimidine-4-carboxamide | |
| | 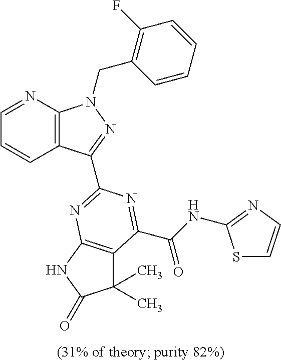 [SMILES: CC1(C)C(=O)Nc2nc(-c3nn(Cc4ccccc4F)c4ncccc34)nc(C(=O)Nc3nccs3)c21] | |
| 73 | 2-[1-(2-fluorobenzyl)-1H-pyrazolo[3,4-b]pyridin-3-yl]-5,5- | LC-MS (Method 9): Rₜ = 1.20 min |
| | dimethyl-6-oxo-N-{2-[(trifluoromethyl)sulfanyl]ethyl}- | MS (ESIpos): m/z = 560 [M + H]⁺ |
| | 6,7-dihydro-5H-pyrrolo[2,3-d]pyrimidine-4-carboxamide | |
| | 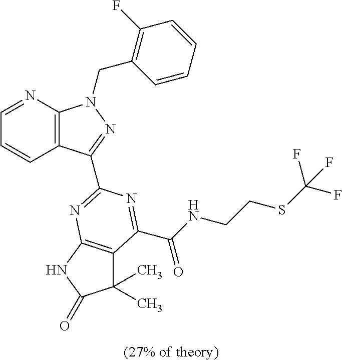 [SMILES: CC1(C)C(=O)Nc2nc(-c3nn(Cc4ccccc4F)c4ncccc34)nc(C(=O)NCCSC(F)(F)F)c21] | |
| 74 | 2-[1-(2-fluorobenzyl)-1H-pyrazolo[3,4-b]pyridin-3-yl]-5,5- | LC-MS (Method 9): Rₜ = 1.16 min |
| | dimethyl-6-oxo-N-(3,3,3-trifluoropropyl)-6,7-dihydro-5H- | MS (ESIpos): m/z = 528 [M + H]⁺ |
| | pyrrolo[2,3-d]pyrimidine-4-carboxamide | |
| | 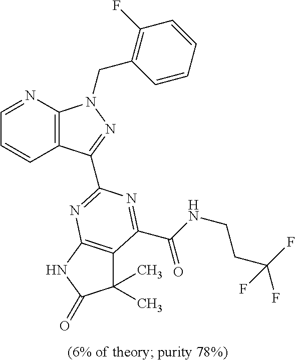 [SMILES: CC1(C)C(=O)Nc2nc(-c3nn(Cc4ccccc4F)c4ncccc34)nc(C(=O)NCCC(F)(F)F)c21] | |
| 75 | N-(2-amino-2-oxoethyl)-2-[1-(2-fluorobenzyl)-1H- | LC-MS (Method 9): Rₜ = 0.94 min |
| | pyrazolo[3,4-b]pyridin-3-yl]-5,5-dimethyl-6-oxo-6,7- | MS (ESIpos): m/z = 489 [M + H]⁺ |
| | dihydro-5H-pyrrolo[2,3-d]pyrimidine-4-carboxamide | |
| | 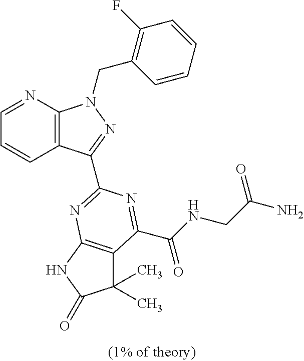 [SMILES: CC1(C)C(=O)Nc2nc(-c3nn(Cc4ccccc4F)c4ncccc34)nc(C(=O)NCC(N)=O)c21] | |
| 76 | N-(3,5-difluorophenyl)-2-[1-(2-fluorobenzyl)-1H- | LC-MS (Method 9): Rₜ = 1.30 min |
| | pyrazolo[3,4-b]pyridin-3-yl]-5,5-dimethyl-6-oxo-6,7- | MS (ESIpos): m/z = 544 [M + H]⁺ |
| | dihydro-5H-pyrrolo[2,3-d]pyrimidine-4-carboxamide | |
| | 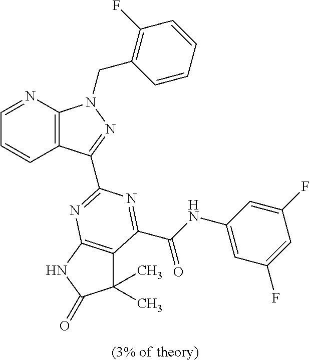 [SMILES: CC1(C)C(=O)Nc2nc(-c3nn(Cc4ccccc4F)c4ncccc34)nc(C(=O)Nc3cc(F)cc(F)c3)c21] | |
| 77 | 2-[1-(2-fluorobenzyl)-1H-pyrazolo[3,4-b]pyridin-3-yl]-5,5- | LC-MS (Method 9): Rₜ = 1.32 min |
| | dimethyl-6-oxo-N-(3,4,5-trifluorophenyl)-6,7-dihydro-5H- | MS (ESIpos): m/z = 562 [M + H]⁺ |
| | pyrrolo[2,3-d]pyrimidine-4-carboxamide | |
| | 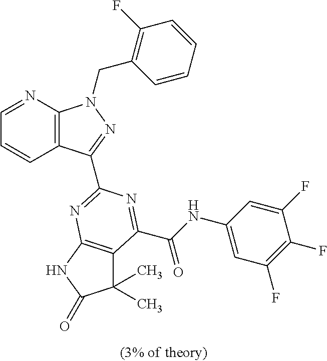 [SMILES: CC1(C)C(=O)Nc2nc(-c3nn(Cc4ccccc4F)c4ncccc34)nc(C(=O)Nc3cc(F)c(F)c(F)c3)c21] | |
| 78 | rac-2-[1-(2-fluorobenzyl)-1H-pyrazolo[3,4-b]pyridin-3- | LC-MS (Method 9): Rₜ = 1.04 min |
| | yl]-5,5-dimethyl-6-oxo-N-(2-oxotetrahydrofuran-3-yl)-6,7- | MS (ESIpos): m/z = 516 [M + H]⁺ |
| | dihydro-5H-pyrrolo[2,3-d]pyrimidine-4-carboxamide | |
| | 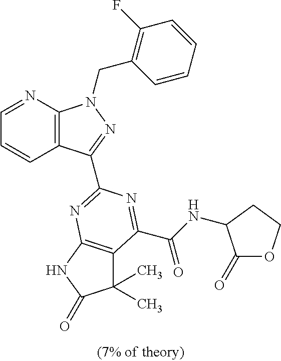 [SMILES: CC1(C)C(=O)Nc2nc(-c3nn(Cc4ccccc4F)c4ncccc34)nc(C(=O)NC3CCOC3=O)c21] | |
| 79 | 2-[1-(2-fluorobenzyl)-1H-pyrazolo[3,4-b]pyridin-3-yl]-N- | LC-MS (Method 9): Rₜ = 1.03 min |
| | methoxy-5,5-dimethyl-6-oxo-6,7-dihydro-5H-pyrrolo[2,3- | MS (ESIpos): m/z = 462 [M + H]⁺ |
| | d]pyrimidine-4-carboxamide | |
| | 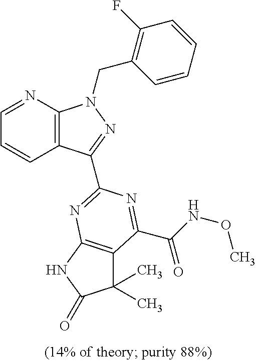 [SMILES: CONC(=O)c1nc(-c2nn(Cc3ccccc3F)c3ncccc23)nc2c1C(C)(C)C(=O)N2] | |
| 80 | N-[(2S)-1-amino-1-oxopropan-2-yl]-2-[1-(2-fluorobenzyl)- | LC-MS (Method 9): Rₜ = 0.97 min |
| | 1H-pyrazolo[3,4-b]pyridin-3-yl]-5,5-dimethyl-6-oxo-6,7- | MS (ESIpos): m/z = 503 [M + H]⁺ |
| | dihydro-5H-pyrrolo[2,3-d]pyrimidine-4-carboxamide | |
| | 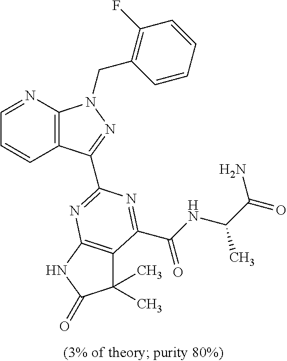 [SMILES: C[C@H](NC(=O)c1nc(-c2nn(Cc3ccccc3F)c3ncccc23)nc2c1C(C)(C)C(=O)N2)C(N)=O] | |
| 81 | 2-[1-(2-fluorobenzyl)-1H-pyrazolo[3,4-b]pyridin-3-yl]-5,5- | LC-MS (Method 9): Rₜ = 1.14 min |
| | dimethyl-6-oxo-N-propyl-6,7-dihydro-5H-pyrrolo[2,3- | MS (ESIpos): m/z = 474 [M + H]⁺ |
| | d]pyrimidine-4-carboxamide | |
| | 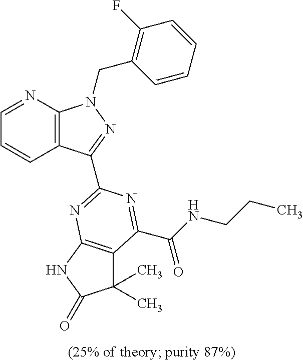 [SMILES: CCCNC(=O)c1nc(-c2nn(Cc3ccccc3F)c3ncccc23)nc2c1C(C)(C)C(=O)N2] | |
| 82 | N-[1,1′-bi(cyclopropyl)-1-yl]-2-[1-(2-fluorobenzyl)-1H- | LC-MS (Method 9): Rₜ = 1.17 min |
| | pyrazolo[3,4-b]pyridin-3-yl]-5,5-dimethyl-6-oxo-6,7- | MS (ESIpos): m/z = 512 [M + H]⁺ |
| | dihydro-5H-pyrrolo[2,3-d]pyrimidine-4-carboxamide | |
| | 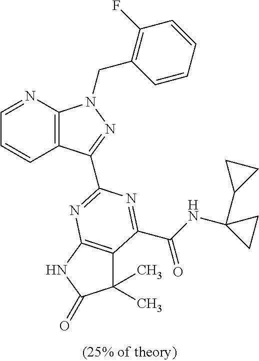 [SMILES: CC1(C)C(=O)Nc2nc(-c3nn(Cc4ccccc4F)c4ncccc34)nc(C(=O)NC3(C4CC4)CC3)c21] | |
| 83 | 2-[1-(2-fluorobenzyl)-1H-pyrazolo[3,4-b]pyridin-3-yl]-N- | LC-MS (Method 9): Rₜ = 1.08 min |
| | (2-fluoroethyl)-5,5-dimethyl-6-oxo-6,7-dihydro-5H- | MS (ESIpos): m/z = 478 [M + H]⁺ |
| | pyrrolo[2,3-d]pyrimidine-4-carboxamide | |
| | 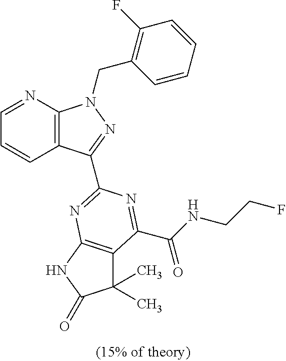 [SMILES: CC1(C)C(=O)Nc2nc(-c3nn(Cc4ccccc4F)c4ncccc34)nc(C(=O)NCCF)c21] | |
| 84 | N-(cyclopropylmethyl)-2-[1-(2-fluorobenzyl)-1H- | LC-MS (Method 9): Rₜ = 1.15 min |
| | pyrazolo[3,4-b]pyridin-3-yl]-5,5-dimethyl-6-oxo-6,7- | MS (ESIpos): m/z = 486 [M + H]⁺ |
| | dihydro-5H-pyrrolo[2,3-d]pyrimidine-4-carboxamide | |
| | 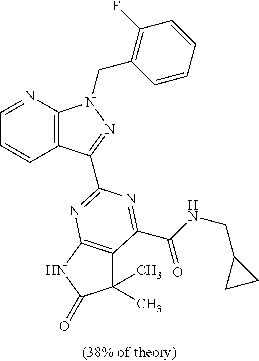 [SMILES: CC1(C)C(=O)Nc2nc(-c3nn(Cc4ccccc4F)c4ncccc34)nc(C(=O)NCC3CC3)c21] | |
| 85 | 2-[1-(2-fluorobenzyl)-1H-pyrazolo[3,4-b]pyridin-3-yl]-5,5- | LC-MS (Method 9): Rₜ = 1.20 min |
| | dimethyl-N-(3-methylbut-2-en-1-yl)-6-oxo-6,7-dihydro- | MS (ESIpos): m/z = 500 [M + H]⁺ |
| | 5H-pyrrolo[2,3-d]pyrimidine-4-carboxamide | |
| | 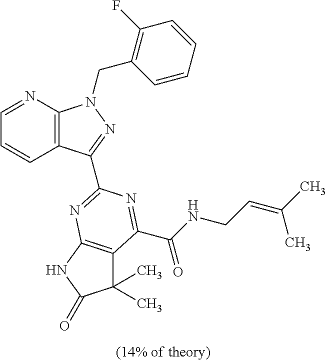 [SMILES: CC(C)=CCNC(=O)c1nc(-c2nn(Cc3ccccc3F)c3ncccc23)nc2c1C(C)(C)C(=O)N2] | |
| 86 | 2-[1-(2-fluorobenzyl)-1H-pyrazolo[3,4-b]pyridin-3-yl]- | LC-MS (Method 9): Rₜ = 1.04 min |
| | N,5,5-trimethyl-6-oxo-6,7-dihydro-5H-pyrrolo[2,3- | MS (ESIpos): m/z = 446 [M + H]⁺ |
| | d]pyrimidine-4-carboxamide | |
| | 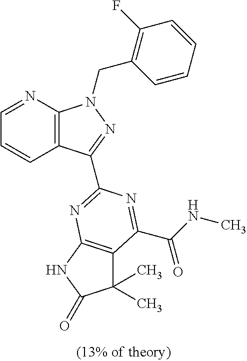 [SMILES: CNC(=O)c1nc(-c2nn(Cc3ccccc3F)c3ncccc23)nc2c1C(C)(C)C(=O)N2] | |
| 87 | 2-[1-(2-fluorobenzyl)-1H-pyrazolo[3,4-b]pyridin-3-yl]-5,5- | LC-MS (Method 9): Rₜ = 1.04 min |
| | dimethyl-6-oxo-N-(1H-pyrazol-3-yl)-6,7-dihydro-5H- | MS (ESIpos): m/z = 498 [M + H]⁺ |
| | pyrrolo[2,3-d]pyrimidine-4-carboxamide | |
| | 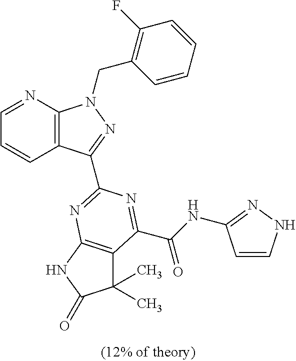 [SMILES: CC1(C)C(=O)Nc2nc(-c3nn(Cc4ccccc4F)c4ncccc34)nc(C(=O)Nc3cc[nH]n3)c21] | |
| 88 | 2-[1-(2-fluorobenzyl)-1H-pyrazolo[3,4-b]pyridin-3-yl]-5,5- | LC-MS (Method 9): Rₜ = 0.99 min |
| | dimethyl-6-oxo-N-(1H-pyrazol-3-yl)-6,7-dihydro-5H- | MS (ESIpos): m/z = 517 [M + H]⁺ |
| | pyrrolo[2,3-d]pyrimidine-4-carboxamide | |
| | 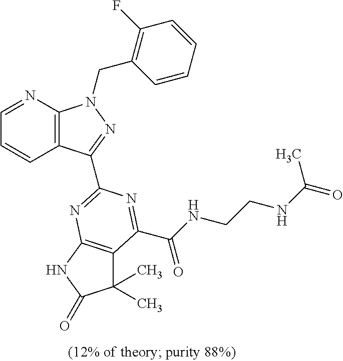 [SMILES: CC(=O)NCCNC(=O)c1nc(-c2nn(Cc3ccccc3F)c3ncccc23)nc2c1C(C)(C)C(=O)N2] | |
| 89 | 2-[1-(2-fluorobenzyl)-1H-pyrazolo[3,4-b]pyridin-3-yl]-5,5- | LC-MS (Method 9): Rₜ = 1.07 min |
| | dimethyl-6-oxo-N-(pyridin-3-yl)-6,7-dihydro-5H- | MS (ESIpos): m/z = 509 [M + H]⁺ |
| | pyrrolo[2,3-d]pyrimidine-4-carboxamide | |
| | 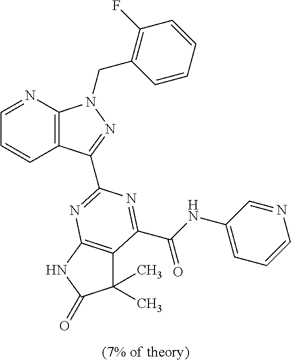 [SMILES: CC1(C)C(=O)Nc2nc(-c3nn(Cc4ccccc4F)c4ncccc34)nc(C(=O)Nc3cccnc3)c21] | |
| 90 | 2-[1-(2-fluorobenzyl)-1H-pyrazolo[3,4-b]pyridin-3-yl]-N- | LC-MS (Method 9): Rₜ = 1.10 min |
| | (3-methoxypropyl)-5,5-dimethyl-6-oxo-6,7-dihydro-5H- | MS (ESIpos): m/z = 504 [M + H]⁺ |
| | pyrrolo[2,3-d]pyrimidine-4-carboxamide | |
| | 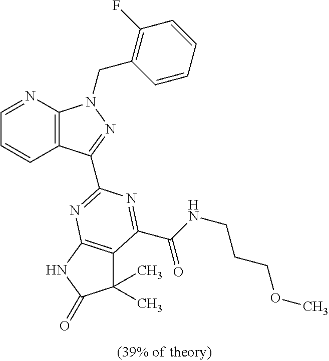 [SMILES: COCCCNC(=O)c1nc(-c2nn(Cc3ccccc3F)c3ncccc23)nc2c1C(C)(C)C(=O)N2] | |
| 91 | 2-[1-(2-fluorobenzyl)-1H-pyrazolo[3,4-b]pyridin-3-yl]-5,5- | LC-MS (Method 9): Rₜ = 0.97 min |
| | dimethyl-N-(6-methylpyridin-3-yl)-6-oxo-6,7-dihydro-5H- | MS (ESIpos): m/z = 523 [M + H]⁺ |
| | pyrrolo[2,3-d]pyrimidine-4-carboxamide | |
| | 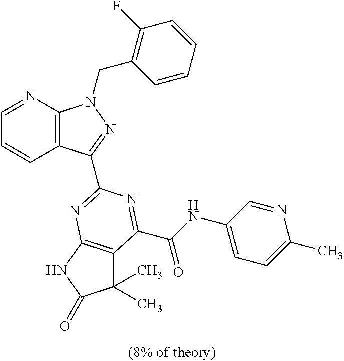 [SMILES: Cc1ccc(NC(=O)c2nc(-c3nn(Cc4ccccc4F)c4ncccc34)nc3c2C(C)(C)C(=O)N3)cn1] | |
| 92 | N-(cyclopentylmethyl)-2-[1-(2-fluorobenzyl)-1H- | LC-MS (Method 9): Rₜ = 1.24 min |
| | pyrazolo[3,4-b]pyridin-3-yl]-5,5-dimethyl-6-oxo-6,7- | MS (ESIpos): m/z = 514 [M + H]⁺ |
| | dihydro-5H-pyrrolo[2,3-d]pyrimidine-4-carboxamide | |
| | 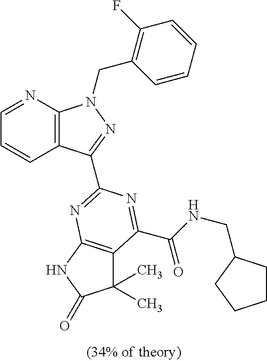 [SMILES: CC1(C)C(=O)Nc2nc(-c3nn(Cc4ccccc4F)c4ncccc34)nc(C(=O)NCC3CCCC3)c21] | |
| 93 | 2-[1-(2-fluorobenzyl)-1H-pyrazolo[3,4-b]pyridin-3-yl]-5,5- | LC-MS (Method 9): Rₜ = 1.11 min |
| | dimethyl-6-oxo-N-(1,3,4-thiadiazol-2-yl)-6,7-dihydro-5H- | MS (ESIpos): m/z = 516 [M + H]⁺ |
| | pyrrolo[2,3-d]pyrimidine-4-carboxamide | |
| | 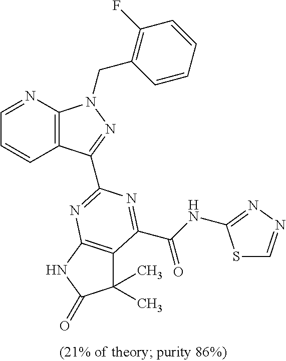 [SMILES: CC1(C)C(=O)Nc2nc(-c3nn(Cc4ccccc4F)c4ncccc34)nc(C(=O)Nc3nncs3)c21] | |
| 94 | 2-[1-(2-fluorobenzyl)-1H-pyrazolo[3,4-b]pyridin-3-yl]-5,5- | LC-MS (Method 9): Rₜ = 0.99 min |
| | dimethyl-6-oxo-N-(4H-1,2,4-triazol-3-yl)-6,7-dihydro-5H- | MS (ESIpos): m/z = 499 [M + H]⁺ |
| | pyrrolo[2,3-d]pyrimidine-4-carboxamide | |
| | 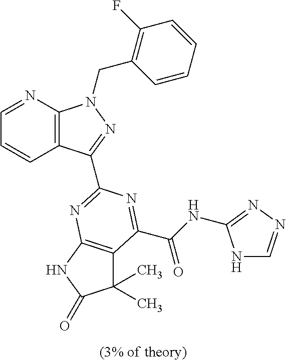 [SMILES: CC1(C)C(=O)Nc2nc(-c3nn(Cc4ccccc4F)c4ncccc34)nc(C(=O)Nc3nnc[nH]3)c21] | |
| 95 | N-(3-amino-3-oxopropyl)-2-[1-(2-fluorobenzyl)-1H- | LC-MS (Method 9): Rₜ = 0.95 min |
| | pyrazolo[3,4-b]pyridin-3-yl]-5,5-dimethyl-6-oxo-6,7- | MS (ESIpos): m/z = 503 [M + H]⁺ |
| | dihydro-5H-pyrrolo[2,3-d]pyrimidine-4-carboxamide | |
| | 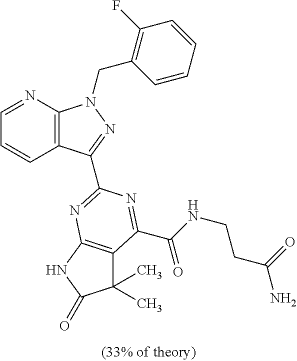 [SMILES: CC1(C)C(=O)Nc2nc(-c3nn(Cc4ccccc4F)c4ncccc34)nc(C(=O)NCCC(N)=O)c21] | |
| 96 | 2-[1-(2-fluorobenzyl)-1H-pyrazolo[3,4-b]pyridin-3-yl]-5,5- | LC-MS (Method 9): Rₜ = 1.15 min |
| | dimethyl-6-oxo-N-[2-(2,2,2-trifluoroethoxy)ethyl]-6,7- | MS (ESIpos): m/z = 558 [M + H]⁺ |
| | dihydro-5H-pyrrolo[2,3-d]pyrimidine-4-carboxamide | |
| | 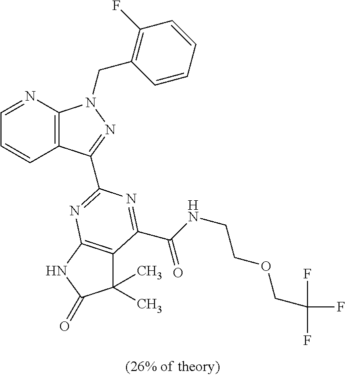 [SMILES: CC1(C)C(=O)Nc2nc(-c3nn(Cc4ccccc4F)c4ncccc34)nc(C(=O)NCCOCC(F)(F)F)c21] | |
| 97 | 2-[1-(2-fluorobenzyl)-1H-pyrazolo[3,4-b]pyridin-3-yl]-5,5- | LC-MS (Method 9): Rₜ = 1.20 min |
| | dimethyl-N-(1-methylcyclobutyl)-6-oxo-6,7-dihydro-5H- | MS (ESIpos): m/z = 500 [M + H]⁺ |
| | pyrrolo[2,3-d]pyrimidine-4-carboxamide | |
| | 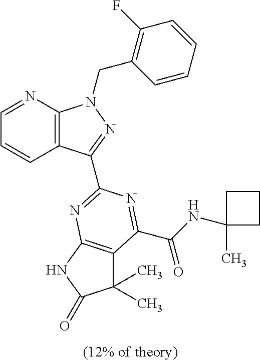 [SMILES: CC1(NC(=O)c2nc(-c3nn(Cc4ccccc4F)c4ncccc34)nc3c2C(C)(C)C(=O)N3)CCC1] | |
| 98 | 2-[1-(2-fluorobenzyl)-1H-pyrazolo[3,4-b]pyridin-3-yl]-5,5- | LC-MS (Method 9): Rₜ = 1.08 min |
| | dimethyl-6-oxo-N-(tetrahydro-2H-pyran-4-yl)-6,7- | MS (ESIpos): m/z = 516 [M + H]⁺ |
| | dihydro-5H-pyrrolo[2,3-d]pyrimidine-4-carboxamide | |
| | 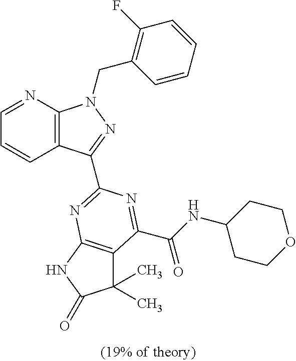 [SMILES: CC1(C)C(=O)Nc2nc(-c3nn(Cc4ccccc4F)c4ncccc34)nc(C(=O)NC3CCOCC3)c21] | |
| 99 | rac-2-[1-(2-fluorobenzyl)-1H-pyrazolo[3,4-b]pyridin-3- | LC-MS (Method 9): Rₜ = 1.08 min |
| | yl]-5,5-dimethyl-6-oxo-N-(tetrahydrofuran-3-ylmethyl)- | MS (ESIpos): m/z = 516 [M + H]⁺ |
| | 6,7-dihydro-5H-pyrrolo[2,3-d]pyrimidine-4-carboxamide | |
| | 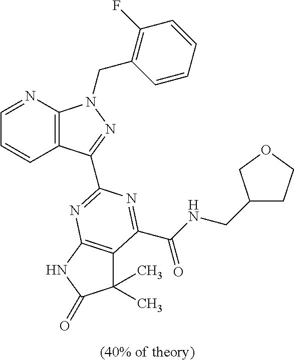 [SMILES: CC1(C)C(=O)Nc2nc(-c3nn(Cc4ccccc4F)c4ncccc34)nc(C(=O)NCC3CCOC3)c21] | |
| 100 | 2-[1-(2-fluorobenzyl)-1H-pyrazolo[3,4-b]pyridin-3-yl]-5,5- | LC-MS (Method 9): Rₜ = 1.03 min |
| | dimethyl-6-oxo-N-(1H-pyrazol-4-yl)-6,7-dihydro-5H- | MS (ESIpos): m/z = 498 [M + H]⁺ |
| | pyrrolo[2,3-d]pyrimidine-4-carboxamide | |
| | 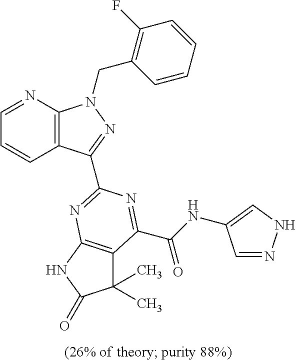 [SMILES: CC1(C)C(=O)Nc2nc(-c3nn(Cc4ccccc4F)c4ncccc34)nc(C(=O)Nc3cn[nH]c3)c21] | |
| 101 | 2-[1-(2-fluorobenzyl)-1H-pyrazolo[3,4-b]pyridin-3-yl]-5,5- | LC-MS (Method 9): Rₜ = 0.92 min |
| | dimethyl-N-(3-methylpyridin-4-yl)-6-oxo-6,7-dihydro-5H- | MS (ESIpos): m/z = 523 [M + H]⁺ |
| | pyrrolo[2,3-d]pyrimidine-4-carboxamide | |
| | 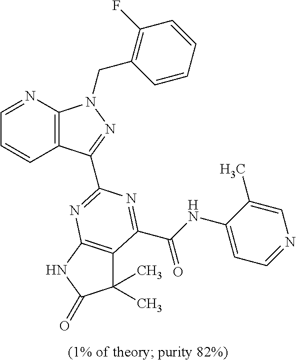 [SMILES: Cc1cnccc1NC(=O)c1nc(-c2nn(Cc3ccccc3F)c3ncccc23)nc2c1C(C)(C)C(=O)N2] | |
| 102 | N-(2,2-dimethylpropyl)-2-[1-(2-fluorobenzyl)-1H- | LC-MS (Method 9): Rₜ = 1.22 min |
| | pyrazolo[3,4-b]pyridin-3-yl]-5,5-dimethyl-6-oxo-6,7- | MS (ESIpos): m/z = 502 [M + H]⁺ |
| | dihydro-5H-pyrrolo[2,3-d]pyrimidine-4-carboxamide | |
| | 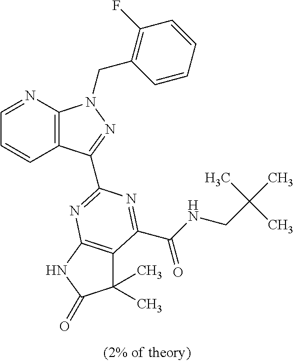 [SMILES: CC(C)(C)CNC(=O)c1nc(-c2nn(Cc3ccccc3F)c3ncccc23)nc2c1C(C)(C)C(=O)N2] | |
| 103 | 2-[1-(2-fluorobenzyl)-1H-pyrazolo[3,4-b]pyridin-3-yl]-5,5- | LC-MS (Method 9): Rₜ = 1.09 min |
| | dimethyl-N-[(3-methyloxetan-3-yl)methyl]-6-oxo-6,7- | MS (ESIpos): m/z = 516 [M + H]⁺ |
| | dihydro-5H-pyrrolo[2,3-d]pyrimidine-4-carboxamide | |
| | 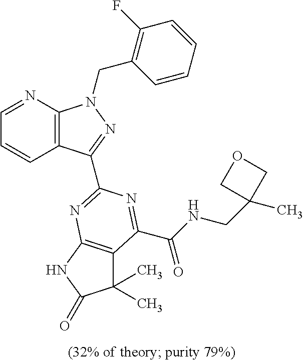 [SMILES: CC1(CNC(=O)c2nc(-c3nn(Cc4ccccc4F)c4ncccc34)nc3c2C(C)(C)C(=O)N3)COC1] | |
| 104 | rac-N-(1-cyclopropylethyl)-2-[1-(2-fluorobenzyl)-1H- | LC-MS (Method 9): Rₜ = 1.18 min |
| | pyrazolo[3,4-b]pyridin-3-yl]-5,5-dimethyl-6-oxo-6,7- | MS (ESIpos): m/z = 500 [M + H]⁺ |
| | dihydro-5H-pyrrolo[2,3-d]pyrimidine-4-carboxamide | |
| | 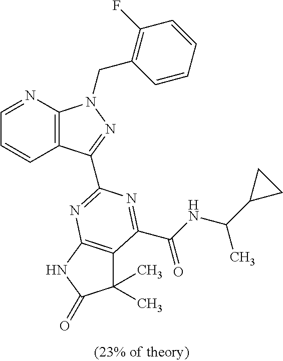 [SMILES: CC(NC(=O)c1nc(-c2nn(Cc3ccccc3F)c3ncccc23)nc2c1C(C)(C)C(=O)N2)C1CC1] | |
| 105 | rac-N-[2-(dimethylamino)propyl]-2-[1-(2-fluorobenzyl)- | LC-MS (Method 9): Rₜ = 0.78 min |
| | 1H-pyrazolo[3,4-b]pyridin-3-yl]-5,5-dimethyl-6-oxo-6,7- | MS (ESIpos): m/z = 517 [M + H]⁺ |
| | dihydro-5H-pyrrolo[2,3-d]pyrimidine-4-carboxamide | |
| | 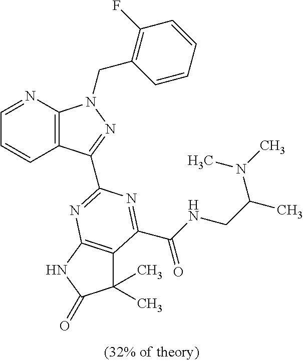 [SMILES: CC(CNC(=O)c1nc(-c2nn(Cc3ccccc3F)c3ncccc23)nc2c1C(C)(C)C(=O)N2)N(C)C] | |
| 106 | 2-[1-(2-fluorobenzyl)-1H-pyrazolo[3,4-b]pyridin-3-yl]-5,5- | LC-MS (Method 9): Rₜ = 0.86 min |
| | dimethyl-N-(2-methylpyridin-4-yl)-6-oxo-6,7-dihydro-5H- | MS (ESIpos): m/z = 523 [M + H]⁺ |
| | pyrrolo[2,3-d]pyrimidine-4-carboxamide | |
| | 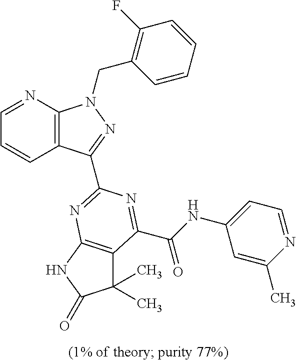 [SMILES: Cc1cc(NC(=O)c2nc(-c3nn(Cc4ccccc4F)c4ncccc34)nc3c2C(C)(C)C(=O)N3)ccn1] | |
| 107 | 2-[1-(2-fluorobenzyl)-1H-pyrazolo[3,4-b]pyridin-3-yl]-N- | LC-MS (Method 9): Rₜ = 1.19 min |
| | (5-fluoropyridin-3-yl)-5,5-dimethyl-6-oxo-6,7-dihydro-5H- | MS (ESIpos): m/z = 527 [M + H]⁺ |
| | pyrrolo[2,3-d]pyrimidine-4-carboxamide | |
| | 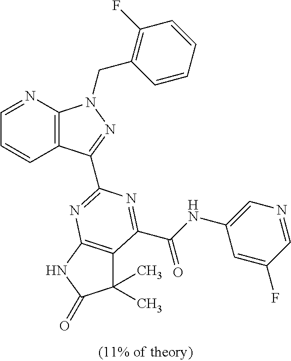 [SMILES: CC1(C)C(=O)Nc2nc(-c3nn(Cc4ccccc4F)c4ncccc34)nc(C(=O)Nc3cncc(F)c3)c21] | |
| 108 | 2-[1-(2-fluorobenzyl)-1H-pyrazolo[3,4-b]pyridin-3-yl]-5,5- | LC-MS (Method 9): Rₜ = 1.19 min |
| | dimethyl-6-oxo-N-(pyrazin-2-yl)-6,7-dihydro-5H- | MS (ESIpos): m/z = 510 [M + H]⁺ |
| | pyrrolo[2,3-d]pyrimidine-4-carboxamide | |
| | 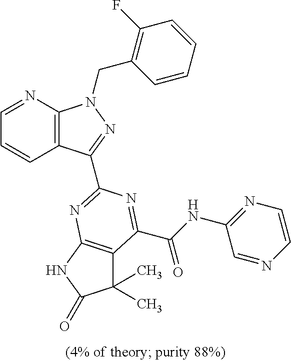 [SMILES: CC1(C)C(=O)Nc2nc(-c3nn(Cc4ccccc4F)c4ncccc34)nc(C(=O)Nc3cnccn3)c21] | |
| 109 | 2-[1-(2-fluorobenzyl)-1H-pyrazolo[3,4-b]pyridin-3-yl]-5,5- | LC-MS (Method 9): Rₜ = 1.03 min |
| | dimethyl-N-(1-methyl-1H-1,2,4-triazol-3-yl)-6-oxo-6,7- | MS (ESIpos): m/z = 513 [M + H]⁺ |
| | dihydro-5H-pyrrolo[2,3-d]pyrimidine-4-carboxamide | |
| | 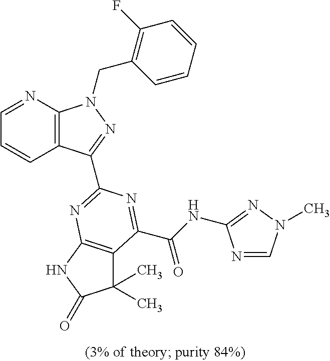 [SMILES: Cn1cnc(NC(=O)c2nc(-c3nn(Cc4ccccc4F)c4ncccc34)nc3c2C(C)(C)C(=O)N3)n1] | |
| 110 | N-(3-amino-3-oxopropyl)-2-[1-(2-fluorobenzyl)-1H- | LC-MS (Method 9): Rₜ = 0.91 min |
| | pyrazolo[3,4-b]pyridin-3-yl]-N,5,5-trimethyl-6-oxo-6,7- | MS (ESIpos): m/z = 517 [M + H]⁺ |
| | dihydro-5H-pyrrolo[2,3-d]pyrimidine-4-carboxamide | |
| | 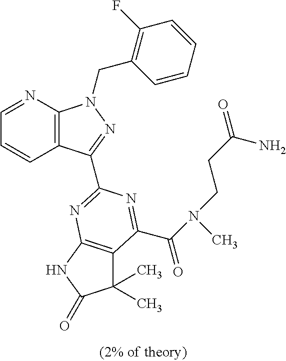 [SMILES: CN(CCC(N)=O)C(=O)c1nc(-c2nn(Cc3ccccc3F)c3ncccc23)nc2c1C(C)(C)C(=O)N2] | |
| 111 | N-(cyclopropylmethyl)-2-[1-(2-fluorobenzyl)-1H- | LC-MS (Method 9): Rₜ = 1.11 min |
| | pyrazolo[3,4-b]pyridin-3-yl]-N,5,5-trimethyl-6-oxo-6,7- | MS (ESIpos): m/z = 500 [M + H]⁺ |
| | dihydro-5H-pyrrolo[2,3-d]pyrimidine-4-carboxamide | |
| | 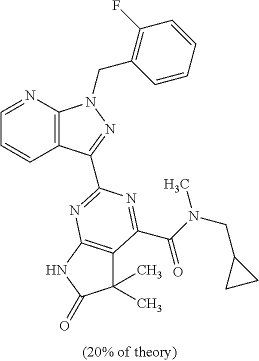 [SMILES: CN(CC1CC1)C(=O)c1nc(-c2nn(Cc3ccccc3F)c3ncccc23)nc2c1C(C)(C)C(=O)N2] | |
| 112 | 2-[1-(2-fluorobenzyl)-1H-pyrazolo[3,4-b]pyridin-3-yl]- | LC-MS (Method 9): Rₜ = 1.10 min |
| | N,5,5-trimethyl-6-oxo-N-(propan-2-yl)-6,7-dihydro-5H- | MS (ESIpos): m/z = 488 [M + H]⁺ |
| | pyrrolo[2,3-d]pyrimidine-4-carboxamide | |
| | 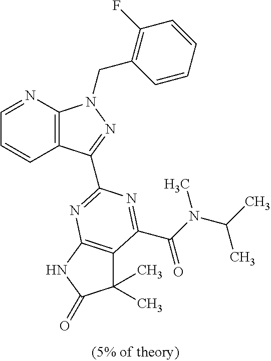 [SMILES: CC(C)N(C)C(=O)c1nc(-c2nn(Cc3ccccc3F)c3ncccc23)nc2c1C(C)(C)C(=O)N2] | |
| 113 | 2-[1-(2-fluorobenzyl)-1H-pyrazolo[3,4-b]pyridin-3-yl]-5,5- | LC-MS (Method 9): Rₜ = 1.09 min |
| | dimethyl-N-(3-methylpyridin-2-yl)-6-oxo-6,7-dihydro-5H- | MS (ESIpos): m/z = 523 [M + H]⁺ |
| | pyrrolo[2,3-d]pyrimidine-4-carboxamide | |
| | 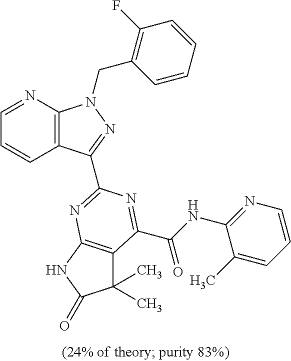 [SMILES: Cc1cccnc1NC(=O)c1nc(-c2nn(Cc3ccccc3F)c3ncccc23)nc2c1C(C)(C)C(=O)N2] | |
| 114 | 2-[1-(2-fluorobenzyl)-1H-pyrazolo[3,4-b]pyridin-3-yl]-N- | LC-MS (Method 9): Rₜ = 0.98 min |
| | (2-hydroxyethyl)-5,5-dimethyl-6-oxo-6,7-dihydro-5H- | MS (ESIpos): m/z = 476 [M + H]⁺ |
| | pyrrolo[2,3-d]pyrimidine-4-carboxamide | |
| | 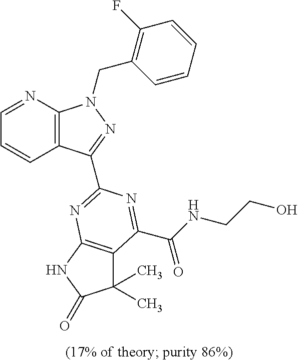 [SMILES: CC1(C)C(=O)Nc2nc(-c3nn(Cc4ccccc4F)c4ncccc34)nc(C(=O)NCCO)c21] | |
| 115 | N-benzyl-2-[1-(2-fluorobenzyl)-1H-pyrazolo[3,4- | LC-MS (Method 9): Rₜ = 1.18 min |
| | b]pyridin-3-yl]-5,5-dimethyl-6-oxo-6,7-dihydro-5H- | MS (ESIpos): m/z = 522 [M + H]⁺ |
| | pyrrolo[2,3-d]pyrimidine-4-carboxamide | |
| | 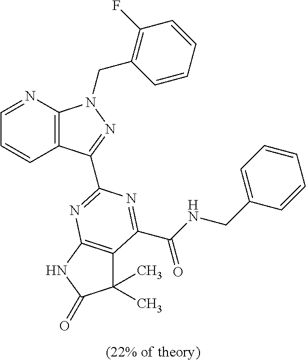 [SMILES: CC1(C)C(=O)Nc2nc(-c3nn(Cc4ccccc4F)c4ncccc34)nc(C(=O)NCc3ccccc3)c21] | |
| 116 | 2-[1-(2-fluorobenzyl)-1H-pyrazolo[3,4-b]pyridin-3-yl]-5,5- | LC-MS (Method 9): Rₜ = 0.88 min |
| | dimethyl-6-oxo-N-(pyridin-4-yl)-6,7-dihydro-5H- | MS (ESIpos): m/z = 509 [M + H]⁺ |
| | pyrrolo[2,3-d]pyrimidine-4-carboxamide | |
| | 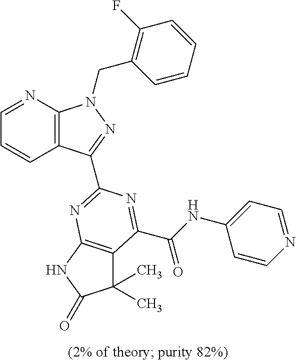 [SMILES: CC1(C)C(=O)Nc2nc(-c3nn(Cc4ccccc4F)c4ncccc34)nc(C(=O)Nc3ccncc3)c21] | |
| 117 | 2-[1-(2-fluorobenzyl)-1H-pyrazolo[3,4-b]pyridin-3-yl]-5,5- | LC-MS (Method 9): Rₜ = 1.05 min |
| | dimethyl-6-oxo-N-(pyridin-2-ylmethyl)-6,7-dihydro-5H- | MS (ESIpos): m/z = 523 [M + H]⁺ |
| | pyrrolo[2,3-d]pyrimidine-4-carboxamide | |
| | 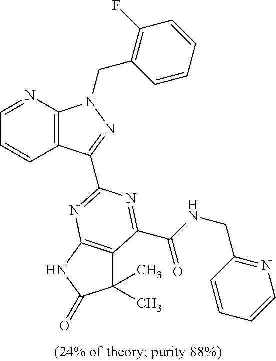 [SMILES: CC1(C)C(=O)Nc2nc(-c3nn(Cc4ccccc4F)c4ncccc34)nc(C(=O)NCc3ccccn3)c21] | |
| 118 | 2-[1-(2-fluorobenzyl)-1H-pyrazolo[3,4-b]pyridin-3-yl]-5,5- | LC-MS (Method 9): Rₜ = 0.91 min |
| | dimethyl-6-oxo-N-(pyridin-4-ylmethyl)-6,7-dihydro-5H- | MS (ESIpos): m/z = 523 [M + H]⁺ |
| | pyrrolo[2,3-d]pyrimidine-4-carboxamide | |
| | 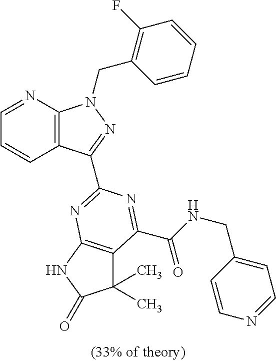 [SMILES: CC1(C)C(=O)Nc2nc(-c3nn(Cc4ccccc4F)c4ncccc34)nc(C(=O)NCc3ccncc3)c21] | |
| 119 | 2-[1-(2-fluorobenzyl)-1H-pyrazolo[3,4-b]pyridin-3-yl]-5,5- | LC-MS (Method 9): Rₜ = 0.99 min |
| | dimethyl-N-(4-methylpyridin-3-yl)-6-oxo-6,7-dihydro-5H- | MS (ESIpos): m/z = 523 [M + H]⁺ |
| | pyrrolo[2,3-d]pyrimidine-4-carboxamide | |
| | 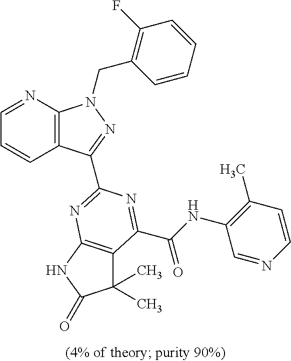 [SMILES: Cc1ccncc1NC(=O)c1nc(-c2nn(Cc3ccccc3F)c3ncccc23)nc2c1C(C)(C)C(=O)N2] | |
| 120 | 2-[1-(2-fluorobenzyl)-1H-pyrazolo[3,4-b]pyridin-3-yl]-N- | LC-MS (Method 9): Rₜ = 0.94 min |
| | (2-hydroxyethyl)-N,5,5-trimethyl-6-oxo-6,7-dihydro-5H- | MS (ESIpos): m/z = 490 [M + H]⁺ |
| | pyrrolo[2,3-d]pyrimidine-4-carboxamide | |
| | 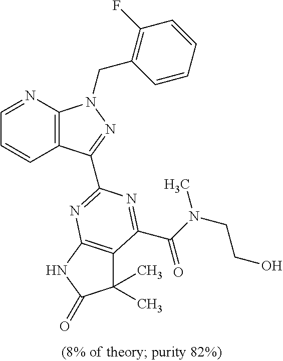 [SMILES: CN(CCO)C(=O)c1nc(-c2nn(Cc3ccccc3F)c3ncccc23)nc2c1C(C)(C)C(=O)N2] | |
| 121 | N-butyl-2-[1-(2-fluorobenzyl)-1H-pyrazolo[3,4-b]pyridin- | LC-MS (Method 9): Rₜ = 1.19 min |
| | 3-yl]-5,5-dimethyl-6-oxo-6,7-dihydro-5H-pyrrolo[2,3- | MS (ESIpos): m/z = 488 [M + H]⁺ |
| | d]pyrimidine-4-carboxamide | |
| | 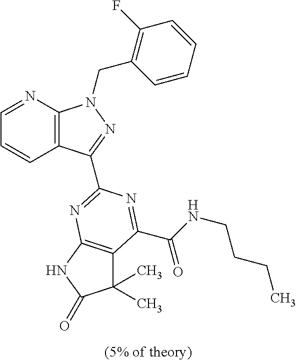 [SMILES: CCCCNC(=O)c1nc(-c2nn(Cc3ccccc3F)c3ncccc23)nc2c1C(C)(C)C(=O)N2] | |
| 122 | 2-[1-(2-fluorobenzyl)-1H-pyrazolo[3,4-b]pyridin-3-yl]-5,5- | LC-MS (Method 9): Rₜ = 1.19 min |
| | dimethyl-N-(2-methylpropyl)-6-oxo-6,7-dihydro-5H- | MS (ESIpos): m/z = 488 [M + H]⁺ |
| | pyrrolo[2,3-d]pyrimidine-4-carboxamide | |
| | 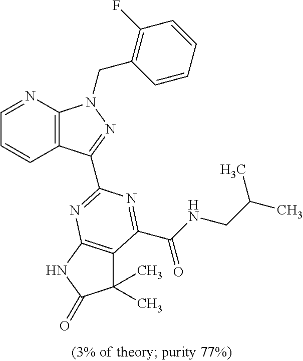 [SMILES: CC(C)CNC(=O)c1nc(-c2nn(Cc3ccccc3F)c3ncccc23)nc2c1C(C)(C)C(=O)N2] | |
| 123 | N-(3-ethoxypropyl)-2-[1-(2-fluorobenzyl)-1H- | LC-MS (Method 9): Rₜ = 1.13 min |
| | pyrazolo[3,4-b]pyridin-3-yl]-5,5-dimethyl-6-oxo-6,7- | MS (ESIpos): m/z = 518 [M + H]⁺ |
| | dihydro-5H-pyrrolo[2,3-d]pyrimidine-4-carboxamide | |
| | 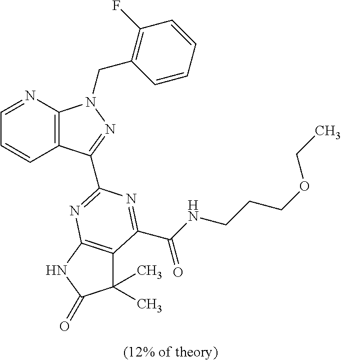 [SMILES: CCOCCCNC(=O)c1nc(-c2nn(Cc3ccccc3F)c3ncccc23)nc2c1C(C)(C)C(=O)N2] | |
| 124 | 2-[1-(2-fluorobenzyl)-1H-pyrazolo[3,4-b]pyridin-3-yl]-N- | LC-MS (Method 9): Rₜ = 1.23 min |
| | hexyl-5,5-dimethyl-6-oxo-6,7-dihydro-5H-pyrrolo[2,3- | MS (ESIpos): m/z = 516 [M + H]⁺ |
| | d]pyrimidine-4-carboxamide | |
| | 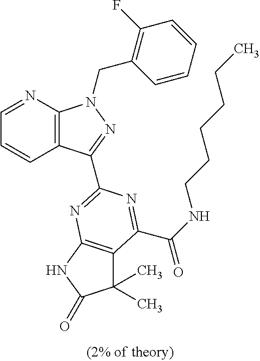 [SMILES: CCCCCCNC(=O)c1nc(-c2nn(Cc3ccccc3F)c3ncccc23)nc2c1C(C)(C)C(=O)N2] | |
| 125 | rac-2-[1-(2-fluorobenzyl)-1H-pyrazolo[3,4-b]pyridin-3- | LC-MS (Method 9): Rₜ = 1.23 min |
| | yl]-5,5-dimethyl-N-(2-methylbutyl)-6-oxo-6,7-dihydro- | MS (ESIpos): m/z = 502 [M + H]⁺ |
| | 5H-pyrrolo[2,3-d]pyrimidine-4-carboxamide | |
| | 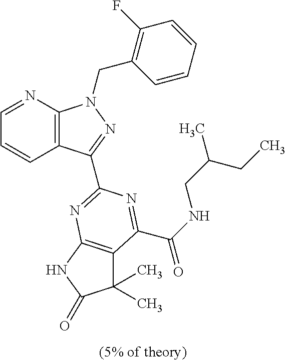 [SMILES: CCC(C)CNC(=O)c1nc(-c2nn(Cc3ccccc3F)c3ncccc23)nc2c1C(C)(C)C(=O)N2] | |
| 126 | N-cyclopentyl-2-[1-(2-fluorobenzyl)-1H-pyrazolo[3,4- | LC-MS (Method 9): Rₜ = 1.20 min |
| | b]pyridin-3-yl]-5,5-dimethyl-6-oxo-6,7-dihydro-5H- | MS (ESIpos): m/z = 500 [M + H]⁺ |
| | pyrrolo[2,3-d]pyrimidine-4-carboxamide | |
| | 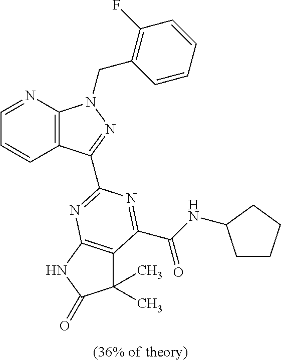 [SMILES: CC1(C)C(=O)Nc2nc(-c3nn(Cc4ccccc4F)c4ncccc34)nc(C(=O)NC3CCCC3)c21] | |
| 127 | 2-[1-(2-fluorobenzyl)-1H-pyrazolo[3,4-b]pyridin-3-yl]-N- | LC-MS (Method 9): Rₜ = 1.09 min |
| | (2-methoxyethyl)-5,5-dimethyl-6-oxo-6,7-dihydro-5H- | MS (ESIpos): m/z = 490 [M + H]⁺ |
| | pyrrolo[2,3-d]pyrimidine-4-carboxamide | |
| | 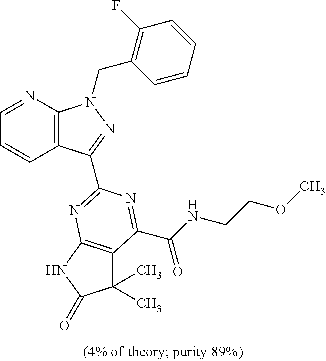 [SMILES: COCCNC(=O)c1nc(-c2nn(Cc3ccccc3F)c3ncccc23)nc2c1C(C)(C)C(=O)N2] | |
| 128 | N-[2-(dimethylamino)ethyl]-2-[1-(2-fluorobenzyl)-1H- | LC-MS (Method 9): Rₜ = 0.79 min |
| | pyrazolo[3,4-b]pyridin-3-yl]-5,5-dimethyl-6-oxo-6,7- | MS (ESIpos): m/z = 503 [M + H]⁺ |
| | dihydro-5H-pyrrolo[2,3-d]pyrimidine-4-carboxamide | |
| | 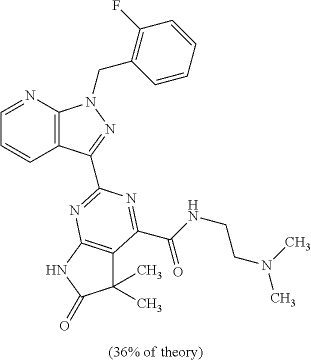 [SMILES: CN(C)CCNC(=O)c1nc(-c2nn(Cc3ccccc3F)c3ncccc23)nc2c1C(C)(C)C(=O)N2] | |
| 129 | N-ethyl-2-[1-(2-fluorobenzyl)-1H-pyrazolo[3,4-b]pyridin- | LC-MS (Method 9): Rₜ = 1.10 min |
| | 3-yl]-5,5-dimethyl-6-oxo-6,7-dihydro-5H-pyrrolo[2,3- | MS (ESIpos): m/z = 460 [M + H]⁺ |
| | d]pyrimidine-4-carboxamide | |
| | 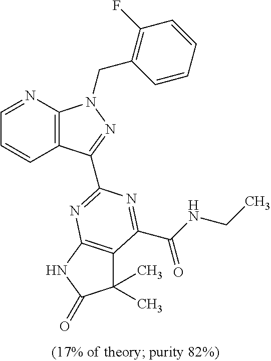 [SMILES: CCNC(=O)c1nc(-c2nn(Cc3ccccc3F)c3ncccc23)nc2c1C(C)(C)C(=O)N2] | |
| 130 | rac-2-[1-(2-fluorobenzyl)-1H-pyrazolo[3,4-b]pyridin-3- | LC-MS (Method 9): Rₜ = 1.13 min |
| | yl]-5,5-dimethyl-6-oxo-N-(tetrahydrofuran-2-ylmethyl)- | MS (ESIpos): m/z = 516 [M + H]⁺ |
| | 6,7-dihydro-5H-pyrrolo[2,3-d]pyrimidine-4-carboxamide | |
| | 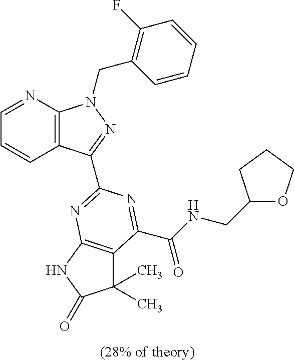 [SMILES: CC1(C)C(=O)Nc2nc(-c3nn(Cc4ccccc4F)c4ncccc34)nc(C(=O)NCC3CCCO3)c21] | |
| 131 | 2-[1-(2-Fluorobenzyl)-1H-pyrazolo[3,4-b]pyridin-3-yl]- | LC-MS (Method 9): Rₜ = 1.23 min |
| | 5,5-dimethyl-6-oxo-N-pentyl-6,7-dihydro-5H-pyrrolo[2,3- | MS (ESIpos): m/z = 502 [M + H]⁺ |
| | d]pyrimidine-4-carboxamide | |
| | 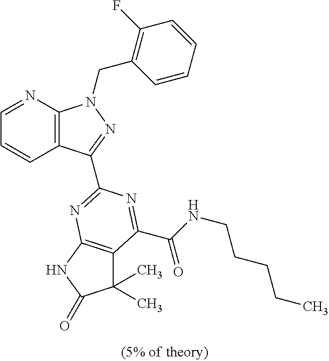 [SMILES: CCCCCNC(=O)c1nc(-c2nn(Cc3ccccc3F)c3ncccc23)nc2c1C(C)(C)C(=O)N2] | |
| 132 | 2-[1-(2-fluorobenzyl)-1H-pyrazolo[3,4-b]pyridin-3-yl]-5,5- | LC-MS (Method 9): Rₜ = 1.22 min |
| | dimethyl-N-(3-methylbutyl)-6-oxo-6,7-dihydro-5H- | MS (ESIpos): m/z = 502 [M + H]⁺ |
| | pyrrolo[2,3-d]pyrimidine-4-carboxamide | |
| | 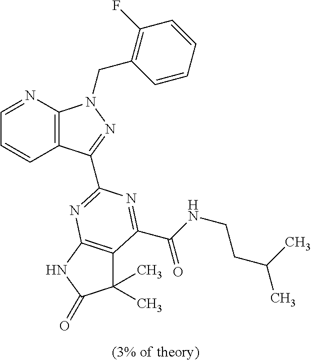 [SMILES: CC(C)CCNC(=O)c1nc(-c2nn(Cc3ccccc3F)c3ncccc23)nc2c1C(C)(C)C(=O)N2] | |
| 133 | rac-2-[1-(2-fluorobenzyl)-1H-pyrazolo[3,4-b]pyridin-3- | LC-MS (Method 9): Rₜ = 1.22 min |
| | yl]-5,5-dimethyl-N-(3-methylbutan-2-yl)-6-oxo-6,7- | MS (ESIpos): m/z = 502 [M + H]⁺ |
| | dihydro-5H-pyrrolo[2,3-d]pyrimidine-4-carboxamide | |
| | 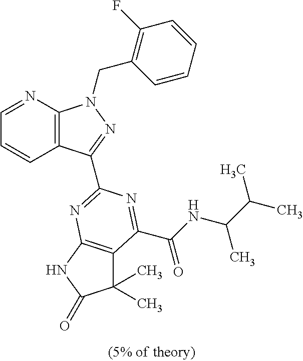 [SMILES: CC(C)C(C)NC(=O)c1nc(-c2nn(Cc3ccccc3F)c3ncccc23)nc2c1C(C)(C)C(=O)N2] | |
| 134 | N-[(2S)-butan-2-yl]-2-[1-(2-fluorobenzyl)-1H- | LC-MS (Method 9): Rₜ = 1.18 min |
| | pyrazolo[3,4-b]pyridin-3-yl]-5,5-dimethyl-6-oxo-6,7- | MS (ESIpos): m/z = 488 [M + H]⁺ |
| | dihydro-5H-pyrrolo[2,3-d]pyrimidine-4-carboxamide | |
| | 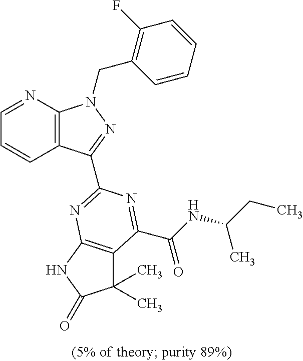 [SMILES: CC[C@H](C)NC(=O)c1nc(-c2nn(Cc3ccccc3F)c3ncccc23)nc2c1C(C)(C)C(=O)N2] | |
| 135 | N-(3,3-dimethylbutyl)-2-[1-(2-fluorobenzyl)-1H- | LC-MS (Method 9): Rₜ = 1.23 min |
| | pyrazolo[3,4-b]pyridin-3-yl]-5,5-dimethyl-6-oxo-6,7- | MS (ESIpos): m/z = 516 [M + H]⁺ |
| | dihydro-5H-pyrrolo[2,3-d]pyrimidine-4-carboxamide | |
| | 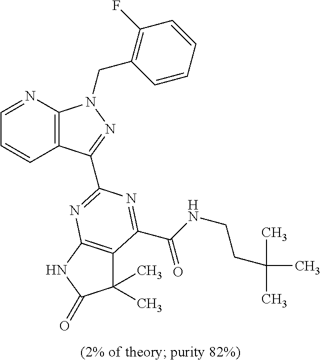 [SMILES: CC(C)(C)CCNC(=O)c1nc(-c2nn(Cc3ccccc3F)c3ncccc23)nc2c1C(C)(C)C(=O)N2] | |
| 136 | rac-N-(2-amino-4,4,4-trifluorobutyl)-2-[1-(2- | LC-MS (Method 9): Rₜ = 0.84 min |
| | fluorobenzyl)-1H-pyrazolo[3,4-b]pyridin-3-yl]-5,5- | MS (ESIpos): m/z = 557 [M + H]⁺ |
| | dimethyl-6-oxo-6,7-dihydro-5H-pyrrolo[2,3-d]pyrimidine- | |
| | 4-carboxamide | |
| | 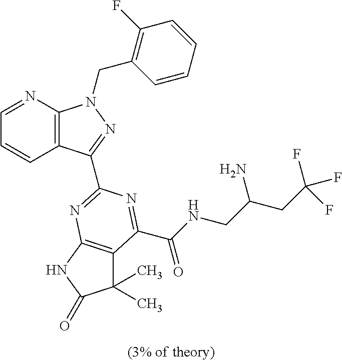 [SMILES: CC1(C)C(=O)Nc2nc(-c3nn(Cc4ccccc4F)c4ncccc34)nc(C(=O)NCC(N)CC(F)(F)F)c21] | |
| 137 | rac-N-(2-amino-4,4,4-trifluorobutyl)-2-[1-(2- | LC-MS (Method 9): Rₜ = 1.01 min |
| | fluorobenzyl)-1H-pyrazolo[3,4-b]pyridin-3-yl]-5,5- | MS (ESIpos): m/z = 557 [M + H]⁺ |
| | dimethyl-6-oxo-6,7-dihydro-5H-pyrrolo[2,3-d]pyrimidine- | |
| | 4-carboxamide | |
| | 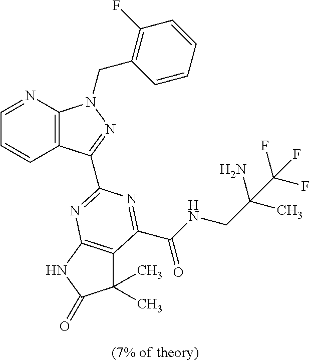 [SMILES: CC1(C)C(=O)Nc2nc(-c3nn(Cc4ccccc4F)c4ncccc34)nc(C(=O)NCC(C)(N)C(F)(F)F)c21] | |
| 138 | 2-[1-(2-fluorobenzyl)-1H-pyrazolo[3,4-b]pyridin-3-yl]-N- | LC-MS (Method 9): Rₜ = 1.03 min |
| | [(1-hydroxycyclopropyl)methyl]-5,5-dimethyl-6-oxo-6,7- | MS (ESIpos): m/z = 502 [M + H]⁺ |
| | dihydro-5H-pyrrolo[2,3-d]pyrimidine-4-carboxamide | |
| | 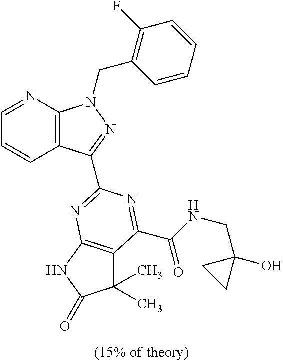 [SMILES: CC1(C)C(=O)Nc2nc(-c3nn(Cc4ccccc4F)c4ncccc34)nc(C(=O)NCC3(O)CC3)c21] | |

### B. Assessment of Pharmacological Efficacy

The pharmacological activity of the compounds of the invention can be demonstrated by in vitro and in vivo studies as known to the person skilled in the art. The application examples which follow describe the biological action of the compounds of the invention, without restricting the invention to these examples.

### Abbreviations and Acronyms

The following abbreviations are used:
AUC area under the curve
BSA bovine serum albumin
Cₘₐₓ peak plasma concentration
Caco-2 epithelial cell line
DMSO dimethyl sulfoxide
EDTA ethylenediaminetetraacetic acid
F bioavailability
Hepes 2-[4-(2-hydroxyethyl)piperazin-1-yl]ethanesulfonic acid
IC inhibition concentration
MEC minimum effective concentration
NADH nicotinamide adenine dinucleotide phosphate
PDE 5 phosphodiesterase 5
PEG polyethylene glycol
Tris tris(hydroxymethyl)aminomethane

### B-1. Vasorelaxant Effect In Vitro

The determination of the relaxant activity of the compounds of the invention on isolated vessels was carried out as described in JP Stasch et al., Br J Pharmacol. 2002; 135, 333-343. Rabbits are stunned by a blow to the neck and exsanguinated. The aorta is removed, freed from adhering tissue and divided into rings of width 1.5 mm, which are placed individually under prestress into 5 ml organ baths with carbogen-sparged Krebs-Henseleit solution at 37° C. having the following composition (each in mM): sodium chloride: 119; potassium chloride: 4.8; calcium chloride dihydrate: 1; magnesium sulfate heptahydrate: 1.4; potassium dihydrogenphosphate: 1.2; sodium bicarbonate: 25; glucose: 10. The contractile force is determined with Statham UC2 cells, amplified and digitalized using A/D transducers (DAS-1802 HC, Keithley Instruments Munich), and recorded in parallel on linear recorders.

To generate a contraction, phenylephrine is added to the bath cumulatively in increasing concentration. After several control cycles, the substance to be studied is added in increasing dosage each time in every further run, and the magnitude of the contraction is compared with the magnitude of the contraction attained in the last preceding run. This is used to calculate the concentration needed to reduce the magnitude of the control value by 50% (IC₅₀ value). The standard administration volume is 5 μl; the DMSO content in the bath solution corresponds to 0.1%.

### B-2. Effect on a Recombinant Guanylate Cyclase Reporter Cell Line

The cellular activity of the compounds of the invention is determined using a recombinant guanylate cyclase reporter cell line, as described in F. Wunder et al., Anal. Biochem. 2005, 339, 104-112. Representative values (MEC=minimum effective concentration) for the compounds of the invention are shown in the table below (Table 1B; in some cases as means of individual determinations):

TABLE 1B

| Example no. | MEC [μM] |
|---|---|
| 1 | 0.065 |
| 2 | 0.03 |
| 3 | 0.03 |
| 4 | 0.2 |
| 5 | 0.3 |
| 6 | 0.2 |
| 7 | 3 |
| 8 | 2 |
| 9 | 0.3 |
| 10 | 0.3 |
| 11 | 0.1 |
| 12 | 0.065 |
| 13 | 0.1 |
| 14 | 0.03 |
| 15 | 0.2 |
| 16 | 0.17 |
| 17 | 0.03 |
| 18 | 0.3 |
| 19 | 10 |
| 20 | 1 |
| 21 | 3 |
| 22 | 2 |
| 23 | 6.5 |
| 24 | 0.1 |
| 25 | 0.3 |
| 26 | 0.3 |
| 27 | 0.1 |
| 28 | 0.03 |
| 29 | 0.3 |
| 30 | 1 |
| 31 | 0.1 |
| 32 | 1 |
| 33 | 0.1 |
| 34 | 1 |
| 35 | 10 |
| 36 | 3 |
| 37 | 0.1 |
| 38 | 0.01 |
| 39 | 0.3 |
| 40 | 0.03 |
| 41 | 0.065 |
| 42 | 0.3 |
| 43 | 0.3 |
| 44 | 0.3 |
| 45 | 0.3 |
| 46 | 0.03 |
| 47 | 0.03 |
| 48 | 0.1 |
| 49 | 0.1 |
| 50 | 0.1 |
| 51 | 0.1 |
| 52 | 0.01 |
| 53 | 0.01 |
| 54 | 0.03 |
| 55 | 3 |
| 56 | 3 |
| 57 | 1 |
| 58 | 1 |
| 59 | 1 |
| 60 | 0.3 |
| 61 | 0.1 |
| 62 | 0.1 |
| 63 | 0.1 |
| 64 | 1 |
| 65 | 1 |
| 66 | 0.3 |
| 67 | 0.3 |
| 68 | 0.1 |
| 69 | 0.1 |
| 70 | 0.3 |
| 71 | 0.3 |
| 72 | 1 |
| 73 | 0.3 |
| 74 | 0.1 |
| 75 | 0.3 |
| 76 | 1 |
| 77 | 1 |
| 78 | 0.3 |
| 79 | 0.1 |
| 80 | 1 |
| 81 | 0.1 |
| 82 | 0.1 |
| 83 | 0.1 |
| 84 | 0.1 |
| 85 | 0.1 |
| 86 | 0.1 |
| 87 | 0.3 |
| 88 | 0.3 |
| 89 | 0.3 |
| 90 | 0.1 |
| 91 | 0.3 |
| 92 | 0.3 |
| 93 | 0.1 |
| 94 | 0.3 |
| 95 | 1 |
| 96 | 0.03 |
| 97 | 0.3 |
| 98 | 1 |
| 99 | 0.3 |
| 100 | 0.3 |
| 101 | 0.3 |
| 102 | 0.3 |
| 103 | 0.3 |
| 104 | 0.1 |
| 105 | 1 |
| 106 | 0.3 |
| 107 | 0.3 |
| 108 | 0.3 |
| 109 | 0.3 |
| 110 | 1 |
| 111 | 0.01 |
| 112 | 0.03 |
| 113 | 0.1 |
| 114 | 0.3 |
| 115 | 0.1 |
| 116 | 1 |
| 117 | 0.1 |
| 118 | 0.1 |
| 119 | 0.3 |
| 120 | 0.1 |
| 121 | 0.3 |
| 122 | 0.1 |
| 123 | 0.1 |
| 124 | 1 |
| 125 | 0.3 |
| 126 | 0.3 |
| 127 | 0.1 |
| 128 | 1 |
| 129 | 0.1 |
| 130 | 0.3 |
| 131 | 0.3 |
| 132 | 0.3 |
| 133 | 1 |
| 134 | 0.3 |
| 135 | 1 |
| 136 | 1 |
| 137 | 0.3 |
| 138 | 1 |

### B-3. Inhibition of Human Phosphodiesterase 5 (PDE 5)

PDE 5 preparations are obtained from human platelets by disruption (Microfluidizer®, 800 bar, 3 passes), followed by centrifugation (75 000 g, 60 min, 4° C.) and ion exchange chromatography of the supernatant on a Mono Q 10/10 column (linear sodium chloride gradient, elution with a 0.2-0.3M solution of sodium chloride in buffer (20 mM Hepes pH 7.2, 2 mM magnesium chloride). Fractions having PDE 5 activity are combined (PDE 5 preparation) and stored at −80° C.

To determine their in vitro action on human PDE 5, the test substances are dissolved in 100% DMSO and serially diluted. Typically, dilution series (1:3) from 200 μM to 0.091 μM are prepared (resulting final concentrations in the test: 4 μM to 0.0018 μM). In each case 2 μl of the diluted substance solutions are placed into the wells of microtitre plates (Isoplate-96/200W; Perkin Elmer). Subsequently, 50 μl of a dilution of the above-described PDE 5 preparation are added. The dilution of the PDE 5 preparation is chosen such that during the later incubation less than 70% of the substrate are converted (typical dilution: 1:100; dilution buffer: 50 mM tris/hydrochloric acid pH 7.5, 8.3 mM magnesium chloride, 1.7 mM EDTA, 0.2% BSA). The substrate, [8-³H] cyclic guanosine-3′,5′-monophosphate (1 μCi/μl; Perkin Elmer), is diluted 1:2000 with assay buffer (50 mM tris/hydrochloric acid pH 7.5, 8.3 mM magnesium chloride, 1.7 mM EDTA) to a concentration of 0.0005 μCi/μl. By addition of 50 μl (0.025 μCi) of the diluted substrate, the enzyme reaction is finally started. The test mixtures are incubated at room temperature for 60 min and the reaction is stopped by adding 25 μl of a suspension of 18 mg/ml yttrium scintillation proximity beads in water (phosphodiesterase beads for SPA assays, RPNQ 0150, Perkin Elmer). The microtitre plates are sealed with a film and left to stand at room temperature for 60 min. Subsequently, the plates are analysed for 30 s per well in a Microbeta scintillation counter (Perkin Elmer). IC₅₀ values are determined using the graphic plot of the substance concentration against percentage PDE 5 inhibition. Representative IC₅₀ values for the compounds of the invention are shown in the table below (Table 2B; in some cases as means of individual determinations):

TABLE 2B

| Example no. | IC50 [nM] |
|---|---|
| 1 | 20 |
| 2 | 13 |
| 3 | 100 |
| 4 | 110 |
| 5 | 86 |
| 6 | 140 |
| 7 | 130 |
| 8 | 81 |
| 9 | 58 |
| 10 | 110 |
| 11 | 91 |
| 12 | 130 |
| 13 | 600 |
| 14 | 130 |
| 15 | 1200 |
| 16 | 170 |
| 17 | 280 |
| 18 | 570 |
| 19 | 220 |
| 20 | 190 |
| 21 | 230 |
| 22 | 170 |
| 23 | 1300 |
| 24 | 3.5 |
| 25 | 2.0 |
| 26 | 930 |
| 27 | 51 |
| 28 | 5.2 |
| 29 | 12 |
| 30 | 310 |
| 31 | 10 |
| 32 | 12 |
| 33 | 110 |
| 34 | 77 |
| 35 | 1300 |
| 36 | 73 |
| 37 | 4 |
| 38 | 770 |
| 39 | 34 |
| 40 | 240 |
| 41 | 88 |
| 42 | 74 |
| 43 | 14 |
| 44 | 110 |
| 45 | 130 |
| 46 | 1000 |
| 47 | 730 |
| 48 | 1100 |
| 49 | 33 |
| 50 | 19 |
| 51 | 87 |
| 52 | 150 |
| 53 | 110 |
| 54 | 1200 |
| 55 | 190 |
| 56 | 120 |
| 57 | 100 |
| 58 | 86 |
| 59 | 91 |
| 60 | 15 |
| 61 | 16 |
| 62 | 18 |
| 63 | 11 |
| 64 | 74 |
| 65 | 6.8 |
| 66 | 87 |
| 67 | 2.6 |
| 68 | 13 |
| 69 | 10 |
| 70 | 24 |
| 71 | 170 |
| 72 | 46 |
| 73 | 52 |
| 74 | 26 |
| 76 | 60 |
| 77 | 87 |
| 78 | 9.3 |
| 79 | 30 |
| 80 | 23 |
| 81 | 15 |
| 82 | 8.6 |
| 83 | 13 |
| 84 | 23 |
| 85 | 38 |
| 86 | 44 |
| 87 | 24 |
| 88 | 46 |
| 89 | 9.0 |
| 90 | 14 |
| 91 | 68 |
| 92 | 35 |
| 93 | 55 |
| 94 | 16 |
| 95 | 48 |
| 96 | 62 |
| 97 | 8.2 |
| 98 | 33 |
| 99 | 31 |
| 100 | 4.3 |
| 103 | 10 |
| 104 | 11 |
| 105 | 620 |
| 107 | 73 |
| 108 | 25 |
| 109 | 39 |
| 111 | 710 |
| 112 | 93 |
| 113 | 44 |
| 114 | 15 |
| 115 | 41 |
| 117 | 25 |
| 118 | 21 |
| 119 | 10 |
| 120 | 220 |
| 121 | 27 |
| 122 | 20 |
| 123 | 26 |
| 125 | 30 |
| 126 | 6 |
| 127 | 16 |
| 128 | 330 |
| 129 | 10 |
| 130 | 28 |
| 131 | 38 |
| 132 | 33 |
| 133 | 30 |
| 134 | 13 |
| 136 | 42 |
| 137 | 42 |
| 138 | 21 |

### B-4. Radiotelemetry Measurement of Blood Pressure in Conscious, Spontaneously Hypertensive Rats

A commercially available telemetry system from DATA SCIENCES INTERNATIONAL DSI, USA, is employed for the blood pressure measurement on conscious rats described below.

The system consists of 3 main components:
implantable transmitters (Physiotel® telemetry transmitter)
receivers (Physiotel® receiver) which are linked via a multiplexer (DSI Data Exchange Matrix) to a
data acquisition computer.

The telemetry system makes it possible to continuously record blood pressure, heart rate and body motion of conscious animals in their usual habitat.

### Animal Material

The studies are conducted on adult female spontaneously hypertensive rats (SHR Okamoto) with a body weight of >200 g. SHR/NCrl from the Okamoto Kyoto School of Medicine, 1963, were a cross of male Wistar Kyoto rats having greatly elevated blood pressure and female rats having slightly elevated blood pressure, and were handed over at F13 to the U.S. National Institutes of Health.

After transmitter implantation, the experimental animals are housed singly in type 3 μMakrolon cages. They have free access to standard feed and water.

The day/night rhythm in the experimental laboratory is changed by the room lighting at 6:00 am and at 7:00 pm.

### Transmitter Implantation

The TA11 PA-C40 telemetry transmitters used are surgically implanted under aseptic conditions in the experimental animals at least 14 days before the first experimental use. The animals instrumented in this way can be used repeatedly after the wound has healed and the implant has settled.

For the implantation, the fasted animals are anesthetized with pentobarbital (Nembutal, Sanofi: 50 mg/kg i.p.) and shaved and disinfected over a large area of their abdomens. After the abdominal cavity has been opened along the linea alba, the liquid-filled measuring catheter of the system is inserted into the descending aorta in the cranial direction above the bifurcation and fixed with tissue glue (VetBonD TM, 3M). The transmitter housing is fixed intraperitoneally to the abdominal wall muscle, and the wound is closed layer by layer.

An antibiotic (Tardomyocel COMP, Bayer, 1 ml/kg s.c.) is administered postoperatively for prophylaxis of infection.

### Substances and Solutions

Unless stated otherwise, the substances to be studied are administered orally by gavage to a group of animals in each case (n=6). In accordance with an administration volume of 5 ml/kg of body weight, the test substances are dissolved in suitable solvent mixtures or suspended in 0.5% tylose.

A solvent-treated group of animals is used as control.

### Experimental Procedure

The telemetry measuring unit present is configured for 24 animals. Each experiment is recorded under an experiment number (Vyear month day).

Each of the instrumented rats living in the system is assigned a separate receiving antenna (1010 Receiver, DSI).

The implanted transmitters can be activated externally by means of an incorporated magnetic switch. They are switched to transmission in the run-up to the experiment. The signals emitted can be detected online by a data acquisition system (Dataquest TM A.R.T. for WINDOWS, DSI) and processed accordingly. The data are stored in each case in a file created for this purpose and bearing the experiment number.

In the standard procedure, the following are measured for 10-second periods in each case:
systolic blood pressure (SBP)
diastolic blood pressure (DBP)
mean arterial pressure (MAP)
heart rate (HR)
activity (ACT).

The acquisition of measurements is repeated under computer control at 5-minute intervals. The source data obtained as absolute values are corrected in the diagram with the currently measured barometric pressure (Ambient Pressure Reference Monitor; APR-1) and stored as individual data. Further technical details are given in the extensive documentation from the manufacturer company (DSI).

Unless indicated otherwise, the test substances are administered at 9:00 am on the day of the experiment. Following the administration, the parameters described above are measured over 24 hours.

### Evaluation

After the end of the experiment, the acquired individual data are sorted using the analysis software (DATAQUEST TM A.R.T. TM ANALYSIS). The blank value is assumed here to be the time 2 hours before administration, and so the selected data set encompasses the period from 7:00 am on the day of the experiment to 9:00 am on the following day.

The data are smoothed over a predefinable period by determination of the average (15-minute average) and transferred as a text file to a storage medium. The measured values presorted and compressed in this way are transferred to Excel templates and tabulated. For each day of the experiment, the data obtained are stored in a dedicated file bearing the number of the experiment. Results and test protocols are stored in files in paper form sorted by numbers.

### REFERENCES

Klaus Witte, Kai Hu, Johanna Swiatek, Claudia Mtissig, Georg Ertl and Bjorn Lemmer: Experimental heart failure in rats: effects on cardiovascular circadian rhythms and on myocardial β-adrenergic signaling. Cardiovasc Res 47 (2): 203-405, 2000; Kozo Okamoto: Spontaneous hypertension in rats. Int Rev Exp Pathol 7: 227-270, 1969; Maarten van den Buuse: Circadian Rhythms of Blood Pressure, Heart Rate, and Locomotor Activity in Spontaneously Hypertensive Rats as Measured With Radio-Telemetry. Physiology & Behavior 55(4): 783-787, 1994

### B-5. Determination of Organ-Protective Effects in a Long-Term Experiment on Rats

The organ-protective effects of the compounds of the invention are shown in a therapeutically relevant “low nitric oxide (NO)/high renin” hypertension model in rats. The study was carried out analogously to the recently published article (Sharkovska Y, et al. J Hypertension 2010; 28: 1666-1675). This involves treating renin-transgenic rats (TGR(mRen2)27) to which the NO synthase inhibitor L-NAME had been administered via drinking water simultaneously with the compound according to the invention or vehicle over several weeks. Hemodynamic and renal parameters are determined during the treatment period. At the end of the long-term study, organ protection (kidney, lung, heart, aorta) is shown by histopathological studies, biomarkers, expression analyses and cardiovascular plasma parameters.

### B-6. Measurements of the Pulmonary Artery Pressure (PAP) in Conscious Dogs Under Hypoxia Conditions

A telemetry system from DATA SCIENCES INTERNATIONAL DSI, USA, for example, is employed for the blood pressure measurement on conscious dogs described below. The system consists of implantable pressure transmitters, receiver and a data acquisition computer. The telemetry system makes it possible to continuously monitor blood pressures and heart rate of conscious animals. The telemetry transmitters used are surgically implanted under aseptic conditions in the experimental animals before the first experimental use. The animals instrumented in this way can be used repeatedly after the wound has healed and the implant has settled. The tests are carried out using adult male beagles. Technical details can be found in the documentation from the manufacturing company (DSI).

### Substances and Solutions

The substances to be tested are each administered to a group of dogs (n=3-6), orally via a gelatine capsule or intravenously in suitable solvent mixtures. A vehicle-treated group of animals is employed as control.

### Experimental Procedure

For the measurements under hypoxia conditions, the animals are transferred to a chamber with a hypoxic atmosphere (oxygen content about 10%). This is established using commercially available hypoxia generators (from Hoehenbalance, Cologne, Germany). In a standard experiment, for example, one hour and five hours after substance administration the dogs are transferred to the hypoxia chamber for 30 min. About 10 min before and after entering the hypoxia chamber, as well as during the stay in the hypoxia chamber, pressures and heart rate are measured by telemetry.

### Evaluation

In healthy dogs, under hypoxia there is a rapid increase in PAP. By substance administration, this increase can be reduced. To quantify the PAP increase and the differences in heart rate and systemic blood pressure, the data before and during the hypoxia period, smoothed by determination of means, are compared. The courses of the measured parameters are presented graphically using the Prism software (GraphPad, USA).

### B-7. Determination of Pharmacokinetic Parameters Following Intravenous and Oral Administration

The pharmacokinetic parameters of the compounds of the invention are determined in male CD-1 mice, male Wistar rats, female beagles and female cynomolgus monkeys. Intravenous administration in the case of mice and rats is effected by means of a species-specific plasma/DMSO formulation, and in the case of dogs and monkeys by means of a water/PEG400/ethanol formulation. In all species, oral administration of the dissolved substance is performed via gavage, based on a water/PEG400/ethanol formulation. The removal of blood from rats is simplified by inserting a silicone catheter into the right Vena jugularis externa prior to substance administration. The operation is carried out at least one day prior to the experiexperiment with isofluran anaesthesia and administration of an analgesic (atropine/rimadyl (3/1) 0.1 ml s.c.). The blood is taken (generally more than 10 time points) within a time window including terminal time points of at least 24 to a maximum of 72 hours after substance administration. The blood is removed into heparinized tubes. The blood plasma is then obtained by centrifugation; if required, it is stored at −20° C. until further processing.

An internal standard (which may also be a chemically unrelated substance) is added to the samples of the compounds of the invention, calibration samples and qualifiers, and there follows protein precipitation by means of acetonitrile in excess. Addition of a buffer solution matched to the LC conditions, and subsequent vortexing, is followed by centrifugation at 1000 g. The supernatant is analysed by LCMS(/MS) using C18 reversed-phase columns and variable mobile phase mixtures. The substances are quantified via the peak heights or areas from extracted ion chromatograms of specific selected ion monitoring experiments or high-resolution LC-MS experiments.

The plasma concentration/time plots determined are used to calculate the pharmacokinetic parameters such as AUC, Cₘₐₓ, F (bioavailability), t_{1/2} (terminal half life), MRT (mean residence time) and CL (clearance), using a validated pharmacokinetic calculation program.

Since the substance quantification is performed in plasma, it is necessary to determine the blood/plasma distribution of the substance in order to be able to adjust the pharmacokinetic parameters correspondingly.

For this purpose, a defined amount of substance is incubated in heparinized whole blood of the species in question in a rocking roller mixer for 20 min. Plasma is obtained by centrifugation at 1000 g. After measurement of the concentrations in plasma and blood (by LC-MS(/MS); see above), the C_{blood}/Cₚₗₐₛₘₐma value is determined by quotient formation.

### B-8. Metabolic Study

To determine the metabolic profile of the compounds of the invention, they are incubated with recombinant human cytochrome P450 (CYP) enzymes, liver microsomes or primary fresh hepatocytes from various animal species (e.g. rats, dogs), and also of human origin, in order to obtain and to compare information about a very substantially complete hepatic phase I and phase II metabolism, and about the enzymes involved in the metabolism.

The compounds of the invention were incubated with a concentration of about 0.1-10 μM. To this end, stock solutions of the compounds of the invention having a concentration of 0.01-1 mM in acetonitrile were prepared, and then pipetted with a 1:100 dilution into the incubation mixture. The liver microsomes and recombinant enzymes were incubated at 37° C. in 50 mM potassium phosphate buffer pH 7.4 with and without NADPH-generating system consisting of 1 mM NADP⁺, 10 mM glucose-6-phosphate and 1 unit glucose-6-phosphate dehydrogenase. Primary hepatocytes were incubated in suspension in Williams E medium, likewise at 37° C. After an incubation time of 0-4 h, the incubation mixtures were stopped with acetonitrile (final concentration about 30%) and the protein was centrifuged off at about 15 000×g. The samples thus stopped were either analyzed directly or stored at −20° C. until analysis.

The analysis is carried out by high-performance liquid chromatography with ultraviolet and mass spectrometry detection (HPLC-UV-MS/MS). To this end, the supernatants of the incubation samples are chromatographed with suitable C18 reversed-phase columns and variable mobile phase mixtures of acetonitrile and 10 mM aqueous ammonium formate solution or 0.05% formic acid. The UV chromatograms in conjunction with mass spectrometry data serve for identification, structural elucidation and quantitative estimation of the metabolites, and for quantitative metabolic reduction of the compound of the invention in the incubation mixtures.

### B-9. Caco-2 Permeability Test

The permeability of a test substance was determined with the aid of the Caco-2 cell line, an established in vitro model for permeability prediction at the gastrointestinal barrier (Artursson, P. and Karlsson, J. “Correlation between oral drug absorption in humans and apparent drug permeability coefficients in human intestinal epithelial (Caco-2) cells” Biochem. Biophys. 1991, 175 (3), 880-885). The Caco-2 cells (ACC No. 169, DSMZ, Deutsche Sammlung von Mikroorganismen und Zellkulturen, Braunschweig, Germany) were sown in 24-well plates having an insert and cultivated for 15 to 16 days. For the permeability studies, the test substance was dissolved in DMSO and diluted to the final test concentration with transport buffer (Hanks Buffered Salt Solution, Gibco/Invitrogen, with 19.9 mM glucose and 9.8 mM HEPES). In order to determine the apical to basolateral permeability (PₐₚₚA-B) of the test substance, the solution comprising the test substance was applied to the apical side of the Caco-2 cell monolayer, and transport buffer to the basolateral side. In order to determine the basolateral to apical permeability (P_{appB}A) of the test substance, the solution comprising the test substance was applied to the basolateral side of the Caco-2 cell monolayer, and transport buffer to the apical side. At the start of the experiment, samples were taken from the respective donor compartment in order to ensure the mass balance. After an incubation time of two hours at 37° C., samples were taken from the two compartments. The samples were analyzed by means of LC-MS/MS and the apparent permeability coefficients (Pₐₚₚ) were calculated. For each cell monolayer, the permeability of Lucifer Yellow was determined to ensure cell layer integrity. In each test run, the permeability of atenolol (marker for low permeability) and sulfasalazine (marker for active excretion) was also determined as quality control.

### C. Working Examples of Pharmaceutical Compositions

The compounds of the invention can be converted to pharmaceutical preparations as follows:

### Tablet:

### Composition:

100 mg of the compound of the invention, 50 mg of lactose (monohydrate), 50 mg of corn starch (native), 10 mg of polyvinylpyrrolidone (PVP 25) (BASF, Ludwigshafen, Germany) and 2 mg of magnesium stearate.

Tablet weight 212 mg. Diameter 8 mm, radius of curvature 12 mm.

### Production:

The mixture of compound of the invention, lactose and starch is granulated with a 5% solution (w/w) of the PVP in water. The granules are dried and then mixed with the magnesium stearate for 5 minutes. This mixture is compressed using a conventional tableting press (see above for format of the tablet). The guide value used for the pressing is a pressing force of 15 kN.

### Suspension for Oral Administration:

### Composition:

1000 mg of the compound of the invention, 1000 mg of ethanol (96%), 400 mg of Rhodigel® (xanthan gum from FMC, Pennsylvania, USA) and 99 g of water.

10 ml of oral suspension correspond to a single dose of 100 mg of the compound of the invention.

### Production:

The Rhodigel is suspended in ethanol; the compound of the invention is added to the suspension. The water is added while stirring. The mixture is stirred for about 6 h until the swelling of the Rhodigel is complete.

### Solution for Oral Administration:

### Composition:

500 mg of the compound of the invention, 2.5 g of polysorbate and 97 g of polyethylene glycol 400. 20 g of oral solution correspond to a single dose of 100 mg of the compound of the invention.

### Production:

The compound of the invention is suspended in the mixture of polyethylene glycol and polysorbate with stirring. The stirring operation is continued until dissolution of the compound of the invention is complete.

i.v. Solution:

The compound of the invention is dissolved in a concentration below the saturation solubility in a physiologically acceptable solvent (e.g. isotonic saline solution, glucose solution 5% and/or PEG 400 solution 30%). The solution is subjected to sterile filtration and dispensed into sterile and pyrogen-free injection vessels.

1. A compound of the general formula (I) 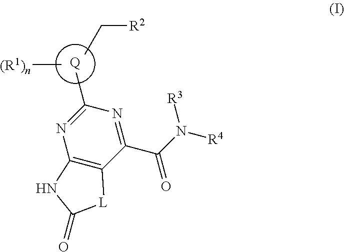
in which
the ring Q represents 5- or 6-membered monocyclic heteroaryl or 8- or 9-membered bicyclic heteroaryl,
L represents a #¹-CR^{5A}R^{5B}—(CR^{6A}R^{6B})ₘ-#² group,
where
#¹ represents the point of attachment to the carbonyl group,
#² represents the attachment site to the pyrimidine ring,
m represents a number 0, 1 or 2,
R^{5A} represents hydrogen, fluorine, (C₁-C₄)-alkyl, hydroxy or amino,
in which (C₁-C₄)-alkyl may be substituted by 1 to 3 substituents independently of one another selected from the group consisting of fluorine, trifluoromethyl, hydroxy, hydroxycarbonyl, (C₁-C₄)-alkoxycarbonyl and amino,
R^{5B} represents hydrogen, fluorine, difluoromethyl, trifluoromethyl, (C₁-C₆)-alkyl, (C₁-C₄)-alkoxycarbonylamino, cyano, (C₃-C₇)-cycloalkyl, difluoromethoxy, trifluoromethoxy, phenyl or a group of the formula -M-R⁷,
in which (C₁-C₆)-alkyl may be substituted by 1 to 3 substituents independently of one another selected from the group consisting of fluorine, cyano, trifluoromethyl, (C₃-C₇)-cycloalkyl, hydroxy, difluoromethoxy, trifluoromethoxy, (C₁-C₄)-alkoxy, hydroxycarbonyl, (C₁-C₄)-alkoxycarbonyl and amino,
and in which
M represents a bond or (C₁-C₄)-alkanediyl,
R⁷ represents —(C═O)ᵣOR⁸, —(C═O)ᵣNR⁹R¹⁰, —C(═S)—NR⁹R¹⁰, —NR—(C═O)—R¹¹, —NR⁸—(C═O)—NR⁹R¹⁰, —NR⁸—SO₂—NR⁹R¹⁰, —NR⁸—SO₂—R¹¹, —S(O)ₛ—R¹¹, —SO₂—NR⁹R¹⁰, 4- to 7-membered heterocyclyl, phenyl or 5- or 6-membered heteroaryl,
in which
r represents the number 0 or 1,
s represents the number 0, 1 or 2,
R⁸, R⁹ and R¹⁰ independently of one another each represent hydrogen, (C₁-C₆)-alkyl, (C₃-C₈)-cycloalkyl, 4- to 7-membered heterocyclyl, phenyl or 5- or 6-membered heteroaryl,
or
R⁹ and R¹⁰ together with the atom(s) to which they are respectively attached form a 4- to 7-membered heterocycle,
R¹¹ represents (C₁-C₆)-alkyl or (C₃-C₇)-cycloalkyl,
or
R⁸ and R¹¹ together with the atom(s) to which they are respectively attached form a 4- to 7-membered heterocycle,
and
in which the (C₁-C₆)-alkyl, (C₃-C₇)-cycloalkyl, (C₃-C₈)-cycloalkyl and 4- to 7-membered heterocyclyl groups mentioned above may each independently of one another additionally be substituted by 1 to 3 substituents independently of one another selected from the group consisting of fluorine, difluoromethyl, trifluoromethyl, (C₁-C₄)-alkyl, (C₃-C₇)-cycloalkyl, hydroxy, difluoromethoxy, trifluoromethoxy, (C₁-C₄)-alkoxy, hydroxycarbonyl, (C₁-C₄)-alkoxycarbonyl, amino, phenyl, 4- to 7-membered heterocyclyl and 5- or 6-membered heteroaryl,
or
R^{5A} and R^{5B} together with the carbon atom to which they are attached form a (C₂-C₄)-alkenyl group, an oxo group, a 3- to 6-membered carbocycle or a 4- to 7-membered heterocycle,
in which the 3- to 6-membered carbocycle and the 4- to 7-membered heterocycle may be substituted by 1 or 2 substituents independently of one another selected from the group consisting of fluorine, hydroxy, methoxy and (C₁-C₄)-alkyl,
R^{6A} represents hydrogen, fluorine, (C₁-C₄)-alkyl or hydroxy,
R^{6B} represents hydrogen, fluorine, (C₁-C₄)-alkyl or trifluoromethyl,
R¹ represents hydrogen, halogen, cyano, difluoromethyl, trifluoromethyl, (C₁-C₄)-alkyl, (C₃-C₇)-cycloalkyl, hydroxy, (C₁-C₄)-alkoxy, phenyl or 5- or 6-membered heterocyclyl,
n represents a number 0, 1, 2 or 3,
R² represents trifluoromethyl, (C₁-C₆)-alkyl, (C₃-C₈)-cycloalkyl, phenyl or 5- or 6-membered heteroaryl,
where (C₁-C₆)-alkyl is substituted by a substituent selected from the group consisting of difluoromethyl and trifluoromethyl and may furthermore be up to trisubstituted by fluorine,
and
where (C₃-C₈)-cycloalkyl may be substituted by 1 or 2 substituents selected independently of one another from the group consisting of fluorine, methyl and methoxy,
and
where phenyl may be substituted by 1 to 3 halogen substituents and furthermore by 1 or 2 substituents independently of one another selected from the group consisting of (C₁-C₄)-alkyl, (C₁-C₄)-alkoxy and cyano,
and
where 5- or 6-membered heteroaryl may be substituted by 1 or 2 substituents selected from the group consisting of trifluoromethyl and methyl and furthermore up to three times by fluorine,
R³ represents hydrogen, (C₁-C₄)-alkyl or (C₃-C₈)-cycloalkyl,
R⁴ represents hydrogen, (C₁-C₁₀)-alkyl, (C₃-C₈)-cycloalkyl, (C₂-C₆)-alkenyl, 4- to 7-membered heterocyclyl, phenyl, 5- or 6-membered heteroaryl, —NR¹²R¹³ or —OR¹⁴,
where (C₁-C₁₀)-alkyl, (C₃-C₈)-cycloalkyl, (C₂-C₆)-alkenyl and 4- to 7-membered heterocyclyl may be substituted by 1 to 3 substituents independently of one another selected from the group consisting of fluorine, difluoromethyl, trifluoromethyl, methyl, ethyl, hydroxy, oxo, (C₁-C₆)-alkyl, (C₃-C₇)-cycloalkyl, difluoromethoxy, trifluoromethoxy, —OR¹⁵, —NR¹⁶—(C═O)—R¹⁷, —NR¹⁶—(C═O)—NR¹⁸R¹⁹, —NR¹⁸R¹⁹, —(C═O)—NR¹⁸R¹⁹, —S(O)ₚ—R²⁰, —NR¹⁸—SO₂—R¹⁹, —SO₂—NR¹⁸R¹⁹, —(C═O)—OR²¹, —NR¹⁶—(C═O)—OR²¹, phenyl, 4- to 7-membered heterocyclyl and 5- or 6-membered heteroaryl,
in which
p represents the number 0, 1 or 2,
R¹⁵ and R²⁰ independently of one another each represent (C₁-C₆)-alkyl, phenyl or (C₃-C₈)-cycloalkyl,
R¹⁶, R¹⁷, R¹⁸ and R¹⁹ independently of one another each represent hydrogen, (C₁-C₆)-alkyl or (C₃-C₈)-cycloalkyl,
or
R¹⁶ and R¹⁷ together with the nitrogen atom to which they are attached form a 4- to 7-membered heterocycle,
or
R¹⁸ and R¹⁹ together with the nitrogen atom to which they are attached form a 4- to 7-membered heterocycle,
R²¹ represents hydrogen, (C₁-C₆)-alkyl or (C₃-C₈)-cycloalkyl,
and
where 5- or 6-membered heteroaryl and phenyl may each be substituted by 1 to 3 substituents independently of one another selected from the group consisting of halogen, difluoromethyl, trifluoromethyl, (C₁-C₄)-alkyl, (C₁-C₄)-alkoxy, difluoromethoxy, trifluoromethoxy, cyano, hydroxy and (C₃-C₇)-cycloalkyl,
and where
R¹² and R¹³ independently of one another represent hydrogen or (C₁-C₄)-alkyl, in which (C₁-C₄)-alkyl may be substituted by 1 to 3 substituents selected from the group consisting of fluorine, hydroxy and (C₁-C₄)-alkoxy,
or
R¹² and R¹³ together with the nitrogen atom to which they are attached form a 4- to 7-membered heterocycle,
and where
R¹⁴ represents (C₁-C₆)-alkyl, (C₃-C₇)-cycloalkyl or (C₃-C₆)-alkenyl,
or
R³ and R⁴ together with the nitrogen atom to which they are attached form a 4- to 7-membered heterocycle,
where the 4- to 7-membered heterocycle may be substituted by 1 to 3 substituents independently of one another selected from the group consisting of fluorine, difluoromethyl, trifluoromethyl, cyano, (C₁-C₄)-alkyl, (C₃-C₇)-cycloalkyl, hydroxy, oxo, (C₁-C₄)-alkoxy, difluoromethoxy, trifluoromethoxy and amino,
and
where the (C₁-C₄)-alkyl, (C₁-C₆)-alkyl, (C₃-C₈)-cycloalkyl, (C₃-C₇)-cycloalkyl, (C₂-C₆)-alkenyl, (C₃-C₆)-alkenyl and 4- to 7-membered heterocyclyl groups mentioned above, unless stated otherwise, may each independently of one another additionally be substituted by 1 to 3 substituents independently of one another selected from the group consisting of fluorine, difluoromethyl, trifluoromethyl, (C₁-C₄)-alkyl, (C₃-C₇)-cycloalkyl, hydroxy, difluoromethoxy, trifluoromethoxy, (C₁-C₄)-alkoxy, hydroxycarbonyl, (C₁-C₄)-alkoxycarbonyl, amino, phenyl, 4- to 7-membered heterocyclyl and 5- or 6-membered heteroaryl,
and the N-oxides, salts, solvates, salts of the N-oxides and solvates of the N-oxides and salts thereof.

2. The compound of the formula (I) as claimed in claim 1 in which the ring Q represents a group of the formula 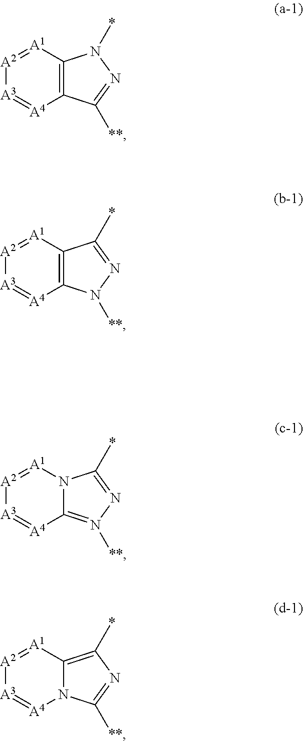
where
* represents the point of attachment to —CH₂—R²,
** represents the point of attachment to the pyrimidine ring,
n represents a number 0, 1 or 2,
A¹, A², A³ and A⁴ independently of one another each represent N, C—H or C—R¹, with the proviso that not more than two of the A¹, A², A³ and A⁴ groups represent N,
L represents a #¹-CR^{5A}R^{5B}—(CR^{6A}R^{6B})ₘ-#² group, where
#¹ represents the point of attachment to the carbonyl group,
#² represents the attachment site to the pyrimidine ring,
m represents a number 0 or 1,
R^{5A} represents hydrogen, fluorine, trifluoromethyl or (C₁-C₄)-alkyl,
R^{5B} represents hydrogen, fluorine, trifluoromethyl, (C₁-C₄)-alkyl, (C₃-C₇)-cycloalkyl or a group of the formula -M-R⁷,
in which (C₁-C₄)-alkyl may be substituted by 1 to 3 substituents independently of one another selected from the group consisting of fluorine, cyano, trifluoromethyl, (C₃-C₇)-cycloalkyl, difluoromethoxy and trifluoromethoxy,
M represents a bond or methylene,
R⁷ represents —(C═O)—OR⁸ or —(C═O)—NR⁹R¹⁰,
in which
R⁸ represents hydrogen, (C₁-C₄)-alkyl, (C₃-C₆)-cycloalkyl or 4- or 7-membered heterocyclyl,
R⁹ and R¹⁰ independently of one another each represent hydrogen, (C₁-C₄)-alkyl, (C₃-C₆)-cycloalkyl, 4- to 7-membered heterocyclyl, phenyl or 5- or 6-membered heteroaryl,
or
R⁹ and R¹⁰ together with the atom(s) to which they are respectively attached form a 4- to 7-membered heterocycle,
or
R^{5A} and R^{5B} together with the carbon atom to which they are attached form a (C₂-C₄)-alkenyl group, a 3- to 6-membered carbocycle or a 4- to 7-membered heterocycle,
where the 3- to 6-membered carbocycle may be monosubstituted by hydroxy and up to disubstituted by fluorine,
R^{6A} represents hydrogen, fluorine, (C₁-C₄)-alkyl or hydroxy,
R^{6B} represents hydrogen, fluorine, (C₁-C₄)-alkyl or trifluoromethyl,
R¹ represents fluorine, chlorine, cyano, difluoromethyl, trifluoromethyl, (C₁-C₄)-alkyl, (C₃-C₅)-cycloalkyl or (C₁-C₄)-alkoxy,
N represents a number 0, 1 or 2,
R² represents (C₁-C₆)-alkyl, phenyl or 5- or 6-membered heteroaryl,
where (C₁-C₆)-alkyl is substituted by a substituent selected from the group consisting of difluoromethyl and trifluoromethyl and may furthermore be up to trisubstituted by fluorine,
and
where phenyl is substituted by 1 to 3 fluorine substituents and may furthermore be substituted by 1 or 2 substituents independently of one another selected from the group consisting of methyl and methoxy,
and
where 5- or 6-membered heteroaryl is up to disubstituted by fluorine,
R³ represents hydrogen, (C₁-C₄)-alkyl or cyclopropyl,
R⁴ represents hydrogen, (C₁-C₁₀)-alkyl, (C₃-C₇)-cycloalkyl, (C₂-C₆)-alkenyl, 4- to 7-membered heterocyclyl, phenyl, 5- or 6-membered heteroaryl, —NR¹²R¹³ or —OR¹⁴,
where (C₁-C₁₀)-alkyl may be substituted by 1 to 3 substituents independently of one another selected from the group consisting of fluorine, difluoromethyl, trifluoromethyl, (C₃-C₇)-cycloalkyl, hydroxy, oxo, —OR¹⁵, —NR¹⁶—(C═O)—R¹⁷, —NR¹⁸R¹⁹, —(C═O)—NR¹⁸R¹⁹, —S(O)ₚ—R²⁰, —NR¹⁸—S₂—R¹⁹, phenyl, 4- to 7-membered heterocyclyl and 5- or 6-membered heteroaryl,
in which (C₃-C₇)-cycloalkyl and 4- to 7-membered heterocyclyl independently of one another may each be substituted by a substituent selected from the group consisting of (C₁-C₄)-alkyl, oxo, hydroxy, amino and furthermore up to tetrasubstituted by fluorine,
and
in which phenyl and 5- or 6-membered heteroaryl independently of one another may each be substituted by (C₁-C₄)-alkyl and furthermore up to trisubstituted by fluorine,
p represents the number 0, 1 or 2,
R¹⁵ and R²⁰ independently of one another represent (C₁-C₄)-alkyl which may be up to pentasubstituted by fluorine, represent phenyl or (C₃-C₇) cycloalkyl,
R¹⁶ and R¹⁷ independently of one another each represent hydrogen, (C₁-C₄)-alkyl or (C₃-C₇)-cycloalkyl,
R¹⁸ and R¹⁹ independently of one another represent hydrogen, (C₁-C₆)alkyl which may be up to pentasubstituted by fluorine, or represent (C₃-C₇)-cycloalkyl,
or
R¹⁸ and R¹⁹ together with the nitrogen atom to which they are attached form a 4- to 7-membered heterocycle,
in which the 4- to 7-membered heterocycle may be up to tetrasubstituted by fluorine,
where (C₃-C₇)-cycloalkyl may be substituted by a substituent selected from the group consisting of (C₁-C₄)-alkyl, hydroxy, amino, cyano and furthermore up to tetrasubstituted by fluorine,
and
where (C₂-C₆)-alkenyl may be substituted by (C₁-C₄)-alkyl and furthermore up to pentasubstituted by fluorine,
and
where 4- to 7-membered heterocyclyl may be substituted by 1 to 4 substituents independently of one another selected from the group consisting of fluorine, trifluoromethyl, oxo, (C₁-C₄)-alkyl, hydroxy and amino,
and
where 5- or 6-membered heteroaryl and phenyl may each be substituted by 1 to 3 substituents independently of one another selected from the group consisting of halogen, (C₁-C₄)-alkyl, (C₁-C₄)-alkoxy, cyano and (C₃-C₅)-cycloalkyl,
and where
R¹² and R¹³ independently of one another represent hydrogen or (C₁-C₄)-alkyl, or
R¹² and R¹³ together with the nitrogen atom to which they are attached form a 4- to 7-membered heterocycle,
and where
R¹⁴ represents (C₁-C₆)-alkyl which may be up to pentasubstituted by fluorine, represents (C₃-C₇)-cycloalkyl or (C₃-C₆)-alkenyl,
or
R³ and R⁴ together with the nitrogen atom to which they are attached form a 4- to 7-membered heterocycle,
where the 4- to 7-membered heterocycle may be substituted by 1 to 3 substituents independently of one another selected from the group consisting of trifluoromethyl, (C₁-C₄)-alkyl, (C₃-C₇)-cycloalkyl, hydroxy, (C₁-C₄)-alkoxy, trifluoromethoxy and amino and furthermore up to tetrasubstituted by fluorine,
and the salts, solvates and solvates of the salts thereof.

3. The compound of the formula (I) as claimed in claim 1 in which
the ring Q represents a group of the formula 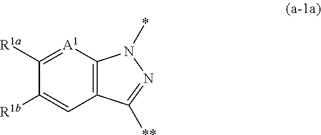
where
* represents the point of attachment to —CH₂—R²,
** represents the point of attachment to the pyrimidine ring,
A¹ represents N or C—H,
R^{1a} represents hydrogen or methyl if A¹ represents nitrogen,
or
R^{1a} represents hydrogen, fluorine or chlorine if A¹ represents C—H,
R^{1b} represents hydrogen or fluorine,
L represents a #¹-CR^{5A}R^{5B}—(CR^{6A}R^{6B})ₘ-#² group, where
#¹ represents the point of attachment to the carbonyl group,
#² represents the attachment site to the pyrimidine ring,
m represents a number 0,
R^{5A} represents hydrogen, methyl or ethyl,
R^{5B} represents hydrogen, fluorine, trifluoromethyl, methyl or ethyl,
where methyl and ethyl may be up to trisubstituted by fluorine,
or
R^{5A} and R^{5B} together with the carbon atom to which they are attached form a cyclopropyl ring,
R² represents 2,2,2-trifluoroeth-1-yl, phenyl or pyridyl,
where phenyl is substituted by 1 to 3 fluorine substituents,
and
where pyridyl is monosubstituted by fluorine,
R³ represents hydrogen, (C₁-C₄)-alkyl or cyclopropyl,
R⁴ represents hydrogen, (C₁-C₆)-alkyl, (C₃-C₆)-cycloalkyl, (C₂-C₆)-alkenyl, 5- or 6-membered heterocyclyl, phenyl, 5- or 6-membered heteroaryl or —OR¹⁴
where (C₁-C₆)-alkyl may be substituted by 1 or 2 substituents independently of one another selected from the group consisting of difluoromethyl, trifluoromethyl, (C₃-C₆)cycloalkyl, hydroxy, oxo, —OR¹⁵, —NR¹⁶—(C═O)—R¹⁷, —NR¹⁸R¹⁹, —(C═O)—NR¹⁸R¹⁹, —S(O)ₚR²⁰, phenyl, 4- to 6-membered heterocyclyl and 5- or 6-membered heteroaryl and furthermore up to trisubstituted by fluorine,
in which (C₃-C₆)-cycloalkyl and 4- to 6-membered heterocyclyl independently of one another may each be substituted by a substituent selected from the group consisting of (C₁-C₄)-alkyl, oxo, hydroxy, amino and furthermore up to tetrasubstituted by fluorine,
and
in which phenyl and 5- or 6-membered heteroaryl independently of one another may each be substituted by (C₁-C₄)-alkyl and furthermore up to trisubstituted by fluorine,
p represents the number 0, 1 or 2,
R¹⁵ and R²⁰ each independently of one another represent (C₁-C₄)-alkyl, in which (C₁-C₄)-alkyl may be substituted up to five times by fluorine,
R¹⁶ represents hydrogen or (C₁-C₄)-alkyl,
R¹⁷ represents (C₁-C₄)-alkyl or (C₃-C₆)-cycloalkyl, R¹⁸ and R¹⁹ independently of one another represent hydrogen or (C₁-C₄)alkyl which may be up to pentasubstituted by fluorine,
or
R¹⁸ and R¹⁹ together with the nitrogen atom to which they are attached form a 5- or 6-membered heterocycle,
where (C₃-C₆)-cycloalkyl may be substituted by a substituent selected from the group consisting of (C₁-C₄)-alkyl, hydroxy, amino, cyano and furthermore up to tetrasubstituted by fluorine,
and
where (C₂-C₆)-alkenyl may be up to trisubstituted by fluorine,
and
where 5- or 6-membered heterocyclyl may be substituted by a substituent selected from the group consisting of oxo, (C₁-C₄)-alkyl, hydroxy and amino and furthermore up to tetrasubstituted by fluorine,
and
where 5- or 6-membered heteroaryl and phenyl independently of one another may each be substituted by a substituent selected from the group consisting of halogen, (C₁-C₄)-alkyl, cyano and (C₃-C₅)-cycloalkyl,
and where
R¹⁴ represents (C₁-C₆)-alkyl which may be up to pentasubstituted by fluorine, or represents (C₃-C₆)-alkenyl,
or
R³ and R⁴ together with the nitrogen atom to which they are attached form a 5- or 6-membered heterocycle,
where the 5- or 6-membered heterocycle may be substituted by a substituent selected from the group consisting of (C₁-C₄)-alkyl, oxo, hydroxy and furthermore up to tetrasubstituted by fluorine,
and the salts, solvates and solvates of the salts thereof.

4. The compound of the formula (I) as claimed in claim 1 in which
the ring Q represents a group of the formula 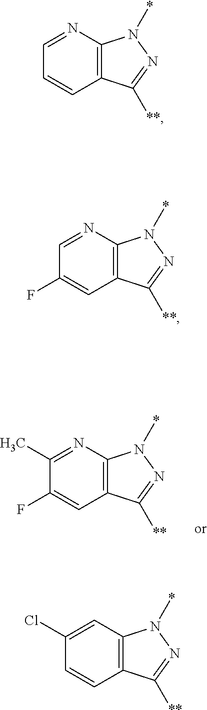
where
* represents the point of attachment to —CH₂—R²,
** represents the point of attachment to the pyrimidine ring, in which
L represents a #¹-CR^{5A}R^{5B}—(CR^{6A}R^{6B})ₘ-#² group, where
#¹ represents the point of attachment to the carbonyl group,
#² represents the attachment site to the pyrimidine ring,
m represents a number 0,
R^{5A} represents methyl,
R^{5B} represents methyl or trifluoromethyl,
R² represents a phenyl group of the formula 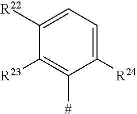
where
# represents the point of attachment to the methylene group,
R²² and R²⁴ independently of one another each represent hydrogen or fluorine,
R²³ represents fluorine,
or
R² represents 3-fluoropyrid-2-yl,
R³ represents hydrogen or methyl,
R⁴ represents hydrogen, (C₁-C₄)-alkyl or cyclopropyl,
where (C₁-C₄)-alkyl may be substituted by a substituent selected from the group consisting of hydroxy, amino, methoxy, 2,2,2-trifluoroethoxy and cyclopropyl, and furthermore up to trisubstituted by fluorine,
and
where cyclopropyl may be substituted by cyano,
and the salts, solvates and solvates of the salts thereof.

5. A process for preparing the compound of the formula (I) according to claim 1, comprising reacting a compound of the formula (II) 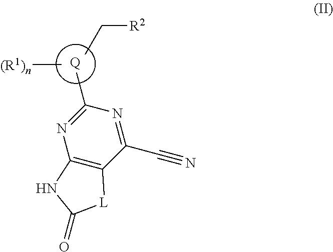
in which n, L, Q, R¹ and R² each have the meanings given above,
in a first step in the presence of a suitable aqueous base or acid to give the carboxamide of the formula (I-A) 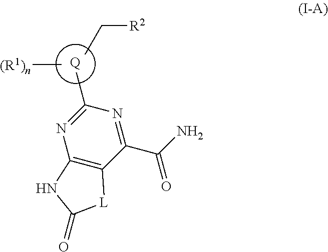
in which n, L, Q, R¹ and R² each have the meanings given above,
and optionally converting the carboxamide (I-A) in a second step in an inert solvent in the presence of a suitable aqueous acid or base into a carboxylic acid of the formula (III) 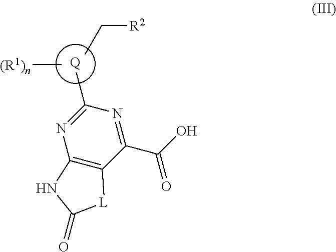
in which n, L, Q, R¹ and R² each have the meanings given above,
and subsequently reacting these in a third step, with activation of the carboxylic acid function, with an amine compound of the formula (IV) 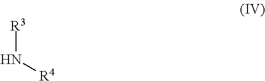
in which R³ and R⁴ each have the meanings given above, to give the carboxamide of the formula (I-B) 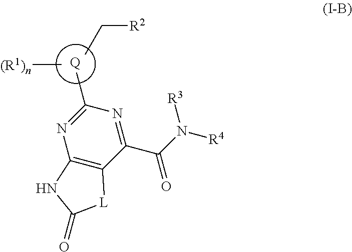
in which n, L, Q, R¹, R², R³ and R⁴ each have the meanings given above,
then detaching any protective groups present, and optionally converting the resulting compounds of the formulae (I-A) and (I-B), optionally with the appropriate (i) solvents and/or (ii) acids or bases, to the solvates, salts and/or solvates of the salts thereof.

6. (canceled)

7. (canceled)

8. A medicament comprising a compound as defined in claim 1 in combination with one or more inert, nontoxic, pharmaceutically suitable excipients.

9. A medicament comprising a compound as defined in claim 1 in combination with one or more further active compounds selected from the group consisting of organic nitrates, NO donors, cGMP-PDE inhibitors, antithrombotic agents, hypotensive agents and lipid metabolism modifiers.

10. (canceled)

11. A method for the treatment and/or prophylaxis of heart failure, angina pectoris, hypertension, pulmonary hypertension, ischemias, vascular disorders, renal insufficiency, thromboembolic disorders, fibrotic disorders, arteriosclerosis, dementia disorders and erectile dysfunction in humans and animals comprising administering an effective amount of at least one compound as defined in claim 1 to a human or animal in need thereof.

12. A method for the treatment and/or prophylaxis of heart failure, angina pectoris, hypertension, pulmonary hypertension, ischemias, vascular disorders, renal insufficiency, thromboembolic disorders, fibrotic disorders, arteriosclerosis, dementia disorders and erectile dysfunction in humans and animals comprising administering an effective amount of the medicament of claim 8 to a human or animal in need thereof.

13. A method for the treatment and/or prophylaxis of heart failure, angina pectoris, hypertension, pulmonary hypertension, ischemias, vascular disorders, renal insufficiency, thromboembolic disorders, fibrotic disorders, arteriosclerosis, dementia disorders and erectile dysfunction in humans and animals comprising administering an effective amount of the medicament of claim 9 to a human or animal in need thereof.

14. A method for the treatment and/or prophylaxis of heart failure, angina pectoris, hypertension, pulmonary hypertension, ischemias, vascular disorders, renal insufficiency, thromboembolic disorders, fibrotic disorders, arteriosclerosis, dementia disorders and erectile dysfunction in humans and animals comprising administering an effective amount of at least one compound as defined in claim 2 to a human or animal in need thereof.

15. A method for the treatment and/or prophylaxis of heart failure, angina pectoris, hypertension, pulmonary hypertension, ischemias, vascular disorders, renal insufficiency, thromboembolic disorders, fibrotic disorders, arteriosclerosis, dementia disorders and erectile dysfunction in humans and animals comprising administering an effective amount of at least one compound as defined in claim 3 to a human or animal in need thereof.

16. A method for the treatment and/or prophylaxis of heart failure, angina pectoris, hypertension, pulmonary hypertension, ischemias, vascular disorders, renal insufficiency, thromboembolic disorders, fibrotic disorders, arteriosclerosis, dementia disorders and erectile dysfunction in humans and animals comprising administering an effective amount of at least one compound as defined in claim 4 to a human or animal in need thereof.
